(19) United States
(12) Patent Application Publication
(10) Pub. No.: US 2026/0031213 A1
(43) Pub. Date: Jan. 29, 2026

(54) METHOD OF RECLAIMING PORTIONS OF SURGICAL INSTRUMENTS FOR REMANUFACTURING AND SUSTAINABILITY

(71) Applicant: Cilag GmbH International, Zug (CH)

(72) Inventors: Frederick E. Shelton, IV, Hillsboro, OH (US); Jacqueline C. Aronhalt, Loveland, OH (US); Matthew E. Derrico, Milford, OH (US); Cameron D. McLain, Deer Park, OH (US); Bruce Brunson, JR., Cincinnati, OH (US); Gregory A. Trees, Loveland, OH (US)

(21) Appl. No.: 19/346,944

(22) Filed: Oct. 1, 2025

Related U.S. Application Data
(63) Continuation of application No. 17/854,065, filed on Jun. 30, 2022.

(51) Int. Cl. G16H 20/40; A61B 34/00; A61B 50/30

(52) U.S. Cl. CPC G16H 20/40; A61B 34/71; A61B 34/76; A61B 50/30

(57) ABSTRACT

A method of reclaiming portions of a surgical kit having a surgical instrument includes disassembling the surgical instrument and determining a disposal methodology of the surgical kit. Furthermore, reclaiming further includes verifying reuse capacity of a portion of the surgical instrument and determining a waste stream for the portion of the surgical instrument. The method also includes disassembling the portion of the surgical instrument from a remainder of the surgical instrument at a predetermined region of the surgical instrument to thereby reclaim the portion of the surgical instrument according to the waste stream.

### CROSS-REFERENCE TO RELATED APPLICATION

This application is a continuation of U.S. patent application Ser. No. 17/854,065, entitled “Method of Reclaiming Portions of Surgical Instruments for Remanufacturing and Sustainability,” filed Jun. 30, 2022, and published as U.S. Pub. No. 2024/0006048 on Jan. 4, 2024.

### BACKGROUND

A variety of ultrasonic surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned, the disclosure of which is incorporated by reference herein, in its entirety; U.S. Pub. No. 2007/0191713, entitled “Ultrasonic Device for Cutting and Coagulating,” published Aug. 16, 2007, now abandoned, the disclosure of which is incorporated by reference herein, in its entirety; and U.S. Pub. No. 2008/0200940, entitled “Ultrasonic Device for Cutting and Coagulating,” published Aug. 21, 2008, now abandoned, the disclosure of which is incorporated by reference herein, in its entirety.

Some instruments are operable to seal tissue by applying radiofrequency (RF) electrosurgical energy to the tissue. Examples of such devices and related concepts are disclosed in U.S. Pat. No. 7,354,440, entitled “Electrosurgical Instrument and Method of Use,” issued Apr. 8, 2008, the disclosure of which is incorporated by reference herein, in its entirety; U.S. Pat. No. 7,381,209, entitled “Electrosurgical Instrument,” issued Jun. 3, 2008, the disclosure of which is incorporated by reference herein, in its entirety.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein, in its entirety; U.S. Pat. No. 8,663,220, entitled “Ultrasonic Electrosurgical Instruments,” issued Mar. 4, 2014, the disclosure of which is incorporated by reference herein, in its entirety; U.S. Pat. No. 10,835,307, entitled “Modular Battery Powered Handheld Surgical Instrument Containing Elongated Multi-Layered Shaft,” issued Nov. 17, 2020, the disclosure of which is incorporated by reference herein, in its entirety; and U.S. Pat. No. 11,229,471, entitled “Modular Battery Powered Handheld Surgical Instrument with Selective Application of Energy Based on Tissue Characterization,” issued Jan. 25, 2022, the disclosure of which is incorporated by reference herein, in its entirety.

In some scenarios, it may be preferable to have surgical instruments grasped and manipulated directly by the hand or hands of one or more human operators. In addition, or as an alternative, it may be preferable to have surgical instruments controlled via a robotic surgical system. Examples of robotic surgical systems and associated instrumentation are disclosed in U.S. Pat. No. 10,624,709, entitled “Robotic Surgical Tool with Manual Release Lever,” published on May 2, 2019, the disclosure of which is incorporated by reference herein, in its entirety; U.S. Pat. No. 9,314,308, entitled “Robotic Ultrasonic Surgical Device With Articulating End Effector,” issued on Apr. 19, 2016, the disclosure of which is incorporated by reference herein, in its entirety; U.S. Pat. No. 9,125,662, entitled “Multi-Axis Articulating and Rotating Surgical Tools,” issued Sep. 8, 2015, the disclosure of which is incorporated by reference herein, in its entirety; U.S. Pat. No. 8,820,605, entitled “Robotically-Controlled Surgical Instruments,” issued Sep. 2, 2014, the disclosure of which is incorporated by reference herein, in its entirety; U.S. Pub. No. 2019/0201077, entitled “Interruption of Energy Due to Inadvertent Capacitive Coupling,” published Jul. 4, 2019, issued as U.S. Pat. No. 11,291,495 on Apr. 5, 2022, the disclosure of which is incorporated by reference herein, in its entirety; U.S. Pub. No. 2012/0292367, entitled “Robotically-Controlled End Effector,” published on Nov. 11, 2012, now abandoned, the disclosure of which is incorporated by reference herein, in its entirety; and U.S. patent application Ser. No. 16/556,661, entitled “Ultrasonic Surgical Instrument with a Multi-Planar Articulating Shaft Assembly,” filed on Aug. 30, 2019, issued as U.S. Pat. No. 11,690,642 on Jul. 4, 2023, the disclosure of which is incorporated by reference herein, in its entirety.

Such instruments and robotic surgical systems may be further be incorporated into a surgical system for performing procedures in a surgical environment, such as surgical operating theaters or rooms in a healthcare facility. A sterile field is typically created around the patient and may include properly attired, scrubbed healthcare professions as well as desired furniture and/or fixtures. Examples of such surgical systems and associated features are disclosed in U.S. Pat. Pub. No. 2019/0201046, entitled “Method for Controlling Smart Energy Devices,” published on Jul. 4, 2019, issued as U.S. Pat. No. 11,589,888 on Feb. 28, 2023, the disclosure of which is incorporated by reference herein, in its entirety; U.S. Pat. Pub. No. 2019/0201080, entitled “Ultrasonic Energy Device Which Varies Pressure Applied by Clamp Arm to Provide Threshold Control Pressure at a Cut Progression Location,” published on Jul. 4, 2019, issued as U.S. Pat. No. 11,419,667 on Aug. 23, 2022, the disclosure of which is incorporated by reference herein, in its entirety; U.S. Pat. Pub. No. 2019/0201091, entitled “Radio Frequency Energy Device for Delivering Combined Electrical Signals,” published Jul. 4, 2019, issued as U.S. Pat. No. 11,364,075 on Jun. 21, 2022, the disclosure of which is incorporated by reference herein, in its entirety; U.S. Pat. Pub. No. 2019/0274717, entitled “Methods for Controlling Temperature in Ultrasonic Device,” published Sep. 12, 2019, issued as U.S. Pat. No. 11,259,830 on Mar. 1, 2022, the disclosure of which is incorporated by reference herein, in its entirety; and U.S. Pat. Pub. No. 2019/0207857, entitled “Surgical Network Determination of Prioritization of Communication, Interaction, or Processing Based on System or Device Needs,” published Jul. 4, 2019, issued as U.S. Pat. No. 10,892,995 on Jan. 12, 2021, the disclosure of which is incorporated by reference herein, in its entirety.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a block diagram of an example a computer-implemented interactive surgical system;

FIG. 2 depicts a top schematic view of an example of a surgical system for performing a surgical procedure in an operating room of a healthcare facility;

FIG. 3 depicts a side schematic view of an example of a surgical hub of the surgical system of FIG. 2;

FIG. 4 depicts a perspective view of a combination generator module with bipolar, ultrasonic, and monopolar contacts of the surgical system of FIG. 2;

FIG. 5 depicts a side schematic view of an exemplary generator and various examples of surgical instruments for use with the surgical system of FIG. 2;

FIG. 6A depicts a side view of a surgical instrument including a plurality of selectively removeable shrouds attached by a plurality of magnetic members in a connected state;

FIG. 6B depicts a partially exploded, side view of the surgical instrument of FIG. 6A with the shrouds separated from one another in a disconnected state;

FIG. 7A depicts a side view of a surgical instrument including a magnetic lock assembly in a locked state and a plurality of selectively removeable shrouds retained by the magnetic lock assembly in a connected state;

FIG. 7B depicts a partially exploded side view of the surgical instrument of FIG. 7A with the magnetic lock assembly in an unlocked state and the shrouds separated from one another in a disconnected state;

FIG. 8A depicts a perspective view of a surgical instrument including a shroud attached by push-pins in a connected state and in electrical communication with a generator;

FIG. 8B depicts a perspective view of the surgical instrument of FIG. 8A with the shrouds separated from one another in a disconnected state;

FIG. 9 depicts an enlarged perspective view of a push-pin being removed from the shroud of the surgical instrument of FIG. 8A;

FIG. 10 depicts a side view of another push-pin for use with surgical instrument of FIG. 8A;

FIG. 11A depicts a perspective view of a portion of a surgical instrument with a shroud removed exposing a main circuit board with a plurality of pluggable sub-boards in an installed position;

FIG. 11B depicts a perspective view of the portion of the surgical instrument of FIG. 11A with the shroud removed and the plurality of pluggable sub-boards in an uninstalled position;

FIG. 12 depicts a perspective view of a portion of a surgical instrument with a shroud removed exposing a main circuit board including a frangible separator in an intact state;

FIG. 13 depicts a perspective view of the main circuit board of the surgical instrument of FIG. 12 with the main circuit board in a separated state;

FIG. 14A depicts a sectional view of a surgical instrument including a latch in a latched position and a memory member in an operable state;

FIG. 14B depicts a sectional view of the surgical instrument of FIG. 14A including the latch in an unlatched position after passing over the memory member rendering the memory member in an inoperable state;

FIG. 15 depicts a sectional view of a surgical instrument including a latch configured to move to an open position and engage a set of contacts to render data on a memory member unreadable;

FIG. 16 depicts a side schematic view of a circuit assembly of a surgical instrument including a memory member connected with a flex circuit to a main circuit board;

FIG. 17 depicts a side schematic view of another circuit assembly of a surgical instrument including a memory member connected with a pin connector to a main circuit board;

FIG. 18 depicts a side schematic view of a circuit assembly of a surgical instrument including a memory member with a frangible notch connected to a main circuit board;

FIG. 19A depicts a sectional view of a portion of a surgical instrument including a selectively removable shroud in an installed position, a support member, and a main circuit board in an operable state;

FIG. 19B depicts a sectional view of the portion of the surgical instrument of FIG. 19A in an uninstalled position with the shroud removed and the support member connecting the main circuit board to the shroud into an inoperable state;

FIG. 20 depicts a side view of an energy drive system of a surgical instrument with an energy coupling;

FIG. 21 depicts an enlarged side view of a portion of the energy drive system of FIG. 20;

FIG. 22 depicts a side view of an energy drive system of a surgical instrument with an energy coupling separating a waveguide from an ultrasonic transducer;

FIG. 23 depicts an enlarged sectional view of a portion of the energy drive system of FIG. 22;

FIG. 24 depicts an enlarged sectional view of a portion of another energy drive system;

FIG. 25 depicts a side view of an energy drive system including an ultrasonic transducer fitted with a cover;

FIG. 26A depicts a perspective view of a body assembly of a surgical instrument with first and second shroud portions in a connected state fitted with a strain relief feature retaining a cable and having portions thereof hidden for greater clarity;

FIG. 26B depicts a perspective view of the body assembly of FIG. 26A with the first and second shroud portions in a disconnected state and the strain relief feature releasing the cable and having portion thereof hidden for greater clarity;

FIG. 27 depicts a perspective view of an exemplary cable assembly connected between a medical device and a generator for use with the surgical system of FIG. 2;

FIG. 28 depicts an enlarged, partially cutaway, perspective view of a cable adapter of the cable assembly of FIG. 27;

FIG. 29 depicts an enlarged, cross-sectional view of the cable adapter of FIG. 28 taken along a centerline thereof receiving an exemplary instrument adapter of the medical device of FIG. 27 in an uncoupled position;

FIG. 30 depicts the enlarged, cross-sectional view of the cable adapter similar to FIG. 29, but showing the instrument adapter received by the cable adapter from a partially coupled, unlocked position to a fully coupled, locked position;

FIG. 30A depicts a cross-sectional view of the cable adapter of FIG. 30 taken along section line 30A-30A of FIG. 30;

FIG. 31 depicts a side schematic view of another exemplary cable assembly receiving another exemplary instrument adapter of a medical device from an uncoupled position to a partially coupled, unlocked position;

FIG. 32 depicts the side schematic view of the cable assembly and instrument adapters similar to FIG. 31, but showing the cable assembly and instrument adapters moved from the partially coupled, unlocked position of FIG. 31 to a fully coupled, locked position;

FIG. 33 depicts a front schematic view of a surgical kit including the surgical instrument of FIG. 5 and an example of an instrument tool assembly;

FIG. 34 depicts a side schematic view of the generator of FIG. 5 and the instrument tool assembly of FIG. 33;

FIG. 35 depicts a front schematic view of the instrument tool assembly of FIG. 33;

FIG. 36A depicts a front schematic view of another example of an instrument tool assembly in a first configuration;

FIG. 36B depicts a front schematic view of the instrument tool assembly of FIG. 36A in a second configuration;

FIG. 37 depicts a schematic front view of an exemplary robotic surgical system that includes a plurality of tools;

FIG. 38 depicts a perspective view of a surgical instrument, where a housing of the surgical instrument is partially removed to expose reclaimable components;

FIG. 39 depicts a top plan view of an exemplary tool that includes a plurality of disassembly features;

FIG. 40 depicts a side elevational view of a first exemplary disassembly feature of the tool of FIG. 39 moving from a first configuration to a second configuration to disassemble a portion of the tool of FIG. 38;

FIG. 41 depicts a side elevational view of the disassembly feature of FIG. 40 moving from a third configuration to a fourth configuration to disassemble a portion of the surgical instrument of FIG. 38;

FIG. 42A depicts a schematic sectional view of first and second housing portions of the surgical instrument of FIG. 38 coupled together in a connected configuration using a mechanical connector prior to disassembly by a second exemplary disassembly feature of the tool of FIG. 39;

FIG. 42B depicts the schematic sectional view of first and second housing portions similar to FIG. 42A, but after the disassembly feature of FIG. 42A moves the mechanical connector to a non-connected configuration;

FIG. 43A depicts a schematic sectional view of first and second housing portions of the surgical instrument of FIG. 38 coupled together in a connected configuration using a magnetic connector;

FIG. 43B depicts the schematic sectional view of first and second housing portions similar to FIG. 43A, but after the magnetic connector is moved to a non-connected configuration by a third exemplary disassembly feature;

FIG. 44A depicts a schematic sectional view of first and second housing portions of the surgical instrument of FIG. 38 coupled together in a connected configuration using an electrical connector;

FIG. 44B depicts the schematic sectional view of first and second housing portions similar to FIG. 44A, but after the electrical connector is moved to a non-connected configuration using a fourth exemplary disassembly feature;

FIG. 45A depicts a schematic sectional view of a portion of the housing of FIG. 38 as a fifth exemplary disassembly feature is activated;

FIG. 45B depicts the schematic sectional view of the portion of the housing similar to FIG. 45A, but after separation using the disassembly feature of FIG. 45A;

FIG. 46A depicts a schematic sectional view of a sixth exemplary disassembly features moving from the first configuration toward the second configuration to separate a frangible portion of the surgical instrument of FIG. 38;

FIG. 46B depicts the schematic sectional view of the disassembly features of FIG. 46A, but with the frangible portion in a separated state;

FIG. 47 depicts a side elevational view of a plurality of tools positioned on a tool dispenser;

FIG. 48 depicts an exemplary surgical kit that includes packaging surrounding another exemplary surgical instrument and a disassembly feature;

FIG. 49 depicts the surgical instrument of FIG. 48 but in a disassembled configuration;

FIG. 50 depicts a diagrammatic view of an exemplary method of operating the robotic surgical system of FIG. 37;

FIG. 51A depicts a schematic view of a surgical theater, where a surgical kit is located within a non-sterile entry room;

FIG. 51B depicts a schematic view of the surgical theater of FIG. 51A, where the surgical kit is transported into the sterile field of the surgical theater;

FIG. 51C depicts a schematic view of the surgical theater of FIG. 51A, where the surgical kit is divided into assembled surgical subcomponents, non-surgical subcomponents, and a plurality of disposal bags;

FIG. 51D depicts a schematic view of the surgical theater of FIG. 51A, where the surgical subcomponents and the non-surgical subcomponents are broken down into post-surgery components organized into categories;

FIG. 51E depicts a schematic view of the surgical theater of FIG. 51A, where the post-surgery components that are organized into categories and placed within a respective disposal bag of the surgical kit of FIG. 51A;

FIG. 51F depicts a schematic view of the surgical theater of FIG. 51A, where the post-surgery components and the disposal bags are transported into a non-sterile exit room;

FIG. 52 depicts a schematic view of an exemplary surgical instrument and a computational device;

FIG. 53 depicts a flowchart of a method for determining disposal instructions for an energized surgical instrument recently used in a surgical procedure;

FIG. 54A depicts a schematic view of a generator having a longer power cord and a surgical instrument having a shorter power cord, wherein the longer power cord is contained within a storage bin of the generator;

FIG. 54B depicts a schematic view of the generator and the surgical instrument of FIG. 54A, where the longer power cord extends away from the storage bin of the generator;

FIG. 54C depicts a schematic view of the generator and the surgical instrument of FIG. 54A, wherein the longer power cord and the shorter power cord are coupled together;

FIG. 55 depicts a perspective schematic view of a first exemplary surgical kit, where an outer packaging of the surgical kit is in a closed configuration;

FIG. 56A depicts a top schematic view of the surgical kit of FIG. 55, but with the outer packaging in a partially open configuration;

FIG. 56B depicts a top schematic view of return packaging of the surgical kit including portions of the surgical instrument of FIG. 56A;

FIG. 57 depicts a perspective schematic view of a second exemplary surgical kit, where the outer packaging of the surgical kit is in a closed configuration;

FIG. 58 depicts a top schematic view of sterile packaging, return packaging, and portions of the surgical instrument of the surgical kit of FIG. 57;

FIG. 59 depicts a diagrammatic view of any exemplary method of using the surgical kits of FIGS. 55 and 57;

FIG. 60 depicts an exploded perspective view of an exemplary proximal body that may be readily incorporated into any of the surgical instruments shown herein;

FIG. 61A depicts a sectional view of the proximal body of FIG. 60, where a first shroud and a second shroud are decoupled from each other;

FIG. 61B depicts an enlarged sectional view of the proximal body of FIG. 60, where the first shroud and the second shroud of FIG. 61A actuated toward each other;

FIG. 61C depicts an enlarged sectional view of the proximal body of FIG. 60, where the first shroud and the second shroud of FIG. 61A are coupled together such that a latching assembly is in a locked configuration;

FIG. 61D depicts an enlarged sectional view of the proximal body of FIG. 60, where the first shroud and the second shroud of FIG. 61A are coupled together, where a user is actuating the latching assembly of FIG. 61C in an unlocked configuration;

FIG. 61E depicts an enlarged sectional view of the proximal body of FIG. 60, where the first shroud and the second shroud of FIG. 61A are initially decoupled from each other;

FIG. 62 depicts an exploded perspective view of another exemplary proximal body that may be readily incorporated into any of the surgical instruments shown herein;

FIG. 63 depicts an exploded perspective view of another exemplary proximal body that may be readily incorporated into any of the surgical instruments shown herein;

FIG. 64A depicts an enlarged perspective view of a coupling assembly of the proximal body of FIG. 63 in a decoupled configuration;

FIG. 64B depicts an enlarged cross-sectional view of the coupling assembly taken along section line 64B-64B of FIG. 64A in a coupled configuration;

FIG. 65A depicts an enlarged sectional view of an alternative coupling assembly in a decoupled configuration;

FIG. 65B depicts an enlarged sectional view of the coupling assembly of FIG. 65A in a coupled configuration;

FIG. 66 depicts an enlarged exploded perspective view of another exemplary proximal body that may be readily incorporated into any of the surgical instruments shown herein;

FIG. 67A depicts an enlarged sectional of a coupling assembly of the proximal body of FIG. 66 in a decoupled configuration;

FIG. 67B depicts an enlarged sectional view of the coupling assembly of FIG. 67A in a coupled configuration;

FIG. 68A depicts an enlarged sectional view of an alternative coupling assembly in a decoupled configuration;

FIG. 68B depicts an enlarged sectional view of the coupling assembly of FIG. 68A in a coupled configuration;

FIG. 69A depicts an enlarged sectional view of another exemplary proximal body that may be readily incorporated into any of the surgical instruments shown herein, where the proximal body is assembled with an electrical component housed therein;

FIG. 69B depicts an enlarged sectional view of the proximal body of FIG. 69A, wherein the proximal body is disassembled with the electrical component biased away from a shroud of the proximal body;

FIG. 70A depicts a sectional view of a shroud of the proximal body of FIG. 69A still coupled with the electrical component in FIG. 69A placed above an exemplary dip tray;

FIG. 70B depicts a sectional view of the shroud of FIG. 70A coupled with the electrical component of FIG. 69A inappropriately placed within the dip tray of FIG. 70A;

FIG. 71A depicts a sectional view of the shroud of FIG. 70A decoupled from the electrical component of FIG. 69A and placed above the dip tray of FIG. 70A;

FIG. 71B depicts a sectional view of the shroud of FIG. 70A decoupled from the electrical component of FIG. 69A and appropriately placed within the dip tray of FIG. 70A;

FIG. 72A depicts an enlarged elevational side view of another exemplary proximal body that may be readily incorporated into any of the surgical instruments shown herein;

FIG. 72B depicts an enlarged elevational side view of the proximal body of FIG. 72A, where a scanning device is scanning a bar code of the proximal body;

FIG. 72C depicts an enlarged elevational side view of the proximal body of FIG. 72A, where a hatch door of the proximal body is removed;

FIG. 73A depicts an enlarged sectional view of another exemplary proximal body that may be readily incorporated into any of the surgical instruments shown herein;

FIG. 73B depicts an enlarged sectional view of the proximal body of FIG. 73A, where a magnet is hovered over the proximal body;

FIG. 73C depicts an enlarged sectional view of the proximal body of FIG. 73A, where a hatch door of the proximal body is removed;

FIG. 74A depicts an enlarged elevational side view of another exemplary proximal body that may be readily incorporated into any of the surgical instruments shown herein;

FIG. 74B depicts an enlarged elevational side view of the proximal body of FIG. 74A, where a heat source is hovered over the proximal body;

FIG. 74C depicts an enlarged elevational side view of the proximal body of FIG. 74A, where a hatch door of the proximal body is removed;

FIG. 75A depicts an enlarged elevational side view of another exemplary proximal body that may be readily incorporated into any of the surgical instruments shown herein;

FIG. 75B depicts an enlarged elevational side view of the proximal body of FIG. 75A, where an interactive is hovered over the proximal body;

FIG. 75C depicts an enlarged elevational side view of the proximal body of FIG. 75A, where a power cord is detached form the proximal body;

FIG. 76A depicts a perspective view of a processing bag assembly in a closed configuration;

FIG. 76B depicts a perspective view of the processing bag assembly of FIG. 76A in an open configuration;

FIG. 76C depicts a perspective view of the processing bag assembly of FIG. 76A in the open configuration with a surgical component loaded therein;

FIG. 76D depicts a perspective view of the processing bag assembly of FIG. 76A in the closed configuration with the surgical component of FIG. 76C loaded therein;

FIG. 77 depicts a schematic view of another exemplary processing bag assembly;

FIG. 78 depicts a schematic view of another exemplary processing bag assembly;

FIG. 79 depicts a schematic view of another exemplary processing bag assembly;

FIG. 80 depicts a schematic view of another exemplary processing bag assembly;

FIG. 81 depicts a flowchart of any exemplary shutdown cycle that may be used with any of the surgical instruments shown herein;

FIG. 82 depicts a schematic view of an exemplary surgical visualization system including an imaging device and a surgical device;

FIG. 83 depicts a schematic diagram of an exemplary control system that may be used with the surgical visualization system of FIG. 82;

FIG. 84 depicts a flow chart of an exemplary method for determining the recovery capacity of at least one feature of a surgical instrument;

FIG. 85 depicts a flow chart of an exemplary assessment method for determining the recovery capacity of at least one feature of a surgical instrument;

FIG. 86 depicts a flow chart of an exemplary assessment method for determining the recovery capacity of at least one feature of a surgical instrument;

FIG. 87 depicts a flow chart of an exemplary assessment method for determining the recovery capacity of at least one feature of a surgical instrument;

FIG. 88 depicts a flow chart of an exemplary assessment method for determining the recovery capacity of at least one feature of a surgical instrument;

FIG. 89 depicts a perspective view of an assessment and cleaning port that may be utilized to assess the recovery capacity of at least one feature of surgical instrument and then clean the at least one feature of the surgical instrument;

FIG. 90A depicts a sectional view of the assessment and cleaning port of FIG. 89 with an end effector partially inserted;

FIG. 90B depicts a sectional view of the assessment and cleaning port of FIG. 89 with the end effector further inserted;

FIG. 91 depicts a perspective view of an end effector sheath;

FIG. 92A depicts a sectional view of the end effector sheath of FIG. 91 with an end effector adjacent to an entry of the sheath;

FIG. 92B depicts a sectional view of the end effector sheath of FIG. 91 with an end effector inserted into the sheath;

FIG. 93 depicts an top plan view of a surgical kit packaging; and

FIG. 94 depicts a perspective view of a removable cleaning kit of the surgical kit packaging of FIG. 93.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” “bottom,” “above,” and “below,” are used with respect to the examples and associated figures and are not intended to unnecessarily limit the invention described herein.

### I. EXAMPLE OF A SURGICAL SYSTEM

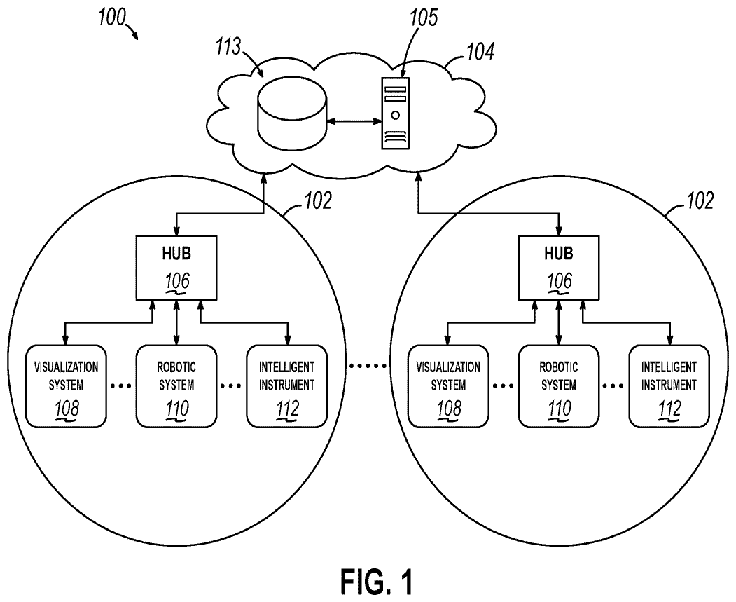

With respect to FIG. 1, a computer-implemented interactive surgical system (100) includes one or more surgical systems (102) and a cloud-based system (e.g., a cloud (104) that may include a remote server (113) coupled to a storage device (105)). Each surgical system (102) of the present example includes at least one surgical hub (106) in communication with cloud (104) that may include a remote server (113). In one example, as illustrated in FIG. 1, surgical system (102) includes a visualization system (108), a robotic system (110), and a handheld intelligent surgical instrument (112), which are configured to communicate with one another and/or hub (106). In some aspects, a surgical system (102) may include an M number of hubs (106), an N number of visualization systems (108), an O number of robotic systems (110), and a P number of handheld intelligent surgical instruments (112), where M, N, O, and P are integers greater than or equal to one. In any case, any suitable combination of features provided below may be incorporated into an exemplary surgical system, such as surgical system (100), and used in the surgical theater in order to perform a desired surgical procedure as would be apparent to one skilled in the art in view of the teachings herein.

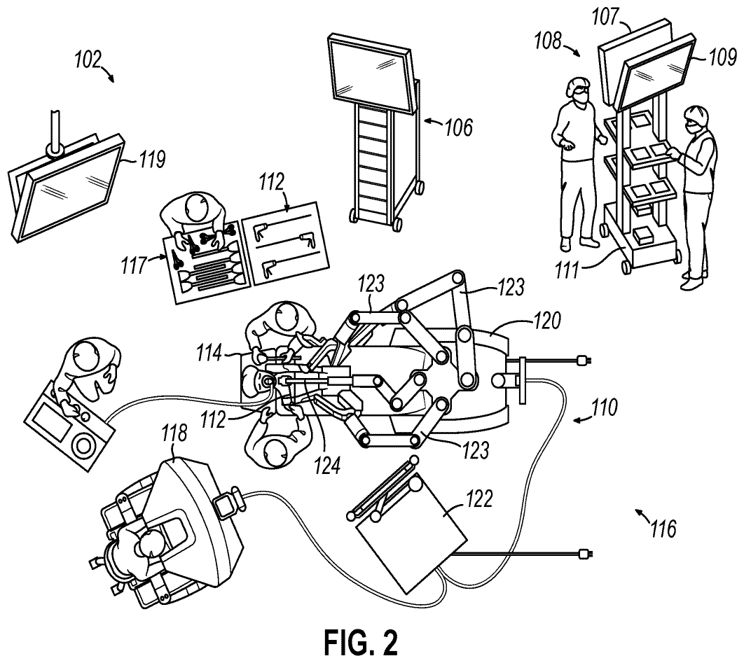

FIG. 2 depicts an example of a surgical system (102) being used to perform a surgical procedure on a patient who is lying down on an operating table (114) in a surgical operating room (116). A robotic system (110) is used in the surgical procedure as a part of surgical system (102). Robotic system (110) includes a surgeon's console (118), a patient side cart (120) (surgical robot), and a surgical robotic hub (122). Patient side cart (120) can manipulate at least one removably coupled surgical tool (117) with any one of a plurality of surgical arms (123) through a minimally invasive incision in the body of the patient while the surgeon views the surgical site through console (118). An image of the surgical site can be obtained by a medical imaging device (124), which can be manipulated by patient side cart (120) to orient imaging device (124). Robotic hub (122) can be used to process the images of the surgical site for subsequent display to the surgeon through console (118).

Other types of robotic systems can be readily adapted for use with surgical system (102). Various examples of robotic systems and surgical tools that are suitable for use with the present disclosure are described in U.S. Provisional Patent Application Ser. No. 62/611,339, entitled “Robot Assisted Surgical Platform,” filed Dec. 28, 2017, the disclosure of which is herein incorporated by reference in its entirety.

Various examples of cloud-based analytics that are performed by cloud (104, and are suitable for use with the present disclosure, are described in U.S. Provisional Patent Application Ser. No. 62/611,340, entitled Cloud-Based Medical Analytics,” filed Dec. 28, 2017, the disclosure of which is herein incorporated by reference in its entirety.

In various aspects, imaging device (124) includes at least one image sensor and one or more optical components. Suitable image sensors include, but are not limited to, Charge-Coupled Device (CCD) sensors and Complementary Metal-Oxide Semiconductor (CMOS) sensors. In various aspects, imaging device (124) is configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope. Some aspects of spectral and multispectral imaging are described in greater detail under the heading “Advanced Imaging Acquisition Module” in U.S. Provisional Patent Application Ser. No. 62/611,341, entitled “Interactive Surgical Platform,” filed Dec. 28, 2017, the disclosure of which is herein incorporated by reference in its entirety.

Strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

In addition to the introduction of any features of surgical system (100), furniture, or fixtures into the sterile field requiring sterilization, additional complications may result from removal of these features from the sterile field, particularly when such features may have contacted, or presumed to have contacted, the patient, including any tissues and/or fluids associated with the surgical procedure. Such contamination of these features from the patient often requires special consideration during or after the surgical procedure, particularly when processing these features for disposal, reuse, or remanufacturing as desired. In one example, surgical system (100) and/or healthcare professionals associated with the surgical procedure may be specifically equipped to address such processing as discussed below in greater detail.

As illustrated in FIG. 2, a primary display (119) is positioned in the sterile field to be visible to an operator at operating table (114). In addition, a visualization tower (111) is positioned outside the sterile field. Visualization tower (111) includes a first non-sterile display (107) and a second non-sterile display (109), which face away from each other. Visualization system (108), guided by hub (106), is configured to utilize displays (107, 109, 119) to coordinate information flow to operators inside and outside the sterile field. For example, hub (106) may cause visualization system (108) to display a snapshot of a surgical site, as recorded by imaging device (124), on a non-sterile display (107) or (109), while maintaining a live feed of the surgical site on the primary display (119). The snapshot on non-sterile display (107) or display (109) can permit a non-sterile operator to perform a diagnostic step relevant to the surgical procedure, for example.

In one aspect, hub (106) is also configured to route a diagnostic input or feedback entered by a non-sterile operator at visualization tower (111) to primary display (119) within the sterile field, where it can be viewed by a sterile operator at the operating table. In one example, the input can be in the form of a modification to the snapshot displayed on non-sterile display (107) or display (109), which can be routed to primary display (119) by hub (106).

Referring to FIG. 2, a surgical instrument (112) is being used in the surgical procedure as part of surgical system (102). Hub (106) is also configured to coordinate information flow to a display of the surgical instrument (112) such as in, for example, U.S. Provisional Patent Application Ser. No. 62/611,341, entitled “Interactive Surgical Platform,” filed Dec. 28, 2017, the disclosure of which is herein incorporated by reference in its entirety. A diagnostic input or feedback entered by a non-sterile operator at visualization tower (111) can be routed by hub (106) to surgical instrument display (115) within the sterile field, where it can be viewed by the operator of surgical instrument (112). Example surgical instruments that are suitable for use with surgical system (102) are described under the heading “Surgical Instrument Hardware” and in U.S. Provisional Patent Application Ser. No. 62/611,341, entitled “Interactive Surgical Platform,” filed Dec. 28, 2017, the disclosure of which is herein incorporated by reference in its entirety, for example.

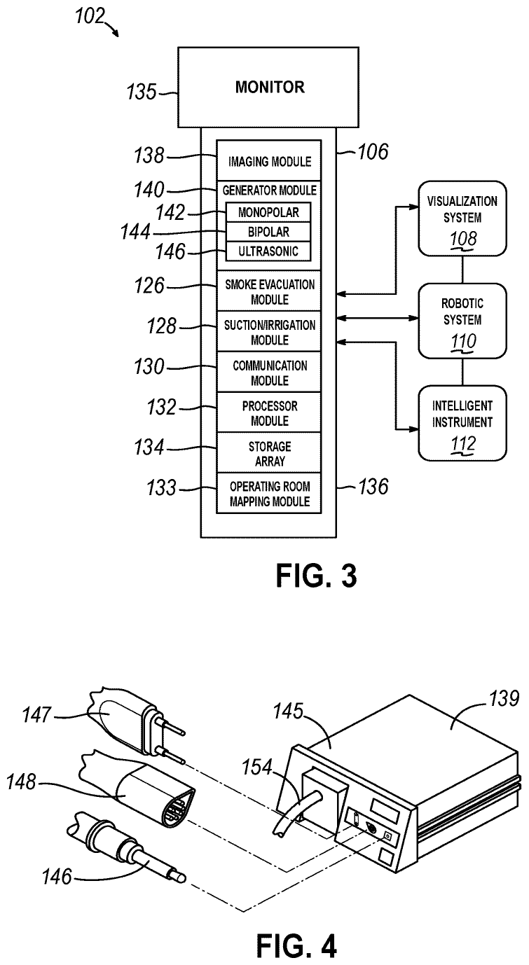

Referring now to FIG. 3, a hub (106) is depicted in communication with a visualization system (108), a robotic system (110), and a handheld intelligent surgical instrument (112). Hub (106) includes a hub display (135), an imaging module (138), a generator module (140), a communication module (130), a processor module (132), and a storage array (134). In certain aspects, as illustrated in FIG. 3, hub (106) further includes a smoke evacuation module (126), a suction/irrigation module (128), and/or an operating room mapping module (133).

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure (136) offers a unified environment for managing the power, data, and fluid lines, which reduces the frequency of entanglement between such lines.

Referring to FIGS. 3-4, aspects of the present disclosure are presented for a hub modular enclosure (136) that allows the modular integration of a generator module (140), a smoke evacuation module (126), and a suction/irrigation module (128). Hub modular enclosure (136) further facilitates interactive communication between modules (140, 126, 128). As shown in FIG. 4, generator module (140) can be a generator module with integrated monopolar, bipolar, and ultrasonic components supported in a single housing unit (139) slidably insertable into hub modular enclosure (136). As illustrated in FIG. 4, generator module (140) can be configured to connect to a monopolar device (146), a bipolar device (147), and an ultrasonic device (148). Alternatively, generator module (140) may comprise a series of monopolar, bipolar, and/or ultrasonic generator modules that interact through hub modular enclosure (136). Hub modular enclosure (136) can be configured to facilitate the insertion of multiple generators and interactive communication between the generators docked into the hub modular enclosure (136) so that the generators would act as a single generator.

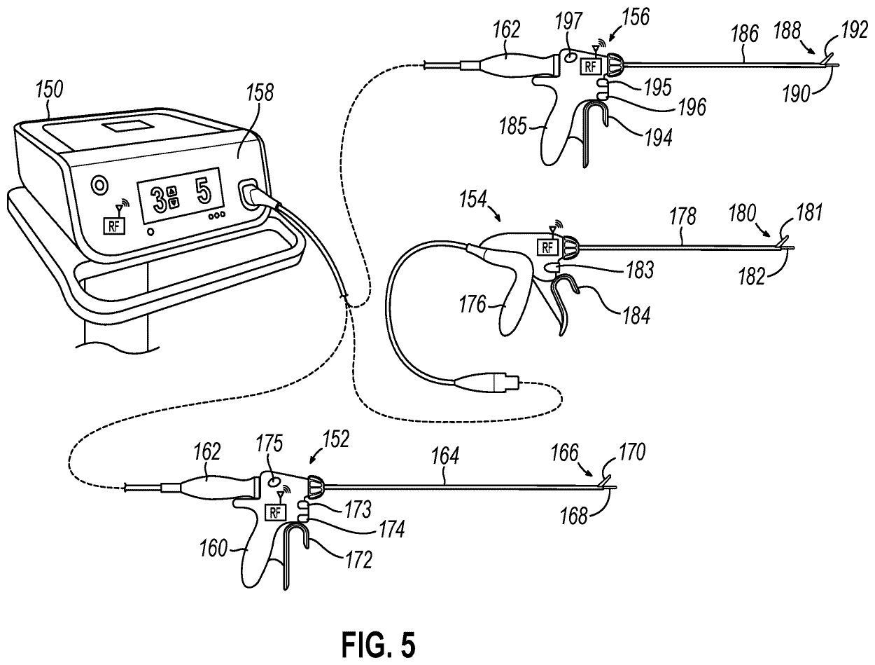

FIG. 5 illustrates one form of a generator (150) and various surgical instruments (152, 154, 156) usable therewith, where surgical instrument (152) is an ultrasonic surgical instrument (152), surgical instrument (154) is an RF electrosurgical instrument (154), and multifunction surgical instrument (156) is a combination ultrasonic/RF electrosurgical instrument (156). Generator (150) is configurable for use with a variety of surgical instruments. According to various forms, generator (150) may be configurable for use with different surgical instruments of different types including, for example, ultrasonic surgical instruments (152), RF electrosurgical instruments (154), and multifunction surgical instruments (156) that integrate RF and ultrasonic energies delivered simultaneously from generator (150). Although generator (150) of the present example in FIG. 5 is shown separate from surgical instruments (152, 154, 156), generator (150) may alternatively be formed integrally with any of surgical instruments (152, 154, 156) to form a unitary surgical system. Generator (150) comprises an input device (158) located on a front panel of generator (150) console. Input device (158) may comprise any suitable device that generates signals suitable for programming the operation of generator (150). Generator (150) may be configured for wired or wireless communication.

Generator (150) of the present example is configured to drive multiple surgical instruments (152, 154, 156). One example of such surgical instrument is ultrasonic surgical instrument (152) and comprises a handpiece (160), an ultrasonic transducer 162, a shaft assembly (164), and an end effector (166). End effector (166) includes an ultrasonic blade (168) acoustically coupled to ultrasonic transducer (162) and a clamp arm (170). Handpiece (160) has a trigger (172) to operate clamp arm (170) and a combination of toggle buttons (173, 174, 175) to energize and drive ultrasonic blade (168) or other function. Toggle buttons (173, 174, 175) can be configured to energize ultrasonic transducer (162) with generator (150).

Generator (150) also is configured to drive another example of surgical instrument (154). RF electrosurgical instrument (154) includes a handpiece (176), a shaft assembly (178), and an end effector (180). End effector (180) includes electrodes in clamp arms (181, 182) and return through an electrical conductor portion of shaft assembly (178).

Electrodes are coupled to and energized by a bipolar energy source within generator (150). Handpiece (176) includes a trigger (183) to operate clamp arms (181, 182) and an energy button (184) to actuate an energy switch to energize electrodes in end effector (180).

Generator (150) also is configured to drive multifunction surgical instrument (156). Multifunction surgical instrument (156) includes a handpiece (185), a shaft assembly (186), and an end effector (188). End effector (188) has an ultrasonic blade (190) and a clamp arm (192). Ultrasonic blade (190) is acoustically coupled to ultrasonic transducer (162). Handpiece (185) has a trigger (194) to operate clamp arm (192) and a combination of toggle buttons (195, 196, 197) to energize and drive ultrasonic blade (190) or other function. Toggle buttons (195, 196, 197) can be configured to energize ultrasonic transducer (162) with generator (150) and energize ultrasonic blade (190) with a bipolar energy source also contained within generator (150). It will be appreciated that handpieces (160, 176, 185) may be replaced with a robotically controlled instrument for incorporating one or more aspects of surgical instruments (152, 154, 156). Accordingly, the term “handpiece” should not be limited to this context and to handheld use.

As used throughout this description, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some aspects they might not. The communication module may implement any of a number of wireless or wired communication standards or protocols, including but not limited to Wi-Fi (IEEE 802.11 family), WMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, Ethernet derivatives thereof, as well as any other wireless and wired protocols that are designated as 3G, 4G, 5G, and beyond. The computing module may include a plurality of communication modules. For instance, a first communication module may be dedicated to shorter range wireless communications such as Wi-Fi and Bluetooth and a second communication module may be dedicated to longer range wireless communications such as GPS, EDGE, GPRS, CDMA, WiMAX, LTE, Ev-DO, and others.

As used herein a processor or processing unit is an electronic circuit which performs operations on some external data source, usually memory or some other data stream. The term is used herein to refer to the central processor (central processing unit) in a system or computer systems (especially systems on a chip (SoCs)) that combine a number of specialized “processors.”

As used herein, a system on a chip or system on chip (SoC or SOC) is an integrated circuit (also known as an “IC” or “chip”) that integrates all components of a computer or other electronic systems. It may contain digital, analog, mixed-signal, and often radiofrequency functions, all on a single substrate. A SoC integrates a microcontroller (or microprocessor) with advanced peripherals like graphics processing unit (GPU), Wi-Fi module, or coprocessor. A SoC may or may not contain built-in memory.

As used herein, a microcontroller or controller is a system that integrates a microprocessor with peripheral circuits and memory. A microcontroller (or MCU for microcontroller unit) may be implemented as a small computer on a single integrated circuit. It may be similar to a SoC; an SoC may include a microcontroller as one of its components. A microcontroller may contain one or more core processing units (CPUs) along with memory and programmable input/output peripherals. Program memory in the form of Ferroelectric RAM, NOR flash or OTP ROM is also often included on chip, as well as a small amount of RAM. Microcontrollers may be employed for embedded applications, in contrast to the microprocessors used in personal computers or other general purpose applications consisting of various discrete chips.

As used herein, the term controller or microcontroller may be a stand-alone IC or chip device that interfaces with a peripheral device. This may be a link between two parts of a computer or a controller on an external device that manages the operation of (and connection with) that device. Modular devices include the modules (as described in connection with FIG. 3, for example) that are receivable within a surgical hub and the surgical devices or instruments that can be connected to the various modules in order to connect or pair with the corresponding surgical hub. The modular devices include, for example, intelligent surgical instruments, medical imaging devices, suction/irrigation devices, smoke evacuators, energy generators, ventilators, insufflators, and displays. The modular devices described herein can be controlled by control algorithms. The control algorithms can be executed on the modular device itself, on the surgical hub to which the particular modular device is paired, or on both the modular device and the surgical hub (e.g., via a distributed computing architecture). In some exemplifications, the modular devices' control algorithms control the devices based on data sensed by the modular device itself (i.e., by sensors in, on, or connected to the modular device). This data can be related to the patient being operated on (e.g., tissue properties or insufflation pressure) or the modular device itself (e.g., the rate at which a knife is being advanced, motor current, or energy levels). For example, a control algorithm for a surgical stapling and cutting instrument can control the rate at which the instrument's motor drives its knife through tissue according to resistance encountered by the knife as it advances.

### II. EXEMPLARY SURGICAL INSTRUMENT INCORPORATING SELECTIVELY SEPARABLE SHROUD

In some instances, it may be desirable to provide a surgical instrument (1000) similar to any one or more of surgical instruments (112, 152, 154, 156) that includes components capable of delivering ultrasonic energy, RF energy, or both ultrasonic and RF energy that easily open to provide access to the internal components for separation into separate waste streams with minimal tools, such as no additional tools. Surgery customarily takes place within the sterile field, as described above. The sterile field, being free of microorganisms, enables the surgical team to decrease the chance of infection by ensuring that only sterilized equipment and tools are used within the sterile field. Surgical instruments are sterilized and packaged within sterile containers that are passed into the sterile field. Health care professionals may be required to disassemble the surgical instruments within the sterile field after a surgical procedure by hand or with tools provided within the sterile containers. For example, a torque wrench provided for assembling a surgical instrument, may have additional features to disassemble the surgical instrument. The surgical instruments include additional features that facilitate disassembly and removal of internal components. These additional features aid in selectively breaking the internal components so the components may be placed into separate waste streams. These separate waste streams are predetermined based on the material of the component or the use of the component. For example, the waste streams may include recycling, disposal, or refurbishing. Components placed in the disposal waste stream would be disposed of in a land fill. Components placed in the recycling waste stream may be further separated, shredded, and melted down into a base component. Components placed in the refurbishing waste stream would be cleaned, tested, repaired, and refitted within another surgical instrument. For example, the plastic and metal components of a shroud may be separated into one waste stream for disposal, heavy metals from an integrated circuit may be separated into a second waste stream for recycling, and ultrasonic transducers may be separated into a third waste stream for refurbishing.

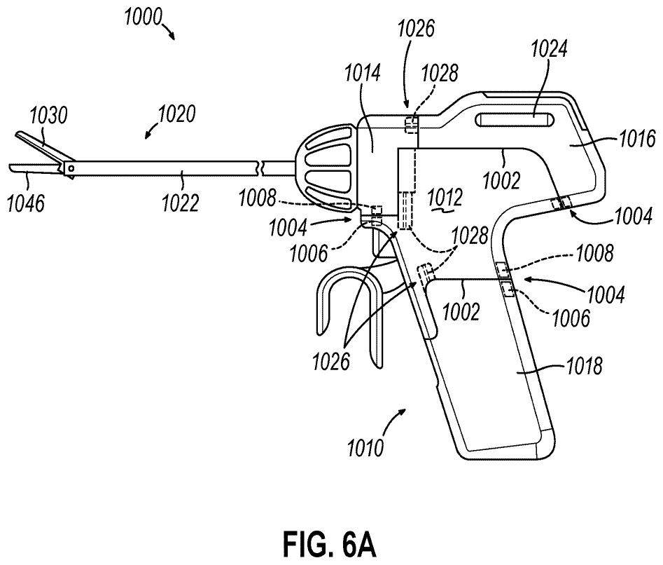

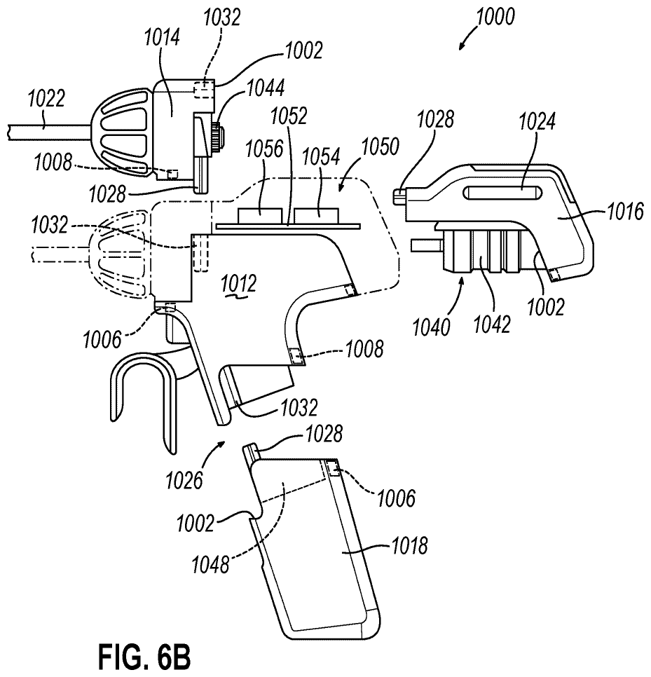

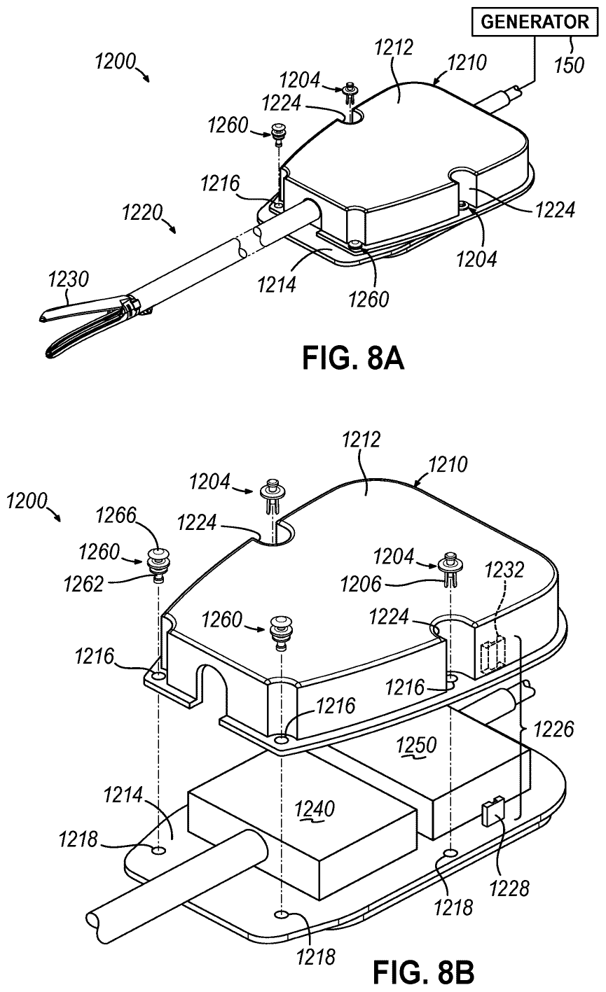

FIGS. 6A-6B illustrate one example of a surgical instrument (1000) similar to surgical instruments (112, 152, 154, 156) configured to treat tissue. Surgical instrument (1000) may be configured to deliver ultrasonic energy, Radio Frequency (“RF”) energy, or both. Additionally, surgical instrument may be configured to be hand-held or fitted with a corresponding portion of a robotic arm (see FIG. 8A). Surgical instrument (1000), like surgical instruments (112, 152, 154, 156), includes a body assembly (1010), a shaft assembly (1020) and an end effector (1030). Shaft (1022) of shaft assembly (1020) extends distally from body assembly (1010) to end effector (1030). Surgical instrument (1000) differs from surgical instruments (112, 152, 154, 156) in that surgical instrument (1000) includes body assembly (1010) configured to be easily disassembled to expose at least one of a plurality of internal components for removal and disposal into separate waste streams.

Surgical instrument (1000) of the present example is configured to deliver ultrasonic energy similar to surgical instrument (152). Body assembly (1010) surrounds a portion of an energy drive system (1040) and a portion of a circuit assembly (1050). Energy drive system (1040), in the present version, includes an ultrasonic transducer (1042), a waveguide (1044), and an ultrasonic blade (1046). Energy drive system (1040) may further include a battery (1048), or a generator (150) (see FIG. 5) configured to supply energy. Ultrasonic transducer (1042) is proximally positioned within body assembly (1010) and extends distally to waveguide (1044). Waveguide (1044) extends distally through shaft assembly (1020) to ultrasonic blade (1046). Circuit assembly (1050) includes a main circuit board (1052), a memory member (1054), and a controller (1056). Circuit assembly (1050) may be in electrical communication with a power supply such as battery (1048) or generator (150) (see FIG. 5) and is operatively connected to energy drive system (1040).

Body assembly (1010) includes a plurality of selectively removeable shroud portions (1012, 1014, 1016, 1018). Shroud portions (1012, 1014, 1016, 1018) are configured to provide support for energy drive system (1040), shaft assembly (1020), and circuit assembly (1050). Shroud portions (1012, 1014, 1016, 1018) additionally inhibit access to a portion of energy drive system (1040) and a portion of circuit assembly (1050). As illustrated, shroud portions (1012, 1014, 1016, 1018) include a first shroud portion (1012), a second shroud portion (1014), a third shroud portion (1016), and a fourth shroud portion (1018), but may include any number of shroud portions (1012, 1014, 1016, 1018) that inhibit access to circuit assembly (1050) and energy drive system (1040). This configuration of shroud portions (1012, 1014, 1016, 1018) is merely one example and not intended to unnecessarily limit the invention. Each shroud portion (1012, 1014, 1016, 1018) is removably affixed to another shroud portion (1012, 1014, 1016, 1018) at a shroud edge (1002). Shroud portions (1012, 1014, 1016, 1018) are mated together at shroud edges (1002) with shroud couplings (1004). Shroud couplings (1004) connect two adjacent shroud edges (1002) during normal operation of surgical instrument (1000) in a connected state. Magnetic fastener in the form of a shroud coupling (1004) includes a first magnetic member (1006) and a second magnetic member (1008). One of first magnetic member (1006) or second magnetic member (1008) includes a rare earth magnet or an electromagnet. Other of first or second magnetic member (1006, 1008) includes rare earth magnet, electromagnet, or a ferromagnetic metal. Ferromagnetic metals include but are not limited to iron, cobalt, or nickel. First magnetic member (1006) is attracted to second magnetic member (1008) by a magnetic field (MF). Magnetic field (MF) includes sufficient force to retain adjacent shroud edges (1002) of shroud portions (1012, 1014, 1016, 1018) in a connected state during operation, but allows for a user to transition shroud portions (1012, 1014, 1016, 1018) to a disconnected state (see FIG. 6B) after operation. Users may remove a shroud portion (1012, 1014, 1016, 1018) to provide access to portions of energy drive system (1040) and portions of circuit assembly (1050) in a disconnected state (see FIG. 6B). Once accessed, portions of energy drive system (1040) and portions of circuit assembly (1050) may be disposed of in separate waste streams. It should be noted that shroud portions (1012, 1014, 1016, 1018) may include gripping features (1024) positioned on exterior of shroud portions (1012, 1014, 1016, 1018) to facilitate opening of shroud portions (1012, 1014, 1016, 1018).

Shroud portions (1012, 1014, 1016, 1018) further include a plurality of alignment features (1026) configured to align each shroud portion (1012, 1014, 1016, 1018) with an adjacent shroud portion (1012, 1014, 1016, 1018). Alignment features (1026) facilitate translating shroud portions (1012, 1014, 1016, 1018) from connected state to the disconnected state (see FIG. 6B) and prevent binding of shroud portions (1012, 1014, 1016, 1018) when removed from one another. Additionally, alignment features (1026) facilitate alignment of first magnetic member (1006) and second magnetic member (1008) when assembling surgical instrument (1000). Alignment features (1026) in one example include a key (1028) positioned on one of first shroud portion (1012) or second shroud portion (1014) and a keyway (1032) positioned on other of first shroud portion (1012) or second shroud portion (1014). Key (1028) is sized to slide within keyway (1032). Key (1028) and keyway (1032) include complementary shapes such as circular, rectangular, square, or triangular. Shroud edges (1002) may overlap to mate key (1028) with keyway (1032) or at least one of key (1028) or keyway (1032) may extend past one of shroud edges (1002) to mate with other of key (1028) or keyway (1032).

FIG. 6B shows surgical instrument (1000) after being transitioned to the disconnected state. In the disconnected state, shrouds (1012, 1014, 1016, 1018) are separated from one another. For example, third shroud (1016) is manually moved horizontally away from first shroud (1012). Second shroud (1014) is manually moved vertically away from first shroud (1012). Fourth shroud (1018) is manually moved slantwise away from first shroud (1012) separating shroud coupling (1004). Alignment features (1026) facilitate the movement of transducer shroud in a horizontal path, second shroud (1014) in a vertical path, and fourth shroud (1018) in a slantwise path. Removal of shrouds (1012, 1014, 1016, 1018) facilitates access to at least a portion of energy drive system (1040) and at least a portion of circuit assembly (1050).

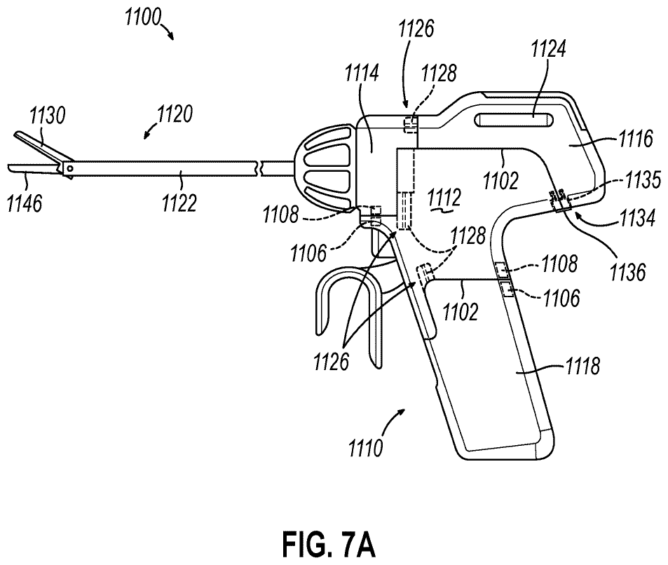

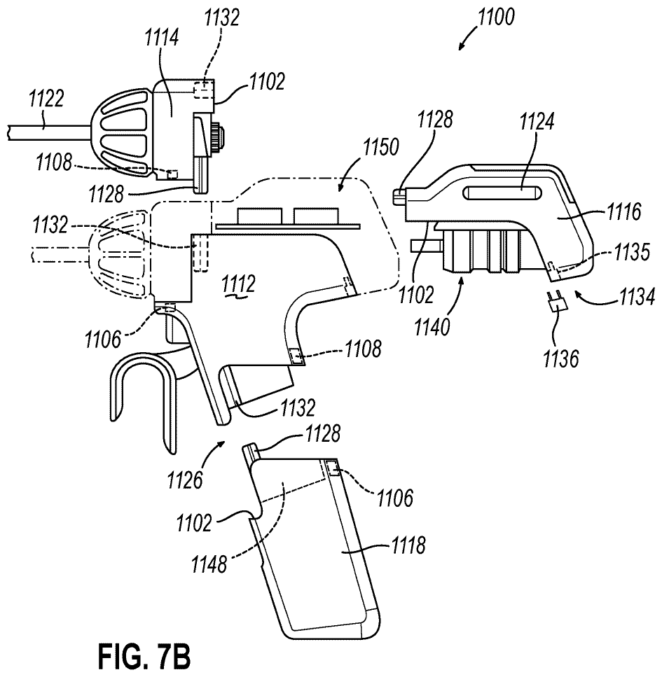

FIG. 7A illustrates one form of a surgical instrument (1100) that is similar to surgical instrument (1000) except as otherwise described herein. Surgical instrument (1100) is shown in a connected state. Surgical instrument (1100), like surgical instrument (1000), includes a body assembly (1110), a shaft assembly (1120) and an end effector (1130). Body assembly (1110) is proximally located relative to shaft assembly (1120). Shaft assembly (1120) includes a shaft (1122) distally extending from body assembly (1110) to an end effector (1130). End effector (1130) includes an ultrasonic blade (1146). Body assembly (1110) includes a plurality of shroud portions (1112, 1114, 1116, 1118) configured to inhibit access to a portion of an energy drive system (1140) and a portion of a circuit assembly (1150). Shroud portions (1112, 1114, 1116, 1118) are connected by a magnetic fastener in the form of a magnetic lock assembly (1134) at complementary shroud edges (1102) of shroud portions (1112, 1114, 1116, 1118). Magnetic lock assembly (1134) includes a first magnetic member (1106), a second magnetic member (1108), a magnetic lock (1135), and a lock key (1136). Magnetic lock assembly (1134) differs from shroud coupling (1004) of surgical instrument (1000) in that magnetic lock assembly (1134) is configured to prevent inadvertently moving shroud portion (1112, 1114, 1116, 1118) away from another shroud portion (1112, 1114, 1116, 1118) without further action by the user. First and second magnetic members (1106, 1108) may include a rare earth magnet, an electromagnet, or a ferromagnetic metal. Magnetic lock (1135) may also be configured as a solenoid (not shown). Solenoid may include an electromagnet and a ferromagnetic metal rod. Solenoid may be positioned on shroud portion (1112, 1114, 1116, 1118) with a strike plate (not shown) having a bore or an aperture on another shroud portion (1112, 1114, 1116, 1118). Ferromagnetic rod is positioned within the strike plate and maintains shroud portions (1112, 1114, 1116, 1118) in a connected state. Solenoid may be configured to move ferromagnetic rod transversely, longitudinally, or slantwise relative to shroud edge (1102) and into strike plate. In some versions, circuit assembly (1150) may be configured to be in electrical communication with magnetic lock (1135). Each shroud edge (1102) of shroud portions (1112, 1114, 1116, 1118) may be fitted with magnetic lock (1135) controlled by circuit assembly (1150). Circuit assembly (1150) in such versions, is able to transition all magnetic locks (1135) from a locked state to an unlocked state and vise-versa.

Magnetic lock assembly (1134) further includes lock key (1136) that in the present example is separable from magnetic lock (1134) and the magnetic members (1106, 1108) are separate and apart from the magnetic lock (1134). In the present versions, lock key (1136) removes electricity from an electromagnet thereby removing magnetic field, which stops the magnetic attraction between first and second magnetic members (1106, 1108) when lock key (1136) is removed from the magnetic lock assembly (1134). In the alternative, lock key (1136) may be inserted into the magnetic lock assembly (1134) to transition magnetic locks (1135) from a locked state to an unlocked state. Lock key (1136) may remove the electricity by shorting an electrical circuit (not shown), breaking an electrical circuit (not shown), or by energizing a switch (not shown).

In some versions, lock key (1136) transitions magnetic lock (1135) from the locked state to the unlocked stated by physically moving one of first or second magnetic members (1106, 1108) away from other of first or second magnetic members (1106, 1108), thereby reducing the magnetic attraction between first and second magnetic members (1106, 1108). In such versions, magnetic lock (1135), first magnetic member (1106), and second magnetic member (1108) are proximate to one another. This reduced magnetic attraction or lack of magnetic attraction allows a user to manually separate shroud portions (1112, 1114, 1116, 1118).

FIG. 7B shows surgical instrument (1100) in the disconnected state after magnetic lock assembly (1134) has been transitioned from the locked state to the unlocked state. Body assembly further includes a gripping feature (1124) configured to facilitate grabbing shroud portions (1112, 1114, 1116, 1118) to separate shroud portions (1112, 1114, 1116, 1118) from one another and an alignment feature (1126) such as a key (1128) and keyway (1132) configured to aid in aligning shroud portions (1112, 1114, 1116, 1118) when removed from one another. In the present version, alignment feature is separate and apart from magnetic lock assembly (1134) and/or magnetic members (1108, 1106). In some versions, alignment feature (1126) is incorporated into magnetic lock assembly (1134) and/or magnetic members (1108, 1106).

FIGS. 8A-10 illustrate one form of a surgical instrument (1200) similar to surgical instrument (1000) configured to deliver electrical energy to treat tissue. Surgical instrument (1200) as illustrated is configured to be fitted to a corresponding portion of a robotic arm but may also be configured as a hand-held surgical instrument as illustrated in FIGS. 6A-7B. Surgical instrument (1200) is shown in electrical communication with a generator (150) (see FIG. 5). Generator (150) is configured to supply energy to surgical instrument (1200). Surgical instrument (1200), like surgical instrument (1000), includes a body assembly (1210), a shaft assembly (1220), and an end effector (1230). Shaft assembly (1220) extends distally from body assembly (1210) to end effector (1230).

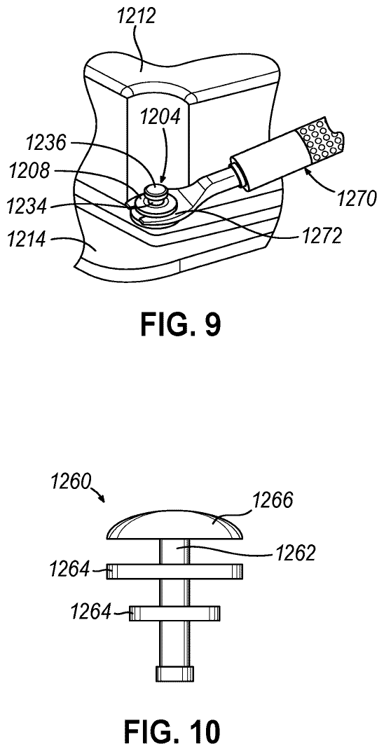

Body assembly (1210) is configured to be disassembled to expose a portion of an energy drive system (1240) and a portion of a circuit assembly (1250). Body assembly (1210) includes a plurality of shroud portions (1212, 1214) having an upper shroud portion (1212) and a lower shroud portion (1214) configured to be coupled together with a shroud coupling in the form of push-pins (1204, 1260). Push-pins (1204, 1260) may be constructed of nylon or some other material known in the art to have resilient properties. More particularly, push-pin (1204) is a two-piece push-pin (1204), whereas push-pin (1260) is a single piece push-pin (1260). Two-piece push-pin (1204) of the present example includes a shank (1206), a shank head (1208) (see FIG. 9), a pin (1234) (see FIG. 9), and a pin head (1236) (see FIG. 9). One-piece push-pin (1260) of the present example includes a shank (1262) and a plurality of resilient ribs (1264) (see FIG. 10) positioned along shank (1262) and a shank head (1266) positioned at a proximal end of shank (1262). Upper shroud portion (1212) defines an upper bore (1216), and lower shroud portion (1214) defines a lower bore (1218). FIG. 8A shows upper bore (1216) aligned with lower bore (1218) and fitted with a push-pins (1204, 1260) in the connected state. Push-pin (1204) forces upper shroud portion (1212) against lower shroud portion (1214) to retain upper shroud portion (1212) against lower shroud portion (1214) in the connected state. It should be noted that surgical instrument (1200) may include all of one type of push-pin (1204, 1206) or include more than one type of push-pin (1204, 1206). In the present version, surgical instrument (1200) includes both two-piece push-pins (1204) and one-piece push-pins (1206).

Two-piece push-pin (1204) is installed by first inserting shank (1206) through upper and lower bores (1216, 1218) so that shank head (1208) rests on a surface adjacent to upper bore (1216). Before inserting, shank (1206) remains in an unexpanded state sized to be fitted within upper and lower bore (1216, 1218). Pin (1234) is pressed within a bore of shank (1206) until pin head (1236) is seated upon a top of shank head (1208). In the installed position, a distal portion of shank (1206) is expanded by pin (1234) to an expanded state and has a diameter that is larger than upper and lower bores (1216, 1218). The expanded shank (1206) axially pulls upper shroud portion (1212) towards lower shroud portion (12140). Installing pin (1234) distally into shank (1206) results in distal portion of shank (1206) having a larger outer diameter than distal portion of shank (1206) before pin (1234) was installed into shank (1206).

One-piece push-pin (1260) is installed by pressing on top of shank head (1266) while directing a distal end of shank (1262) into upper and lower bores (1216, 1218). One-piece push-pin (1260) is installed with shank head (1266) being positioned on a first side of upper bore (1216) and a proximal-most rib (1264) being positioned on a distal side of lower bore (1218) such that push-pin (1260) resists removal of upper shroud portion (1212) from lower shroud portion (1214).

Other versions of push-pins (not shown) may be configured for manual operation without additional tools. These other versions operate similar to a blind rivet but having an actuator (not shown), a spring (not shown), a pin (not shown) and a shank (not shown). Actuator is moved by a user translating pin within shank (not shown). An outside diameter of shank is reduced so that push-pin may be retracted through upper and lower bores (1216, 1218) and upper and lower shroud portions (1216, 1218) are transitioned from a connected state to a disconnected state.

FIG. 8B shows surgical instrument (1200) in the disconnected state with push-pins (1204, 1260) removed from upper and lower bores (1216, 1218). In the disconnected state, body assembly (1210) provides access to a portion of energy drive system (1240) and a portion of circuit assembly (1250) for removal and disposal in separate waste streams. Upper and lower shroud portions (1212, 1214) may include gripping features (1224) that aid in removal of upper shroud portion (1212) from lower shroud portion (1214) and alignment features (1226) that provide alignment of upper and lower bores (1216, 1218). Alignment features (1226) such as a key (1228) positioned on one of the upper or lower shroud portions (1212, 1214) and a keyway (1232) positioned on the other of the upper or lower shroud portions (1212, 1214).

FIG. 9 shows push-pin (1204) being removed from upper and lower shroud portions (1212, 1214) by a torque wrench (1270) including a forked member (1272). Push-pin (1204) may be constructed of nylon or some other material known in the art to have resilient properties.

FIG. 10 shows one-piece push-pin (1260) including shank (1262) and the plurality of resilient ribs (1264) positioned along shank (1262) and shank head (1266) positioned at a proximal end of shank (1262). Push-pin (1260) is also constructed of nylon or some other material known in the art to have resilient properties. Push-pin (1260) may also be removed with torque applied to push-pin (1260) by prying upon shank head (1266) with forked member (1272).

### III. EXEMPLARY SURGICAL INSTRUMENT INCORPORATING SELECTIVELY SEPARABLE CIRCUIT ASSEMBLIES

In some instances, it may be desirable to provide a surgical instrument that includes components capable of delivering ultrasonic energy, RF energy, or both ultrasonic and RF energy that is easily opened so that the internal components may be separated into separate waste streams with minimal tools, such as no tools, within the sterile field. These surgical instruments are configured to stay intact during normal use but facilitate disassembly of internal components and/or selectively break the internal components. One such internal component is a circuit assembly that is configured to be separated by a hand or hands of the user into separate portions having different properties. The separate portions of the circuit assembly are placed in predetermined separate waste streams. These waste streams include but are not limited to recycling, disposal, or refurbishing.

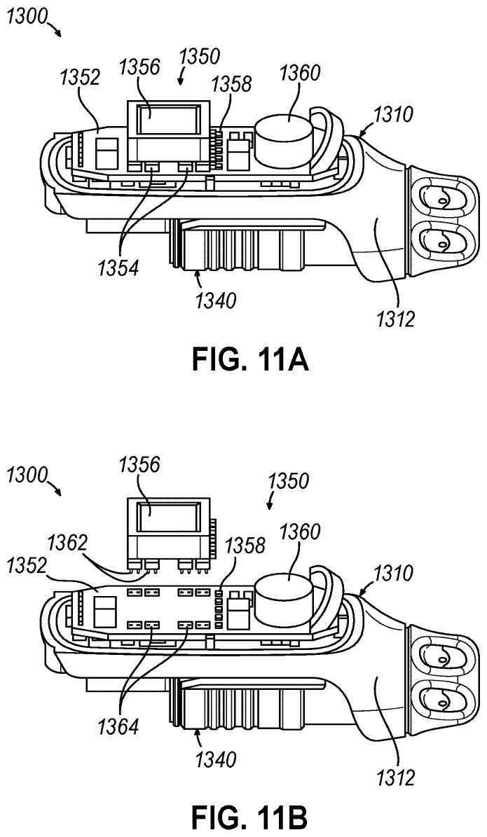

FIGS. 11A-11B show a portion of surgical instrument (1300) similar to surgical instrument (1000) except as otherwise described herein. Surgical instrument (1300) includes a shaft assembly (not shown) that extends distally from a body assembly (1310) to an end effector (not shown). Body assembly (1310) includes a plurality of shroud portions (1312) similar to surgical instrument (1000). Top shroud portion (not shown) has been removed from lower shroud portion (1312) to expose a portion of circuit assembly (1350). Circuit assembly (1350) includes a main circuit board (1352) and a plurality of sub-boards (1354). Lower shroud portion (1312) provides support for circuit assembly (1350) and energy drive assembly (1340). Main circuit board (1352) includes an integrated circuit (1358) configured to provide electrical communication between a memory member (1356), a controller (1360), inputs (not shown), and outputs (not shown). In some versions, sub-boards (1354) include memory member (1356) and controller (1360). In the present version, main circuit board (1352) includes an integrated controller (1360) and sub-boards (1354) include memory member (1356). FIG. 11A shows sub-boards (1354) plugged into main circuit board (1352) in an installed position in communication with controller (1360) via integrated circuit (1358).

FIG. 11B shows sub-boards (1354) having memory member (1356) shown in an uninstalled position. Memory member (1356) in the uninstalled position is unplugged from main circuit board (1352). Memory member (1356) may include electrically erasable programmable read-only memory (“EEPROM”), erasable programable read-only memory (“EPROM”), programable read-only memory (“PROM”), read only memory (“ROM”), random access memory (“RAM”), or other suitable forms of memory known in the art for use with circuit assemblies. Sub-boards (1354) include a plurality of prongs (1362) configured to removably couple with respective receptacles (1364) defined by main circuit board (1352). Placement of prongs (1362) and receptacles (1364) may be reversed with prongs (1362) being located on main circuit board (1352) and receptacle (1364) being located on sub-board (1354). Prongs (1362) provide electrical communication between sub-boards (1354) and main circuit board (1352). Sub-boards (1354) may be removed from main circuit board (1352) for disposal in a separate waste stream than main circuit board (1352). For example, sub-boards (1354) may be refurbished and reused, and main circuit board (1352) may be recycled, although such distribution is merely one example and not intended to unnecessarily limit the invention.

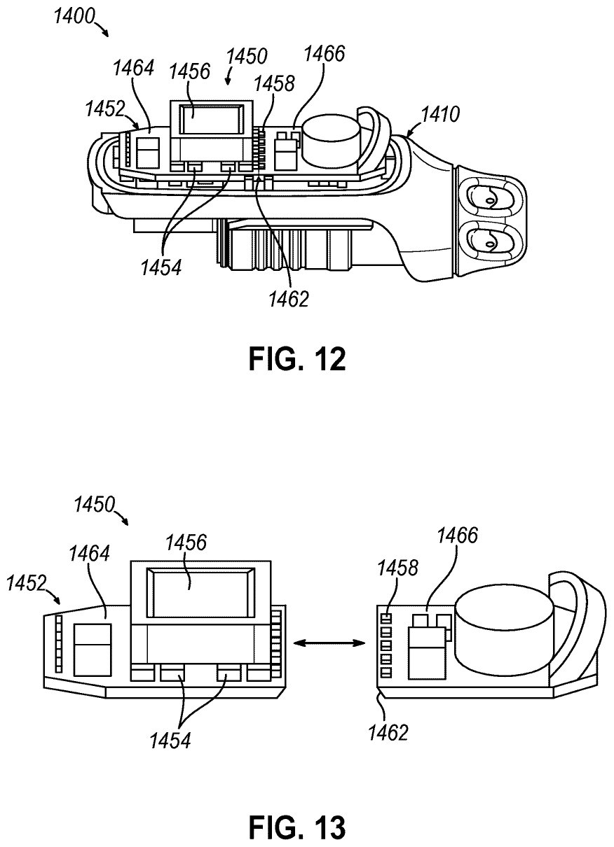

FIGS. 12-13 show a portion of surgical instrument (1400) similar to surgical instrument (1300) except as otherwise described herein. Surgical instrument (1400) like surgical instrument (1300) includes a circuit assembly (1450) configured to be broken into separate portions for disposal in separate waste streams. FIG. 12 shows a portion of body assembly (1410) with a top shroud portion (not shown) removed to expose circuit assembly (1450). Circuit assembly (1450) includes a main circuit board (1452) and sub-boards (1454). Main circuit board (1452) and sub-boards (1454) are shown in an intact state. Surgical instrument (1400) differs from surgical instrument (1300) in that sub-boards (1454) are fixedly coupled to main circuit board (1452) and main circuit board (1452) includes frangible separators (1462) configured to connect a first circuit portion (1464) to a second circuit portion (1466) in an operable state. Frangible separators (1462) may include perforations, consecutive holes, weakened portions, or any other separation feature know in the art that would facilitate breaking a circuit board along a predetermined path. In the operable state, frangible separators (1462) allow for electrical communication along circuit assembly (1450), such as electrical communication across frangible separators (1462).

FIG. 13 shows circuit assembly (1450) in a separated state with first circuit portion (1464) separated from second circuit portion (1466). First circuit portion (1464) may be separated from second circuit portion (1466) by breaking frangible separator (1462), such as with a user's hand or hands. First circuit portion (1464) may include components that require first circuit portion (1664) to be placed in a separate waste stream than second circuit portion (1466). For example, sub-board (1454) may have memory member (1456) unsuitable for reconditioning or recycling that is placed in the disposal waste stream and second circuit portion (1466) may include integrated circuit (1458) including heavy metals that require reconditioning or recycling, although such arrangement is merely one example and not intended to unnecessarily limit the invention.

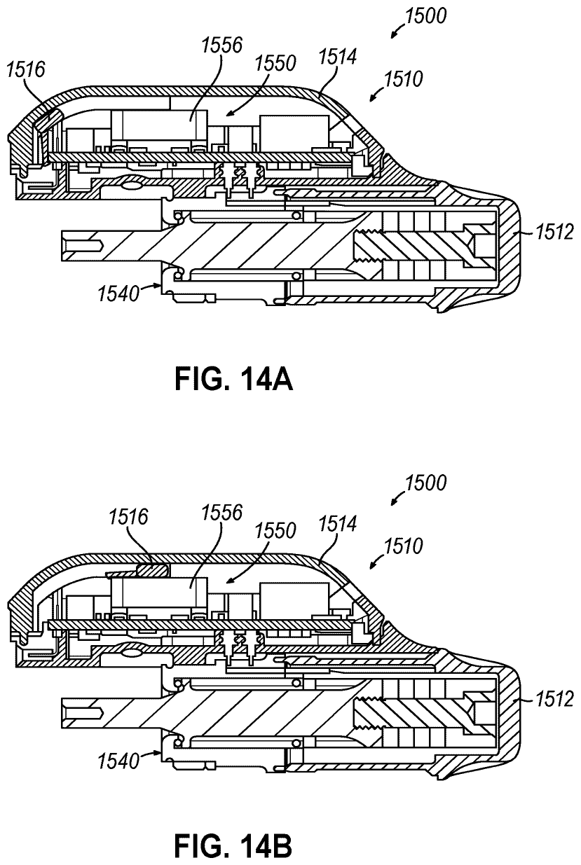

FIGS. 14A-14B show a portion of surgical instrument (1500) similar to surgical instrument (1400) except as otherwise described herein. A body assembly (1510) of the present example includes a first shroud portion (1512), a second shroud portion (1514), and a latch (1516). First shroud portion (1512) provides support for a portion of energy drive system (1540) and a portion of a circuit assembly (1550). Second shroud portion (1514) inhibits access to circuit assembly (1550). Second shroud portion (1514) is removably affixed to first shroud portion (1512) with latch (1516) in a secured position.

FIG. 14A shows circuit assembly (1550) having a memory member (1556) in an operable state with latch (1516) in a closed position with second shroud portion (1514) connected to first shroud portion (1512). Memory member (1556) as mentioned above may include RAM, ROM, PROM, EPROM, EEPROM, or any other suitable forms of memory known in the art.

FIG. 14B shows the circuit assembly (1550) in an inoperable state after latch (1516) was transitioned to an open position. Latch (1516) includes a rare earth magnet that uses a magnetic field to causes a disruption of electrical signals or magnetic pulse that damages or scrambles memory within memory member (1556). Latch (1516) transitions from a secured position to an unsecured position. Before fully opening second shroud portion (1514), latch (1516) passes in close proximity to memory member (1556) thereby rendering data contained in memory member (1556) unreadable. By way of example, latch (1516) may have a hall effect sensor with an integrated magnet that allows circuit assembly (1550) initiate a sequence rendering the data unreadable, such as a reset, rewrite, or scramble, which erases or destroys memory. In other versions, the latch (1516) includes a magnet that passed a magnetic field close to the memory member (1556) that erases or destroys the memory.

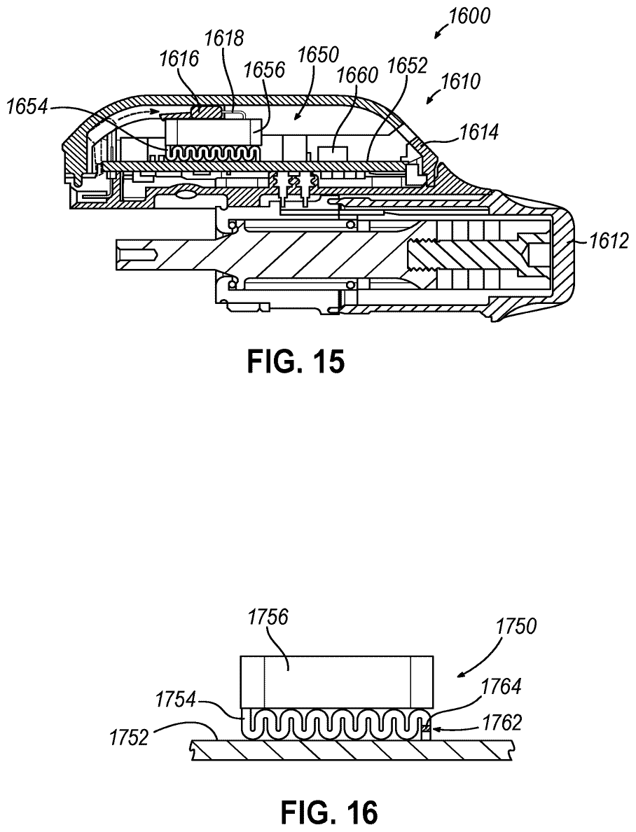

FIG. 15 shows a portion of surgical instrument (1600) similar to surgical instrument (1500) except as otherwise described herein. Like surgical instrument (1500), surgical instrument (1600) includes a body assembly (1610) having a first shroud portion (1612) connected to a second shroud portion (1614) by a latch (1616). Body assembly (1610) houses, supports, and inhibits access to circuit assembly (1650). Circuit assembly (1650) includes a memory member (1656), a main circuit board (1652), and a flexible circuit (1654). Memory member (1656) as mentioned above may include a memory such as RAM, ROM, PROM, EPROM, EEPROM, or any other suitable forms of memory known in the art. Memory member (1656) and/or controller (1660) is in electrical communication with main circuit board (1652) via flexible circuit (1654). Flexible circuit (1654) allows memory member (1656) to be placed separate and apart from main circuit board (1652). In some versions, memory member (1656) may be connected directly to main circuit board (1652) by soldering or with a plug and receptacle. Surgical instrument (1600) includes latch (1616) that is in electrical communication with memory member (1656) via a cable (1618). Latch (1616) is capable of transitioning from a closed position to an open position thereby transitioning first and second shroud portions (1612, 1614) from a connected state to a disconnected state. Additionally, latch (1616) in the open position engages a set of contacts that renders data stored on memory member (1656) unreadable, such as by electrically resetting, rewriting, or scrambling memory, by providing electrical communication with one of the follow reset elements: an integrated capacitor to supply voltage or current into memory member (1656), a current reverser to apply reverse current (i.e., reverse polarity), a power source capable of producing an electrical pulse that damages data stored on memory member (1656), and/or another memory (not shown) that releases unstable data (i.e., noise) to render memory member (1656) inoperable. When reverse polarity is used to destroy memory member (1656), a greater voltage than the voltage used in normal operation may be applied with low amperage to negative terminal or ground rather than positive terminal. For example, 10 volts supplied with low amperage is supplied to ground connections of a memory member (1656) that during normal operation uses 6 volts, although such configuration is merely one example and not intended to unnecessarily limit the invention.

FIG. 16 shows a circuit assembly (1750) for incorporation into any of surgical instrument (112, 152, 154, 156, 1000, 1100, 1200, 1400, 1500, 1600). Circuit assembly (1750) includes a main circuit board (1752), a memory member (1756), a controller (not shown), and a flexible circuit (1754). Memory member (1756), as mentioned above, may include a memory such as RAM, ROM, PROM, EPROM, EEPROM, or any other suitable forms of memory known in the art. Memory member (1756) is positioned within a body assembly (not shown) that houses, supports, and inhibits access to circuit assembly (1750). Flexible circuit (1754) includes a failure region (1762) that allows for removal of memory member (1756) and/or destruction of flexible circuit (1754) during the sterilization process. In the present version, failure region (1762) includes a conductive epoxy (1764) that bridges failure region (1762) to provide electrical communication between memory member (1556) and main circuit board (1752). When circuit assembly (1750) is heated to a temperature suitable to sterilize surgical instrument (1700), conductive epoxy (1764) destructs, such as by melting, in a manner that separates flexible circuit (1754) at failure region (1762) into one or more portions. Flexible circuit (1754) in a separated state disallows electrical communication between memory member (1756) and main circuit board (1752). In other versions, failure region (1762) is perforated or otherwise weakened so that a user may manually remove memory member (1756) from main circuit board (1752) with a hand or hands of the user. Additionally, controller may be attached to main circuit board (1752) with flexible circuit (1754) having failure region (1762) for ease of removal to be placed in a separate waste stream than main circuit board (1752). In other versions, controller may be similarly attached with a flexible circuit (1754) including a failure region (1762). Once removed, memory member (1756) and/or controller is inhibited from being used again in another circuit assembly.

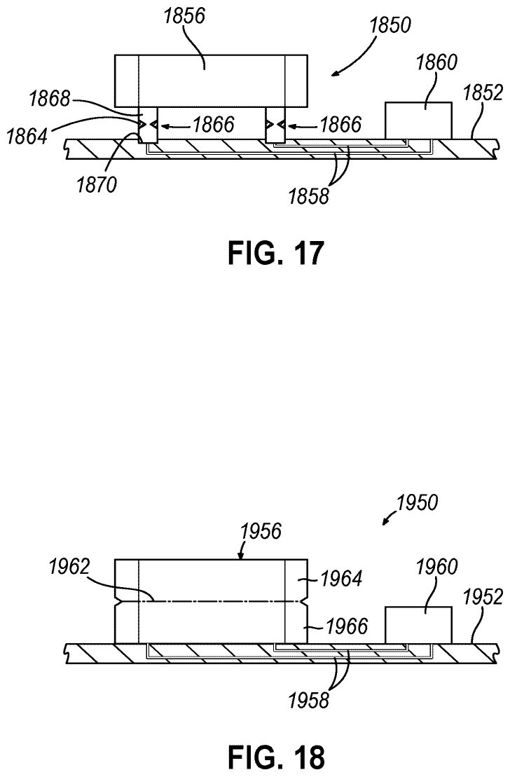

FIG. 17 shows a circuit assembly (1850) for incorporation into any of surgical instruments (112, 152, 154, 156, 1000, 1100, 1200, 1400, 1500, 1600). Circuit assembly (1850) is similar to circuit assembly (1750) except as otherwise described herein. Circuit assembly (1850) includes a main circuit board (1852), an integrated circuit (1858), a memory member (1856), and a controller (1860). Circuit assembly (1850) differs from circuit assembly (1750) in that memory member (1856) is attached to main circuit board (1852) by a pin connector (1868). Pin connector (1868) is a rigid connector constructed of metal capable of being soldered. Pin connector (1868) is directly soldered into a pin bore (1870). Pin bore (1870) is defined by main circuit board (1852). Pin connector (1868) includes a failure region (1866). Failure region (1866) allows for the removal of memory member (1856) manually prior to or during the sterilization process. As shown, failure region (1866) includes a conductive epoxy (1864) that bridges a gap in pin connector (1868). During the sterilization process, conductive epoxy (1864) reaches its predetermined melting temperature and derogates, such as by melting, when heated to a temperature suitable to sterilize a surgical instrument, such as any of surgical instruments (112, 152, 154, 156, 1000, 1100, 1200, 1400, 1500, 1600). Once conductive epoxy (1864) melts, two portions of pin connectors (1868) separate such that memory member (1856) from main circuit board (1852) disallows communication by integrated circuit (1858) to controller (1860).

In other versions, failure region (1866) includes a portion of pin connector (1868) that has a reduced diameter relative to a remaining portion of pin connector (1868). Failure region (1866) provides a location from which memory member (1856) may be broken away from main circuit board (1852) by a user's hand or hands with a reduced force (relative to the force to remove a memory member (1856) without a failure region (1866)) rendering the instrument inoperable and/or to provide for separate disposal in a separate waste stream than the other components of main circuit board (1852).

FIG. 18 shows a circuit assembly (1950) for incorporation into any of surgical instruments (112, 152, 54, 156, 1000, 1100, 1200, 1400, 1500, 1600). Circuit assembly (1950) is similar to circuit assembly (1850) except as otherwise described herein. Circuit assembly (1950) includes a main circuit board (1952), a memory member (1956), an integrated circuit (1958), and a controller (1960). Circuit assembly (1950) differs from circuit assembly (1850) in that main circuit board (1952), controller (1960) and/or memory member (1956) includes a frangible notch (1962) defining a weak section on main circuit board (1952), controller (1960), and/or memory member (1956). In the present version, memory member (1956) includes a first memory portion (1964), a second memory portion (1966), and frangible notch (1962). First memory portion (1964) is separated from second memory portion (1966) by frangible notch (1962). Second memory portion (1966) is rigidly attached to main circuit board (1952). First memory portion (1964) is removably coupled to second memory portion (1966) so that if main circuit board (1952) is removed from a body assembly (not shown), first memory portion (1964) is configured to engage a portion of body assembly and break memory member (1956) to thereby render memory member (1956) inoperable. In this respect, frangible notch (1962) is configured to inhibit memory member (1956) from being removed from main circuit board (1952) intact for reuse. Additionally, if a user attempts to install circuit assembly (1950) or inadvertently loaded circuit assembly (1950) into a sterilized or improper instrument, a portion of body assembly engages a first memory portion (1964) and damages memory member (1956) by separating first memory portion (1964) from second memory portion (1966) preventing reuse.

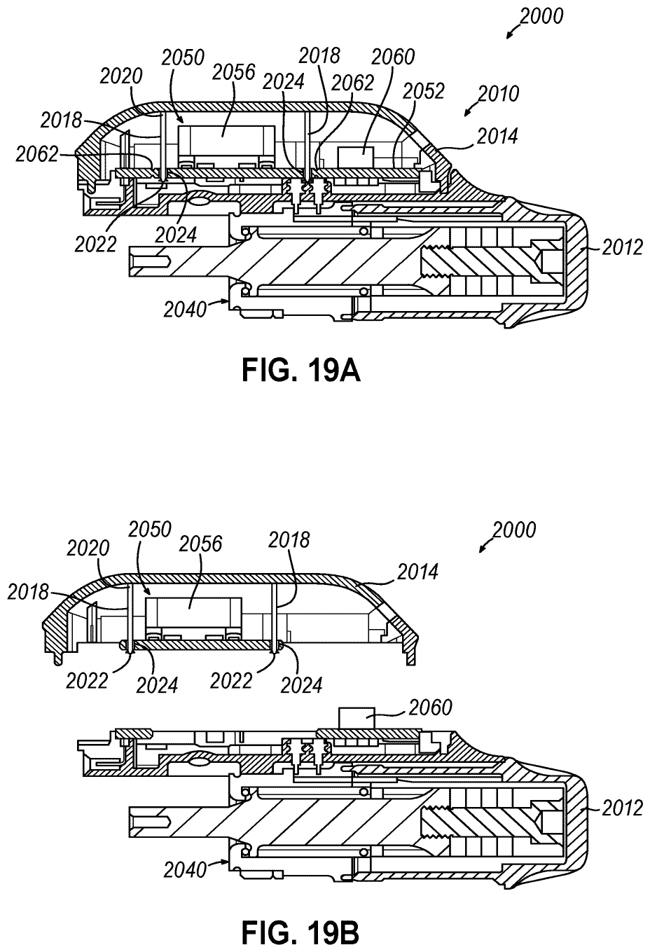

FIG. 19A shows a portion of surgical instrument (2000) similar to surgical instrument (1500) except as otherwise described herein. Body assembly (2010) includes a first shroud portion (2012), a second shroud portion (2014), and one or more support members (2018). First shroud portion (2012) provides support for a portion of energy drive system (2040) and a portion of circuit assembly (2050). Second shroud portion (2014) is affixed to first shroud portion (2012) in the connected state. Support members (2018) are rigid members that include a first support end (2020) and a second support end (2022). First support end (2020) is affixed to one of first shroud portion (2012) or second shroud portion (2014), and second support end (2022) attaches to circuit assembly (2050). Support members (2018) of the present example pass through a bore (2024) defined by circuit assembly (2050) and attach to the other of first shroud portion (2012) or second shroud portion (2014). Support members (2018) include a second support end (2022) that has a catch, such as a barb or a hook, configured for one-way installation into bore (2024). In this respect, the term “one-way installation” refers to catch being configured to install easily without damage, but to cause damage upon deinstallation. Circuit assembly (2050) includes a main circuit board (2052), a memory member (2056), and a controller (2060) and is shown in the operable state. Circuit assembly (2050) of the present example further includes a frangible separator (2062) proximate to support member (2018). Also in the present example, second shroud portion (2014) is configured to be transitioned to a disconnected state by moving second shroud portion (2014) relative to first shroud portion (2012), such as by moving second shroud portion (2014) horizontally. However, such movement is merely one exemplary direction and is not intended to unnecessarily limit the invention. By way of further example, second shroud portion (2014) may be removed vertically, slantwise, or another direction relative to first shroud portion (2012).

FIG. 19B shows portion of surgical instrument (2000) with second shroud portion (2014) removed from first shroud portion (2012). Support members (2018) move with second shroud portion (2014) away from first shroud portion (2012). Circuit assembly (2050) is held in place by first shroud portion (2012). Support members (2018) being transitioned away from first shroud portion (2012), cause damage to circuit assembly (2050) to render circuit assembly (2050) inoperable by breaking circuit assembly (2050) along frangible separators (2062), thus preventing reuse of circuit assembly (2050) and/or providing for placement in separate waste streams.

### IV. EXEMPLARY SURGICAL INSTRUMENT INCORPORATING SELECTIVELY SEPARABLE ENERGY DRIVE SYSTEM

In some instances, it may be desirable to provide a surgical instrument that includes components capable of delivering ultrasonic energy, RF energy, or both ultrasonic and RF energy. It may be desirable that these surgical instruments be easily openable so that the internal components may be separated into separate waste streams with minimal tools, such as no tools, within the sterile field. These surgical instruments are configured to stay intact during normal use but facilitate disassembly of internal components and/or selectively break the internal components. One such internal component is an energy drive system that is configured to be removed, separated by a hand or hands of the user, and placed into separate waste streams. These waste streams include but are not limited to recycling, disposal, or refurbishing.

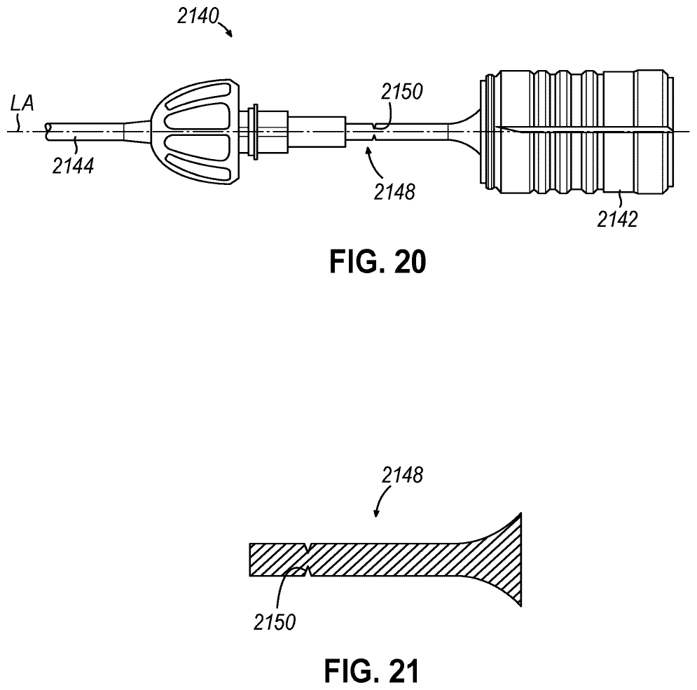

FIGS. 20-21 show a portion of an energy drive system (2140) for incorporation into any of surgical instruments (112, 152, 154, 156, 1000, 1100, 1200, 1400, 1500, 1600, 1700). Energy drive system (2140) includes an ultrasonic transducer (2142), a waveguide (2144), an energy coupling (2148), and an ultrasonic blade (not shown). Ultrasonic transducer (2142) extends along a longitudinal axis (LA), and energy coupling (2148) is configured to removably couple ultrasonic transducer (2142) to waveguide (2144). Waveguide (2144) further extends along longitudinal axis (LA) and is operatively affixed to ultrasonic blade. Energy coupling (2148) has a frangible division (2150) that is capable of being broken by a hand or hands of the user. Frangible division (2150) may include drilled holes, serrations, notches, or any other weakening for breaking in a predetermined manner. Once energy coupling (2148) is broken, ultrasonic transducer (2142) cannot be joined with waveguide (2144) without further reconditioning. By way of example, additional frangible divisions (2150) may be positioned between waveguide (2144) and ultrasonic blade. Ultrasonic transducer (2142) may thus be sanitized, refurbished, and reused in a similar surgical instrument while waveguide (2144) and ultrasonic blade are placed in another waste stream, such as disposal or recycling.

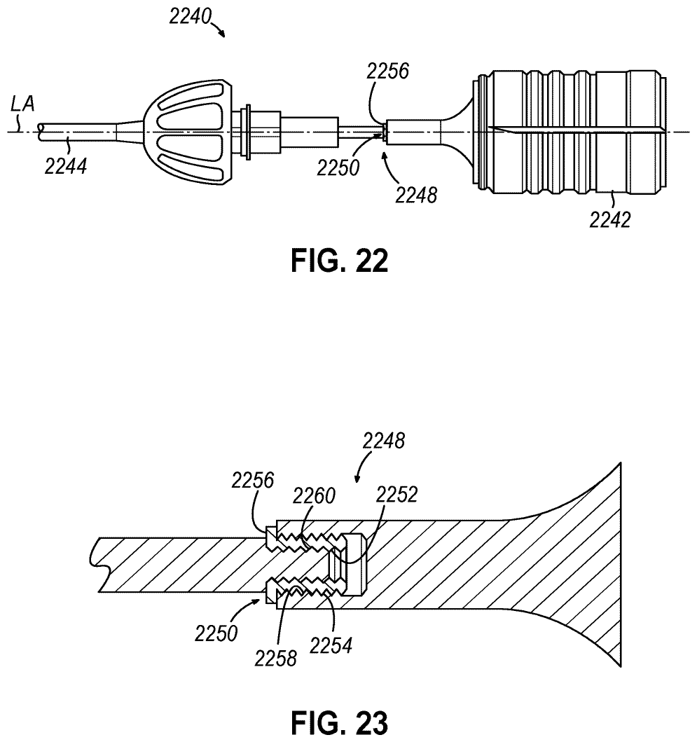

FIGS. 22-23 show another portion of an energy drive system (2240) for incorporation into any of surgical instruments (112, 152, 154, 156, 1000, 1100, 1200, 1400, 1500, 1600, 1700) similar to energy drive system (2140) except as otherwise described herein. Like energy drive system (2140), energy drive system (2240) includes an ultrasonic transducer (2242), a waveguide (2244), an energy coupling (2248), and an ultrasonic blade (not shown). Energy drive system (2240) differs from energy drive system (2140) in that energy drive system (2240) includes energy coupling (2248) configured to uncouple and recouple waveguide (2244) with ultrasonic transducer (2242). Energy coupling (2248) in the present example is in the form of a bushing (2250) configured to be threaded between ultrasonic transducer (2242) and waveguide (2244). Bushing (2250) includes an internal bushing thread (2252) and an external bushing thread (2254). Bushing (2250) also includes a torque feature (2256) in the form of a hex head configured to allow a user to tighten bushing (2250) within ultrasonic transducer (2242) to a specified torque, such as with a torque wrench (not shown) provided within a sterile packaging (not shown). As shown, external bushing thread (2254) is threaded into an internal transducer thread (2258), and external waveguide thread (2260) is threaded into internal bushing thread (2252), although this configuration may be reversed in other examples. Additionally, bushing (2250) may include two sets of internal threads, or two sets of external threads to be mated with complementary threads positioned on waveguide (2244) and ultrasonic transducer (2242). For example, external bushing threads (2254) threadedly couple with an internal waveguide thread (not shown) and an external transducer thread (not shown). Bushing (2250) is configured to transmit ultrasonic energy between ultrasonic transducer (2242) and waveguide (2244). Bushing (2250) of the present example is constructed of a less durable material than waveguide (2244) and/or ultrasonic transducer (2242) so that waveguide (2244) and/or ultrasonic transducer (2242) is preserved thereby eliminating the preference to refurbish waveguide (2244) and/or ultrasonic transducer (2242).

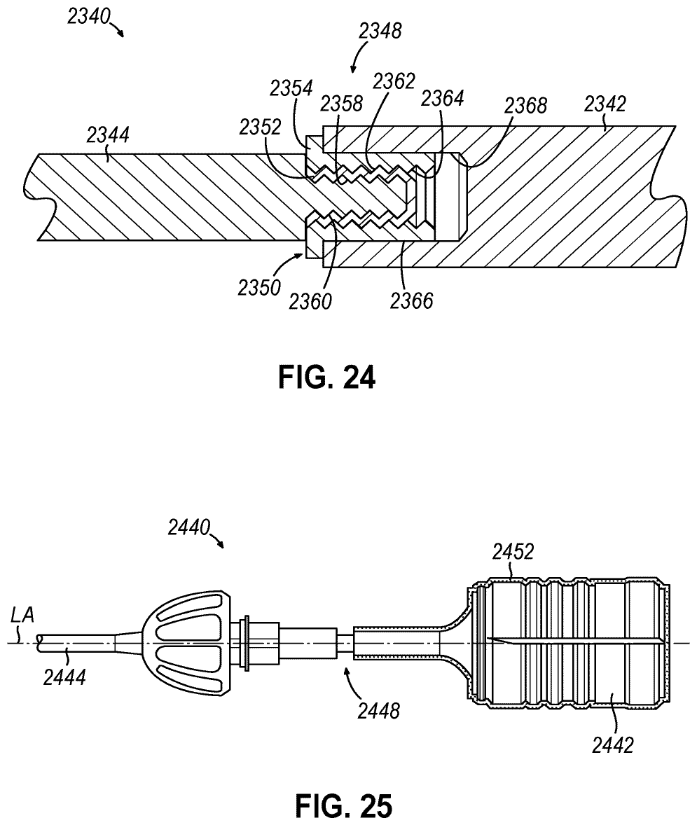

FIG. 24 shows a portion of an energy drive system (2340) including another energy coupling (2348) to be incorporated into any of surgical instruments (112, 152, 154, 156, 1000, 1100, 1200, 1400, 1500, 1600, 1700). Energy coupling (2348) is similar to energy coupling (2248) except as otherwise described herein. Energy coupling (2348), like energy coupling (2248), is capable of threadedly coupling a waveguide (2344) with an ultrasonic transducer (2342). Energy coupling (2348) more particularly includes a bushing assembly (2350) with a screw thread insert (2352) and a bushing (2354).

Waveguide (2344) includes a proximal end having an external waveguide thread (2360), and ultrasonic transducer (2343) including an internal transducer surface (2368). Screw thread insert (2352) of the present example is constructed of a helically wound wire, but may alternatively be a solid, machined piece. Screw thread insert (2352) includes an internal insert thread (2358) configured to engage an external waveguide thread (2360) and an external insert thread (2362) configured to engage internal bushing threads (2364). Bushing (2354) includes an internal bushing thread (2364) configured to be threaded around outer insert thread (2362). Bushing (2354) further includes an external bushing surface (2366) having a smooth bore sized for an interference fit relative to an internal transducer surface (2368). Bushing (2354) is press fit within ultrasonic transducer (2342). In some versions, external waveguide thread (2360) is mated with inner insert thread (2358), external insert thread (2362) is mated with internal bushing thread (2364), and external bushing surface (2366) is press fit within ultrasonic transducer (2342). Screw thread insert (2352) is separable from bushing (2354) so that, after use, ultrasonic transducer (2342) or waveguide (2344) may be removed for recycling or refurbishment and either screw thread insert (2352) and/or bushing (2350) may be disposed or recycled. Screw thread insert (2352) and bushing (2354) may be constructed of materials that conduct ultrasonic energy, but are less wear resistant than waveguide (2344) and/or ultrasonic transducer (2342) so screw thread insert (2352) and/or bushing (2354) may be replaced after a predetermined number of uses or after a predetermined amount of wear develops to prevent ultrasonic transducer (2342) and/or waveguide (2344) from developing wear.

FIG. 25 shows a portion of an energy drive system (2440) for incorporation into any of surgical instruments (112, 152, 154, 156, 1000, 1100, 1200, 1400, 1500, 1600, 1700). Energy drive system (2440) is similar to energy drive system (2240) except as otherwise described herein. Energy drive system (2440) includes an ultrasonic transducer (2442), a waveguide (2444), and an ultrasonic blade (not shown). Energy drive system (2440) may further include an energy coupling (2448) to further aid a user in disassembling energy drive system (2440) with a hand or hands of a user. Ultrasonic transducer (2442) extends along a longitudinal axis (LA) and may be removably coupled to waveguide (2444) by energy coupling (2448). Waveguide (2444) further extends along longitudinal axis (LA) and is operatively affixed to ultrasonic blade.

Ultrasonic transducer (2442) may be removed and refurbished for reuse. More particularly, ultrasonic transducer (2442) includes a protective coating, such as a cover (2452), to inhibit damage to ultrasonic transducer (2442) when removed from body assembly (2410). Cover (2452) includes a non-conductive base such as rubber, plastic, or ceramic. Cover (2452) of the present example also includes a conductive base installed by electroplating or by being topically applied to the surface of ultrasonic transducer (2442). Such protective coating may be applied as a liquid and allowed to dry. In other versions, cover (2452) is in a solid state and is fastened over ultrasonic transducer (2442).

### V. EXEMPLARY SURGICAL INSTRUMENT INCORPORATING SELECTIVELY SEPARABLE HOUSING AND STRAIN RELIEF FEATURE

In some instances, it may be desirable to provide a surgical instrument that includes components capable of delivering ultrasonic energy, RF energy, or both ultrasonic and RF energy that is easily opened so that the internal components may be separated into separate waste streams with minimal tools, such as no tools, within the sterile field. These surgical instruments are configured to stay intact during normal use but facilitate disassembly of internal components and/or selectively break the internal components. One such internal component is an electrical cable that is configured to be removed by a hand or hands of the user after separating a housing for access to the electrical cable. The electrical cable is configured to be placed into a waste stream that may include, but are not limited to, recycling, disposal, or refurbishing. Other components may be placed in waste streams other than the waste stream desired for electrical cable.

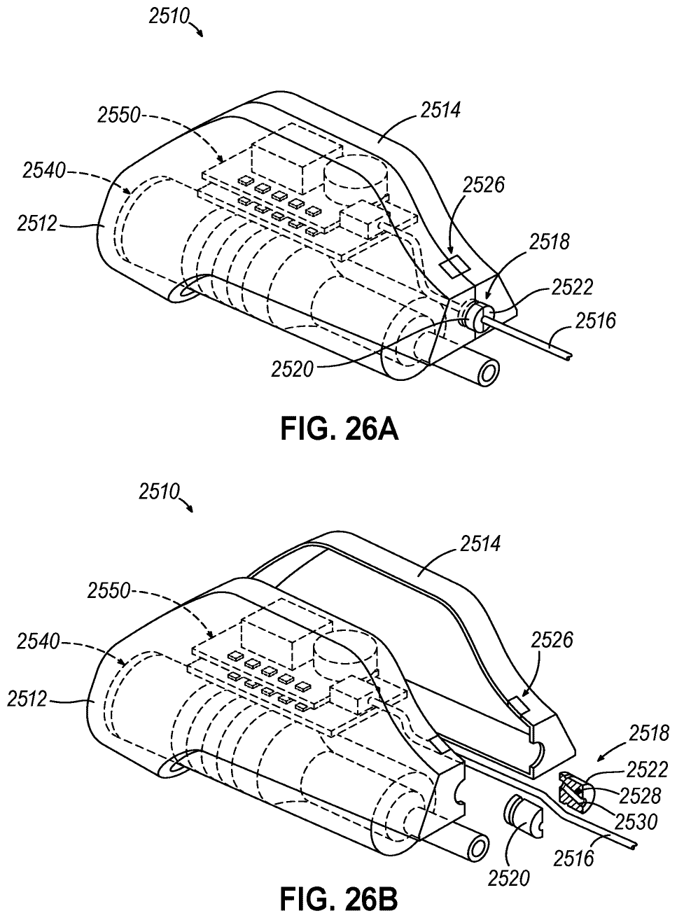

FIG. 26A shows a portion of a body assembly (2510) for incorporation into any of surgical instruments (112, 152, 154, 156, 1000, 1100, 1200, 1400, 1500, 1600, 1700). Body assembly (2510) includes a first shroud portion (2512), a second shroud portion (2514), a lateral coupling (2526), and an electrical cable (2516). First and second shroud portions (2512, 2514) more particularly are in the form of first and second cover portions. Body assembly (2510) is configured to support a circuit assembly (2550) and an energy drive system (2540) while also inhibiting access to circuit assembly (2550) and energy drive system (2540). Electrical cable (2516) provides electrical communication between a generator (150) (see FIG. 5) and energy drive system (2540) and/or circuit assembly (2550). With first shroud portion (2512) and second shroud portion (2514) retain electrical cable (2516) in a connected state. Lateral coupling (2526) is positioned between first and second shroud portions (2512, 2514) and is configured to retain first and second shroud portions (2512, 2514) in the connected state when positioned in a locked position. Lateral coupling (2526) may include any of the aforementioned shroud couplings (1004, 1204, 1206), magnetic lock assemblies (1134) and latches (1516, 1616) as previously discussed. Lateral coupling (2526) may be locked and unlocked with a key, electrically, or have magnetic members that are separated from one another. Body assembly (2510) further includes a strain relief assembly (2518) including a first relief portion (2520) and a second relief portion (2522). In the present version, strain relief assembly (2518) is proximate to lateral coupling (2526). Strain relief assembly (2518) may be operatively attached to first and second shroud portions (2512, 2514), or integrally formed with first and second shroud portions (2512, 2514), or a separate component installed within a bore (2524) positioned between first and second shroud portions (2512, 2514). As shown in the present example, strain relief assembly (2518) is a separate component. Strain relief assembly (2518) includes a recess configured to mate with bore (2524). Recess axially locates strain relief assembly (2518) within first and second shroud portions (2512, 2514). Strain relief assembly (2518) is configured to retain electrical cable (2516) when first shroud portion (2512) and second shroud portion (2514) are in the connected state. Strain relief assembly (2518) includes an angled surface (2528) positioned on the first relief portion (2520) with a complementary surface (2530) on the second relief portion (2522) configured to axially bind electrical cable (2516) therebetween.

FIG. 26B shows strain relief assembly (2518) in a disconnected state with first shroud portion (2512) laterally spaced apart from second shroud portion (2514). In order to transition body assembly from the connected state to the disconnected state, a user transitions lateral coupling (2526) from the locked state to an unlocked state. First shroud portion (2512) is laterally spaced away from second shroud portion (2514) thereby releasing electrical cable (2516) from strain relief assembly (2518). First relief portion (2520) is separated from second relief portion (2522) and fully releases electrical cable (2516) when first and second shroud portions (2512, 2514) are in the disconnected state. First shroud portion (2512) is operatively attached to first relief portion (2520) and separates from second shroud portion (2514) that is operatively attached to second relief portion (2522). Once first relief portion (2520) is spaced apart from second relief portion (2522), electrical cable (2516) is configured to be removed from body assembly (2510) with a hand or hands of the user with reduced manual force.

### VI. SURGICAL INSTRUMENT WITH REMOVABLE CABLE AND ASSOCIATED COUPLINGS

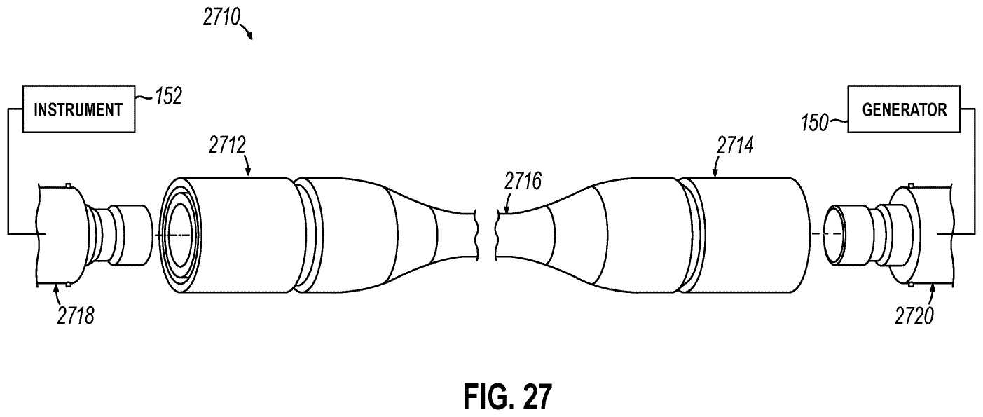

FIG. 27 illustrates an exemplary surgical device as a first example of a cable assembly (2710) used to connect to a medical device, such as surgical instrument (152), to a generator, such as generator (150) for transmitting electrical power and electrical communication signals. Cable assembly (2710) includes at least one cable adapter (2712, 2714) for connecting to at least one device, but may also include multiple adapters (2712, 2714) for connecting to multiple devices as shown in the present example and a cable (2716) which collectively act as a jumper between devices. Surgical instrument (152) may be reusable as a whole or may be disassembled to reuse only a portion of cable assembly (2710). Reusable portions may be determined based on the cost of particular portions, the case of removing and replacing particular portions, the environmental impact of not reusing particular portions, or the visual aesthetics of particular portions. Surgical instrument (152) may also be disassembled for cleaning and sterilization purposes. Such sterilization purposes may include autoclaving and ethylene oxide sterilization of either a select portion of surgical instrument (125) or an entirety surgical instrument (152). Surgical instrument (152) may help to alleviate the cost of procedures by allowing for a reusable electrical conduit rather than using a traditional one-time use disposable electrical conduit. Cable adapters (2712, 2714) and cable (2716) may also shield the electrical conduits from external signals which would affect the performance of surgical instrument (152) and/or generator (150) when conducting electrical communications. While cable assembly (2710) is shown more particularly in the present example as connecting surgical instrument (152) to generator (150) respectively via an instrument adapter (2718) and a generator adapter (2720), cable assembly (2710) may alternatively connect any such devices and/or generators discussed herein such that the invention is not intended to be unnecessarily limited to use with surgical instrument (152) and generator (150).

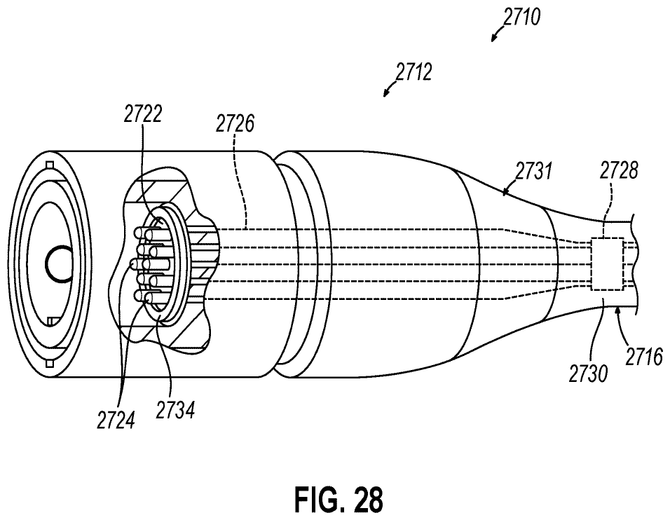

FIG. 28 shows cable adapter (2712) having a terminal seat (2722) with a plurality of electrical contacts (2724) supported thereon. Each electrical contact (2724) in the present example connects to an electrical conduit (2726), which extends to an integrated circuit (2728). As further shown in the present example, integrated circuit (2728) is removably connected to a cable body (2730) of cable (2716) although integrated circuit (2778) in another example may be integrated into any one of adapters (2712, 2714) (see FIG. 27) or even not included at all in other examples. Integrated circuit (2778) is configured to track various parameters and/or metrics of past use of surgical instrument (152), cable assembly (2710), and/or generator (150).

A strain relief (2731) is coupled between cable adapter (2712) and cable body (2730) and configured to inhibit damage to cable body (2730) and/or electrical conduits (2726) when cable assembly (2710) is pulled and/or bent with a damaging force or a damaging angle. In one example, strain relief (2731) may be adhered to cable body (2730) or may simply surround cable body (2730) similar to a shroud. Strain relief (2731) may be permanently or removably secured to cable adapter (2712) and/or cable body (2730) for replacement. Strain relief (2731) may vary in size in order to accommodate differing diameters and to offer more or less strain protection as desired.

At least one electrical conduit (2726), such as a wire, extends from integrated circuit (2728) and traverses cable body (2730) to opposing cable adapter (2714) (see FIG. 27) for connection with generator (150) (see FIG. 27). By way of example, electrical contacts (2724) may be externally coated or made of a corrosion resistant material such as gold or dielectric grease to prohibit corrosion and to promote electrical communication. Electrical contacts (2724) and electrical conduits (2726) may be shielded by adapters (2712, 2714, 2718, 2720) as applicable to form a female type fitting, such as in cable adapters (2712, 2714) of the present example, or may be external to adapters (2712, 2714, 2718, 2720) to form a male type fitting, such as in instrument and generator adapters (2718, 2720).

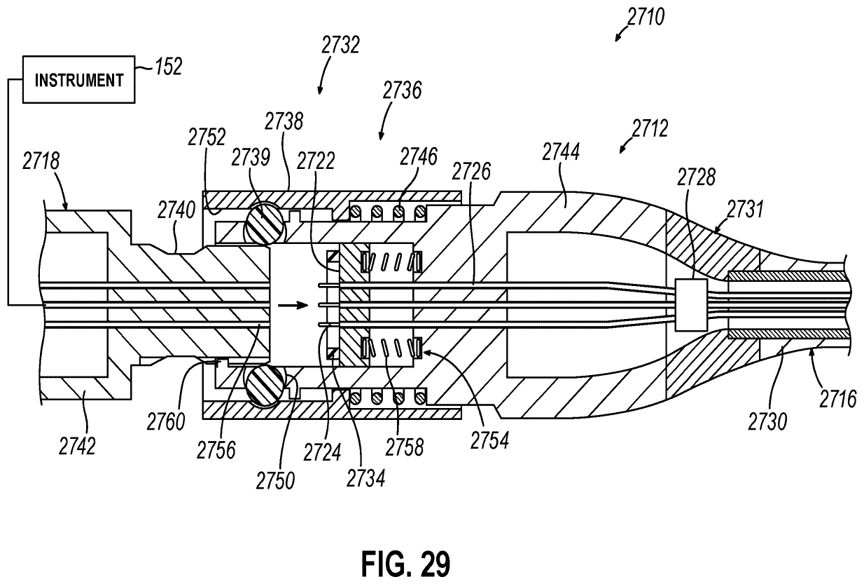

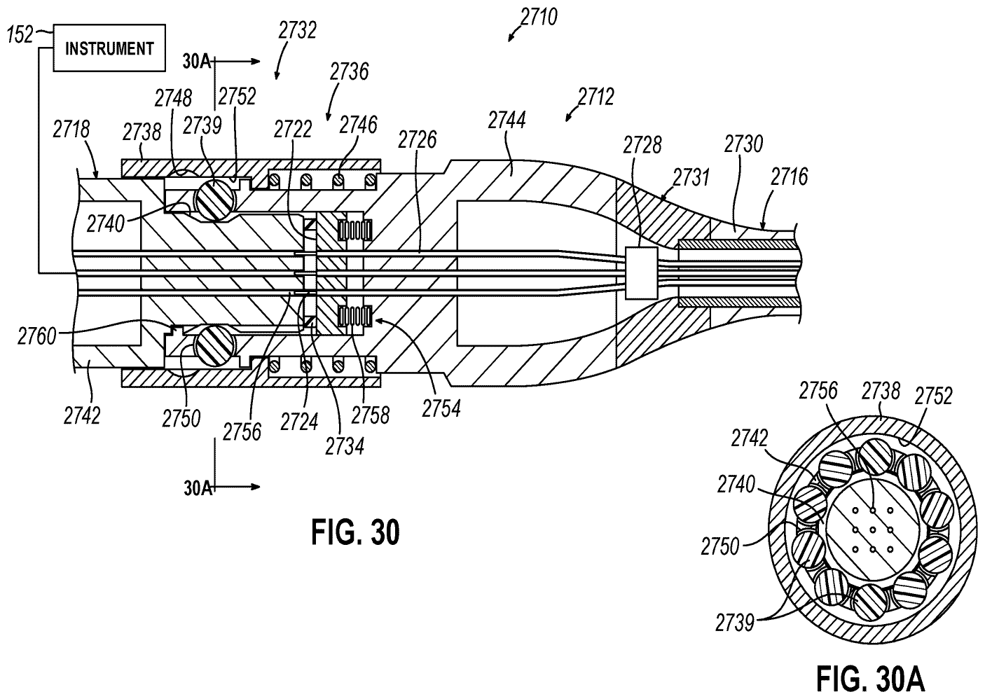

To this end, with respect to FIG. 29, cable adapter (2712) and instrument adapter (2718) define an electrical coupling (2732), whereas cable adapter (2714) (see FIG. 27) and generator adapter (2720) (see FIG. 27) similarly define another such electrical coupling (2732), albeit on opposing ends of cable (2716). Cable adapter (2712) of electrical coupling (2732) includes a seal (2734) configured to inhibit ingress of foreign materials into electrical coupling (2732) when coupled as shown in FIG. 30. Seal (2734) may also be located around all electrical contacts (2724) within electrical coupling (2732) to inhibit the ingress of foreign materials to electrical contacts (2724) when cable assembly (2710) is coupled to surgical instrument (152) (see FIG. 27) and/or generator (150) (see FIG. 27).

Referring to each of FIG. 29, FIG. 30, and FIG. 30A, electrical coupling (2732) includes a latch coupling (2736), which has a selectively translatable sleeve (2738), a plurality of catch members (2739), and an annular groove (2740). More particularly, annular groove (2740) is positioned on an instrument adapter body (2742) to surround a longitudinal axis of electrical coupling (2732), whereas catch members (2739) are angularly positioned about the longitudinal axis of electrical coupling (2732) surrounding the longitudinal axis about a majority of a circumference of a cable adapter body (2744) of cable adapter (2714). Catch members (2739) are further selectively movably secured to cable adapter body (2744). Sleeve (2738) is also movably secured to cable adapter body (2744) to radially surround the longitudinal axis selectively translate in the longitudinal direction.

To this end, sleeve (2738) of the present example is configured to move from the locked position, which mechanically couples cable adapter (2712) to instrument adapter (2718), to the unlocked position, which mechanically uncouples cable adapter (2712) from instrument adapter (2718). In the present example, sleeve (2738) is biased toward the locked position by a sleeve spring (2746) secured in compression between cable adapter body (2744) and sleeve (2738) thereby pushing sleeve (2738) toward the locked position.

In order to enable mechanical coupling and uncoupling from the unlocked position, sleeve (2738) further includes an inner annular recess (2748) positioned to longitudinally align with catch members (2739), which in the present example are more particularly shown as ball-bearings (2739). Cable adapter (2712) further includes at least one receptacle (2750) configured to retain ball-bearings (2739), although it will be appreciated that any member configured to be so retained, such as pins, may be similarly used. Receptacle (2750) longitudinally secures ball-bearings (2739), while simultaneously allowing for limited inner and outer radial movement and permitted by surrounding structures. For example, an inner sidewall (2752) generally urges ball-bearings (2739) radially inward when sleeve is not in the unlocked position; however, inner annular recess (2748) receives ball-bearings (2739) in the unlocked position to allow ball-bearings (2739) to move radially outward. Thus, with sleeve (2738) in the unlocked position, instrument adapter body (2742) urges ball-bearings (2739) radially outward into inner annular recess (2748) of sleeve (2738) upon insertion into cable adapter body (2744). Once annular groove (2740) longitudinally aligns with ball-bearings (2739) as instrument adapter (2718) and cable adapter (2712) are partially coupled, sleeve (2738) selectively returns to the locked position such that inner sidewall (2752) of sleeve (2738) urges ball-bearings (2739) radially inward toward annular groove (2740) in instrument adapter body (2742). Ball-bearings (2739) are effectively captured in annular groove (2740) between inner sidewall (2752) of sleeve (2738) and instrument adapter body (2742) such that instrument adapter (2718) and cable adapter (2712) are fully coupled in the locked position for communication along electrical conduits (2726).

While mechanically coupling instrument adapter body (2742) relative to cable adapter body (2744) generally inhibits inadvertent uncoupling of cable and instrument adapters (2712, 2718), electrical coupling (2732) of the present example further includes a communication coupling (2754) configured to urge direct engagement between electrical contacts (2724) on terminal seat (2722) of cable adapter (2712) and electrical contacts (2756) of instrument adapter (2718). By way of example, terminal seat (2722) of communication coupling (2754) with electrical contacts (2724) is resiliently biased away from cable body (2730) toward instrument adapter (2718) to promote engagement with electrical contacts (2756) in instrument adapter (2718). Communication coupling (2754) thereby allows the electrical contacts (2724) with terminal seat (2722) to longitudinally translate within a predetermined range, which in the present example is a longitudinal predetermined stroke, by pushing against cable adapter body (2744). On one end of the predetermined stroke with a seat spring (2758) in an extended state, electrical contacts (2724) with terminal seat (2722) are fully advanced at a distance away from cable (2716), prior to insertion of instrument adapter body (2742). At the other end of the predetermined stroke, with instrument adapter body (2742) coupled to cable adapter (2712) and seat spring (2758) in a retracted state, electrical contacts (2756) on instrument adapter body (2742) urge terminal seat (2722) toward cable (2716) such that seat spring (2758) is compressed between terminal seat (2722) and cable adapter body (2744). Seat spring (2758) will continue to push electrical contacts (2724) on terminal seat (2722) into adjoining electrical contacts (2756) of instrument adapter (2718) to promote engagement and electrical communication therebetween. In one example, in the event of uncoupling of cable adapter (2712) and instrument adapter (2718) while unlocking latch coupling (2736), seat spring may further aid in the separation of cable adapter (2712) and instrument adapter (2718).

In order to further aid alignment between cable adapter (2712) and instrument adapter (2718), cable adapter (2712) further includes an alignment feature, such as an alignment key (2760), for angularly aligning cable adapter (2712) and instrument adapter (2718) about the longitudinal axis relative to each other. Holding angular alignment will allow the respective electrical contacts (2724, 2756) to properly align as applicable and engage with one another to maintain engagement while coupling and during use of surgical instrument (152) (see FIG. 27).

While the above description is generally directed toward cable adapter (2712) and instrument adapter (2718), it will be appreciated that cable adapter (2714) and generator adapter (2720) are generally identical to cable adapter (2712) and instrument adapter (2718), respectively, unless otherwise described herein. The above description of cable adapter (2712) and instrument adapter (2718) thus similarly applies to cable adapter (2714) and generator adapter (2720) in the present example, although the invention should not be limited to cable adapter (2712) being like cable adapter (2714) and instrument adapter (2718) being like generator adapter (2720). It will be further appreciated that any adapters (2712, 2714, 2718, 2720) may be interchanged with related features on any devices as desired so long as adapters (2712, 2714, 2718, 2720) are configured to effectively mate for communication therebetween during use. In other words, in one example one or more instrument or generator adapters (2718, 2720) may be incorporated into cable assembly (2710) and/or one or more of cable adapters (2712, 2714) may be incorporated into surgical instrument (152) (see FIG. 27) and/or generator (150) (see FIG. 27). The invention is thus not intended to be limited to the particular arrangement of adapters (2712, 2714, 2718, 2720) as shown in the present example.

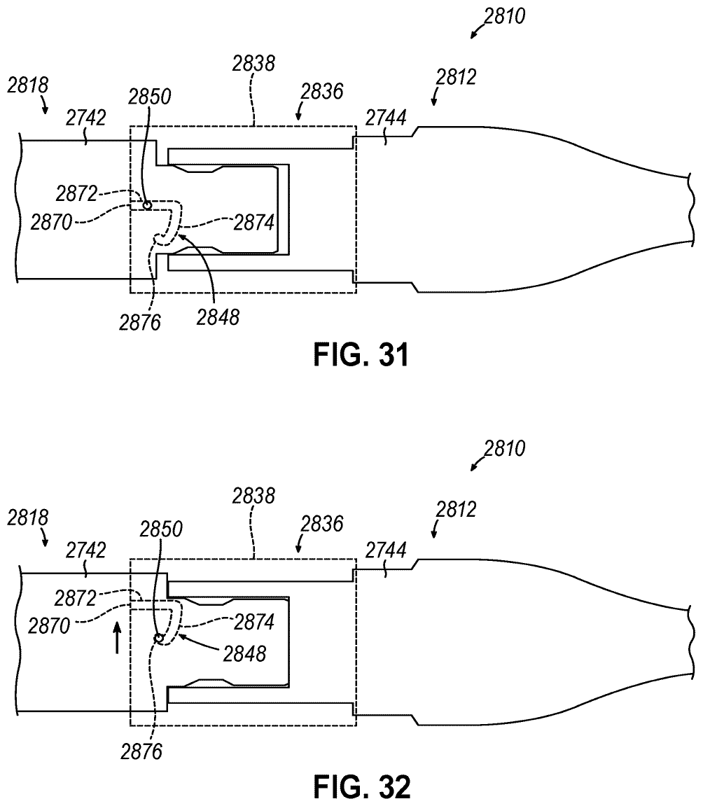

FIGS. 31 and 32 illustrate another exemplary cable assembly (2810) with a cable adapter (2812), an instrument adapter (2818), and an alternative example of a latch coupling (2836) having a rotatable sleeve (2838) for mechanically coupling instrument adapter body (2742) and cable adapter body (2744) together. In this respect, cable and instrument adapters (2812, 2818) are like cable and instrument adapters (2712, 2718) discussed above unless otherwise explicitly noted herein with like numbers indicating like features. More particularly, sleeve (2838) includes a bayonet slot (2848) extending radially therethrough and configured to receive an accompanying bayonet pin (2850) radially extending from instrument adapter body (2742). Sleeve (2838) with bayonet slot (2848) is configured to rotate relative to cable adapter body (2744), whereas bayonet pin (2850) rigidly extends from and is fixed relative to instrument adapter body (2742).

Bayonet slot (2848) more particularly includes an opening (2870), a longitudinally extending linear portion (2872), a longitudinally and angularly extending arcuate portion (2874), and a locking, terminal cavity (2876). Sleeve (2838) is rotatable about cable adapter body (2844) from a partially coupled, unlocked position with bayonet pin (2850) received through opening (2870) into linear portion (2872) as shown in FIG. 31 to a fully coupled, locked position with bayonet pin (2850) rotated through arcuate portion (2874) against an end of bayonet slot (2848) in terminal cavity (2876). Once bayonet pin (2850) reaches the rounded portion, a sleeve spring (2846) advances sleeve (2838) distally in relation to cable (2716) such that bayonet pin (2850) is captured terminal cavity (2876) to thereby secure cable adapter (2812) to instrument adapter (2818) in the locked position. In one example, as terminal cavity (2876) receives bayonet pin (2850), bayonet pin (2850) and sleeve (2838) collectively generate an audible noise and/or tactile feedback to indicate the locked position.

While the above description is generally directed toward cable adapter (2812) and instrument adapter (2818), it will be appreciated that adapters (2812, 2818) may be cooperatively interchanged or included together with any of adapters (2712, 2714, 2718, 2720) with related features on any devices as desired so long as adapters (2712, 2714, 2718, 2720) are configured to effectively mate for communication therebetween during use. Again, the invention is thus not intended to be limited to the particular arrangement of adapters (2812, 2818) as shown in the present example.

### VII. SURGICAL SYSTEM AND METHODS OF ASSEMBLY AND DISASSEMBLY OF SURGICAL INSTRUMENT

Manufacturers of medical devices often offer a wide array of products tailored to fit the individual preferences of the surgeon, hospital, and patient. In order to address these preferences, manufacturers may distribute surgical kits of individual components that require assembly prior to use. In many cases, this assembly occurs immediately prior to use and can be performed by hand without tools. Such assembly may include connecting two electrical connections together or connecting a vacuum line to a vacuum canister. In other cases, this assembly may require specialized tools, such as a wrench to secure a nut.

As with assembly, it may be desirable to disassemble components after use. Disassembly may be advantageous for reasons such as reusing certain components, analyzing internal wear items, and recycling or refurbishing high value components. Disassembly may require use of certain tools and instruments which are specialized to access and remove these components without damaging them.

Surgical instruments, such as instruments (152, 154, 156) and other instruments shown and described herein, s often the most valuable component of surgical kits, but, in many cases, are one-time use components and thus not reusable. If any of such instruments (152, 154, 156) are reusable, these instruments (152, 154, 156) may require at least partial disassembly and cleaning after use to remove any biological material that may have attached to the instrument (152, 154, 156). Whether instrument (152, 154, 156) is one-time use or reusable, instrument (152, 154, 156) will often contain high value components, such as transducers, circuit boards, and sensors that can be either reused or recycled rather than be disposed of.

Accessing and harvesting these high value components can often be a laborious task and in many scenarios is cost prohibitive with little to no design consideration to component harvesting after use. For example, certain non-reversible assembly procedures may be used in the assembly of such instruments (152, 154, 156) to discourage them from being taken apart. These non-reversible procedures may include over-molding, use of adhesives, soldering, use of conformal coatings, one-time fittings, and non-reversible fasteners. Surgical kits, including instruments (152, 154, 156), may have one or more features configured to encourage harvesting of high value and recyclable components. Furthermore, instruments (152, 154, 156) may include the use of assembly practices such as reversible adhesives, standard fasteners, special access ports, and electrical connectors rather than solder joints.

To this end, FIG. 33 depicts a surgical kit (3000) for use in a surgical setting with one or more such features configured to encourage harvesting of high value and recyclable components. Kit (3000) includes a tray (3016) designed to hold and present components and, in the present example, includes all of the items needed to perform a particular operation, although alternative examples may have more or less components as desired for all or part of a procedure. Such surgical kits (3000) can simplify ordering by using a single part number for the entire kit (3000) rather than ordering each component separately, which may result in inadvertently omitting a desired component for a surgical procedure. Surgical kit (3000) may be shipped sterile, can be sterilized on site, or can be used non-sterile if desired. Components in surgical kit (3000) of the present example include instrument (152), replacement parts (3012) for instrument (152), a parts compartment (3018) built into tray (3016) for capturing spare or discarded parts, a parts container (3020), such as a TYVEK® bag described herein, for capturing spare or discarded parts that may need to be fluidly sealed, an instrument tool assembly (3022), and instructions for use (not shown). While surgical kit (3000) incorporates instrument (152) therein, any surgical instrument, such instruments (154, 156) or other such instruments described herein, may be incorporated into an alternative kit such that the invention is not intended to be unnecessarily limited to instrument (152).

More particularly, instrument tool assembly (3022), which may also be refereed to herein as tool (3022), is configured to act upon instrument (152) to gain access to the high value and recyclable components of instrument (152). In this case, tool (3022) is provided in kit (3000) along with instrument (1520 to ensure availability for use with instrument (152), such as upon disassembly of instrument (152).

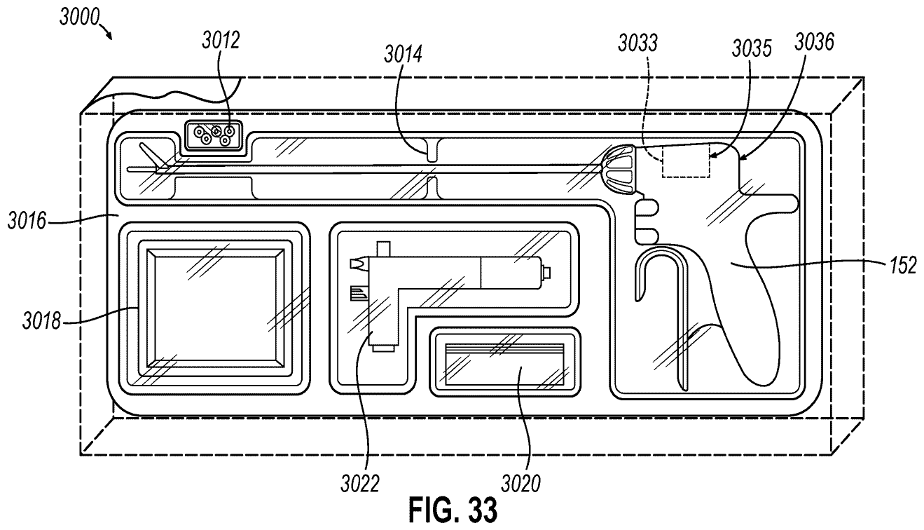

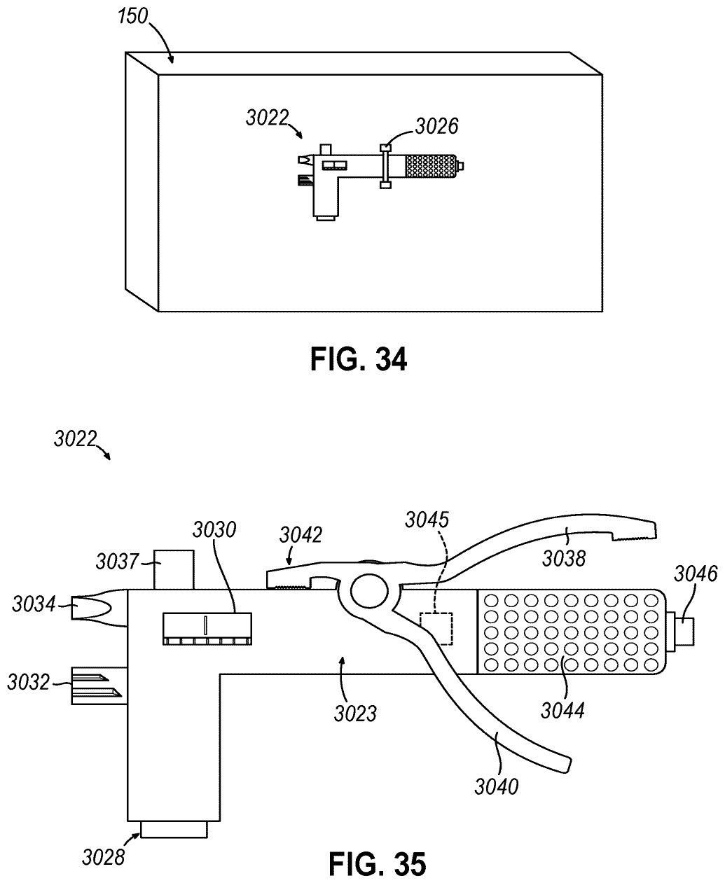

Rather than inclusion of tool (3022) in kit (3000), FIG. 34 shows an alternative example with tool (3022) provided or attached to generator (150) associated with instrument (152) (see FIG. 33). In one example, generator (150) includes a mount (3026) for tool (3022) on generator (150), such as on a sidewall of generator (150). Mount (3026) of the present example is configured to support multiple attachments and detachments of tool (3022). Mount (3026) may also include instructions for using tool (3022), a tool part number of tool (3022), a handpiece part number that tool (3022) is associated with, and/or a website where operators may refer for use of generator (150) and tool (3022). Tool (3022), more particularly, also includes electronics (3045) (see FIG. 35) and labels operable to communicate with nearby instrument (152) (see FIG. 33) and/or generator (150). Electronics (3045) includes a central processing unit (CPU), a memory, a communication unit such as a wireless receiver and transmitter, and/or a feedback system such as a haptic button or display. Electronics (3045) are operable with generator (150) and instrument (152) to communicate usage and function data either wired or wirelessly.

FIG. 35 shows one example of tool (3022) in greater detail for assembling or disassembling instrument (152) (see FIG. 33). Tool (3022) may include a body (3023), a torque limiting aspect, such as a torque wrench coupler (3028), affixed to body (3023). Coupler (3028) may fit a traditional hexagonal head faster, a flat-head, Philips-head screw, or any other proprietary or non-proprietary fittings. In one example, tool (3022) includes a predefined torque limit associated with coupler (3028) that is set and calibrated when tool (3022) is produced and is not adjustable by the user. In another example, tool (3022) includes an adjustable torque limit associated with coupler (3028) such that a user is able to set a torque limit from a plurality of available torque limits to change the torque applied as desired by the user. A torque limit indicator (3030) is presented on body (3023) of the present example to indicate the torque limit that the tool (3022) has been adjusted.

Adjusting the torque limit may be performed by twisting handle (3044) to an indicated torque limit, such as a click-type torque wrench, by twisting a rotation shaft, such as a T-Handle torque wrench, by pressing a button, such as an electronic torque wrench, or by manipulating some portion of tool (3022) according to a predetermined amount. Upon use, tool (3022) indicates that a desired torque has been reached with an audible noise, a tactile indication, and/or a visual indication. Tool (3022) is also be capable of indicating a current torque being applied without limiting the torque, such as a beam style torque wrench. Torque limiting aspect, such as that associated with coupler (3028), may be selectable to either allow torque limiting or to disable torque limiting, thereby using tool (3022) as a wrench or a ratchet. All torque limiting and torque indicating features may be used in clockwise and/or counter-clockwise rotation and may be presented electronically and/or mechanically.

Tool (3022) further includes a pry portion (3034), which is more particularly shown as a wedge (3034) in the present example, intended to separate and pry apart two components of instrument (152) by wedging pry portion (3034) at a predetermined access portion (3035) between the two components, such as two portions of housing. Separation of the two components at predetermined access portion (3035) may be achieved by either using pry portion (3034) as a lever once it has been wedged between the two components or may also be completed by wedging pry portion (3034) deeper between two components and thus forcing them apart. Pry portion (3034) may be located anywhere on tool (3022) capable of manipulating tool (3022) adequately to achieve the desired separation of the two components. Pry portion (3034) may be used to separate components that have been coupled together, such as with a clip or press pin, adhesive, magnets, fracture areas, or by any other securement. Pry portion (3034) also functions as a scraper to remove biological material or adhesive on the instrument (152) or generator (150).

Tool (3022) of the present example also include a key (3032) intended to unlock portions of instrument (152) and/or the generator (150). Key (3032) may be device specific or generic as to apply to several devices. Key (3032) may be operable to disassemble instrument (152) for component harvesting or may be operable to unlock or access features of instrument (152) that would not otherwise be available. By way of example, key is configured to be received within a lock assembly (not shown) of a hatch (3033) configured to transition from a locked state to an unlocked state. In the locked state, hatch (3033) is secured to inhibit access therethrough to an interior (3036) of instrument (152). In the unlocked state, hatch (3033) is configured to selectively move to allow access to interior (3036) and to remove and/or replace components such as an ultrasonic transducer from the interior (3036).

Tool (3022) of the present example also includes a cleaning tube (3037) configured to scrub and clean instrument (152). More particularly, tool (3022) has a lumen cleaner configured to clean a lumen (not shown) on instrument (152). Cleaning tube (3037) and lumen cleaner may include bristles and contact surfaces configured to clean surfaces.

By way of further example, tool (3022) has a plier (3040) and a spreader (3038). Plier (3040) and spreader (3038) may be separate components or may be included as one component hinged against body (3023) of tool (3022). Plier (3040) and spreader (3038) have handles configured to respectively pinch and spread an arm (3042) against body of tool (3022), as shown in the present example. Plier (3040) and spreader (3038) may also feature a single handle with multiple arms (not pictured), one arm being configured to pinch against body (3023) of tool (3022) and the other arm being configured to spread against body (3023) of tool (3022). Plier (3040) is configured to compress components together, such as assembling multiple components of instrument (152) or may be used to crush components of instrument (152), thereby gaining access to portions of instrument (152). In contrast, spreader (3038) is configured to separate components of instrument (152) and thereby gain access to portions of instrument (152), such as through predetermined access portion (3035).

In one example, tool (3022) may be incorporated into a robotic surgical system. In addition to the following teachings, tool (3022) may be constructed and operable in accordance with at least some of the teachings of U.S. Pat. No. 9,125,662, the disclosure of which is incorporated by reference herein, in its entirety; and/or various other references cited herein. By way of further example, tool (3022) is configured to couple at a robotic coupling (3046) with a complementary component of a robotic arm (123) (see FIG. 2). While tool (3022) is coupled to robotic arm (123), robotic arm (123) can use tool (3022) to disassembly, assembly, and/or clean instrument (152) and generator (152). Robotic arm (123) can use tool (123) on instrument (152) while instrument (152) is in tray (3016), is mounted on generator (150), is freely movable, such as on a table, or is coupled to a complementary robotic arm (123). Robotic arm (123) can use all of the previously mentioned functions of tool (3022) such as changing the torque limit and using torque limiting aspect, using key (3032), using pry portion (3034), and using plier (3040) and spreader (3038). When robotic arm (123) is in proximity to tool (3022), robotic arm (123) can communicate with electronics (3045) to learn how tool (3022) has previously been used and to disable tool (3022) from future use. Communication between robotic arm (123) and tool (3022) can be completed wirelessly or through a wired link at robotic coupling (3046).

As noted above, tool (3022) in one example includes electrical features and/or electrically conductive mechanical features. Such features may provide a risk of power or signals undesirably crossing from one electrical feature to another electrical feature and/or from one electrical feature to an electrically conductive mechanical feature. In addition, tool (3022) may include electrical features and/or electrically conductive mechanical features that may provide a risk of generating electrical potentials between proximate components or creating capacitive couplings between electrical features and/or between an electrical feature and an electrically conductive mechanical feature. In the context of tool (3022), such risks may occur with respect to the components that tool (3022) are coupled with. Other components of tool (3022) that may present the above-described risks will be apparent to those skilled in the art in view of the teachings herein.

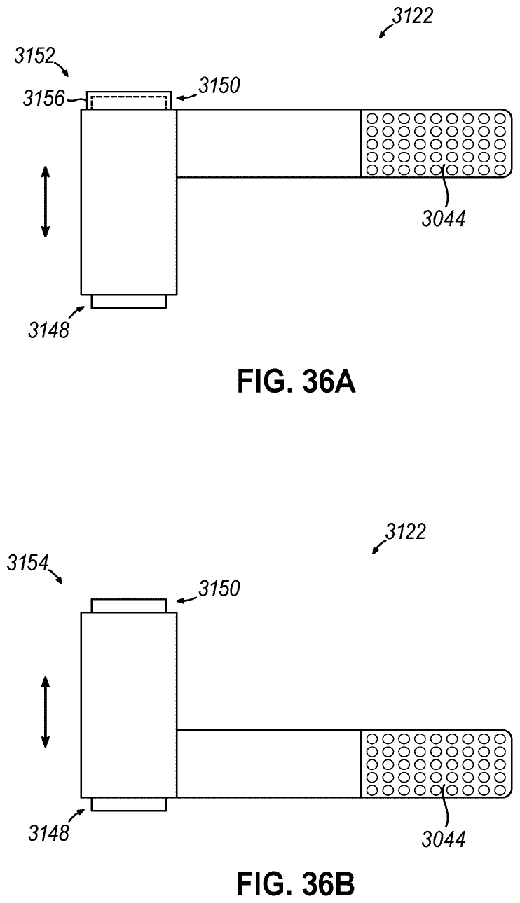

Referring to FIGS. 36A and 36B shows another example of a tool (3122) similar to tool (3022) (see FIG. 35) except as otherwise discussed below. FIG. 36A shows tool (3122) in a first configuration (3152) such that tool (3122) may be used in a particular manner with a first feature (3048) enabled. First feature (3148) may include any of the above features of tool (3022) (see FIG. 35) described above, such as torque wrench coupler (3028), key (3032), pry portion (3034), cleaning tube (3037), spreader (3038), plier (3040), handle (3044), linking with electronics (3045), and robotic coupling (3046) (see FIG. 35). Tool (3122) is configured such that once first feature (3148) is utilized for its intended purpose, a second feature (3150) of tool (3122) becomes operable after use of first feature (3148), and in one example, only after use of first feature (3148). Again, second feature (3150) may include any of the above features of tool (3022) described above, such as torque wrench coupler (3028), key (3032), pry portion (3034), cleaning tube (3037), spreader (3038), plier (3040), handle (3044), linking with electronics (3045), and robotic coupling (3046) (see FIG. 35). While any combination of features of tool (3022) (see FIG. 35) may be incorporated into tool (3122), in the present example, first feature (3148) includes torque wrench coupler (3028) and second feature (3150) includes pry portion (3034), although the invention is not intended to be unnecessarily limited to this particular combination of present example. In the present example, the tool (3122) can be automatically transitioned from first feature (3148) to second feature (3150) once torque wrench coupler (3028) has reached a predefined torque. Transitioning tool (3122) from first feature (3148) to second feature (3150) can change the way a user holds and operates tool (3122).

To enable second feature (3150), tool (3122) transitions from first configuration (3152) to a second configuration (3154) as shown in FIG. 36B. Tool (3122) also includes a feature inhibitor (3156), such as a cap. To this end, when in first configuration (3152), feature inhibitor (3156) is configured to inhibit access to and/or function of second feature (3150) prior to use of first feature (3148). Feature inhibitor (3156) may further include a warning indicia thereon, such as an instruction that first feature (3148) is to be used or that tool (3122) is to be transitioned to the second configuration (3154) prior to second feature (3150) becoming accessible and/or used. Tool (3122) may require removal or destruction of feature inhibitor (3156) to make second feature (3150) operable or accessible. When in the second configuration (3154), tool (3022) is configured to disable first feature (3148) such that first feature (3148) is no longer accessible and/or operable. For example, feature inhibitor (3156) as cap may be placed to cover and/or hide first feature (3148) so that first feature (3148) is no longer accessible and/or visible. Tool (3122) in one example is required to be reset to further make operable first feature (3148) by resetting tool (3122) to first configuration (3152). In one example, when in the second configuration (3154), both the first feature (3048) and second feature (3050) of tool (3022) operable. In this respect, the invention is not intended to be unnecessarily limited to successive operation and access of first and second features (3148, 3150) as shown in the present example.

### VIII. ROBOTIC SURGICAL SYSTEM WITH REMOVABLE PORTION

In some instances, it may be desirable to provide a surgical instrument that includes components capable of delivering ultrasonic energy, RF energy, or both ultrasonic and RF energy that easily open to provide access to the internal components for separation into separate waste streams with minimal tools. Surgery customarily takes place within the sterile field, as described above. The sterile field, being free of microorganisms, enables the surgical team to decrease the chance of infection by ensuring that only sterilized equipment and tools are used within the sterile field. Surgical instruments are sterilized and packaged within sterile containers that are passed into the sterile field. Health care professionals may be required to disassemble the surgical instruments within the sterile field after a surgical procedure by hand or with tools provided within the sterile containers. For example, a torque wrench provided for assembling a surgical instrument, may have additional features to disassemble the surgical instrument.

The surgical instruments include additional features that facilitate disassembly and removal of internal components. These separate waste streams are predetermined based on the material of the component or the use of the component. For example, the waste streams may include recycling, disposal, or refurbishing. Components placed in the disposal waste stream would be disposed of in a land fill. Components placed in the recycling waste stream may be further separated, shredded, and melted down into a base component. Components placed in the refurbishing waste stream would be cleaned, tested, repaired, and refitted within another surgical instrument. For example, the plastic and metal components may be separated into one waste stream for disposal, heavy metals from an integrated circuit may be separated into a second waste stream for recycling, and ultrasonic transducers may be separated into a third waste stream for refurbishing.

### A. Overview of Robotic Surgical System

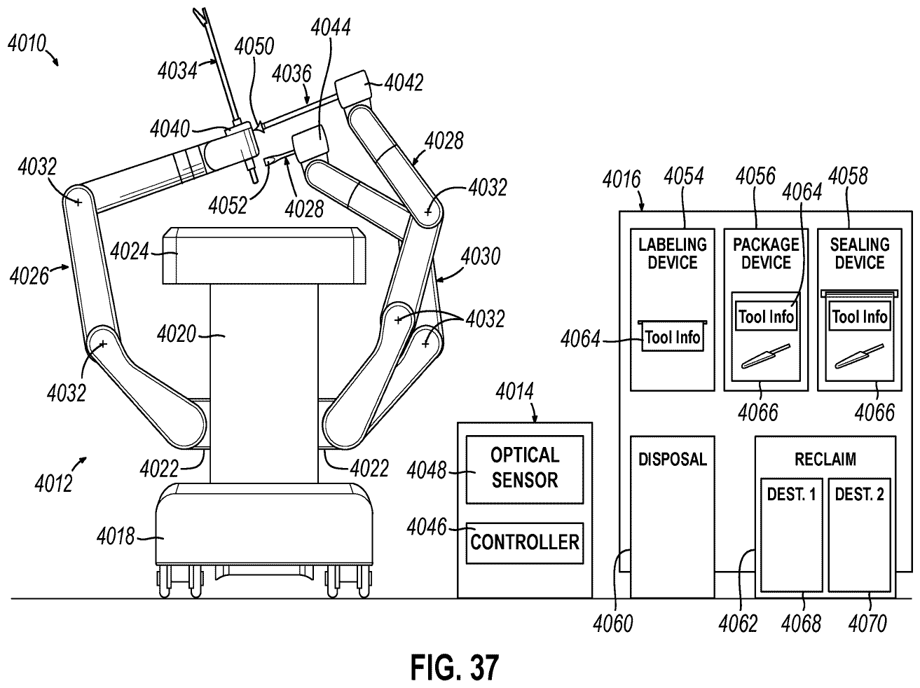

FIG. 37 shows an exemplary robotic surgical system (4010) that includes a patient side cart (4012) (surgical robot), a surgical robotic hub (4014), and a packaging system (4016). Patient side cart (4012) may be similar to patient side cart (120), and surgical robotic hub (4014) may be similar to surgical robotic hub (122) (see FIG. 2), except where otherwise indicated below. While not shown, robotic surgical system (4010) may include additional features similar to robotic surgical system (110) (e.g., a surgeon's console etc.).

As shown in FIG. 37, patient side cart (4012) includes a base (4018), a column (4020), vertical carriages (4022), and an operating table (4024). Base (4018) and column (4020) collectively support operating table (4024). Vertical carriages (4022) are configured to move up or down along, or relative to, column (4020). Operating table (4024) is configured to support a patient thereon, and may be similar to operating table (114). Vertical carriages support plurality of robotic arms, which may be similar to surgical arms (123) that are shown in FIG. 2. While the plurality of robotic arms are shown as including first, second, and third robotic arms (4026, 4028, 4030), more robotic arms (e.g., fourth, fifth, sixth arms etc.) or fewer robotic arms (e.g., first and second arms) are also envisioned. First, second, and third robotic arms (4026, 4028, 4030) extend outwardly from column (4020). As shown, each of first, second, and third robotic arms (4026, 4028, 4030) include joints (4032) allowing for multiple degrees of freedom (e.g., seven or eight degrees of freedom). First robotic arm (4026) is operatively coupled with a surgical instrument (4034) at a first interface (4040). Surgical instrument (4034) is configured to interact with the patient. Surgical instrument (4034) may be similar to surgical instruments (112, 152, 154, 156) described above.

Robotic surgical system (4010) includes a plurality of tools. While FIG. 37 schematically shows the plurality of robotic tools as including tools (4036, 4038), more tools (e.g., third, fourth, fifth, sixth tools etc.) or fewer tools (e.g., first tool) are also envisioned. As schematically shown, second robotic arm (4028) is operatively coupled with tool (4036) at a second interface (4042), and third robotic arm (4030) is operatively coupled with tool (4038) at a third interface (4044).

With continued reference FIG. 37, hub (4014) includes a controller (4046) and an optional sensor (4048). Tools (4036, 4038) are operatively coupled with controller (4046). Disassembly feature (4050) of tool (4036) and/or disassembly feature (4052) of tool (4038) is configured to disconnect at least a portion of surgical instrument (4034) from robotic surgical system (4010) in response to instructions from controller (4046). Controller (4046) may autonomously instruct tool (4036) to disconnect at least a portion of surgical instrument (4034) from robotic surgical system (4010). As used herein, autonomously is intended to mean being capable of performing an operation without requiring additional user instruction once commenced. Controller (4046) is configured to instruct disassembly feature (4050) of tool (4036) to disconnect surgical instrument (4034) in response to feedback received from sensor (4048). In some versions, sensor (4048) may include an optical sensor configured to determine whether people are present in operating room (116).

With continued reference to FIG. 37, packaging system (4016) includes a labeling device (4054), a packaging device (4056) (e.g., a bagging device), a sealing device (4058), a disposal apparatus (4060), and a reclamation apparatus (4062). Labeling device (4054) is configured to dispense labels (4064). Labels (4064) may include tool information (e.g., serial numbers, manufacturing information, use information, etc.). Packaging device (4056) is configured to dispense a package (4066). Label (4064) may be affixed to an exterior surface of package (4066). In some versions, label (4064) may be already attached to package (4066). Package (4066) is configured to receive portion(s) of surgical instrument (4034) in response to instructions from controller (4046). Package (4066) may have a variety of shapes, sizes, and forms. For example, package (4066) may include flexible bags, rigid containers, and/or semi-rigid containers. Controller (4046) is configured to instruct tool (4036) and/or tool (4038) to insert portion of surgical instrument (4034) into package (4066). This may provide personnel within operating room (116) with bagging capability alongside patient side cart (4012).

Packaging system (4016) functions in coordination with patient side cart (4012) so that robotic arms (4026, 4028, 4030) may locate package (4066), allow package (4066) to be automatically dispensed, open package (4066), and close package (4066) when surgical instrument (4034) is located within an interior of package (4066). Scaling device (4058) may seal package (4066) after portion(s) of surgical instrument (4034) is received by package (4066) in response to instructions from controller (4046). Reclamation apparatus (4062) may include first and second shipping containers (4068, 4070). First shipping container (4068) may be used to ship package (4066) to a first location, and second shipping container (4070) may be used to ship package (4066) to a second location that is different from the first location.

At least one of tools (4036, 4038) includes a disassembly feature (4050, 4052). Robotic surgical system (4010) utilizes the interaction of at least surgical instrument (4034) and tools (4036) to achieve the desired disassembly. As will be described in greater detail below, tools (4036, 4038) may utilization discrete strokes or forces for disassembly. For example, tool (4036) may disassemble a first portion of surgical instrument (4034) and tool (4038) may disassemble a different second portion of surgical instrument (4034). Alternatively, disassembly features (4050, 4052) of tools (4036, 4038) may be used in combination (or even in combination with other tools (not shown)) to disassemble surgical instrument (4034). In some versions, disassembly features (4050, 4052) of tools (4036, 4038) may be used in combination simultaneously. In some versions, only tool (4036) includes a disassembly feature (4050), and tool (4038) includes a surgical instrument (e.g., surgical instruments (112, 152, 154, 156)). While not shown, in some versions, surgical instrument (4034) may be used to disassemble at least a portion of tools (4036, 4038).

### B. Exemplary Surgical Instrument

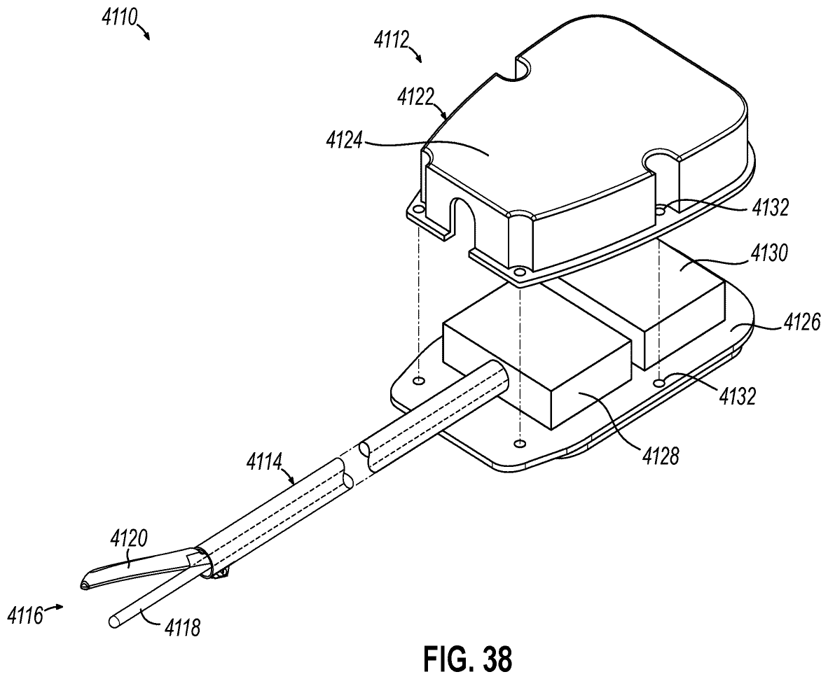

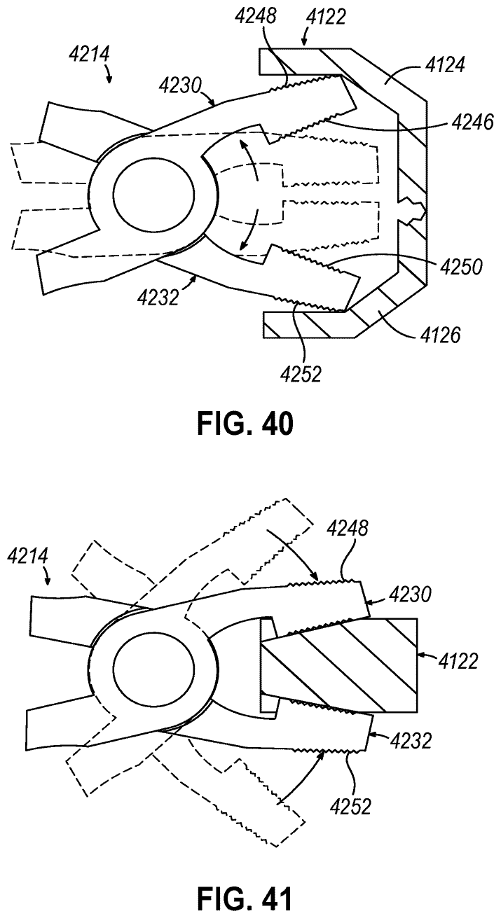

FIG. 38 shows a perspective view of an exemplary surgical instrument (4110) that may be used instead of surgical instrument (112, 152, 154, 156, 4034). Surgical instrument (4110) may be configured to deliver ultrasonic energy, Radio Frequency (“RF”) energy, or both. While surgical instrument (4110) is configured to be operatively coupled with first robotic arm (4026) (see FIG. 37) at first interface (4040) (see FIG. 37), surgical instrument (4110) may alternatively be hand-held. Surgical instrument (4110) includes a body (4112), a shaft assembly (4114), and an end effector (4116). End effector (4116) includes an ultrasonic blade (4118) disposed on a first jaw and a clamp arm (4120) disposed on an opposing second jaw. Clamp arm (4120) is configured to pivot relative to ultrasonic blade (4118). Body (4112) includes a housing (4122) and a plurality of reclaimable components. Housing (4122) is shown as including first and second housing portions (4124, 4126), which may also be referred to as shroud portions. As shown and described below with reference to FIGS. 40-44B, first and second housing portions (4124, 4126) may be coupled using a variety of different coupling structures.

Referring back to FIG. 37 as well as FIG. 38, first housing portion (4124) is separated from second housing portion (4126). While the plurality of reclaimable components are shown as first and second reclaimable components (4128, 4130), more reclaimable components (e.g., third reclaimable component, fourth reclaimable component, etc.) or fewer reclaimable components are envisioned. Controller (4046) may autonomously instruct disassembly feature (4050) and/or disassembly feature (4052) to disconnect first and second reclaimable components (4128, 4130). In some versions, surgical instrument (4110) may be in the form of an ultrasonic surgical instrument that includes ultrasonic components. . . . First reclaimable component (4124) includes an ultrasonic waveguide (which may include ultrasonic blade (4118)). Second reclaimable component (4130) includes an ultrasonic transducer. Controller (4046) may autonomously instruct disassembly feature (4050) and/or disassembly feature (4052) to disconnect the ultrasonic waveguide and the ultrasonic transducer from patient side cart (4012). With first and second housing portions (4124, 4126) released, second reclaimable component (4130) (e.g., the ultrasonic transducer) is exposed for subsequent removal. Surgical instrument (4110) includes markers (4132) to indicate to controller (4046) the predetermined disassembly location.

### C. Exemplary Surgical Tool

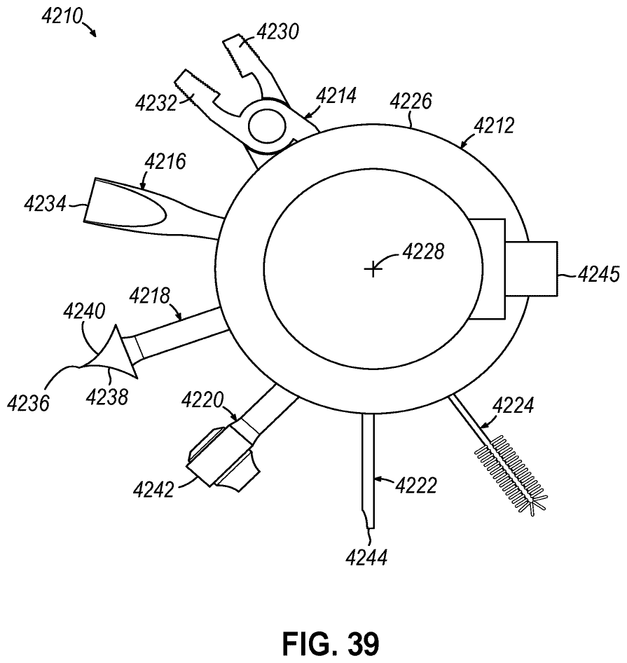

FIG. 39 shows a view of second exemplary tool (4210) that may be used place of tools (4036, 4038). Tool (4210) may be also referred to as a multitool. Tool (4210) includes a body (4212) and a plurality of disassembly features. Body (4212) may rotate about a center point (4228). While the plurality of disassembly features is shown as disassembly features (4214, 4216, 4218, 4220, 4222, 4224), more or fewer disassembly features are envisioned. Disassembly features (4214, 4216, 4218, 4220, 4222, 4224) are shown as extending outwardly from a periphery (4226) of body (4212). Disassembly features (4214, 4216, 4218, 4220, 4222, 4224) may have a variety of shapes and sizes. Disassembly features (4214, 4216, 4218, 4220, 4222, 4224) are configured to remove at least a portion of housing (4122) of surgical instrument (4110). In some versions, disassembly features (4214, 4216, 4218, 4220, 4222, 4224) may be removed, and different disassembly features (4214, 4216, 4218, 4220, 4222, 4224) inserted allowing for a variety of disassembly features depending on surgical instrument (4110) to be disassembled. Once controller (4046) receives the disassembly instructions (e.g., a device code), controller (4046) obtains desired disassembly features (4214, 4216, 4218, 4220, 4222, 4224), and then performs disassembly instructions based on that surgical instrument (4110).

Disassembly feature (4214) is shown as a pair of pliers, but may also function as a pair of reverse pliers. Disassembly feature (4214) includes opposing first and second jaws (4230, 4232) that are configured to move relative to each other as shown and described below with reference to FIGS. 40-41. Disassembly feature (4216) is shown as a scraper. While disassembly feature (4216) is shown as having a distal most end (4234) that is planar and blunt, distal most end (4234) may alternatively be pointed. Disassembly feature (4218) is shown as a wedge. While disassembly feature (4218) is shown as having a distal most end (4236) that is pointed, distal most end (4234) may alternatively be blunt. First and second lateral surfaces (4238, 4240) of disassembly feature (4218) extend outwardly and away from distal most end (4236). Disassembly feature (4220) is shown as a torque wrench. Distal most end (4242) may rotate a fastener for disassembly. Disassembly feature (4222) is shown as a screwdriver having a distal most end (4244) configured to rotate a fastener for disassembly. Disassembly feature (4224) is shown as a pipe cleaner. Disassembly feature (4224) may be used to remove debris prior to disassembly features (4214, 4216, 4218, 4220, 4222) be utilized.

Tool (4210) is shown as including a coupling portion (4245) configured to couple with second interface (4042) or third interface (4044). In some versions, disassembly features (4214, 4216, 4218, 4220, 4222, 4224) may be removable from body (4212). In some versions, disassembly features (4214, 4216, 4218, 4220, 4222, 4224) may be injection molded from a single unitary piece. In some versions, disassembly features (4214, 4216, 4218, 4220, 4222, 4224) may be manufactured using exclusively metal or exclusively plastic. In some versions, robotic arms (4028, 4030), tools (4036, 4038) and accompanying disassembly features (4214, 4216, 4218, 4220, 4222, 4224) are not utilized in the normal operation of the surgical instrument (4110), but allow for tools (4036, 4038) to obtain different orientations and access various portions of patient side cart (4012) and/or switch tools (4036, 4038) and disassembly features (4214, 4216, 4218, 4220, 4222, 4224).

### 1. First Exemplary Disassembly Feature

FIGS. 40-41 show an enlarged view of disassembly feature (4214) of FIG. 39. Particularly, FIG. 40 shows disassembly feature (4214) being used as a reverse pliers to fracture first and second housing portions (4124, 4126) of housing (4122). Opposing first and second jaws (4230, 4232) move from a first configuration (shown in dashed lines) to a second configuration (shown in solid lines) to disassemble housing (4122) of surgical instrument (4110) of FIG. 38. In some versions, only one of first and second jaws (4230, 4232) moves, while the other jaw of first and second jaws (4230, 4232) remains stationary. While distal most ends (4234) of first and second jaws (4230, 4232) are shown as being planar, first and second jaws (4230, 4232) may alternatively terminate to a point. The point may further enable distal most ends (4234) of first and second jaws (4230, 4232) to pry open housing (4122). First jaw (4230) includes inner and outer surfaces (4246, 4248). Similarly, second jaw (4232) includes inner and outer surfaces (4250, 4252). As shown, outer surfaces (4248 4252) of first and second jaws (4230, 4232) push against housing (4122) to fracture housing (4122) or otherwise manipulate housing (4122) as desired.

FIG. 41 shows disassembly feature (4214) of FIG. 40 moving from a third configuration to a fourth configuration to grasp a portion of surgical instrument (4110) of FIG. 38. As shown, inner surfaces (4246, 4250) of first and second jaws (4230, 4232) collectively grab a portion of surgical instrument (4110) as desired. For example, outer surfaces (4248 4252) of first and second jaws (4230, 4232) may be first used to access the desired reclaimable components, then inner surfaces (4246, 4250) of first and second jaws (4230, 4232) may be used to remove and orient the reclaimable components for packaging system (4016). Disassembly feature (4214) may be used to autonomously engage a mechanical key on first robotic arm (4026).

### 2. Second Exemplary Disassembly Feature

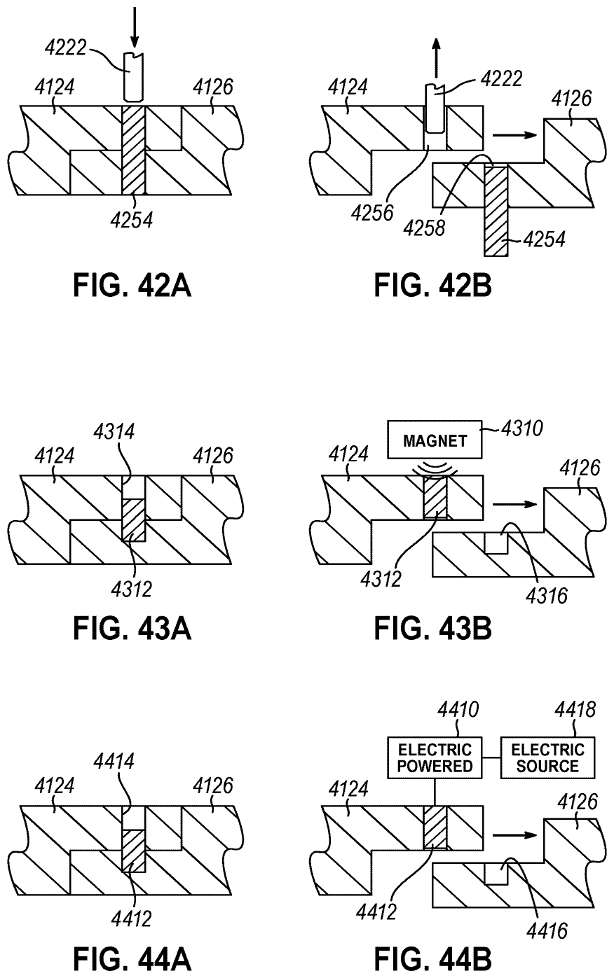

FIGS. 42A-42B show first and second housing portions (4124, 4126) of surgical instrument (4110) being disassembled using disassembly feature (4222) of FIG. 39. Particularly, FIG. 42A shows first and second housing portions (4124, 4126) of FIG. 38 coupled together in a connected configuration using a mechanical connector (4254) prior to separation using disassembly feature (4222). FIG. 42B shows first and second housing portions (4124, 4126) of FIG. 42A, but after disassembly feature (4222) of FIG. 42A moves mechanical connector (4254) to a non-connected configuration. Disassembly feature (4222) functions as a mechanical key to release first and second housing portions (4124, 4126) of surgical instrument (4110) to enable self-disassembly. Mechanical connector (4254) slides within slots (4256, 4258) of first and second housing portions (4124, 4126). While disassembly feature (4222) is shown as translating mechanical connector (4254) to disengage first and second housing portions (4124, 4126), disassembly feature (4222) may rotate and/or translate mechanical connector (4254) to disengage first and second housing portions (4124, 4126). Small predefined housing features may prevent inadvertent disassembly, while still allowing tools (4036, 4038) suitable access to first and second housing portions (4124, 4126).

### 3. Third Exemplary Disassembly Feature

FIGS. 43A-43B show first and second housing portions (4124, 4126) of surgical instrument (4110) being disassembled using an exemplary disassembly feature (4310), which is shown as a magnet. Disassembly feature (4310) may be included as a standalone tool or may be included in tool (4210). Particularly, FIG. 43A shows first and second housing portions (4124, 4126) of FIG. 38 coupled together in a connected configuration using a magnetic connector (4312) prior to separation by disassembly feature (4310). FIG. 43B shows first and second housing portions (4124, 4126) of FIG. 43A, but after disassembly feature (4310) moves magnetic connector (4312) to a non-connected configuration.

Disassembly feature (4310) functions as a magnetic key to release first and second housing portions (4124, 4126) of surgical instrument (4110) to enable self-disassembly. Magnetic connector (4312) slides within slots (4314, 4316) of first and second housing portions (4124, 4126) between the connected configuration and the non-connected configuration. While disassembly feature (4310) is shown as translating magnetic connector (4312) to disengage first and second housing portions (4124, 4126), disassembly feature (4310) may rotate and/or translate magnetic connector (4312) to disengage first and second housing portions (4124, 4126). While disassembly feature (4310) is shown as attracting magnetic connector (4312), disassembly feature (4310) may alternatively repel magnetic connector (4312).

### 4. Fourth Exemplary Disassembly Feature

FIGS. 44A-44B show first and second housing portions (4124, 4126) of surgical instrument (4110) being disassembled using an exemplary disassembly feature (4410), which is shown as an electrically powered disassembly feature (4410). Disassembly feature (4410) may be included as a standalone tool or may be included in tool (4210). Particularly, FIG. 44A shows first and second housing portions (4124, 4126) of FIG. 38 coupled together in a connected configuration using an electrically movable connector (4412) prior to separation using disassembly feature (4410). FIG. 44B shows first and second housing portions (4124, 4126) of FIG. 44A, but after the disassembly feature (4410) of FIG. 44A moves electrically movable connector (4412) to a non-connected configuration. Electrically movable connector (4412) slides within slots (4414, 4416) of first and second housing portions (4124, 4126).

Disassembly feature (4410) functions as an electrically powered key to release first and second housing portions (4124, 4126) of surgical instrument (4110) to enable self-disassembly. Disassembly feature (4410) is operatively connected to a power source (4418) to provide power to disassembly feature (4410). While disassembly feature (4410) is shown as translating electrically movable connector (4412) to disengage first and second housing portions (4124, 4126), disassembly feature (4410) may rotate and/or translate electrically movable connector (4412) to disengage first and second housing portions (4124, 4126).

### 5. Fifth Exemplary Disassembly Feature

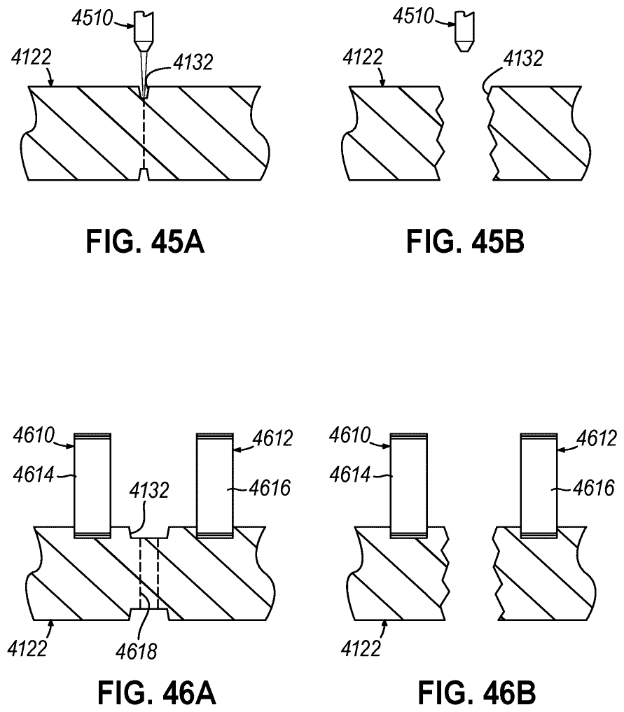

FIGS. 45A-45B show housing (4122) of surgical instrument (4110) being separated using an exemplary disassembly feature (4510), which is shown as a laser. Disassembly feature (4510) may be included as a standalone tool or may be included in tool (4210). Particularly, FIG. 45A shows housing (4122) in a connected configuration prior to separation by disassembly feature (4510). Housing (4122) includes marker (4132) shown as a recessed portion to direct disassembly feature (4510) to the desired location. FIG. 45B shows housing (4122) already being disconnected after disassembly feature (4510) penetrates completely through housing (4122). Disassembly feature (4510) may cut through specific predetermined areas to release first and second reclaimable components (4128, 4130). In some versions, housing (4122) of surgical instrument (4110) may be formed from nitinol, so that disassembly feature (4510) may apply heat to transform the nitinol material for disassembly.

### 6. Sixth Exemplary Disassembly Feature

FIGS. 46A-46B show disassembly features (4610, 4612), shown as first and second end effectors configured to interact with tissue of a patient. Disassembly features (4610, 4612) may be operatively coupled with second and third robotic arms (4028, 4030). Disassembly features (4610, 4612) are shown moving from the first configuration toward the second configuration. Disassembly features (4610, 4612) may be similar to end effectors (166, 180, 188, 4116) of surgical instruments (152, 154, 156, 4110). Disassembly features (4610, 4612) each include a pivotable clamp arm (4614, 4616) for grasping. Marker (4132) of housing (4122) highlights a frangible portion (4618). Disassembly features (4610, 4612) are configured to separate frangible portion (4618) of housing (4122) of surgical instrument (4034) of FIG. 38. FIG. 46B shows a front schematic view of the frangible portion (4618) in a severed state.

In some versions, disassembly feature (4610) is configured to provide a first predetermined force, a first predetermined motion, and/or first predetermined task to disconnect at least portion of surgical instrument (4110). In some versions, underapplication of a first predetermined force or overapplication of the first predetermined force does not release housing (4122). The first predetermined force is greater than a maximum force capable of being provided manually by a user. In other words, the first predetermined force may exceed the force that the user is capable of manually exerting to remove housing (4122). In some versions, both underapplication and overapplication prevent housing (4122) from sufficiently opening.

Disassembly feature (4612) is configured to provide a second predetermined force, a second predetermined motion, and/or second predetermined task independent from first predetermined force, first predetermined motion, and/or first predetermined task applied by disassembly feature (4610) to disconnect at least portion of surgical instrument (4034) in response to instructions from controller (4046). Utilization of multiple separate independently applied forces, motions, or tasks collectively allow portions of surgical instrument (4034) to disassemble. For example, disassembly features (4610, 4612) of respective tools (4036, 4038) may produce synchronized motions that cooperatively unlock two or more separate portions simultaneously to open or remove a portion of surgical instrument (4034) that the user would not be able to manually produce the cooperative forces.

### D. Exemplary Tool Dispenser

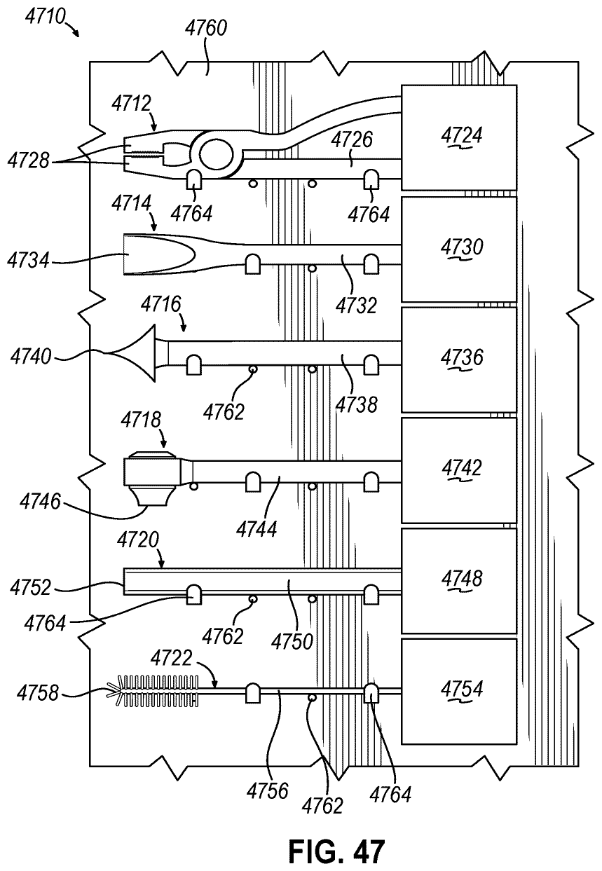

FIG. 47 shows an exemplary tool dispenser (4710) configured to retain a plurality of tools (shown as tools (4712, 4714, 4716, 4718, 4720, 4722)). Tools (4712, 4714, 4716, 4718, 4720, 4722) may be individually wrapped in sterile packaging. Tool (4712) includes a coupling portion (4724), a shaft (4726), and a disassembly feature (4728), which is similar to disassembly feature (4214) shown in FIGS. 39-41. Tool (4714) includes a coupling portion (4730), a shaft (4732), and a disassembly feature (4734), which is similar to disassembly feature (4216) shown in FIG. 39. Tool (4716) includes a coupling portion (4736), a shaft (4738), and a disassembly feature (4740), which is similar to disassembly feature (4218) shown in FIG. 39. Tool (4718) includes a coupling portion (4742), a shaft (4744), and a disassembly feature (4746), which is similar to disassembly feature (4220) shown in FIG. 39. Tool (4720) includes a coupling portion (4748), a shaft (4750), and a disassembly feature (4752), which is similar to disassembly feature (4222) shown in FIG. 39. Tool (4722) includes a coupling portion (4754), a shaft (4756), and a disassembly feature (4758), which is similar to disassembly feature (4224) shown in FIG. 39.

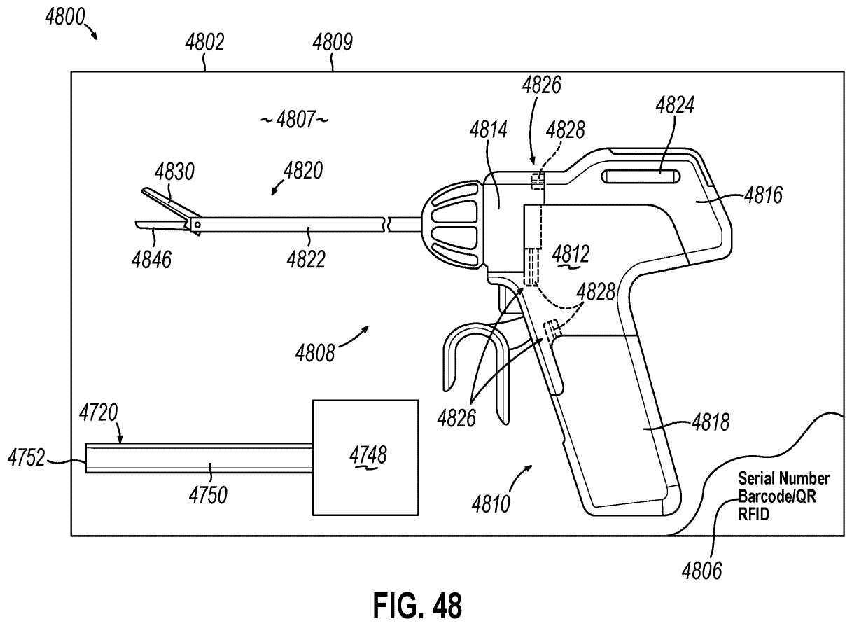

Coupling portions (4724, 4730, 4736, 4742, 4748, 4754) are configured to couple with second interface (4042) of robotic arm (4028) or third interface (4044) of robotic arm (4030) shown in FIG. 37. In some versions, coupling portions (4724, 4730, 4736, 4742, 4748, 4754) may have a base in the shape of an X cross-sectional pattern or a T cross-sectional pattern. The user may attach coupling portions (4724, 4730, 4736, 4742, 4748, 4754) of tools (4712, 4714, 4716, 4718, 4720, 4722) to second interface (4042) or third interface (4044) based on surgical instrument (4034) to be disassembled. Tools (4712, 4714, 4716, 4718, 4720, 4722) may hang on tool dispenser (4710) for subsequent retrieval. As shown, tool dispenser (4710) includes a pegboard (4760) that includes recessed portions or apertures (4762) that support projections (4764). Projections (4764) support tools (4712, 4714, 4716, 4718, 4720, 4722). However, a variety of suitable tool dispensers are also envisioned. While tools (4712, 4714, 4716, 4718, 4720, 4722) are shown as extending horizontally, tools (4712, 4714, 4716, 4718, 4720, 4722) may alternatively be arranged at a variety of other angles to be received by robotic arms (4028, 4030). Tools (4712, 4714, 4716, 4718, 4720, 4722) may be selected by controller (4046) for disassembly of surgical instrument (4034) based disassembly instructions. Disassembly instructions may be transmitted to controller (4046) or another portion of robotic surgical system (4010). Disassembly instructions may be disposed on packaging materials as shown and described below with reference to FIG. 48.

### E. Exemplary Surgical Kit

FIG. 48 shows an exemplary surgical kit (4800) that includes a packaging (4802), tool (4720), instrument information (4806), and a surgical instrument (4808). Packaging defines an interior (4807) and an exterior (4809). For example, surgical kit (4800) may contain both a surgical instrument (4110) as well as tool (4036, 4038) for coupling and/or decoupling surgical instrument (4808) from robotic surgical system (4010). Surgical kit (4800) may include a variety of surgical instruments (112, 152, 154, 156, 4034, 4110) and tools (4036, 4038, 4210, 4712, 4714, 4716, 4718, 4720, 4722). Instrument information (4806) may be in the form disassembly instructions to indicate to the desired position and/or orientation of surgical instrument (4808) for disassembly and/or a computer readable code that may be read and interpreted by controller (4046).

Surgical instrument (4808) may be similar to surgical instruments (112, 152, 154, 156). Surgical instrument (4808) may be configured to deliver ultrasonic energy, Radio Frequency (“RF”) energy, or both. Surgical instrument (4808) may be configured to be hand-held or fitted with a corresponding portion of a robotic arm (see FIG. 38). Surgical instrument (4808), like surgical instruments (112, 152, 154, 156), includes a body assembly (4810), a shaft assembly (4820), and an end effector (4830). Shaft (4822) of shaft assembly (4820) extends distally from body assembly (4810) to end effector (4830). Surgical instrument (4808) differs from surgical instruments (112, 152, 154, 156) in that surgical instrument (4808) includes body assembly (4810) configured to be easily disassembled to expose at least one of a plurality of internal components for removal into separate waste streams. Surgical instrument (4808) is configured to deliver ultrasonic energy similar to surgical instrument (152). Body assembly (4810) surrounds a portion of an energy drive system (4840) and a portion of a circuit assembly (4850). Energy drive system (4840) includes an ultrasonic transducer (4842), a waveguide (4844), and an ultrasonic blade (4846). Energy drive system (4840) may further include a battery (4848), or a generator (150) (see FIG. 5) configured to supply energy. Ultrasonic transducer (4842) is proximally positioned within body assembly (4810) and extends distally to waveguide (4844). Waveguide (4844) extends distally through shaft assembly (4820) to ultrasonic blade (4846). Circuit assembly (4850) includes a main circuit board (4852), a memory member (4854), and a controller (4856). Reclaimable components may include portions of body assembly (4810), shaft assembly (4820) and/or end effector (4830), including components of energy drive system (4840).

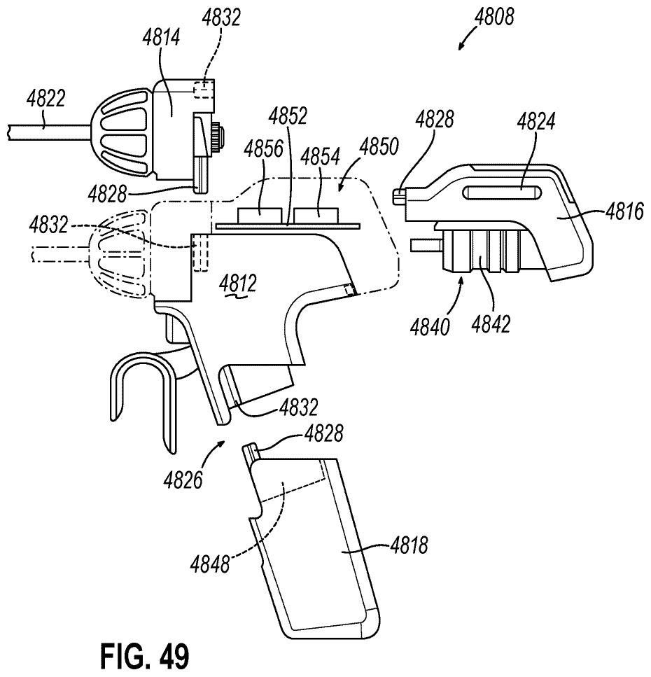

Body assembly (4810) includes a plurality of selectively removeable shroud portions (4812, 4814, 4816, 4818). Shroud portions (4812, 4814, 4816, 4818) are configured to provide support for energy drive system (4840), shaft assembly (4820), and circuit assembly (4850). As illustrated, shroud portions (4812, 4814, 4816, 4818) include a first shroud portion (4812), a second shroud portion (4814), a third shroud portion (4816), and a fourth shroud portion (4818), but may include any number of shroud portions (4812, 4814, 4816, 4818) that inhibit access to circuit assembly (4850) and energy drive system (4840). Each shroud portion (4812, 4814, 4816, 4818) is removably affixed to another shroud portion (4812, 4814, 4816, 4818). Users may remove a shroud portion (4812, 4814, 4816, 4818) to provide access to portions of energy drive system (4840) and portions of circuit assembly (4850) in a disconnected state (see FIG. 49). Once accessed, portions of energy drive system (4840) and portions of circuit assembly (4850) may be disposed of in separate waste streams. Shroud portions (4812, 4814, 4816, 4818) may include gripping features (4824). Shroud portions (4812, 4814, 4816, 4818) further include a plurality of alignment features (4826) configured to align each shroud portion (4812, 4814, 4816, 4818) with an adjacent shroud portion (4812, 4814, 4816, 4818). Alignment features (4826) in one example include a key (4828) and a keyway (4832). Key (4828) is sized to slide within keyway (4832).

FIG. 49 shows surgical instrument (4808) of FIG. 48, but after being removed from packaging (4802) of surgical kit (4800) and after disassembly using disassembly feature (4804) that was included in the same packaging (4802) of surgical kit (4800). In the disconnected state, shroud portions (4812, 4814, 4816, 4818) are separated from one another. Removal of shroud portions (4812, 4814, 4816, 4818) facilitates access to and removal at least a portion of energy drive system (4840) and/or at least a portion of circuit assembly (4850).

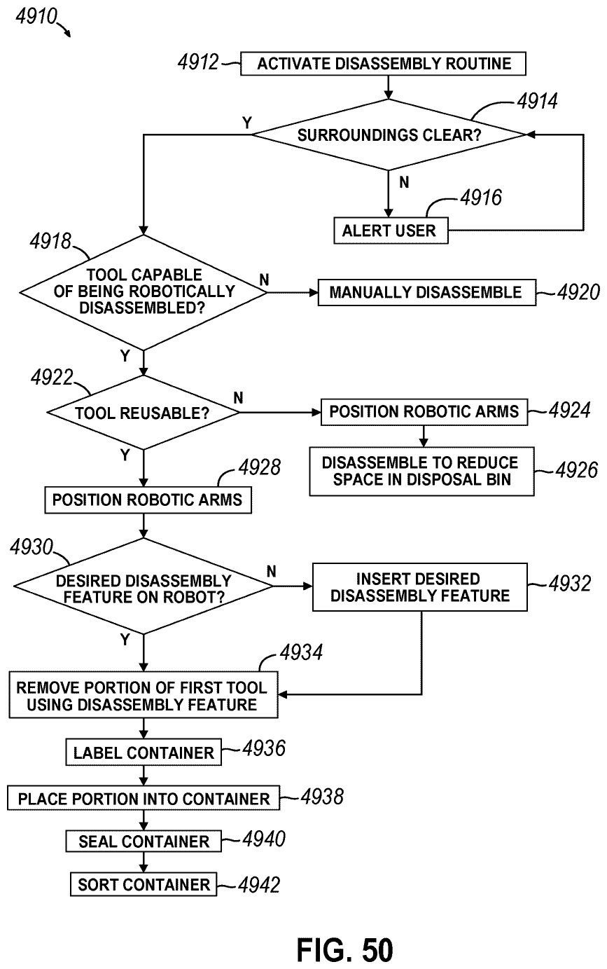

FIG. 50 shows a diagrammatic view of an exemplary method (4910) of disassembling the robotic surgical system (4010) of FIG. 37. Method (4910) may include steps (4912, 4914, 4916, 4918, 4920, 4922, 4924, 4926, 4928, 4930, 4932, 4934, 4936, 4938, 4940, 4942). However, more or fewer steps are also envisioned.

### F. Exemplary Method

At step (4912), method (4910) includes activating the disassembly routine. In some versions, once the disassembly routine is activated, the remainder of the steps may be performed autonomously without any interaction by the user. For example, at the end of a procedure, the autonomous system may react to user input to disassemble surgical instrument (4034). Particularly, a device code may be received by controller (4046), controller (4046) interprets disassembly instructions based on surgical instrument (112, 152, 154, 156, 4034, 4110, 4808). Markers (4132) of surgical instrument (4110) may be used to aid patient side cart (4012) in locating the predetermined disassembly location. For example, at least one of first and second housing portions (4124, 4126) may include markers (4132) to indicate to robot the position/orientation of surgical instrument (112, 152, 154, 156, 4034, 4110, 4808). In some versions, controller (4046) may sense information pertaining to surgical instrument (112, 152, 154, 156, 4034, 4110, 4808), such that the act of disassembling is performed based on sensing with or without subsequent user intervention.

At step (4914), method (4910) includes determining if the surroundings are clear. Controller (4046) checks available contextual information prior to disassembly. For example, controller (4046) may verify the patient is off the operating table (4024) and staff are located a safe position away from robotic surgical system. This determination may be performed using at least one of sensor (4048) in operating room (116), badge proximity scanners, a laparoscopic camera, and a weight sensor on operating table (4024). Badge proximity scanners may assess whether users are present within operating room (116) and where they are located within operating room (116). If the surroundings are not clear, at step (4916), controller (4046) may alert the user. The user may manually clear the alert or robotic surgical system (4010) may continuously or periodically assess whether the surroundings (e.g., within operating room (116)) are clear.

If the surroundings are clear, at step (4918), method (4910) may determine if surgical instrument (4034) is capable of being robotically disassembled. Robotic surgical system (4010) provides feedback for which surgical instruments (112, 152, 154, 156, 4034, 4110, 4808) may be disassembled and instructs the user to attach any of surgical instruments (112, 152, 154, 156, 4034, 4110, 4808) and tools (4036, 4038, 4210, 4712, 4714, 4716, 4718, 4720, 4722) to first, second and third robotic arms (4026, 4028, 4030). If surgical instrument (112, 152, 154, 156, 4034, 4110, 4808) is not capable of being robotically disassembled, at step (4920), surgical instrument (4034) may be manually disassembled. For components of surgical instruments that cannot be disassembled by tools (4036, 4038, 4210, 4712, 4714, 4716, 4718, 4720, 4722) of second and third robotic arms (4028, 4030), manual instructions may be displayed through monitors located within operating room (116).

In some versions, a first surgical instrument (112, 152, 154, 156, 4034, 4110, 4808) has a first keying and a second surgical instrument (112, 152, 154, 156, 4034, 4110, 4808) has a second keying; controller (4046) of robotic surgical system (4010) accesses a lookup table that informs controller (4046) which of the key patterns to use for surgical instrument (112, 152, 154, 156, 4034, 4110, 4808). For example, a first-generation device may utilize a first disassembly protocol or program. A second-generation device has an architecture that differs from the first generation device. Robotic surgical system (4010) may identify tools (4036, 4038, 4210, 4712, 4714, 4716, 4718, 4720, 4722) uniquely and select the desired disassembly method for the desired generation of surgical instrument (112, 152, 154, 156, 4034, 4110, 4808).

If surgical instrument (112, 152, 154, 156, 4034, 4110, 4808) is capable of being robotically disassembled, at step (4922), controller (4046) may determine if surgical instrument (112, 152, 154, 156, 4034, 4110, 4808) is reusable. During disassembly, robotic surgical system (4010) recognizes the appropriate reclamation and disposal methods for surgical instrument (112, 152, 154, 156, 4034, 4110, 4808) and components contained therein. Robotic surgical system (4010) may optionally perform mechanical and/or electrical tests to determine if surgical instrument (112, 152, 154, 156, 4034, 4110, 4808) is capable of to be reused or recertified. Controller (4046) is configured to perform at least one mechanical or electrical test on surgical instrument (112, 152, 154, 156, 4034, 4110, 4808) to determine reusability of surgical instrument (112, 152, 154, 156, 4034, 4110, 4808). Sensor (4048) is configured to sense an area around robotic surgical system (4010). If surgical instrument (112, 152, 154, 156, 4034, 4110, 4808) is not reusable, at step (4924), controller (4046) may instruct first, second, and third robotic arms (4026, 4028, 4030) to desired position. At step (4926), if surgical instrument (112, 152, 154, 156, 4034, 4110, 4808) is not able to be reused, patient side cart (4012) upon instruction from controller (4046) dismantles surgical instrument (4110) to reduce space in disposal apparatus (4060). This may assist in environmentally friendly disposal of components as instructed.

If surgical instrument (112, 152, 154, 156, 4034, 4110, 4808) is reusable, at step (4928), controller (4046) may instruct first, second, and third robotic arms (4026, 4028, 4030) to move the desired position. This predetermined position may aid in the removal of surgical instrument (112, 152, 154, 156, 4034, 4110, 4808). Robotic surgical system (4010) properly positions tools (4036, 4038, 4210, 4712, 4714, 4716, 4718, 4720, 4722) so that first, second, and third robotic arms (4026, 4028, 4030) do not collide, and so that surgical instrument (112, 152, 154, 156, 4034, 4110, 4808) and tools (4036, 4038, 4210, 4712, 4714, 4716, 4718, 4720, 4722) are properly positioned to interact with one another.

After positioning first, second, and third robotic arms (4026, 4028, 4030), at step (4930), controller (4046) may identify the desired disassembly features (4050, 4052, 4214, 4216, 4218, 4220, 4222, 4224, 4310, 4410, 4510, 4610, 4612, 4728, 4734, 4740, 4746, 4752, 4758) and determine if the desired disassembly features (4050, 4052, 4214, 4216, 4218, 4220, 4222, 4224, 4310, 4410, 4510, 4610, 4612, 4728, 4734, 4740, 4746, 4752, 4758) is currently coupled with robotic arms (4028, 4030). If the desired disassembly feature (4050, 4052, 4214, 4216, 4218, 4220, 4222, 4224, 4310, 4410, 4510, 4610, 4612, 4728, 4734, 4740, 4746, 4752, 4758) is not currently coupled with robotic arm (4028, 4030), at step (4932), controller (4046) may instruct second and third robotic arms (4028, 4030) to couple with desired tool (4036, 4038, 4210, 4712, 4714, 4716, 4718, 4720, 4722) that include the desired disassembly features (4050, 4052, 4214, 4216, 4218, 4220, 4222, 4224, 4310, 4410, 4510, 4610, 4612, 4728, 4734, 4740, 4746, 4752, 4758).

If the desired disassembly feature(s) (4050, 4052, 4214, 4216, 4218, 4220, 4222, 4224, 4310, 4410, 4510, 4610, 4612, 4728, 4734, 4740, 4746, 4752, 4758) is currently coupled with robotic arms (4028, 4030) of patient side cart (4012), at step (4934), controller (4046) may instruct robotic arms (4028, 4030) to remove a portion of surgical instrument (112, 152, 154, 156, 4034, 4110, 4808) using disassembly features (4050, 4052, 4214, 4216, 4218, 4220, 4222, 4224, 4310, 4410, 4510, 4610, 4612, 4728, 4734, 4740, 4746, 4752, 4758). Regarding surgical instrument (4808), reclaimable components may include portions of body (4112), shaft assembly (4114) and/or end effector (4116), including ultrasonic components. Regarding surgical instrument (4808), reclaimable components may include portions of body assembly (4810), shaft assembly (4820) and/or end effector (4830), including components of energy drive system (4840). Tool (4036) (and optionally tool (4038)) may apply a predetermined force, motion, or stroke to overcome a connection bias of surgical instrument (112, 152, 154, 156, 4034, 4110, 4808). A portion of surgical instrument (112, 152, 154, 156, 4034, 4110, 4808) may be disassembled using disassembly features (4050, 4052, 4214, 4216, 4218, 4220, 4222, 4224, 4310, 4410, 4510, 4610, 4612, 4728, 4734, 4740, 4746, 4752, 4758) of tool (4036, 4038, 4210, 4712, 4714, 4716, 4718, 4720, 4722) in response to instructions from controller (4046).

At step (4936), controller (4046) may label package (4066) using labeling device (4054). In some versions, controller (4046) and may assign and labeling device (4054) print label (4064) denoting one or more characteristics of reclaimable portion (4124, 4126). Package (4066) may be pre-labeled, or labels (4064) may be printed from labeling device (4054), which may be part of packaging system (4016) or hub (4014). In some versions, hub (4014) may recognize reclaimable components (4128, 4130) being bagged and dispense appropriate bags and labels.

At step (4938), controller (4046) may instruct second robotic arm (4028) and tool (4036, 4038, 4210, 4712, 4714, 4716, 4718, 4720, 4722) to place portion into a predetermined container. Robotic surgical system (4010) may place reclaimable components (4128, 4130) into the appropriate package (4066). Reclamation containers of different types are positioned alongside patient side cart (4012) to provide operating room personnel with ease of use and increased efficiency. Robotic surgical system (4010) may autonomously detect disposal apparatus (4060) and first and second shipping containers (4068, 4070). In some versions, an algorithm of controller (4046) may identify a bag station location and autonomously move reclaimable components (4128, 4130) to appropriate position. Packaging device (4056), which may include a bag dispenser configured to dispense flexible bags, interacts with controller (4046) such that robotic arms (4028, 4030) locate package (4066) that is automatically dispensed, opened, and closed when reclaimable components (4128, 4130) are positioned therein. Autonomously bagging and scaling may aid in proper disposition post-surgery.

At step (4940), method (4910) may also include sealing package (4066) that contains portion (e.g., reclaimable components (4128, 4130)) of surgical instrument (112, 152, 154, 156, 4034, 4110, 4808) using sealing device (4058) in response to instructions from controller (4046). For example, controller (4046) may instruct second robotic arm (4028) and tool (4036, 4038, 4210, 4712, 4714, 4716, 4718, 4720, 4722) and/or third robotic arm (4030) and tool (4036, 4038, 4210, 4712, 4714, 4716, 4718, 4720, 4722)) to seal package (4066). Sealing device (4058) may vacuum seal and/or heat seal reclaimable components (4128, 4130) of surgical instrument (112, 152, 154, 156, 4034, 4110, 4808) post procedure within packages (4066) to prevent cross contamination and reduce space around packaging system (4016).

At step (4942), controller (4046) may sort packages (4066) by desired location/destination. Robotic surgical system (4010) may utilize location-based information to incorporate different disposal and shipping instructions based on country or regional differences and/or specific medical facility capabilities. Reclaimable components (4128, 4130) in packages (4066) and disposable components in disposal apparatus (4060) may be sorted and disposed in different ways depending on the local regulations.

### IX. EXEMPLARY INTRODUCTION, ASSEMBLY, USE, AND DISPOSAL OF A SURGICAL KIT IN A SURGICAL THEATER

As mentioned above, objects intended to penetrate a sterile field of the surgical theater during a surgical procedure need to be suitably sterilized; while objects leaving the sterile field after a surgical procedure often require special consideration when processing for disposal, reuse, or remanufacturing. In some instances, surgical instrument/tool (112, 117, 152, 154, 156) may require at least some degree of assembly within the surgical theater prior to exemplary use in accordance with the description herein; and then require some degree of disassembly after exemplary use such that selective components may be disposed, reused, and/or remanufactured. For example, ultrasonic blade (168) may need to be suitably coupled to ultrasonic transducer (162) and/or shaft assembly (164) may need to be suitably coupled to handpiece (160) within the surgical theater prior to exemplary use of instrument (152). Additionally, after exemplary use, ultrasonic blade (168) may need to be decoupled from ultrasonic transducer (162) for suitable processing in accordance with the description herein.

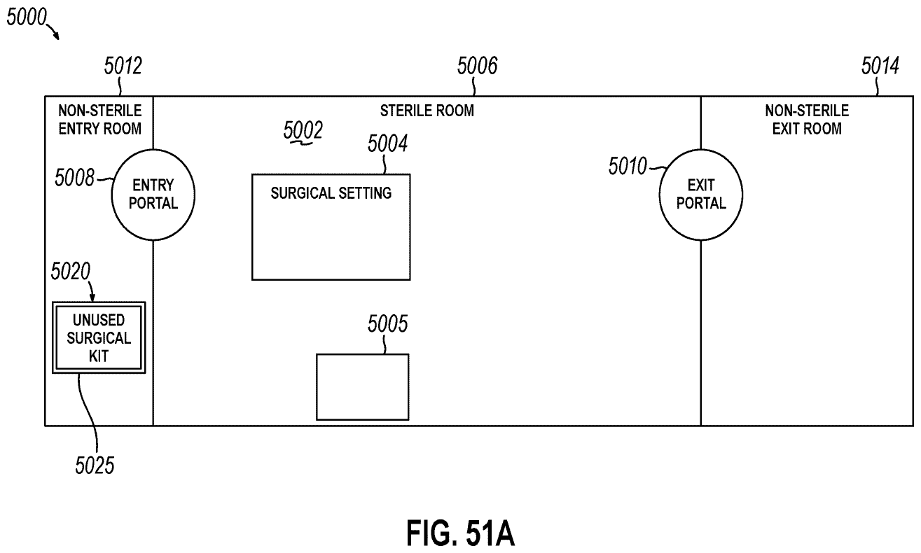

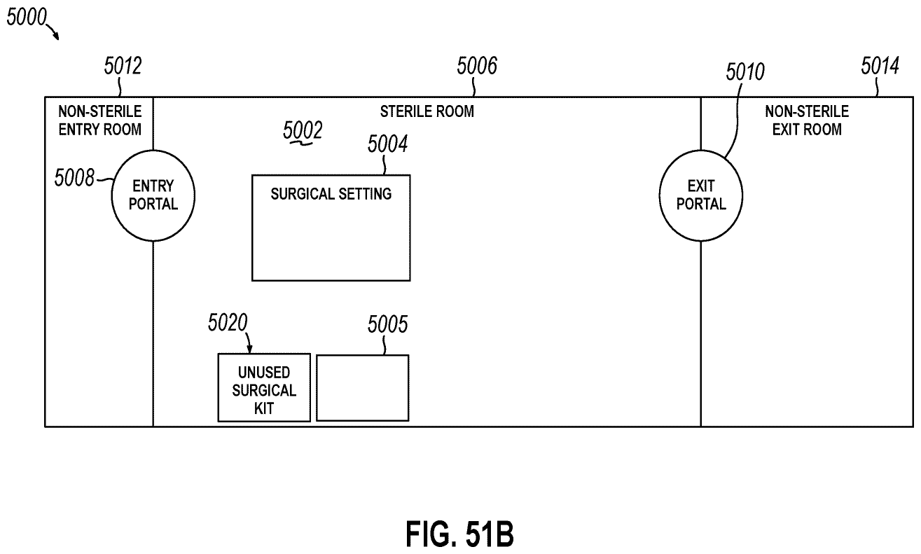

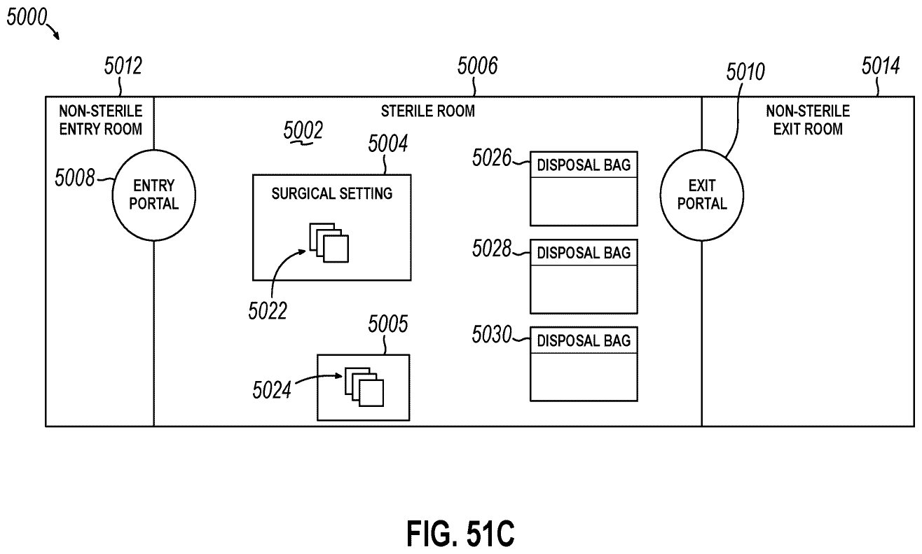

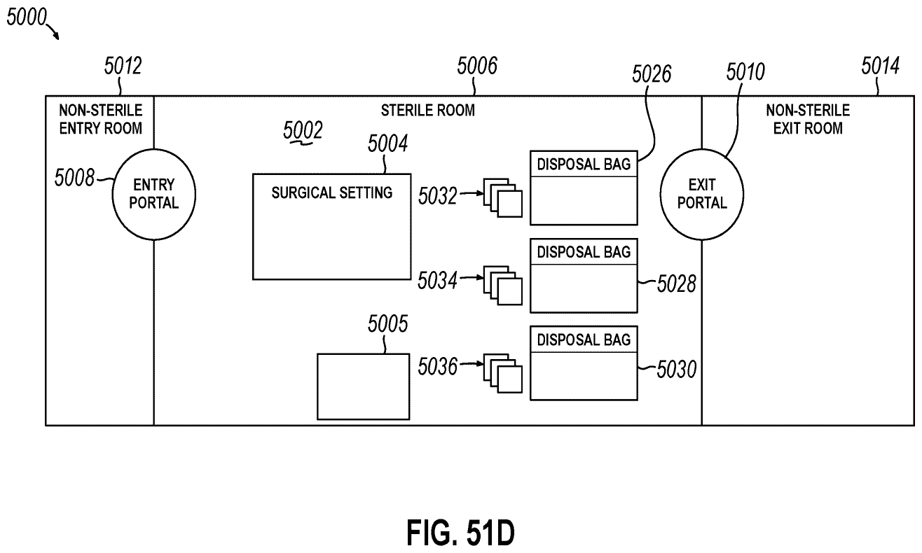

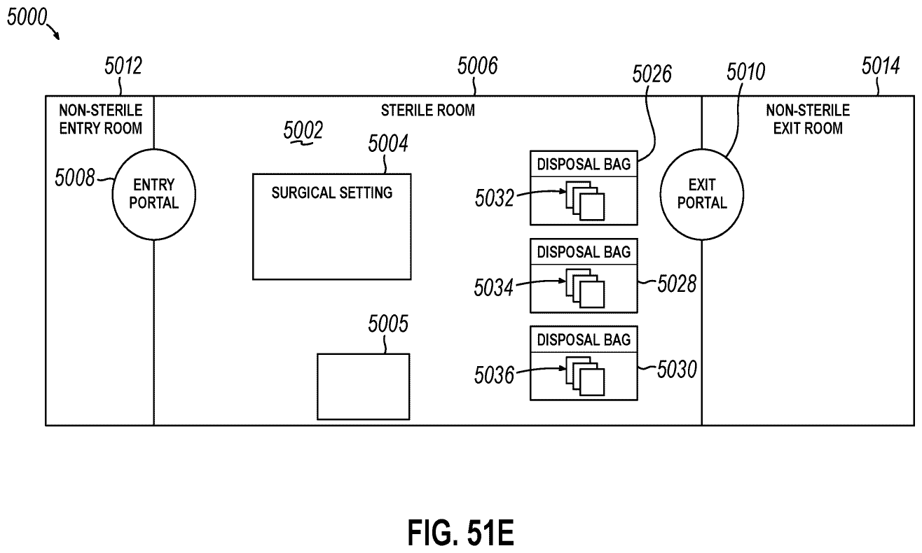

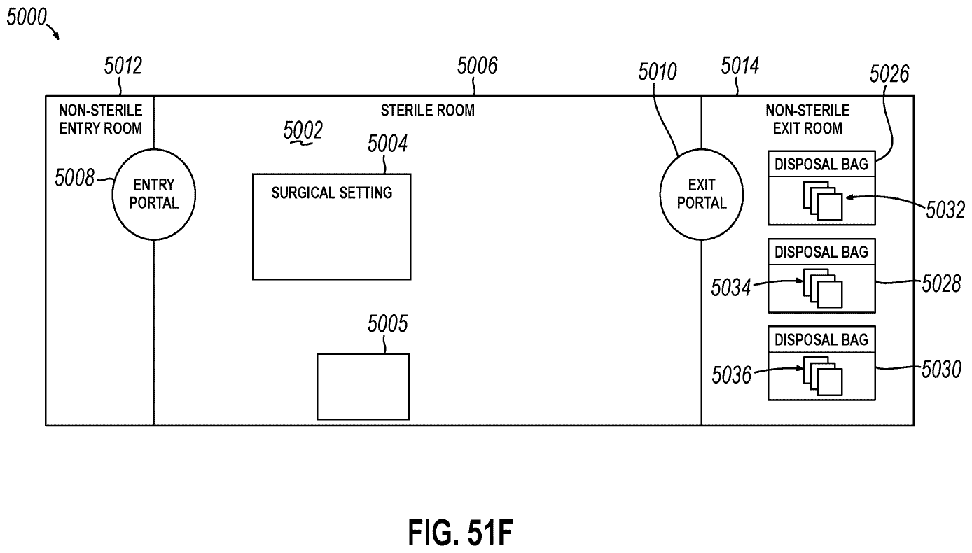

Therefore, in some instances, a surgical instrument/tool (112, 117, 152, 154, 156) may be introduced into the sterile field as a surgical kit with subcomponents requiring at least some degree of assembly prior to exemplary use. In addition to the subcomponents used to form surgical instrument/tool (112, 117, 152, 154, 156), such a surgical kit may contain the necessary tools for assembly, disassembly, and suitable processing for disposal, reuse, or remanufacturing of instrument/tool (112, 117, 152, 154, 156) and its subcomponents. FIGS. 51A-51F show an exemplary introduction, assembly, use, disassembly, and disposal of a surgical kit (5020).

Initially, as shown in FIG. 51A, prior to a surgical procedure, surgical kit (5020) may be contained within a sealed and sterilized interior of a storage bag (5025). Storage bag (5025) may have a non-sterile exterior such that non-sterilized persons may grasp the exterior of storage bag (5025) in order to transport surgical kit (5020) into non-sterile room (5012) in preparation for a surgical procedure as would be apparent to one skilled in the art in view of the teachings herein. Additionally, the sealed and sterilized nature of the interior of storage bag (5025) may help ensure surgical kit (5020) remains suitably sterilized while being transported within storage bag (5025).

In preparation for a surgical procedure, surgical kit (5020) may be transferred from non-sterile entry room (5012) into a sterile field (5006) of a surgical theater (5000), as shown in FIG. 51B. Surgical kit (5020) may be transferred into sterile field (5006) via a suitable entry portal (5008) as would be apparent to one skilled in the art in view of the teachings herein. During such a transfer, a person within non-sterile entry room (5012) may place kit (5020) and bag (5025) into entry portal (5008) and provide suitable access to, while maintaining the sterile nature of, the interior of bag (5025). Next, a sterilized person within sterile field (5006) may suitably access the sterilized interior of storage bag (5025) and remove surgical kit (5020) from storage bag (5025) such that kit (5020) is suitably transferred into both sterile room (5002) and sterile field (5006) as shown in FIG. 51B. Therefore, kit (5020) may remain suitably sterilized after being transferred from non-sterile entry room (5012) into surgical theater (5000).

Sterile room (5002) may be substantially similar to surgical operating room (116) described above. Sterile room (5002) includes a surgical setting (5004), which may be substantially similar to operating table (114) described above. Additionally, sterile room (5002) also includes an assembly station (5005), which may include a sterile table and/or any other suitable structures as would be apparent to one skilled in the art in view of the teachings herein.

Turning to FIG. 51C, once surgical kit (5020) is suitably transferred into sterile room (5002), the contents of surgical kit (5020) may be accessed and placed on assembly station (5005). In the current example, surgical kit (5020) includes surgical subcomponents (5022), non-surgical subcomponents (5024), and a plurality of disposal bags (5026, 5028, 5030). Surgical subcomponents (5022) are intended to be assembled in order to form a surgical instrument substantially similar to instrument/tool (112, 117, 152, 154, 156) described above. Non-surgical subcomponents (5024) may include portions of surgical kit (5020) that are not intended to be directly used in the surgical procedure, but for ancillary purposes to the surgical procedure. For example, non-surgical subcomponents (5024) may include tool(s) for assembling and disassembling surgical subcomponents (5022), a tray that may organize other components (5022, 5024, 5026, 5028, 5030), or other features as desired for exemplary use in accordance with the description herein. As will be described in greater detail below, disposal bags (5026, 5028, 5030) are configured to store respective post-surgery components (5032, 5034, 5036) (see FIG. 51D) such that post-surgery components (5032, 5034, 5036) may be removed from sterile room (5002) while being suitably sealed from exposure to a non-sterile environment (5012, 5014).

As also shown in FIG. 51C, after being suitably assembled (either pre-assembled, assembled by hand within sterile room (5002), and/or utilizing tools from non-surgical subcomponents (5024)), surgical subcomponents (5022) may be utilized in the surgical setting (5004) to perform a suitable surgical procedure on a patient as would be apparent to one skilled in the art in view of the teachings herein. Surgical subcomponents (5022) may form any suitable instrument/tool (112, 117, 152, 154, 156) described herein. Therefore, surgical subcomponents (5022) may be configured as a component of computer-implemented interactive surgical system (100). For example, in instances where surgical subcomponents (5022) form a removably coupled surgical tool (117) intended for use with patient side cart (120), surgical subcomponents (5022) may be used in conjunction with robotic system (110). As another example, in instances where surgical subcomponents (5022) form a handheld intelligent surgical instrument (112), surgical subcomponents (5022) may be handled by a surgeon and suitably coupled with hub (106) during exemplary use in accordance with the description herein.

Turning to FIG. 51D, after suitable use, surgical subcomponents (5022) may have been exposed to the patient such that subcomponents (5022) require special consideration. Therefore, surgical subcomponents (5022) and non-surgical subcomponents (5024) may be broken down and compartmentalized into post-surgery components (5032, 5034, 5036). Any suitable means of disassembling subcomponents (5022, 5024) may be utilized as would be apparent to one skilled in the art in view of the teachings herein. Post-surgery components (5032, 5034, 5036) may be categorized based on the type of post-surgery processing intended for each category. For example, broken down subcomponents (5022, 5024) intended to be reused or remanufactured may be sorted into a first category of post-surgery components (5032); while broken down subcomponents (5022, 5024) containing electrical components intended to be disposed of may be sorted into a second category of post-surgery components (5034); and broken down subcomponents (5022, 5024) that are intended to be disposed of, but do not contain electrical components, may be sorted into a third category of post-surgery components (5036). At the moment shown in FIG. 51D, certain disposable components may be further processed in preparation for being disposed of properly. For example, an internal power source (e.g., battery) utilized as a surgical subcomponent (5022) may be required to be fully discharged at this moment.

Next, as shown in FIG. 51E, while still in sterile room (5002), categorized post-surgery components (5032, 5034, 5036) may then be placed within a respective disposal bag (5026, 5028, 5030). Disposal bags (5026, 5028, 5030) may then be sealed such that post-surgery components (5032, 5034, 5036) are sealed from the external environment outside of respective bags (5026, 5028, 5030). Disposal bags (5026, 5028, 5030) may utilize any suitable sealing means as would be apparent to one skilled in the art in view of the teachings herein.

Next, as shown in FIG. 51D, post-surgery components (5032, 5034, 5036) and respective disposal bags (5026, 5028, 5030) may be transported out of room (5002) and sterile field (5006) into a non-sterile room (5014) via an exit portal (5010). Therefore, post-surgery components (5032, 5034, 5036) may then be transported to a suitable location for disposal, reprocessing, and/or remanufacturing while remaining sealed off from the external environment. In instances where components (5032, 5034, 5036) may have contacted the patient during a surgical procedure, such components (5032, 5034, 5036) will be inhibited from undesirable exposure in non-sterile rooms (5012, 5014).

While in the current example, a non-sterile entry room (5012) and a non-sterile exit room (5014) are used in conjunction with entry portal (5008) and exit portal (5010), this is merely optional. In some instances, surgical kit (5020) and post-surgery components (5032, 5034, 5036) are transported from/to the same non-sterile room (5012, 5014) and utilizing the same portal (5008, 5010).

While in the current example disposal bags (5026, 5028, 5030) are used, and suitably disposal structure configured to suitably support respective components (5032, 5034, 5036) while sealing such components (5032, 5034, 5036) from the external environment during transport may be used as would be apparent to one skilled in the art in view of the teachings herein.

### X. EXEMPLARY DEVICE AND METHOD FOR DETERMINING AND EXTRACTING DISPOSAL INSTRUCTIONS FOR A USED ENERGIZED SURGICAL INSTRUMENT

As mentioned above, after suitable use of surgical subcomponents (5022), surgical subcomponents (5022) (e.g., the portions of surgical kit (5020) used to form an energized surgical instrument/tool (112, 117, 152, 154, 156)) may be broken down into post-surgery components (5032, 5034, 5036) for purposes of disposal, reprocessing, and/or remanufacturing. As one example, such post-surgery components (5032, 5034, 5036) may be categorized as components (5032) intended to be reused/remanufactured, electrical components (5034) intended for disposal, and non-electrical components (5036) intended for disposal.

In some instances, there may be various standards for classifying which used surgical subcomponents (5022) may be reusable/manufacturable and which surgical subcomponents (5022) are disposable. Additionally, there may be various standards for suitably processing/disposing such reusable, manufacturable, and disposable surgical subcomponents; either within the sterile environment as surgical subcomponents (5022) are originally being disassembled, or outside the sterile environment while surgical subcomponents (5022) are being suitably reprocessed, remanufactured, and/or disposed of as would be apparent to one skilled in the art in view of the teachings herein. The standards and/or suggested guidelines for classifying subcomponents (5022) as either reusable or disposable may vary between jurisdictions. Additionally, the reprocessing, remanufacturing, and/or disposal standards and/or suggested guidelines may also vary between jurisdictions. Such standards/guidelines for classifying, reprocessing, remanufacturing, disposal, etc., of used surgical subcomponents (5022) may be referred to as disposal methodology.

For example, in a first jurisdiction, an energized component (e.g., electrodes, transducers, ultrasonic blades, batteries, etc.) may only be certified for a first number of surgical uses (such as one surgical procedure); while in a second jurisdiction, that same energized component may be certified for reprocessing and/or remanufacturing for a different number of surgical uses (such as multiple surgical procedures). As another example, in a first jurisdiction, an electrical component may require being completely discharged before being transferred to a respective disposal bag (5026, 5028, 5030); while in a second jurisdiction, the same electrical component may not require being completely discharged before being transferred to a respective disposal bag (5026, 5028, 5030).

Therefore, it may be desirable to retrieve a set of disposal instructions for processing the components of a surgical kit (5020) after an exemplary surgical procedure in accordance with the description herein. Further, it may be desirable to retrieve a specific set of disposal instructions for a surgical kit (5020) based any number of suitable variables that would be apparent to one skilled in the art in view of the teachings herein. For example, such variables may include, but are not limited to, the geographic location of the surgical procedure, the use of other surgical products in conjunction with surgical kit (5020), and/or performance data of surgical subcomponents (5022).

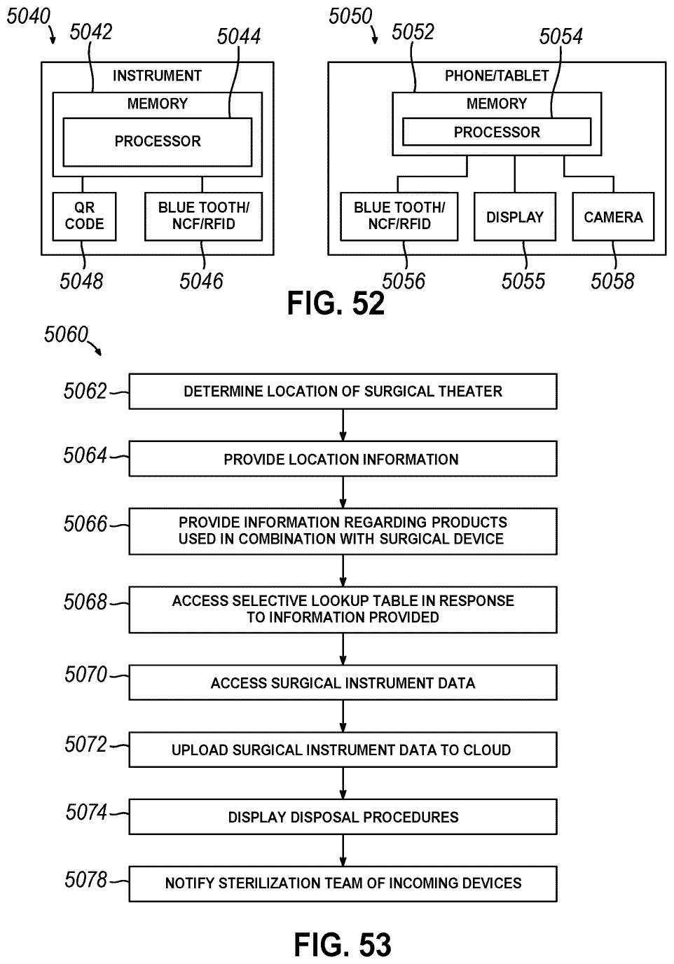

FIG. 52 shows an exemplary surgical instrument (5040) and an exemplary disposal assistance device (5050) that may be utilized in the determination procedure (5060) for disposal instructions shown in FIG. 53. It should be understood that while exemplary surgical instrument (5040) and disposal assistance device (5050) are utilized in the current example to perform determination procedure (5060) for disposal instructions as shown in FIG. 52, any suitable surgical instrument may be utilized, alone or in conjunction with any suitable disposal assistance device, as would be apparent to one skilled in the art in view of the teachings herein.

Surgical instrument (5040) may be assembled from a surgical kit (5020) such that surgical instrument (5040) is substantially similar to surgical subcomponents (5022) described above. Therefore, surgical instrument (5040) may be assembled within the surgical theater (5000) prior to being used in a surgical procedure, and then disassembled and categorized into various post-surgery components (5032, 5034, 5036). In some instances, such disassembly may occur within the surgical theater (500) after being used in a surgical procedure.

Surgical instrument (5040) may be substantially similar to any surgical instrument/tool described herein, including surgical instrument/tool (112, 117, 152, 154, 156) described above. Therefore, surgical instrument (5040) is configured to be used in conjunction with computer-implemented interactive surgical system (100) by coupling with suitable components of hub (106), such as generator module (140), communication module (130), storage array (134), etc. In some instances, surgical instrument (5040) may be handheld; while in other instances, surgical instrument (5040) may be configured to suitably couple with robotic system (110). In some instances, surgical instrument (5040) may be utilized without being connected to hub (1060), such that surgical instrument (5040) includes its own power source or such that surgical instrument (5040) is coupled to a generator other than one associated with hub (1060).

Surgical instrument (5040) includes a memory unit (5042) and a processing unit (5044); which may together function as a control unit for surgical instrument (5040). Memory unit (5042) may be operable to store various information related to the performance of surgical instrument (5040) during an exemplary surgical procedure in accordance with the description herein. Memory unit (5042) may also store data related to the various components used to form surgical instrument (5040). For example, memory unit (5042) of surgical instrument (5040) may store data related to what geographical location instrument (5040) was sold and sent to. Memory unit (5042) may include data containing the serial number identifying the specific instrument (5040). Surgical instrument (5040) may also include a QR code (5048). QR code (5048) may contain suitable data required to identify the specific instrument (5040) along with various suitable information related to the specific instrument (5040) (such as a geographical location in which instrument (5040) was sold and/or sent).

Instrument (5040) also includes a communication module (5046) in communication with processor (5044). Communication module (5046) is configured to establish communication with a corresponding communication module (5056) of disposal assistance device (5050) such that data may be shared between instrument (5040) and disposal assistance device (5050). Communication module (5046) may include any suitable communication means as would be apparent to one skilled in the art in view of the teachings herein. For example, communication module (5046) may be configured to communicate via Bluetooth technology, Near Field Communication “NCF”, Radio Frequency Identification “RFID”, etc.

Disposal assistance device (5050) may take any suitable form, such as a smart phone, a tablet, etc. Disposal assistance device (5050) may be used within the sterile field (5006) during disassembly of surgical subcomponents (5022) in accordance with the description herein. Additionally, or alternatively, disposal assistance device (5050) may be used outside the sterile field (5006) during reprocessing, remanufacturing, and/or disposal of surgical subcomponents (5022) in accordance with the description herein.

Disposal assistance device (5050) includes a memory unit (5052) and a processing unit (5054); which may together function as a control unit for disposal assistance device (5050). Memory unit (5052) may include a suitable application (e.g., software product) that includes download and uploading capabilities which may be used to access cleaning and sterilization protocols in accordance with the description herein. Disposal assistance device (5050) includes corresponding communication module (5056) configured to communicate with communication module (5046) described above. Communication module (5056) may include any suitable communication means as would be apparent to one skilled in the art in view of the teachings herein. For example, communication module (5056) may be configured to communicate via Bluetooth technology, Near Field Communication “NCF”, Radio Frequency Identification “RFID”, etc.

Disposal assistance device (5050) also includes a display (5055) in communication with processing unit (5054). Processing unit (5054) may instruct display (5055) to show suitable information to a user, such as information provided by running suitable applications described herein. Display (5055) may take any suitable form as would be apparent to one skilled in the art in view of the teachings herein. For example, display (5055) may be a touch screen. Disposal assistance device (5050) also includes a camera (5058) in communication with processing unit (5054). Camera (5058) may be utilized to capture and communicate images to processing unit (5054) such that suitable applications may utilize images in accordance with the description herein. Camera (5058) may include any suitable components as would be apparent to one skilled in the art in view of the teachings herein.

In some instances, the application running disposal assistance device (5050) may be configured to connect to a hub (106) that was used in conjunction with instrument (5040) during a surgical procedure. Such an application may have limited permissions with hub (106) such that the staff utilizing disposal assistance device (5050) and the corresponding application do not have access to any portion of hub (106) aside from relevant disposal instructions, cleaning, and/or sterilization information. Such connection between hub (106) and the application of device (5050) may allow device (5050) to communicate with interconnected hospital systems for operating room system/inventory tracking, etc. Therefore, once instrument (5040) or other suitable instruments/components are used in a surgery and scanned by device (5050), hub (106) may update the inventory of the hospital system to recognize use of instruments (5040) and/or other components. It should be understood that being “scanned” by device (5050) may include device (5050) utilizing camera (5056) to scan QR code (5048); establishing a connection between communication modules (5046, 5056); or any other suitable form of device (5050) obtaining data from instrument (5040) as would be apparent to one skilled in the art in view of the teachings herein.

In instances where disposal assistance device (5050) is utilized within surgical theater (5000), the application being utilized by disposal assistance device (5050) may communicate with a sterilization group located outside surgical theater (5000). Therefore, as disposal assistance device (5050) scans instrument (5040) and/or various subcomponents of instrument (5040) in order to obtain suitable reprocessing/disposal instructions in accordance with the description herein, the application running on device (5050) may inform the sterilization group that suitable portions of instrument (5040) intended for sterilization will be transported to the sterilization group.

Similarly, the application being utilized by disposal assistance device (5050) may communicate with a maintenance group to notify the maintenance group when equipment is ready for service. Therefore, as disposal assistance device (5050) scans instrument (5040) and/or various subcomponents of instrument (5040) in order to obtain suitable reprocessing/disposal instructions in accordance with the description herein, the application running on device (5050) may inform the maintenance group that suitable portions of instrument (5040) intended for maintenance are ready for service.

In instances where disposal assistance device (5050) is in communication with hub (106), disposal assistance device (5050) and hub (106) may be configured to confirm any lost or missing devices, such as instrument (5040), used during a procedure. For example, hub (106) may be configured to track what instruments (5040) or other devices are used during a procedure. During the disposal process, instruments (5040) used in the procedure may be scanned or otherwise processed utilizing disposal assistance device (5050). Communication between device (5050) and hub (106) may allow disposal assistance device (5050) and/or hub (106) to compare which instruments (5040) were used during a procedure and which instruments (5040) were scanned for suitable reprocessing/disposal. This comparison allows device (5050) and/or hub (106) to ensure all devices have been accounted for. In instances where a smart disposal system is used, disposal assistance device (5050) may confirm proper disposal via communication with a smart disposal system.

In some instances, the application running on disposal assistance device (5050), may connect with either remote server (113) and/or cloud (104) instead of hub (106). As one example, some hospital systems may not have access to a hub (106). In such instances, cloud (104) and/or remote service (113) may coordinate with the suitable application on device (5050) to provide cleaning, sterilization, and/or disposal protocols and provide step-by-step instruction in accordance with the teachings herein. In examples where application connects with either remote server (113) and/or cloud (104), such an application may auto-connect disposal assistance device (5050) with a suitable manufacturer call center for help.

In some instances, the application running on device (5050) may utilize location information to determine the country and/or region in which device (5050) is being used. For example, the application running on device (5050) may have application location services that determine the location of use by utilizing any suitable means as would be apparent to one skilled in the art in view of the teachings herein. In some instances, the application running on device (5050) may request the user to input a location of use when setting up the application account on device (5050).

In some instances, instrument (5040) may not be connected to hub (106) during a surgical procedure such that hub (106) does not collect performance data of surgical instrument (5040) during exemplary use. As mentioned above, memory (5042) of instruments (5040) may be operable to store various information related to the performance of surgical instrument (5040) during an exemplary surgical procedure in accordance with the description herein. For example, such information may include device motor data, sensor data, failures experienced by instrument (5040) during use, error codes, etc. In some instances, the application running on device (5050) may be configured to extract performance data stored on memory (5042) of instrument (5040) via communication modules (5046, 5056). Application running on device (5050) may then be configured to upload the extracted performance data onto hub (106), cloud (104), remote server (113), etc. In instances where no hub (106) is used, a gateway to device (5050) may be utilized to extract performance data. This extraction and uploading of data could be invisible to the operator utilizing device (5050) and could occur automatically once device (5050) establishes communication with instrument (5040) via communication modules (5046, 5056).

FIG. 53 shows a determination procedure (5060) that is configured to allow instrument (5040), generator module (140), and/or hub (106) to identify relevant factors of instrument (5040) usage and then access or determine a suitable disposal methodology based on the identified relevant factors. Determination procedure (5060) may be utilized with any suitable equipment as would be apparent to one skilled in the art in view of the teachings herein. For example, determination procedure (5060) may be utilized in conjunction with instrument (5040) and/or disposal assistance device (5050) described above. As another example, determination procedure (5060) may be utilized in conjunction with instrument (5040) and hub (106). As yet another example, determination procedure (5060) may be utilized in conjunction with instrument (5040) hub (106), and disposal assistance device (5050). While determination procedure (5060) is shown and described in a particular order of steps, it should be understood that some steps may be entirely optional. Further, it should be understood that the order in which steps are performed may have any suitable order as would be apparent to one skilled in the art in view of the teachings herein.

As mentioned above, standards and/or guidelines for disposal methodology may deviate depending on the location which instrument (5040) is used. Therefore, one step (5062) in the determination procedure (5060) may include determining the location of the surgical theater (5000) in which the procedure is being performed. Any suitable components or means may be utilized in order to determine the location of the surgical theater (5000) as would be apparent to one skilled in the art in view of the teachings herein. For example, the application running on disposal assistance device (5050) may be configured to determine the country and/or region in which device (5050) is being used; which may then be used to infer the location of the surgical theater (5000).

As another example, instrument (5040) and/or generator module (140) may have a device code and/or serial number that contains or provides access to the specified location to which instrument (5040) was shipped to for intended use in accordance with the description herein. In instances where the device code and/or serial number is associated with instrument (5040), when instrument (5040) couples with generator module (140)/hub (106)/cloud (104), generator module (140)/hub (106)/cloud (104) may read the device code provided by instrument (5040) in order to determine the location of intended use. In some instances, generator (140) may be pre-programmed by the manufacturer with the intended location in which generator (140) is to be shipped; such as utilizing program EEPROM code. In such instances, generator (140) may determine the location of use by referencing this pre-programmed information during exemplary use. Further, when generator (140) is activated, generator (140) may perform an initialization check in order to identify location of use. Such location of use may be stored in the field service settings.

As another example, rather than a device code and/or serial number, generator module (140) and/or instrument (5040) may include another identifier, such and an RFID chip that would be able to track the sale of the product to determine its location of intended use. In some instances, the location of intended use, as determined by the manufacturer, may be cross checked with a suitable means of identifying actual use (some examples of identifying actual use are provided below). In instances where the location of intended use does not match with the identified location of actual use, hub (106) and/or cloud (104) may be notified to allow special processing of the device tracking by the manufacturer.

As another example, a GPS device may be associated with instrument (5040), device (5050), hub (106), generator module (140), or any other suitable device that would be apparent to one skilled in the art in view of the teachings herein. Once activated, the GPS device may determine the location of use and communicate that information to any suitable component, such as hub (106), generator module (140), device (5050), and/or instrument (5040). As another example, hub (106) and/or device (5050) may analyze the network in which they are connected to in order to determine the location of use. As another example, generator, hub (106) and/or device (5050) may have a hospital identifier that may determine the location of use. As another example, hub (106) and/or device (5050) may determine location of use by connecting to remote server (113), and/or cloud (104). After connecting to the remote server (113) and/or cloud (104), hub (106) and/or device (5050) may access manufacturer-generated regional codes that specify a location of use. As another example, hub (106), device (5050), and/or any other suitable component of surgical system (100) may provide an interface allowing a user to select their region of use. As another example, hub (106) and/or device (5050) may utilize its associated IP address to determine the location of use. As another example, hub (106) and/or device (5050) may utilize its software license to determine the location of use.

After suitably determining the location of use (5062), instrument (5040), hub (106), and/or device (5050) may then provide location information (5064) to the suitable applications or devices that are used in determining and/or accessing the suitable disposal methodology. The provided location information (5064) may be utilized by disposal assistance device (5050), remote server (113), cloud (104) and/or hub (106). It should be understood that providing location information (5064) may occur at any suitable time during determination procedure (5060) as would be apparent to one skilled in the art in view of the teachings herein. For example, providing location information (5064) may occur simultaneously with other steps in determination procedure (5060). Information may be provided automatically or in response to a specific action; such as device (5050) scanning instrument (5040) or communicating with instrument (5040) via communication modules (5046, 5056).

During a surgical procedure in accordance with the description herein, instrument (5040) and/or hub (106) may track if any other products were used in combination with instrument (5040). For example, instrument (5040) and/or hub (106) may track if any drugs, controlled substances, adjuncts, or any other suitable medical products as would be apparent to one skilled in the art in view of the teachings herein. Use of such medical products may affect the standards and/or guidelines for disposal methodology used for processing instrument (5040) after exemplary use. Therefore, one step (5066) in the determination procedure (5060) may include providing information regarding the various relevant medical products used in combination with instrument (5040). Instrument (5040), hub (106), and/or device (5050) may then provide such medical product information (5066) to the suitable applications or devices that are used in determining and/or accessing the suitable disposal methodology. For example, the provided medical product information (5066) may be utilized by disposal assistance device (5050), remote server (113), cloud (104) and/or hub (106). Information may be provided automatically or in response to a specific action; such as device (5050) scanning instrument (5040) or communicating with instrument (5040) via communication modules (5046, 5056).

While in the current example, location information (5064) and relevant medical product information (5066) is provided to help determine standards and/or guidelines for the disposal methodology in some instances just the location information (5064), or just the medical product information (5066) may be utilized to help determine standards and/or guidelines for disposal methodology. Additionally, any other suitable information may also be provided as well, in combination with the other parameters or alone. For example, performance data accumulated during use of surgical instrument (5040) that is accessed (5070) and uploaded (5072) in accordance with the description herein may be provided to the suitable applications or devices that are used in determining and/or accessing the suitable disposal methodology.

Hub (106) and/or disposal assistance device (5050) may access a selective lookup table (5068) that includes the various local disposal methodologies. Utilizing the suitable information provided (5064, 5066, 5070, 5072), hub (106) and/or disposal assistance device (5050) selects the suitable local disposal methodology from the various methodologies provided on the selective lookup table. The various standards and/or guidelines for local disposal methodology may be stored on any suitable device as would be apparent to one skilled in the art in view of the teachings herein. For example, lookup tables may be stored on cloud (104), remote server (113), hub (106), disposal assistance device (5050), etc. Accessing the selective lookup table (5068) may occur automatically or in response to a specific action; such as device (5050) scanning instrument (5040) or communicating with instrument (5040) via communication modules (5046, 5056).

As mentioned above, instrument (5040) may store performance data on memory (5042). In some examples, as also mentioned above, when instrument (5040) is scanned by disposal assistance device (5050) or communication between instrument (5040) and device (5050) is established via communication module (5046, 5056), such information may be accessed (5070) and uploaded (5072) to cloud (104). Cloud (104) may store uploaded performance data for various purposes. In some instances, uploading performance data to cloud (104) via communication between instrument (5040) and device (5050) may be beneficial when a hub (106) is not utilized during the surgical procedure.

With the proper standards and/or guidelines for local disposal methodology accessed, determination procedure (5060) may then display (5074) the local disposal procedure to the staff in charge of performing the disposal methodology. The disposal procedure may include displaying chronological instructions of the disposal methodology. Such instructions may include how to disassemble instrument (5040); what components of instrument (5040) are reusable and which components of instrument (5040) are disposable; how to properly prepare such components for disposal or reuse; how to properly store such components for disposal or reuse; etc. Such a display (5074) may be shown on device (5050) or suitable components of hub (106). Display (5074) may allow a user to scroll through the chronological instructions of the disposal methodology and indicate when each instruction is accomplished.

While the staff performs the disposal methodology in accordance with the displayed standards and/or guidelines displayed, hub (106) and/or device (5050) may notify sterilization team of incoming device (5078) expected to be transported to the sterilization team in light of the displayed disposal methodology. It should be understood that in instances where the sterilization staff is utilizing determination procedure (5060), such a notification (5078) may be omitted.

### XI. EXEMPLARY GENERATOR STORAGE BIN WITH REUSABLE CORD AND STERILIZATION MEANS

In some instances, surgical kit (5020) described above comes with a power cord configured to electrically couple surgical subcomponents (5022) to generator module (140) such that generator module (140) may electrically power surgical subcomponents (5022) during exemplary use in accordance with the description herein. Cords may also enable data to communicate between surgical subcomponents (5022) and hub (106). In some instances, it may be desirable to have at least a portion of cord be reusable with generator module (140), rather than be associated with surgical kit (5020). Therefore, after a procedure, the portion of cord associated with generator module (140) may remain associated with generator module (140), and the portion of cord associated with surgical kit (5020) may be categorized as a post-surgery component (5032, 5034, 5036) for reuse or disposal as would be apparent to one skilled in the art in view of the teachings herein.

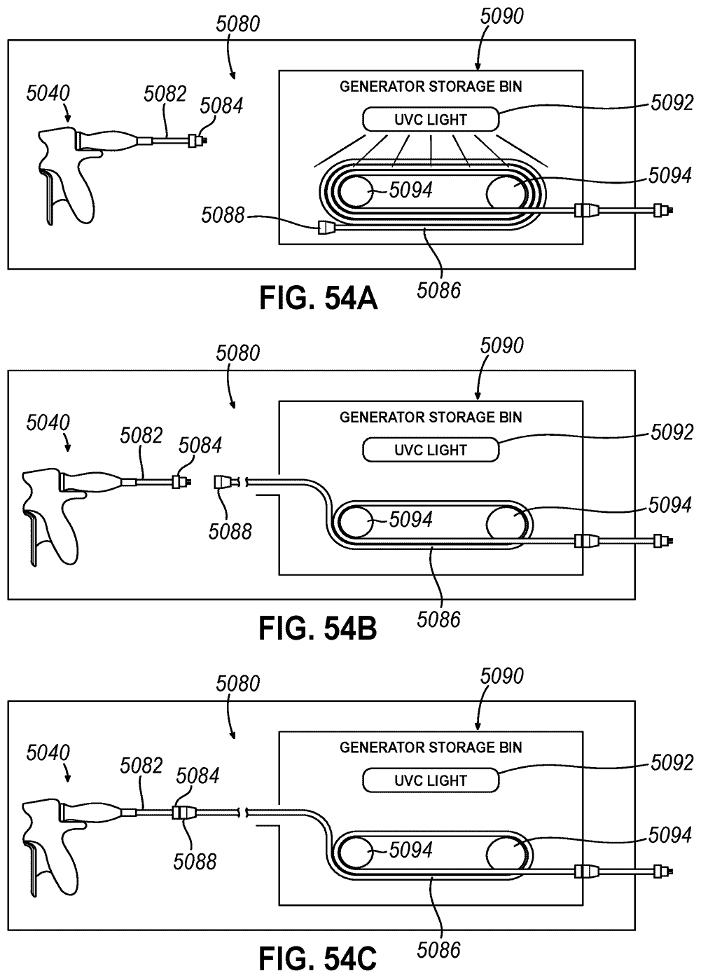

FIGS. 54A-54C show an exemplary power coupling assembly (5080) and generator storage bin (5090) that may be readily incorporated into surgical kit (5020) and generator module (140). Power coupling assembly (5080) includes a short cord (5082) and a long cord (5086). Short cord (5086) may be associated with instrument (5040) formed by surgical kit (5020) such that short cord (5082) comes packaged with surgical kit (5020). Short cord (5082) contains a coupling member (5084) configured to selectively establish communication with long cord (5086) and generator module (140) via coupling member of long cord (5088). When short cord (5082) is coupled with long cord (5086), generator module (140) may actively power instrument (5040) in accordance with the description herein.

Long cord (5086) is attached to generator module (140) and is wrapped and stored internally within generator storage bin (5090) of generator module (140) via couplings posts (5094). Since long cord (5086) remains associated with generator module (140) and is configured to couple with short cord (5082), long cord (5086) may reduce the amount of disposal from surgical kit (5020). In some instances, no short cord (5082) is present such that long cord (5086) is coupled directly with instrument (5040). In some instances, device contains a detachable, reusable power cord with a connection junction positioned at the end of handpiece. The connection can be severed with a physical unique key that may require multiple actions. The connection can alternatively be severed by an electronically activated release mechanism that could be initiated via the user or the generator module (140).

Storage bin (5090) may include a pop top with a bin for cord storage and multi-tool storage. Rather than posts (5094), storage bin (5090) may have a hook with cord (5086) wrapped around hook.

Storage bin (5090) includes a UV light (5092). UV light (5092) may be utilized to sterilize long cord (5086) after exemplary use and while long cord (5086) is stored within storage bin (5090). FIGS. 54A-54C show an exemplary coupling of cords (5082, 5086). First, as shown in FIG. 54A, long cord (5086) and coupling member (5088) is within storage bin (5090). Next, coupling member (5088) may be removed from storage bin (5090), as shown in FIG. 54B. Finally, coupling members (5084, 5088) may be coupled to power instrument (5040) as shown in FIG. 54C.

### XII. EXEMPLARY SURGICAL KITS AND METHOD OF PROCESSING A MEDICAL DEVICE

In some instances, it may be desirable to reclaim a portion of a surgical instrument (e.g., surgical instruments (152, 154, 156)) to reduce medical waste, which may reduce the environmental impact and the associated cost. It may be beneficial to separate various components of surgical instrument (152, 154, 156) for added efficiencies. For example, it may be beneficial to process different components differently (e.g., send different components to different locations). For components that are not capable of being reclaimed, it may be desirable to dispose of the component in an environmentally friendly manner. To assist the personnel of a medical facility (e.g., a hospital), it is desirable to make the reclamation process and disposal process as streamlined and straightforward as possible. This may improve the process workflow within an operating room of the medical facility.

### A. First Exemplary Surgical Kit

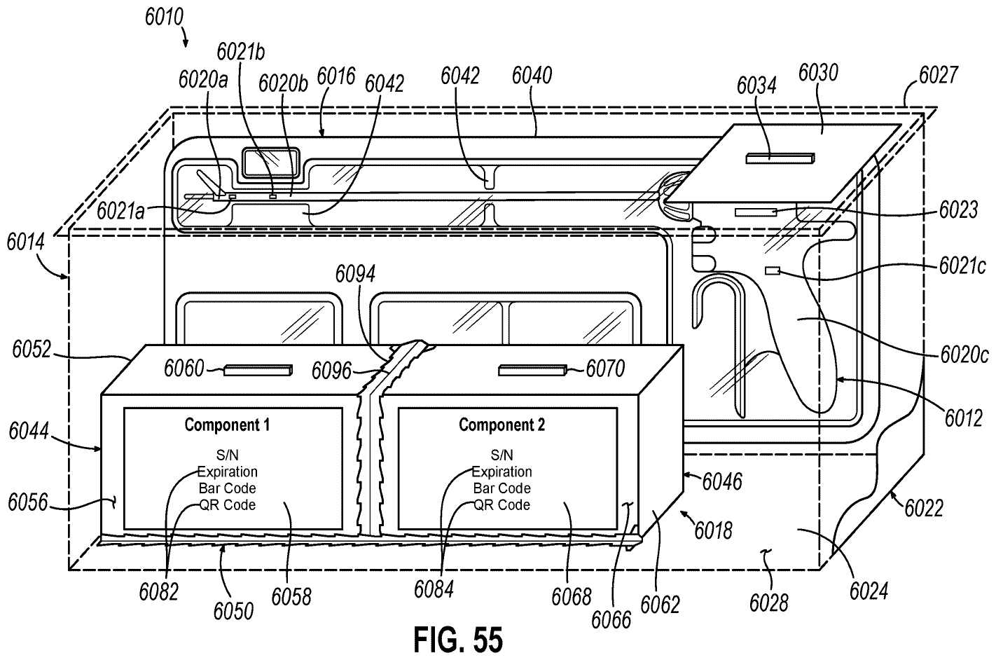

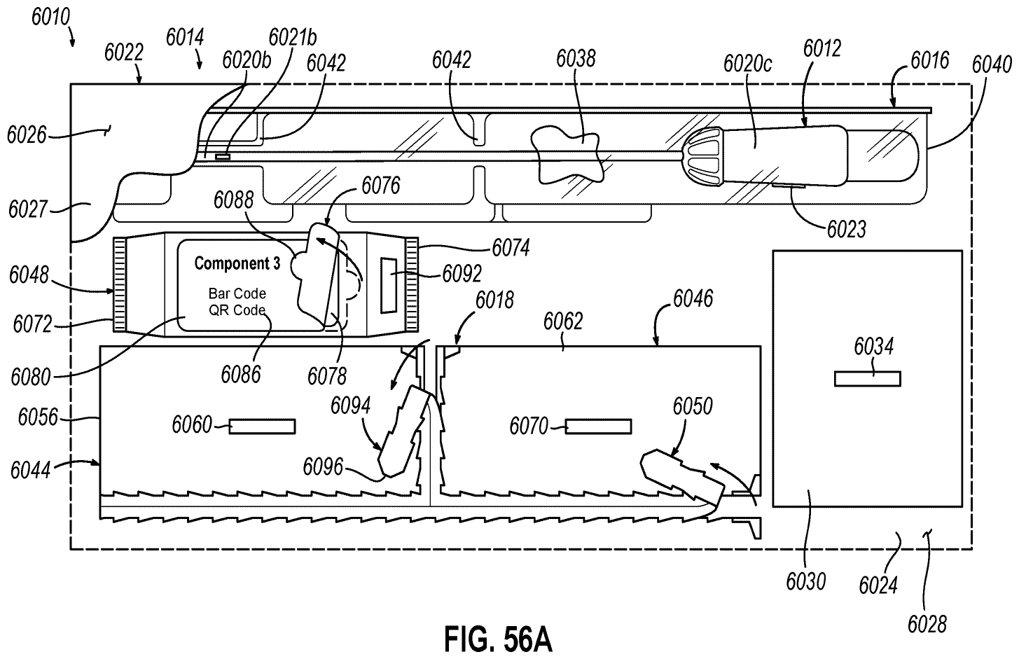

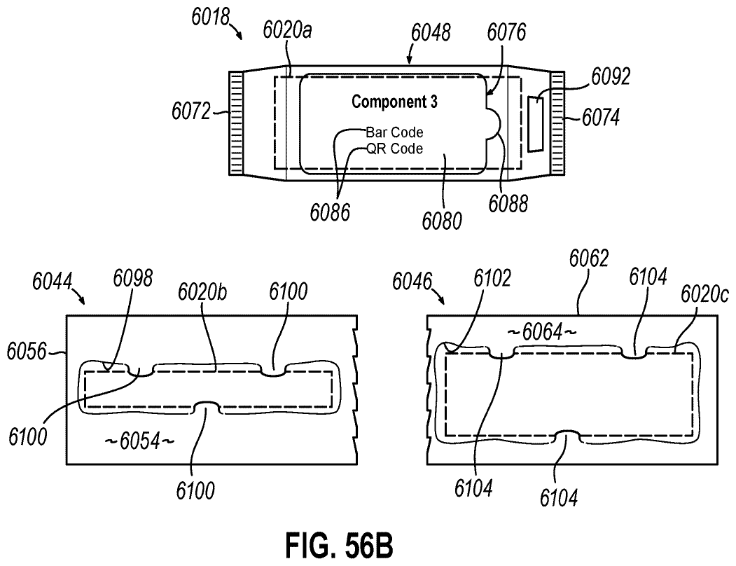

FIGS. 55-56A show a first exemplary surgical kit (6010) including a surgical instrument (6012), an outer packaging (6014), a sterile packaging (6016), and a return packaging (6018). Surgical kit (6010) may provide initial sterile delivery of surgical instrument (6012) and also packaging for reclamation or disposal of surgical instrument (6012). In some versions, sterile packaging (6016), or even outer packaging (6014), may be reclaimed to minimize contamination during return transport and handling.

Surgical instrument (6012) may be similar to surgical instruments (152, 154, 156) shown in FIG. 5. For example, surgical instrument (6012) may be similar to ultrasonic surgical instrument (152), RF electrosurgical instrument (154), combination ultrasonic/RF electrosurgical instrument (156), or a surgical stapler (not shown). Surgical instrument (6012) may be attachable to a surgical arm (123) of robotic system (110) or may be similar to handheld intelligent surgical instrument (112). Surgical instrument (6012) includes a plurality of portions, shown as first, second, and third portions (6020a-c), configured to separate from each other. As used herein, portions may refer to different locations of the same component or entirely different components. While first portion (6020a) is shown as including the end effector, second portion (6020b) is shown as including the shaft, and third portion is shown as including the handle, these are not exclusive. First, second, and third portions (6020a-c) may refer to a variety of different portions of surgical instrument (6012). While first, second, and third portions (6020a-c) are shown, more or fewer portions are envisioned. It is also envisioned that certain portions of surgical instrument (6012) may still be disposed of as medical waste, while other portions are reclaimed for subsequent use in a surgical instrument.

First, second, and third portions (6020a-b) may separate using a variety of different connection structures (e.g., mechanical, electrical, and/or magnetic connection structures). In some versions, first, second, and third portions (6020a-c) of surgical instrument (6012) may be destructively disassembled (e.g., using a laser or a breaking tool) after use of surgical instrument (6012). For example, breaking tool (not shown) may break first and second portions (6020a-b) of surgical instrument (6012) along a predetermined break point. As shown, first, second, or third portions (6020a-c) of surgical instrument (6012) each include a machine readable tag (6021a-c). As used herein, a machine readable tag is intended to encompass a physical chip (e.g., an RFID chip) as well as an optical code (e.g., a QR code or bar code). As shown, surgical instrument (6012) includes a master machine readable tag (6023), which may be a separate machine readable tag from machine readable tags (6021a-c). However, one of machine readable tags (6021a-c) may serve as master machine readable tag (6023). Master machine readable tag (6023) may include information (e.g., the serial number, production date, model number, etc.) pertaining to an entirety of surgical instrument (6012) and/or of specific included portions (e.g., first, second, and third portions (6020a-c)).

Outer packaging (6014) includes a body (6022) and an optional lid (6027) that define an interior (6024) and an exterior (6026). In the closed configuration of FIG. 55, interior (6024) is completely enclosed. Outer packaging (6014) moves from the closed configuration of FIG. 55 to the open configuration of FIG. 56A when outer packaging (6014) is opened up. In the open configuration, the user may remove sterile packaging (6016) containing surgical instrument (6012). In other words, outer packaging (6014) is opened by the user to expose interior (6024) in the open configuration to allow for access of surgical instrument (6012) for use in the surgical procedure. Interior (6024) of outer packaging (6014) is collectively formed from inner surfaces (6028) of body (6022) and lid (6027).

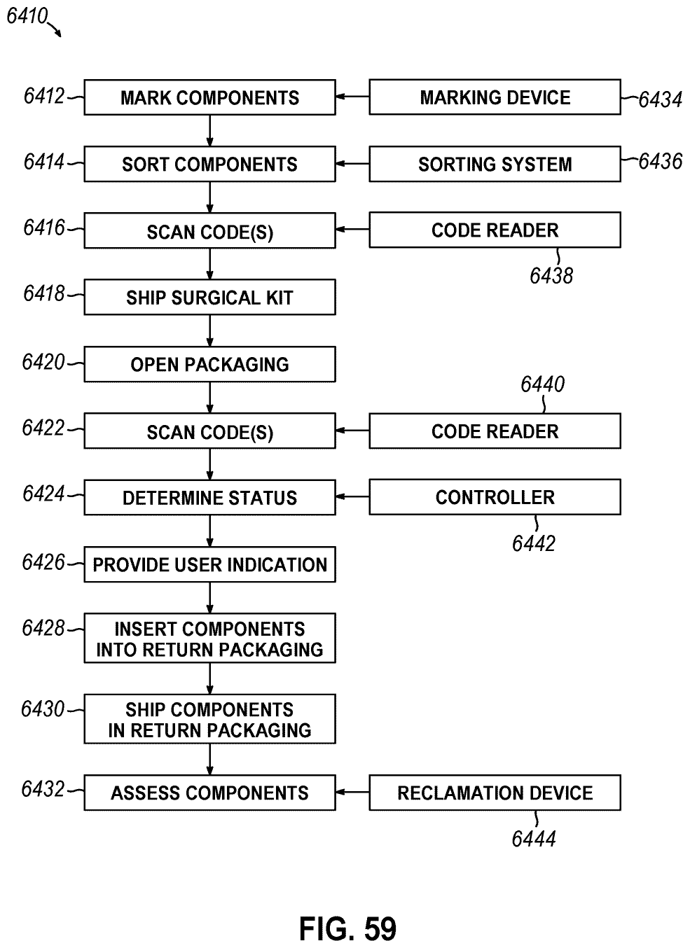

As shown in FIG. 55, inner surface (6028) of lid (6027) includes a repositionable label (6030) configured to couple with return packaging (6018). Outer packaging (6014) may include a signal blocking feature layer to prevent machine readable tag (6021a-c, 6023, 6034) from being read by code reader (6438, 6440) (see FIG. 59) when outer packaging (6014) is in the closed configuration. A user may simply remove repositionable label (6030) from lid (6027) of outer packaging (6014) and place repositionable label (6030) on return packaging (6018) which may reduce the time for user to locate, remove, and position repositionable label (6030). Alternatively, repositionable label (6030) may be disposed freely within interior (6024) of outer packaging (6014).

Sterile packaging (6016) is configured to surround surgical instrument (6012). Sterile packaging (6016) includes a body (6036) that defines an interior (6038) (see FIG. 56A) and an exterior (6040). Sterile packaging (6016) is disposed within interior (6024) of outer packaging (6014) in the closed configuration. In some versions, outer packaging (6014) may be omitted, such that return packaging (6018) is coupled with sterile packaging (6016) or is disposed within interior (6038) of sterile packaging (6016). As shown, sterile packaging (6016) includes recessed portions (6042) that complement the size and/or shape of the components.

Return packaging (6018) is configured to receive at least one of first portion (6020a), second portion (6020b), or third portion (6020c) of surgical instrument (6012) after the surgical procedure. Return packaging (6018) may be disposed completely within interior (6024) of outer packaging (6014) in the closed configuration. Optionally, return packaging (6018) may be coupled with outer packaging (6014), coupled with sterile packaging (6016), and/or disposed within interior (6038) of sterile packaging (6016) in the closed configuration. Return packaging (6018) may connect to a specific location of surgical instrument (6012) to improve disassembly. As shown in FIG. 55, return packaging (6018) may be coupled with outer packaging (6014) using a frangible portion (6050) configured to be severed by a user. In some versions, frangible portion (6050) may include a piece of string, which, when pulled, weakens scores frangible portion (6050).

Return packaging (6018) may be segmented to contain separate portions of reclaimed components. Return packaging (6018) may include a plurality of individual return packages. As shown in FIG. 56A, the plurality of individual return packages include first, second, and third return packages (6044, 6046, 6048). However, more or fewer return packages (6044, 6046, 6048) are also envisioned. First return package (6044) includes a body (6052) that defines an interior (6054) (see FIG. 56B) and an exterior (6056). Exterior (6056) includes a repositionable label (6058) and a machine readable tag (6060). Similarly, second return package (6046) includes a body (6062) that defines an interior (6064) (see FIG. 56B) and an exterior (6066). Exterior (6066) includes a repositionable label (6068) and a machine readable tag (6070). Third return package (6048) is shown as a bag with opposing first and second ends (6072, 6074) being scaled. Third return package (6048) includes a resealable flap (6076) configured to allow the user to insert third portion (6020c) into an interior (6078). Resealable flap (6076) may include a label (6080).

Each machine readable tag (6060, 6070, 6092) is coupled with at least one of surgical instrument (6012), outer packaging (6014), or return packaging (6018). Code readers (6434, 6436) (see FIG. 59) are configured to read machine readable tag (6060, 6070, 6092) from surgical instrument (6012), outer packaging (6014), or return packaging (6018). For example, return packaging (6018) may include a machine readable tag (6060, 6070, 6092) configured to identify surgical instrument (6012) using code readers (6434, 6436). For example, first, second, and third return packages (6044, 6046, 6048) may also include machine readable tags (6060, 6070, 6092) readable by code readers (6438, 6440). Machine readable tags (6060, 6070, 6092) may be used of in addition to or instead of machine readable tags (6021a-c, 6023, 6034).

Repositionable labels (6058, 6068) and label (6080) may include an optical code, such as a bar code or a QR code, that is optically readable by code readers (6438, 6440). Repositionable labels (6058, 6068) and label (6080) may include indicia (6082, 6084, 6086) to identify surgical instrument (6012) or first portion (6020a), second portion (6020b), or third portion (6020c) of surgical instrument (6012). For example, indicia (6082, 6084, 6086) may include at least one of a serial number, a model number, and/or an expiration date. Other indicia (6082, 6084, 6086) is also envisioned. For third return package (6048), label (6080) may be attached at one end of resealable flap (6076) and be closable by coupling terminal end (6088) of resealable flap (6076) to third return package (6048).

In some versions, first return package (6044) may be initially coupled with second return package (6046). As shown in FIGS. 55 and 56A, first and second return packages (6044, 6046) are coupled using a frangible portion (6094). Frangible portion (6094) may be manually severed by a user to maintain sterility. In some versions, frangible portion (6094) may include a piece of string (6096), which, once pulled, weakens and scores frangible portion (6094) along a break line. As shown, third return package (6048) is disposed within interior (6024) of outer packaging (6014), and is not coupled with first and second return packages (6044, 6046).

As shown in FIG. 56B, first return package (6044) portion is configured to receive first portion (6020a) of surgical instrument (6012). First return package (6044) includes a recessed portion (6098) and at least one projection, shown as projections (6100). Projections (6100) are configured to positively engage first portion (6020a) of surgical instrument (6012). Similarly, second return package (6046) includes a recessed portion (6102) and at least one projection, shown as projections (6104). Projections (6104) are configured to positively engage second portion (6020b) of surgical instrument (6012). Projections (6100, 6104) may be integrally formed with respective return packages (6044, 6046), or projections (6100, 6104) may be a separate component coupled with respective return packages (6044, 6046). Third return package (6048) is configured to receive third portion (6020c) of surgical instrument (6012). Recessed portions (6098, 6102) of return packaging (6018) may serve a variety of functions. For example, recessed portions (6098, 6102) may provide rigidity to return packaging (6018). Recessed portions (6098, 6102) and projections (6100, 6104) may be shaped and sized to retain first and second portions (6020a-b) upon disassembly of surgical instrument (6012) after the surgical procedure. Recessed portions (6098, 6102) and projections (6100, 6104) may improve the ability to separate first and second portions (6020a-b) in a controlled manner.

### B. Second Exemplary Surgical Kit

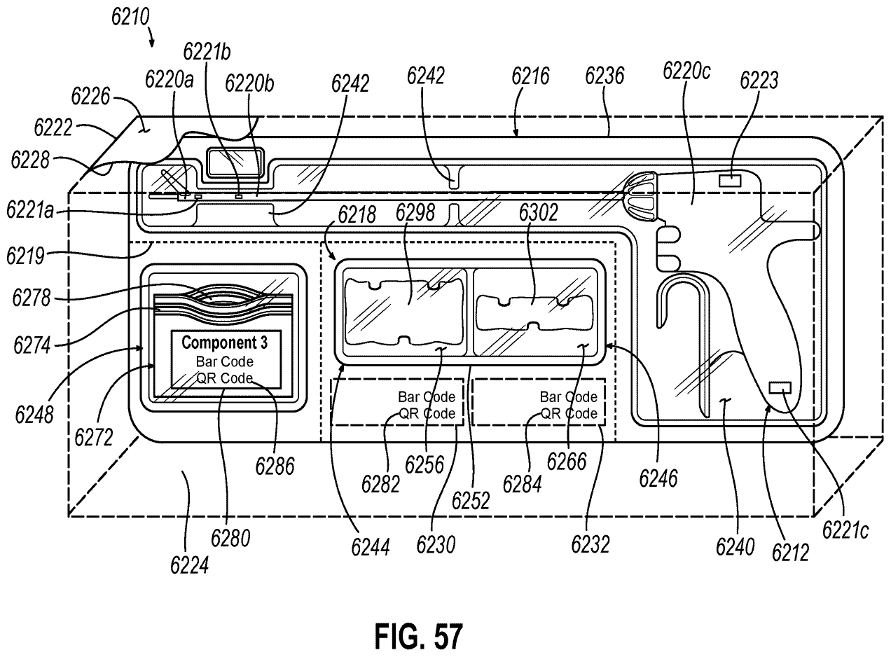

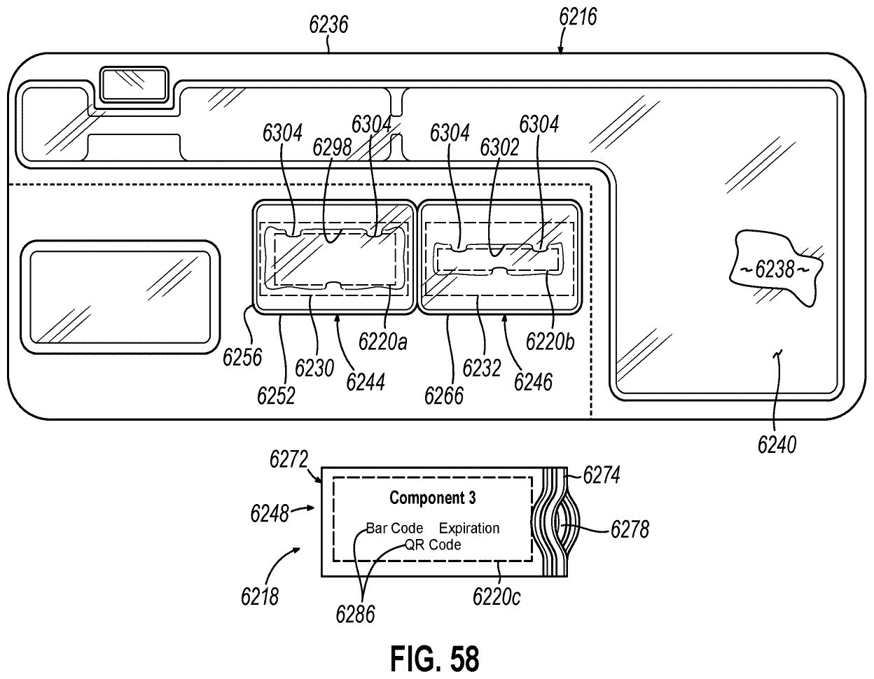

FIG. 57 shows a second exemplary surgical kit (6210) that includes a surgical instrument (6212), an outer packaging (6214), a sterile packaging (6216), and a return packaging (6218). Surgical kit (6210) is similar to surgical kit (6010) described above with reference to FIGS. 55-56B with differences described in detail below. Unlike sterile packaging (6016), a portion of return packaging (6218) is coupled with sterile packaging (6216) and a portion of return packaging (6218) is disposed within interior (6238) (see FIG. 58) of sterile packaging (6216). In some versions, outer packaging (6214) may be omitted.

Surgical instrument (6212) is similar to surgical instrument (6012). Surgical instrument (6212) includes a plurality of portions, including first, second, and third portions (6220a-c), configured to separate from each other along a frangible portion (6219). In some versions, at least one of first, second, or third portions (6220a-c) of surgical instrument (6212) includes a machine readable tag (6221a-c). Surgical instrument (6212) includes a master machine readable tag (6223) similar to master machine readable tag (6023).

Outer packaging (6214) is similar to outer packaging (6014). Outer packaging (6214) includes a body (6222) that defines an interior (6224) and an exterior (6226). In the closed configuration of FIG. 57, interior (6224) is completely enclosed. Outer packaging (6214) moves from the closed configuration of FIG. 57 to the open configuration of (similar to FIG. 56A) when outer packaging (6214) is opened. Interior (6224) of outer packaging (6214) is formed from inner surfaces (6228) of body (6222).

Sterile packaging (6216) is similar to sterile packaging (6016). Sterile packaging (6216) is configured to surround surgical instrument (6212). Sterile packaging (6216) includes a body (6236) that defines an interior (6238) and an exterior (6240). Sterile packaging (6216) is disposed within interior (6224) of outer packaging (6214) in the closed configuration. As shown, sterile packaging (6216) may include recessed portions (6242) that complement the size and/or shape of the components. Repositionable labels (6230, 6232) may be used to cover and label first and second portions (6220a-b) for transport. While not shown, computer readable code may also be included for tracking. A portion of surgical instrument (6212) may be placed back into sterile packaging (6216) after usage.

Return packaging (6218) is configured to receive at least one of first portion (6220a), second portion (6220b), or third portion (6220c) of surgical instrument (6212) after the surgical procedure. Return packaging (6218) may be segmented to contain separate portions of the returned or recovered components. Return packaging (6218) includes a plurality of individual return packages. As shown in FIG. 57, the plurality of individual return packages includes first, second, and third return packages (6244, 6246, 6248). However, more or fewer return packages are envisioned. First return package (6244) includes a body (6252) that defines an interior (not shown) and an exterior (6256). Similarly, second return package (6246) includes a body (6262) that defines an interior (not shown) and an exterior (6266). FIG. 58 shows repositionable label (6230) affixed to exterior (6256) and repositionable label (6268) affixed to exterior (6266). Repositionable labels (6230, 6232) may include indicia (6282, 6284) similar to indicia (6082, 6084) that act as machine readable tags as described above. While not shown, exteriors (6256, 6266) may include additional machine readable tags.

Third return package (6248) includes a flexible bag (6272). Flexible bag (6272) is configured to allow the user to insert third portion (6220c) into an interior (6278) of flexible bag (6272). As shown, flexible bag (6272) includes a label (6280) containing indicia (6286). Return packaging (6218) includes a fluid proof seal (6274) configured to prevent fluid from passing therethrough. Fluid proof seal (6274) is configured to transition from an open configuration to receive surgical instrument (6212) after the surgical procedure to a closed configuration to prevent fluid from exiting return packaging (6218). Indicia (6286) may include one or more of a serial number, an expiration data, and/or a unique identifier that may be marked by surgical robotic hub (122) or recorded by surgical robotic hub (122) to indicate usage, time of use, location of use, etc.

As shown in FIG. 58, first return package (6244) is configured to receive first portion (6220a) of surgical instrument (6212). First return package (6244) includes a recessed portion (6298) and at least one projection, shown as projections (6300). Projections (6300) are configured to positively engage first portion (6220a) of surgical instrument (6212). Similarly, second return package (6246) includes a recessed portion (6302) and at least one projection shown as projections (6304). Projections (6300) are configured to positively engage second portion (6220b) of surgical instrument (6212). Recessed portions (6298, 6302) retain first and second portions (6220a-b) upon disassembly of surgical instrument (6212). Third return package (6248) portion is configured to receive third portion (6220c) of surgical instrument (6212), so that third portion (6220c) may be handled while minimizing user contamination.

### C. Exemplary Method

A method (6410) of processing a medical device (e.g., surgical instrument (6012, 6212)) is shown and described with reference to FIG. 59. As shown, method (6410) includes steps (6412, 6414, 6416, 6418, 6420, 6422, 6424, 6426, 6428, 6430, 6432). However, more or fewer steps are also envisioned.

At step (6412), method (6410) includes applying at least one machine readable tag (6021a-c, 6221a-c) to components (e.g., first, second, and third portions (6020a-c, 6220a-c)) of surgical instruments (6012, 6212) using a marking device (6430). In some versions, marking device (6430) may apply machine readable tag (6021a-c, 6221a-c) using an indenting process or a laser etching process. While machine readable tag (6021a-c, 6221a-c) is described as being applied prior to surgical instrument (6012, 6212) being assembled, in some instances, machine readable tag (6021a-c, 6221a-c) may be applied after surgical instrument (6012, 6212) is assembled. For example, master machine readable tag (6023, 6223) may be applied after surgical instrument (6012, 6212) is assembled.

At step (6414), method (6410) includes sorting and assembling the components for surgical instrument (6012, 6212). For example, the sorting may be performing using a sorting system (6436). Matching reusable components may optimize performance and longevity of surgical instrument (6012, 6212). Sorting system (6436) may include a vision system that may read machine readable tag (6021a-c, 6221a-c) during this sorting step. An indication may be provided regarding the usability status of the component. The indication may be an alert in the form of audible tone or visual indication. For example, the visual indication may include one or more colored lights on assembly equipment. Machine readable tags (6021a-c, 6221a-c) indicate specific components (e.g., first, second, and third portions (6020a-c, 6220a-c)) have been assembled into surgical instrument (6012, 6212). Specific internal components have an electronic chip that identifies specific components that can be read by master machine readable tag (6023). Master machine readable tag (6023, 6223) recognize the components associated with the installation and usage of that surgical instrument (6012, 6212). For example, master machine readable tag (6023) may recognize proximity or at each station of a manufacturing facility there is a camera that recognizes machine readable tag (6021a-c, 6221a-c) and groups component to surgical instrument (6012, 6212).

At step (6416), method (6410) includes scanning machine readable tags (6021a-c, 6023, 6221a-c) prior to surgical kits (6010, 6210) leaving the manufacturer. At step (6418), method (6410) includes shipping surgical kit (6010, 6210) to the medical facility for subsequent use.

At step (6420), method (6410) includes opening outer packaging (6014, 6214) and/or sterile packaging (6016, 6216) to transform surgical kit (6010, 6210) from the closed configuration to the open configuration. For example, a user at a medical facility may manually open outer packaging (6014, 6214) and/or sterile packaging (6016, 6216). Sterile packaging (6216) may be removed from outer packaging (6214) in preparation for storage or sterile packaging (6216) may be removed from outer packaging (6214) in prior to being used for a medical procedure. Surgical system (102) may recognize when packaging is opened. For example, after outer packaging (6014, 6214) is opened and surgical instrument (6012, 6212) is removed from outer packaging (6014, 6214), the signal from machine readable tag (6021a-c, 6023, 6034, 6221a-c, 6223) is no longer blocked by outer packaging (6014, 6214). Until lid (6027) is removed, the signal is blocked or otherwise contained. Once lid (6027) of outer packaging (6014) is removed, signals from machine readable tags (6021a-c, 6023, 6034, 6221a-c, 6223) may reach surgical robotic hub (122).

At step (6422), method (6410) includes scanning machine readable tag (6021a-c, 6023, 6221a-c, 6223) using a code reader (6440). Surgical robotic hub (122) or another part of surgical system (102) may include code reader (6440). Code reader (6440) may determine information pertaining to the entirety of surgical instrument (6012, 6212) or a component thereof. This scanning may be manually performed by the user or automatically by surgical robotic hub (122). Machine readable tag (6021a-c, 6221a-c) may be scanned once surgical instrument (6012, 6212) is assembled into robotic system (110).

At step (6424), method (6410) includes determining the status of the various components. The status may include a coupling status (e.g., whether surgical instrument (6012, 6212) is coupled with robotic system (110)), the condition of the components of surgical instrument (6012, 6212), and their availability to be used. Controller (6442), which may be located in surgical robotic hub (122), may determine use information of first, second, and third portions (6020a-c, 6220a-c) using the scan from step (6422). Controller (6442) may record this use information on at least one of cloud (104), a surgical robotic hub (122), controller (6442), or machine readable tag (6021a-c, 6023, 6034, 6221a-c, 6223) for later retrieval. This use data may be transmitted to a central system of the manufacturer of surgical instrument (6012, 6212) and/or a database at the return facility for enhanced tracking.

At step (6426), method (6410) includes providing an indication to the user regarding the coupling status of surgical instrument (6012, 6212). Surgical system (102) may recognize when first, second, and third portions (6020a-c, 6220a-c) have been used. Surgical system (102) may recognize that the component make of the specific surgical instrument (6012, 6212) is acceptable or unacceptable and may prevent installation if unacceptable. The indication may be an alert in the form of audible tone or visual indication.

At step (6428), method (6410) includes inserting the components into return packaging (6018, 6218). In some versions, a packing tray (which may be formed from a portion of sterile packaging (6216) or a separate tray) may be used to assist with filling of first, second, and third return packages (6044, 6046, 6048, 6244, 6246, 6248) and/or prevent spillage of contents (e.g., first, second, and third portions (6020a-c, 6220a-c)) placed within first, second, and third return packages (6044, 6046, 6048, 6244, 6246, 6248). The packing tray may improve organization and reduce the overall time to prepare for subsequent shipping of first, second, and third return packages (6044, 6046, 6048, 6244, 6246, 6248) at step (6430).

At step (6432), method (6410) includes assessing returned portions of surgical instrument (6012, 6212). Assessing returned components (e.g., first, second, and third portions (6020a-c, 6220a-c)), improves process flow so that the components may be assessed and possibly reused. For example, reclamation system (6444) may mark surgical instrument (6012, 6212) to track usages. Tracking may allow for more straightforward sorting of components (e.g., first, second, and third portions (6020a-c, 6220a-c)) at the return location (e.g., manufacturing facility or other return facility). The return location may scan machine readable tag (6021a-c, 6221a-c) and sort the component (e.g., first, second, and third portions (6020a-c, 6220a-c)) according to a variety of criteria, including the number of uses. Reclamation system (6444) may read usage information from surgical instrument (6012, 6212). This usage data may be on a memory (e.g., an EEPROM). Reclamation system (6444) may determine whether surgical instrument (6012, 6212) has been used or not. The memory may store record data pertaining to the use of surgical instrument (6012, 6212) and record component serial numbers. In some versions, surgical instrument (6012, 6212) may be physical marked as described in step (6412).

### XIII. EXEMPLARY SURGICAL INSTRUMENT WITH ALIGNMENT FEATURES FOR IMPROVED ASSEMBLY AND DISASSEMBLY

As mentioned above, objects intended to penetrate a sterile field of the surgical theater during a surgical procedure need to be suitably sterilized; while objects leaving the sterile field after a surgical procedure often require special consideration when processing for disposal, reuse, or remanufacturing. In some instances, surgical instrument/tool (112, 117, 152, 154, 156) may require at least some degree of assembly within the surgical theater prior to exemplary use in accordance with the description herein; and then require some degree of disassembly after exemplary use such that selective components may be harvested for disposal, reuse, and/or remanufacturing. Therefore, in some instances, a surgical instrument/tool (112, 117, 152, 154, 156) may be introduced into the sterile field as a surgical kit with subcomponents requiring at least some degree of assembly prior to exemplary use. In addition to the subcomponents used to form surgical instrument/tool (112, 117, 152, 154, 156), such a surgical kit may contain tools for assembly, disassembly, and suitable processing for disposal, reuse, or remanufacturing of instrument/tool (112, 117, 152, 154, 156) and its subcomponents.

In some instances, a proximal body of surgical instrument/tool (112, 117, 152, 154, 156), such as handpiece (160, 176, 185), may contain internal components (such as electronics and/or batteries) that need be processed for disposal, reuse, or remanufacturing separately from the rest of handpiece (160, 176, 185). Therefore, such internal components may need to be accessed and removed from handpiece (160, 176, 185) after a surgical procedure, but within the sterile surgical theater.

It may be desirable to provide a proximal body, such as handpiece (160, 176, 185), with internal components that are accessible within the surgical theater. However, it is also desirable to ensure the proximal body, such as handpiece (160, 176, 185), is structurally robust enough to maintain its assembled formation during exemplary use of surgical instrument/tool (112, 117, 152, 154, 156). In other words, it may be desirable to selectively access internal components of handpiece (160, 176, 185) after exemplary use in a surgical procedure; yet also ensure handpiece (160, 176, 185) does not inadvertently disassemble during exemplary use in a surgical procedure.

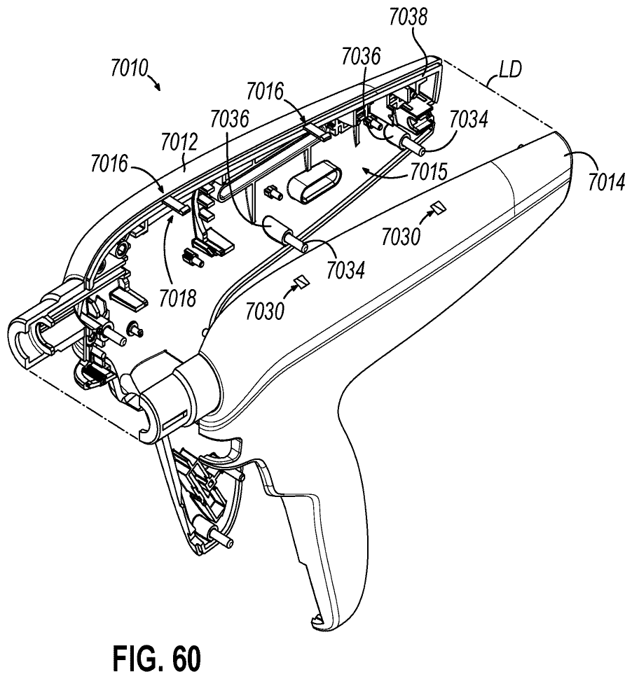

FIG. 60 shows an exemplary proximal body (7010) that may be readily incorporated into surgical instrument/tool (112, 117, 152, 154, 156). Proximal body (7010) includes a first shroud (7012) and a second shroud (7014) that are configured to couple together in order to form proximal body (7010). As will be described in greater detail below, first shroud (7012) and second shroud (7014) contain complementary coupling features configured to resist inadvertent disassembly of proximal body (7010) during exemplary use, but also allow a user to separate shroud (7012, 7014) after exemplary use in order to harvest internal components for processing.

In the current example, proximal body (7010) is shown as a handpiece, such that proximal body (7010) may be used in replacement of handpiece (160, 176, 185) described above. Proximal body (7010) is configured to suitably couple with a shaft assembly and end effector, such as shaft assembly (164, 178, 186) and end effector (166, 180, 188) described above. Together, shrouds (7012, 7014) define a hollow interior (7015) which may, when suitably assembled, house suitable components of surgical instrument/tool (112, 117, 152, 154, 156) as would be apparent to one skilled in the art in view of the teachings herein. For example, proximal body (7010) may be configured to suitably house circuit boards, control units, batteries, ultrasonic transducer (162), toggle buttons (173, 174, 175, 195, 196, 197), trigger (183, 194), etc.

In the current example, shrouds (7012, 7014) include a plurality of aligned coupling sleeves (7036) that may receive friction fitting coupling bodies (7034). Complementary coupling sleeves (7036) of each shroud (7012, 7014) may receive a respective coupling body (7034) such that one coupling body (7034) is inserted into each complementary coupling sleeve (7036) of each shroud (7012, 7014). Coupling bodies (7034) may help inhibit shrouds (7012, 7014) from decoupling in the lateral direction (LD) by a frictional braking force generated between coupling body (7034) and respective coupling sleeves (7036). Coupling bodies (7034) and coupling sleeves (7036) may include any suitable geometry as would be apparent to one skilled in the art in view of the teachings herein. In one aspect of the disclosure, coupling bodies (7034) may contain a plurality of circumferentially extending ribs that may further promote engagement between the interior surfaces of coupling sleeves (7036) and coupling bodies (7034). Coupling bodies (7034) may be formed from any suitable material as would be apparent to one skilled in the art in view of the teachings herein.

Shrouds (7012, 7014) also include complementary support ribs (7038) lining a perimeter of the interior surface of shrouds (7012, 7014). Complementary support ribs (7038) of each shroud (7012, 7014) are configured to receive each other in a nested fashion in order to resist shrouds (7012, 7014) from vertically and longitudinally actuating relative to each other while coupled together. Therefore, ribs (7038) engage each other to inhibit shrouds (7012, 7014) from moving relative to each other in directions that are perpendicular to the to the lateral direction (LD) shown in FIG. 60.

Shrouds (7012, 7014) also include at least one latching assembly (7016). As will be described in greater detail below, while shrouds (7012, 7014) are suitably coupled to each other, latching assemblies (7016) are configured to move between a locked configuration and an unlocked configuration. While in the locked configuration, latching assemblies (7016) are configured to assist coupling bodies (7024) and sleeves (7036) in resisting shrouds (7012, 7014) from laterally decoupling. While in the unlocked configuration, latching assembly (7016) is configured to allow a user to at least initiate lateral separation of shrouds (7012, 7014) such that the user may overcome the frictional braking force inhibiting lateral separation of shrouds (7012, 7014).

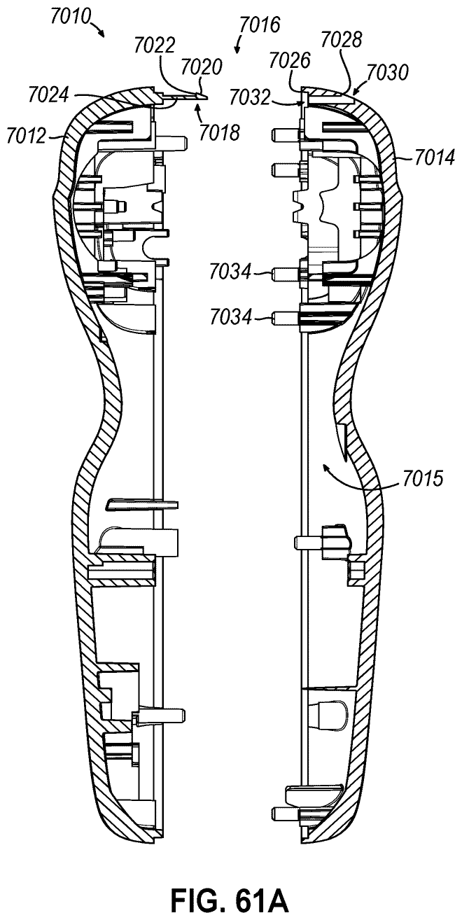

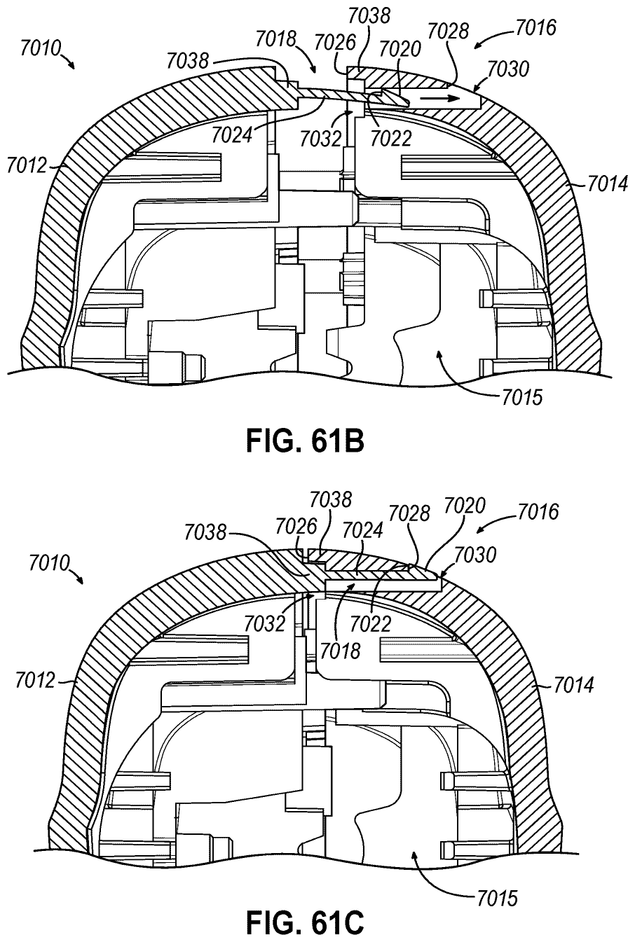

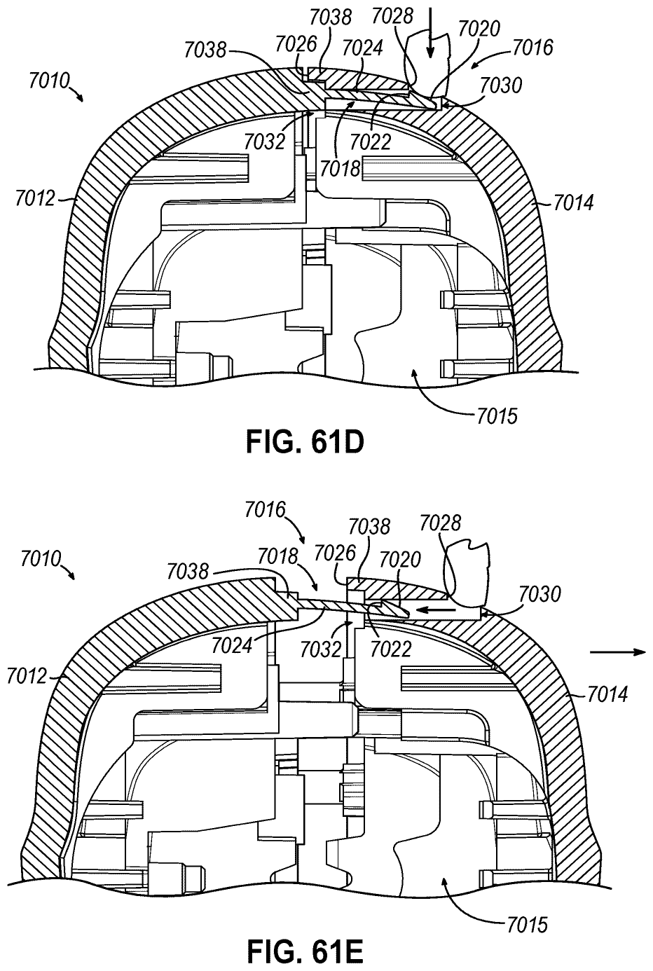

Each latching assembly (7016) includes a resilient latch (7018) extending laterally from one shroud (7012), while the other shroud (7014) defines channel (7032) (see FIGS. 61A-61E) and an access hole (7030) in communication with each other. Resilient latch (7018) includes a resilient leg (7024) extending laterally away from its respective shroud (7012). As will be described in greater detail below, cam surface (7020) is configured to engage a corresponding cam surface (7026) of shroud (7014) as shrouds (7012, 7014) are laterally coupled together in order to drive resilient leg (7024) of latch (7018) from the relaxed position (see FIG. 61A) into the flexed position (see FIG. 61B). As will also be described in greater detail below, locking shoulder (7022) is configured to engage a corresponding locking shoulder (7028) defined by shroud (7014) when shrouds (7012, 7014) are laterally coupled together in order to inhibit shrouds (7012, 7014) from laterally decoupling.

Resilient leg (7024) terminates into a respective camming surface (7020) and locking shoulder (7022). Resilient leg (7024) is sufficiently flexible such that leg (7024) may deflect from a relaxed position (see FIG. 61A) into a flexed position (see FIG. 61B) in response to an external force. Additionally, resilient leg (7024) is sufficiently resilient such that leg (7024) may return to the relaxed position (see FIG. 61C) once the external force is sufficiently removed.

FIGS. 61A-61E show an exemplary coupling and decoupling of shrouds (7012, 7014) utilizing latching assembly (7016). First, as shown in FIG. 61A, a user may align shrouds (7012, 7014) such that resilient latch (7018) is vertically and longitudinally aligned with channel (7032). It should be understood that while latch (7018) and channel (7032) are suitably aligned to couple shrouds (7012, 7014) together, complementary coupling sleeves (7036) of each shroud are suitably aligned, as well as complementary support ribs (7038) of each shroud (7012, 7014).

Next, as shown in FIG. 61B, with latch (7018) and channel (7032) aligned, a user may move shrouds (7012, 7014) toward each other such that contact between camming surfaces (7020, 7026) drives resilient leg (7024) from the relaxed position into a flexed position. As shrouds (7012, 7014) actuate further toward each other, camming surface (7020) of resilient latch (7018) may remain engaged with channel (7032) such that channel (7032) keeps latch (7018) in the flexed position.

Next, as shown in FIG. 61C, once shrouds (7012, 7014) are fully coupled, camming surface (7020) is advanced laterally past the portion of channel (7032) forcing resilient latch (7018) into the flexed position such that resilient leg (7024) returns to the relaxed position and camming surface (7020) enters access hole (7030). With camming surface (7020) within access hole (7030), locking shoulder (7022) is directly adjacent to locking surface (7028), which defines a portion of access hole (7030). Locking shoulder (7022) and locking surface (7028) are directly adjacent to each other such that if shrouds (7012, 7014) attempt to laterally disengage each other, contact between shoulder (7022) and surface (7028) inhibits lateral movement of shrouds (7012, 7014) away from each other. Therefore, resilient latch (7018) is in the locked position while locking shoulder (7022) and locking surface (7028) are directly adjacent to each other, as shown in FIG. 61C. It should be understood that latching assembly (7016) also assists in aligning shrouds (7012, 7014) as shrouds (7012, 7014) are initially being coupled together, as well as keeping shrouds (7012, 7014) aligned during exemplary use.

While in the locked position, a user may utilize proximal body (7010) in any suitable manner as would be apparent to one skilled in the art in view of the teachings herein. For example, a user may manipulate proximal body (7010) in order to suitably control surgical instrument/tool (112, 117, 152, 154, 156) in which proximal body (7010) is incorporated into. With latch (7018) in the locked position, latching assemblies (7016) enhance the structural robustness of proximal body (7010) by further inhibiting shrouds (7012, 7014) from inadvertently disassociating from each other.

After a user is finished utilizing proximal body (7010) in accordance with the description herein, it may be desirable to access various components housed within hollow interior (7015) for further processing (e.g., disposal, reuse, remanufacturing, etc.). If a user desires to access interior (7015) of proximal body (7010), the user may press down on terminating ends of resulting latch (7018) via access hole (7030) as shown in FIG. 61D. In particular, the user may press on resilient latch (7018) to flex resilient leg (7014) such that locking shoulder (7022) is no longer directly adjacent to locking surface (7028), thereby driving resilient latch (7018) into the unlocked configuration. With locking shoulder (7022) and locking surface (7028) separated from each other in the unlocked configuration, latching assembly (7016) may no longer inhibit lateral separation of shrouds (7012, 7014). Therefore, while a user presses downward on resilient latch (7018) via access hole (7030) as shown in FIG. 61D, the user may simultaneously pull the portions of shrouds (7012, 7014) directly adjacent to latching assembly (7016) apart from each other, as shown in FIG. 61E.

Once shrouds (7012, 7014) are suitably separated while latching assembly (7016) is in the unlocked configuration, camming surface (7020) of resilient latch (7018) reengages camming surface (7026) defined by channel (7032). Engagement between camming surfaces (7020, 7026) keeps resilient latch (7018) in the flexed position, thereby allowing a user to further pull shrouds (7012, 7014) apart. A user may further pull shrouds (7012, 7014) apart such that resilient latch (7018) exits channel (7032), thereby allowing latch (7018) to return to the relaxed position.

It should be understood that the resistance to lateral decoupling of shrouds (7012, 7014) provided by latching assembly (7016) may be controlled depending on whether latching assembly (7016) is in the locked configuration or the unlocked configuration. Therefore, if a user desires to decouple shrouds (7012, 7014) in accordance with the teachings herein, user may drive resilient latch (7018) into the flexed position such that locking shoulder (7022) and locking surface (7028) disassociate from each other. Otherwise, resilient latch (7018) will remain in the locked configuration, as shown in FIG. 61C, such that locking shoulder (7022) and locking surface (7028) inhibit inadvertent decoupling of shrouds (7012, 7014). In other words, latching assembly (7016) allows a user to easily decouple shrouds (7012, 7014) from one another; where shrouds (7012, 7014) remain structurally robust in the locked configuration, while shrouds (7012, 7014) are susceptible to separation in the unlocked configuration.

If shrouds (7012, 7014) of proximal body were strictly coupled together utilizing the frictional braking force provided by coupling bodies (7034) and coupling sleeves (7036), the lateral decoupling force required to pull shrouds (7012, 7014) apart may be substantially constant, regardless if the user desires to keep shrouds (7012, 7014) together, or separate shrouds (7012, 7014) to access internal components. In such instances, one may have to choose a frictional braking force structurally robust enough to keep shrouds (7012, 7014) together during exemplary use, but difficult to access internally; or choose a frictional braking force that allows easy decoupling of shrouds (7012, 7014), but also leaving shrouds (7012, 7014) susceptible to laterally decoupling during exemplary use.

While two latching assemblies (7016) are shown in the current example, any suitable number of latching assemblies (7016) may be utilized as would be apparent to one skilled in the art in view of the teachings herein. For example, a single latching assembly (7016) may be utilized. Additionally, while latching assemblies (7016) are shown positioned on the top of proximal body (7010); latching assemblies (7016) may be placed at any suitable location, or combination of locations, on proximal body (7010) as would be apparent to one skilled in the art in view of the teachings herein.

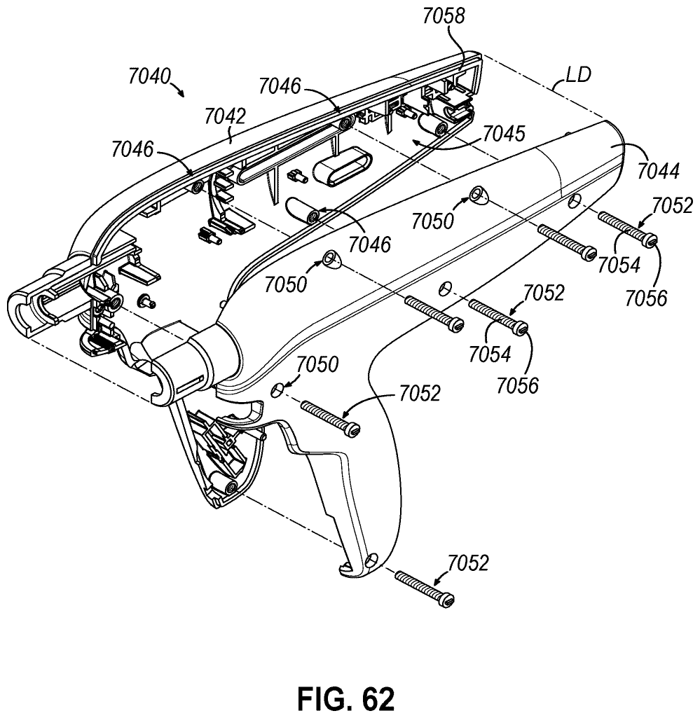

While latching assembly (7016) is utilized to provide both structural robustness during exemplary use and internal access to harvest internal components after exemplary use, any other suitable structures may be utilized as would be apparent to one skilled in the art in view of the teachings herein. FIG. 62 shows another exemplary proximal body (7040) that may be readily incorporated into surgical instrument/tool (112, 117, 152, 154, 156). Proximal body (7040) includes a first shroud (7042) and a second shroud (7044) that are configured to couple together in order to form proximal body (7040). As will be described in greater detail below, first shroud (7042) and second shroud (7044) contain complementary coupling features configured to resist inadvertent disassembly of proximal body (7040) during exemplary use, but also allow a user to separate shrouds (7042, 7044) after exemplary use in order to harvest internal components for processing.

In the current example, proximal body (7040) is shown as a handpiece, such that proximal body (7040) may be used in replacement of handpiece (160, 176, 185) described above. Proximal body (7040) is configured to suitably couple with a shaft assembly and end effector, such as shaft assembly (164, 178, 186) and end effector (166, 180, 188) described above. Together, shrouds (7042, 7044) define a hollow interior (7045) which may, when assembled, house suitable components of surgical instrument/tool (112, 117, 152, 154, 156) as would be apparent to one skilled in the art in view of the teachings herein. For example, proximal body (7040) may be configured to suitably house circuit boards, control units, batteries, ultrasonic transducer (162), toggle buttons (173, 174, 175, 195, 196, 197), trigger (183, 194), etc.

Similar to shrouds (7012, 7014) described above, shrouds (7042, 7044) also include complementary support ribs (7058) lining a perimeter of the interior surface of shrouds (7042, 7044). Complementary support ribs (7058) of each shroud (7042, 7044) are configured to receive each other in a nested fashion in order to resist shrouds (7042, 7044) from vertically and longitudinally actuating relative to each other while coupled together. Therefore, ribs (7058) engage each other to inhibit shrouds (7042, 7044) from moving relative to each other in directions that are perpendicular to the to the lateral direction (LD) shown in FIG. 62.

Rather than latching assemblies (7016), shroud (7042) includes a plurality of female threaded coupling sleeves (7046); while shroud (7044) defines a plurality of corresponding through holes (7050). Through holes (7050) and corresponding female threaded coupling sleeves (7046) are configured to receive a corresponding threaded twist screw (7052). Through holes (7050) are dimensioned large enough to receive threaded shafts (7054) of a corresponding twist screw (7052), but not large enough such that head (7056) of twist screw (7052) may travel past through holes (7050). Additionally, threaded shaft (7054) is configured to mesh with the threading of female threaded coupling sleeve (7046) such that rotation of threaded shaft (7054) relative to female threaded coupling sleeve (7046) longitudinally actuates threaded shaft (7054) relative the female threaded coupling sleeve (7046) and shroud (7042). Twist screws (7052) are dimensioned such that when suitably coupled, head (7056) abuts against a surface of shroud (7044), thereby compressing shrouds (7042, 7044) together; while the threaded engagement between threaded shaft (7054) and female threaded coupling sleeves (7046) inhibits twist screw (7052) from disassociating with shroud (7042). Therefore, a user may insert threaded shafts (70454) into a corresponding through hole (7050) until threaded shaft (7054) engages female threaded coupling sleeve (7046). Next, the user may then rotate twist screw (7052) with suitable torque at head (7056) until twist screw (7052) suitably couples shrouds (7042, 7044) together. Twist screws (7052) may therefore inhibit shrouds (7042, 7044) from disassociating with each other in the lateral direction (LD) during exemplary use.

After exemplary use, a user may remove threaded twist screws (7052) from shrouds (7042, 7044) in order to allow easy decoupling of shrouds (7042, 7044) in the lateral direction (LD) to harvest internal components for processing. A user may apply torque to head (7056) of each twist screw (7052) until threaded shaft (7054) decouples with female threaded coupling sleeve (7046). After all twist screws (7052) are decoupled from their respective female threaded coupling sleeve (7046), the user may laterally decouple shrouds (7042, 7044) to provide access to internal components. Therefore, twist screws (7052), female threaded coupling sleeves (7046), and through holes (7050) allow a user to easily decouple shrouds (7042, 7044) from one another; where shrouds (7042, 7044) remain structurally robust when twist screws (7052) are suitably assembled, while shrouds (7042, 7044) are susceptible to separation when twist screws (7052) suitably detached.

In some aspects of the disclosure, shrouds (7042, 7044) may include suitable through holes (7056) and coupling bodies (7034); along with use of female threaded coupling sleeves (7046), through holes (7050), and twist screws (7052). Any suitable combination of coupling bodies (7034) and twist screws (7052) may be utilized as would be apparent to one skilled in the art in view of the teachings herein.

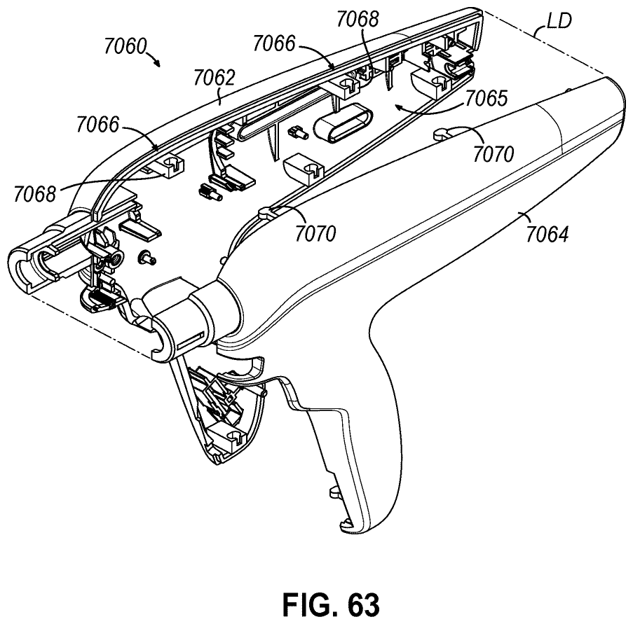

FIG. 63 shows another exemplary proximal body (7060) that may be readily incorporated into surgical instrument/tool (112, 117, 152, 154, 156). Proximal body (7060) includes a first shroud (7062) and a second shroud (7064) that are configured to couple together in order to form proximal body (7060). As will be described in greater detail below, first shroud (7062) and second shroud (7064) contain complementary coupling features configured to resist inadvertent disassembly of proximal body (7060) during exemplary use, but also allow a user to separate shrouds (7062, 7064) after exemplary use in order to harvest internal components for processing.

In the current example, proximal body (7060) is shown as a handpiece, such that proximal body (7060) may be used in replacement of handpiece (160, 176, 185) described above. Proximal body (7060) is configured to suitably couple with a shaft assembly and end effector, such as shaft assembly (164, 178, 186) and end effector (166, 180, 188) described above. Together, shrouds (7062, 7064) define a hollow interior (7065) which may, when assembled, house suitable components of surgical instrument/tool (112, 117, 152, 154, 156) as would be apparent to one skilled in the art in view of the teachings herein. For example, proximal body (7060) may be configured to suitably house circuit boards, control units, batteries, ultrasonic transducer (162), toggle buttons (173, 174, 175, 195, 196, 197), trigger (183, 194), etc.

Rather than having latching assembly (7016) or twist screws (7052), shrouds (7062, 7064) contain vertical direction coupling assemblies (7066). Vertical direction coupling assemblies (7066) are configured to allow shrouds (7062, 7064) to actuate vertically relative to each other in order to suitably couple and decouple. Vertical direction coupling assembly (7066) includes a plurality of first coupling bodies (7068) extending from first shroud (7062) and a plurality of corresponding second coupling bodies (7070) extending from second shroud (7064) toward first shroud (7062).

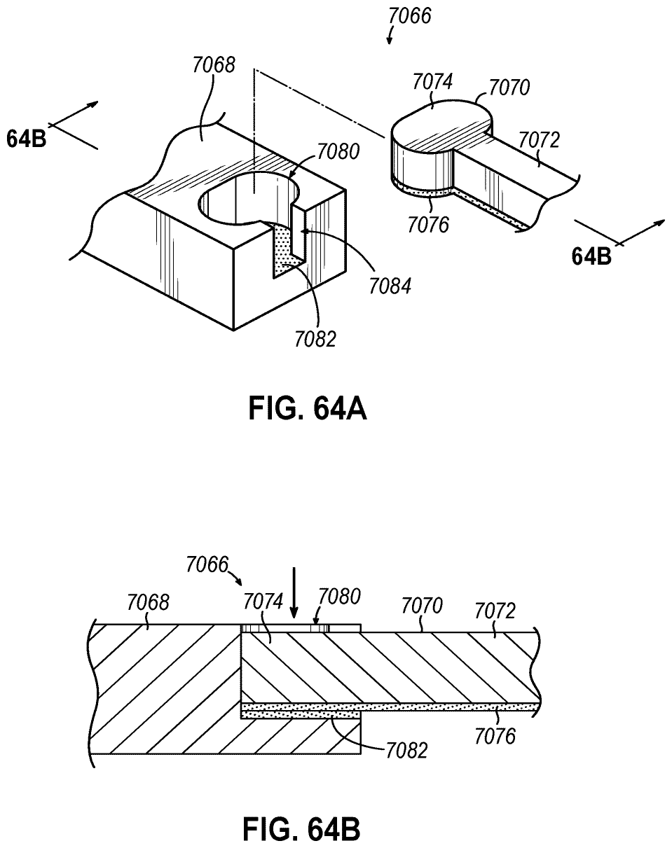

As best shown in FIG. 64A, first coupling body (7068) defines a complementary channel (7080) extending from a top surface of first coupling body (7068) and terminating into a magnetic floor (7082). First coupling body (7068) also defines a slot (7084) in communication with channel (7080) such that slot (7084) and channel (7080) are dimensioned to receive a corresponding second coupling body (7070).

Second coupling body (7070) includes a narrow portion (7072) terminating into a widened portion (7074). Second coupling body (7070) also includes a magnetic surface (7076) configured to be directly adjacent and/or in contract with magnetic floor (7082) of first coupling body (7068). Narrow portion (7072) extends away from sheath (7064) and is dimensioned to suitably fit within slot (7084) defined by first coupling body (7068). Widened portion (7074) is dimensioned to fit within complementary channel (7080) defined by first coupling body (7068). When suitably coupled as shown in FIG. 64B, the complementary geometries of slot (7084) and channel (7080) respectively with narrow portion (7072) and widened portion (7074) are configured to inhibit relative movement between shrouds (7062, 7064) while suitably coupled in every direction except the vertical direction. Additionally, magnetic surface (7076) and magnetic floor (7082) are magnetically attracted toward each other, such that while first and second coupling bodies (7068, 7070) are coupled to each other, the magnetic attraction between surface (7076) and floor (7082) inhibits second coupling body (7070) from actuating vertically out of the confines of first coupling body (7068); thereby inhibiting relative movement between shrouds (7062, 7064) in the vertical direction as well.

It should be understood that the magnetic attraction between floor (7082) and surface (7076) is suitably strong enough such that sheaths (7062, 7064) do not inadvertently disassociate from each other during exemplary use in accordance with the description herein. However, the magnetic attraction between floor (7082) and surface (7076) may be overcome with a sufficient amount of force in the vertical direction when a user desires to intentionally decouple sheaths (7072, 7064) from each other to access hollow interior (7065) in order to harvest internal components for processing. In other words, shrouds (7062, 7064) remain structurally robust during exemplary use, while shrouds (7062, 7064) are susceptible to separation in response to a sufficient force in the vertical direction via coupling assembly (7066), which allows a user to easily decouple shrouds (7062, 7064) from one another. It should be understood that, in the vertical direction, coupling assemblies (7066) also assist in aligning shrouds (7062, 7064) as shrouds (7062, 7064) are initially being coupled together, as well as keeping shrouds (7062, 7064) aligned during example use.

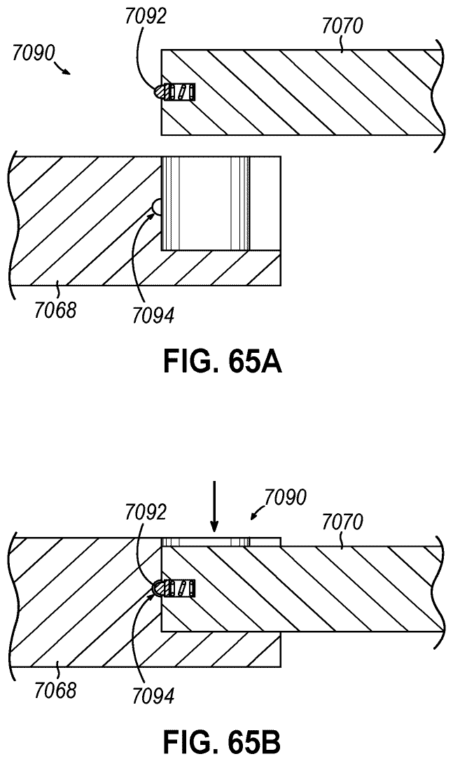

While magnetic attraction is used in the current example to inhibit relative movement between shrouds (7062, 7064) in the vertical direction, any other suitable structures may be utilized to inhibit relative vertical movement as would be apparent to one skilled in the art in view of the teachings herein. FIGS. 65A-65B show an alternative coupling assembly (7090) that may be readily incorporated into shroud (7062, 7064) in replacement of coupling assembly (7066) described above. Therefore, coupling assembly (7090) is substantially similar to coupling assembly (7066) described above, with differences elaborated below.

In particular, rather than magnets, coupling assembly (7090) includes a resilient nub (7092) associated with an exterior surface of second body (7070); while coupling assembly (7090) also includes a corresponding recess (7094) defined by an interior surface of first coupling body (7068). Resilient nub (7092) and recess (7094) are dimensioned to interact with each other in a snap-fit fashion such that while coupled together, a frictional braking force between resilient nub (7092) and recess (7094) inhibits relative movement between first body (7068) and second body (7070). However, the frictional braking force between resilient nub (7092) and recess (7094) may be overcome with a sufficient amount of force in the vertical direction when a user desires to intentionally decouple sheaths (7072, 7064) from each other to access hollow interior (7065) in order to harvest internal components for processing. In other words, coupling assembly (7090) allows a user to easily decouple shrouds (7062, 7064) from one another; where shrouds (7062, 7064) remain structurally robust during exemplary use, while shrouds (7062, 7064) are susceptible to separation in response to a sufficient force in a the vertical direction.

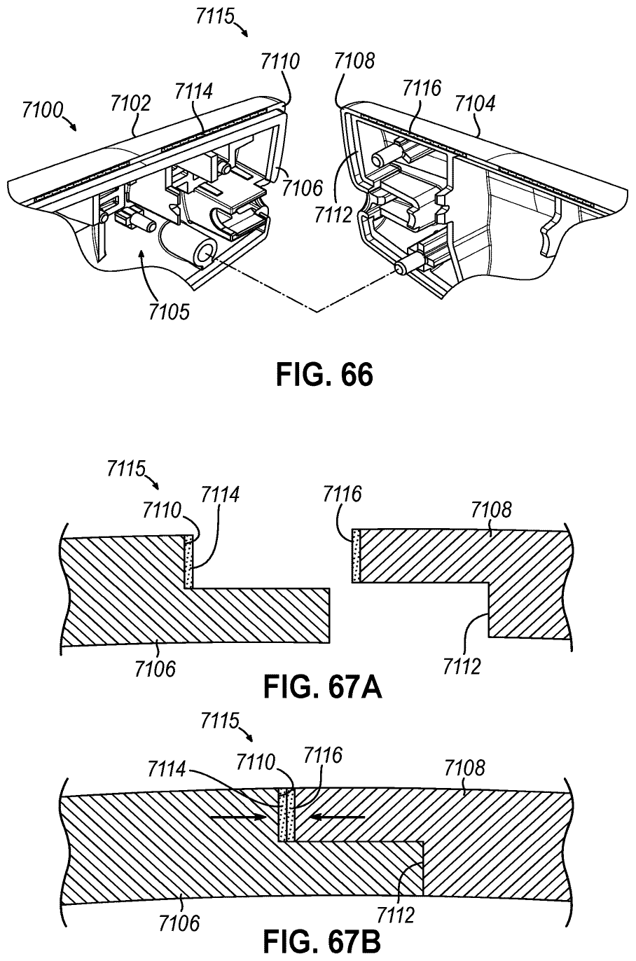

FIG. 66 shows another exemplary proximal body (7100) that may be readily incorporated into surgical instrument/tool (112, 117, 152, 154, 156). Proximal body (7100) includes a first shroud (7102) and a second shroud (7104) that are configured to couple together in order to form proximal body (7100). As will be described in greater detail below, first shroud (7102) and second shroud (7104) contain complementary coupling features configured to resist inadvertent disassembly of proximal body (7100) during exemplary use, but also allow a user to separate shrouds (7102, 7104) after exemplary use in order to harvest internal components for processing.

In the current example, proximal body (7100) is shown as a handpiece, such that proximal body (7100) may be used in replacement of handpiece (160, 176, 185) described above. Proximal body (7100) is configured to suitably couple with a shaft assembly and end effector, such as shaft assembly (164, 178, 186) and end effector (166, 180, 188) described above. Together, shrouds (7102, 7104) define a hollow interior (7105) which may, when assembled, house suitable components of surgical instrument/tool (112, 117, 152, 154, 156) as would be apparent to one skilled in the art in view of the teachings herein. For example, proximal body (7100) may be configured to suitably house circuit boards, control units, batteries, ultrasonic transducer (162), toggle buttons (173, 174, 175, 195, 196, 197), trigger (183, 194), etc.

Each shroud (7102, 7104) in the current embodiment includes a respective complementary support rib (7106, 7108), which may be substantially similar to complementary support ribs (7038) described above. Each complementary support rib (7106, 7108) lines a perimeter of the interior surface of respective shrouds (7102, 7104) and extend away from a respective recessed surface (7110, 7112). Shrouds (7102, 7104) are configured to couple together via lateral movement relative to each other. During coupling, as shown in FIGS. 67A-67B, complementary support ribs (7106, 7108) of each shroud (7102, 7104) are configured to receive each other in a nested fashion such that support rib (7106) abuts against recessed surface (7112), and such that support rib (7108) abuts against recessed surface (7110). The nested engagement between support ribs (7106, 7108), while shrouds (7102, 7104) are suitably coupled, inhibits shrouds (7102, 7104) from vertically and longitudinally actuating relative to each other while coupled together. Therefore, ribs (7106, 7108) engage each other to inhibit shrouds (7102, 7104) from moving relative to each other in directions that are perpendicular to the lateral direction (LD).

Shrouds (7102, 7104) also include a respective magnet (7114, 7116) located on surface (7110) and support rib (7108) of respective shrouds (7102, 7104). While in the current aspect of the disclosure, magnets (7114, 7116) are shown on surface (7110) and support rib (7108), this is merely optional, as magnets (7114, 7116) may be located on any suitable components of shrouds (7102, 7104) as would be apparent to one skilled in the art in view of the teachings herein. Magnets (7114, 7116) are magnetically attracted toward each other, such that while support ribs (7106, 7108) are nested with each other, the magnetic attraction between magnets (7114, 7116) inhibits support ribs (7106, 7108) from actuating laterally out of engagement with each other; thereby inhibiting relative movement between shrouds (7102, 7104) in the lateral direction as well.

It should be understood that the magnetic attraction between magnets (7114, 7116) is suitably strong enough such that sheaths (7102, 7104) do not inadvertently disassociate from each other during exemplary use in accordance with the description herein. However, the magnetic attraction between magnets (7114, 7116) may be overcome with a sufficient amount of force in the lateral direction when a user desires to intentionally decouple sheaths (7102, 7104) from each other to access the hollow interior (7105) in order to harvest internal components for processing. In other words, magnets (7114, 7116) allows a user to easily decouple shrouds (7102, 7104) from one another; where shrouds (7102, 7104) remain structurally robust during exemplary use, while shrouds (7102, 7104) are susceptible to separation in response to a sufficient force in the vertical direction.

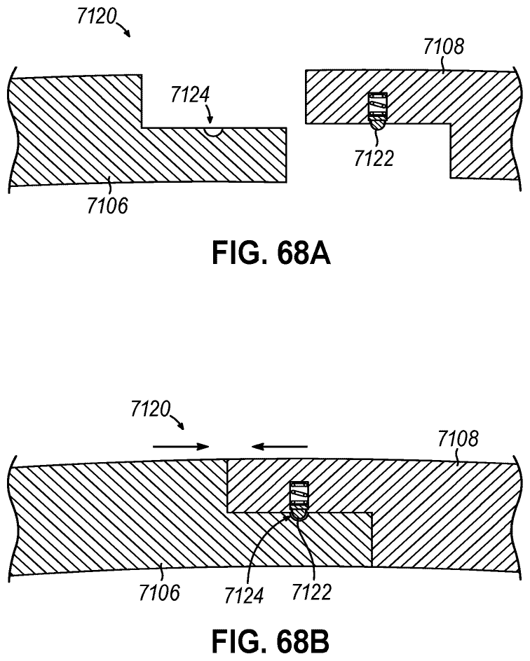

While magnetic attraction is used in the current example to inhibit relative movement between shrouds (7102, 7104) in the lateral direction, any other suitable structures may be utilized to inhibit relative vertical movement as would be apparent to one skilled in the art in view of the teachings herein. FIGS. 68A-68B show an alternative coupling assembly (7120) that may be readily incorporated into shroud (7102, 7104) in replacement of magnets (7114, 7116) described above.

Rather than magnets, coupling assembly (7120) includes a resilient nub (7122) associated with an exterior surface of support rib (7108); while coupling assembly (7120) also includes a corresponding recess (7124) defined by a complementary surface of support rib (7106). Resilient nub (7122) and recess (7124) are dimensioned to interact with each other in a snap-fit fashion such that while coupled together, a frictional braking force between resilient nub (7122) and recess (7124) inhibits relative movement between support ribs (7106, 7108). However, the frictional braking force between resilient nub (7122) and recess (7124) may be overcome with a sufficient amount of force in the lateral direction when a user desires to intentionally decouple sheaths (7102, 7104) from each other to access the hollow interior (7105) in order to harvest internal components for processing. In other words, coupling assembly (7120) allows a user to easily decouple shrouds (7102, 7104) from one another; where shrouds (7102, 7104) remain structurally robust during exemplary use, while shrouds (7102, 7104) are susceptible to separation in response to a sufficient force in the lateral direction.

In some instances, where proximal body (7010, 7040, 7060, 7100) is intended to be processed for reuse or remanufacturing, it may be desirable to prevent re-assembly of shrouds (7012, 7014, 7042, 7044, 7062, 7064, 7102, 7104) with each other or with other suitable components of surgical instrument/tool (112, 117, 152, 154, 156) coupled to proximal body (7010, 7040, 7060, 7100) if critical parts forming surgical instrument/tool (112, 117, 152, 154, 156) are out of shape or out of a specified tolerance. For example, one or more features of shrouds (7012, 7014, 7042, 7044, 7062, 7064, 7102, 7104) or other suitable components could be used as a blocking means to prevent re-assembly if such features become distorted or damaged outside a tolerance range that is acceptable. As one example, a clamp trigger, similar to trigger (183) described above, may be coupled to proximal body (7010) via coupling body (7034) and couplings sleeves (7036). During exemplary use, forces acting on trigger (183) during pivotal movement of trigger (183) may result in damage to coupling sleeves (7036) and or trigger (183), which would then prevent reassembly once sheaths (7012, 7014) are suitably processed for reuse (e.g., sterilized).

As mentioned above, in some instances after exemplary use of proximal body (7010, 7040, 7060, 7100), shrouds (7012, 7014, 7042, 7044, 7062, 7064, 7102, 7104) may be processed for reuse and or remanufacturing. As also mentioned above, hollow interior (7015, 7045, 7065, 7105) may house suitable components of surgical instrument/tool (112, 117, 152, 154, 156) as would be apparent to one skilled in the art in view of the teachings herein, such as circuit board and control units. Therefore, in instances where electrical components are contained within hollow interior (7015, 7045, 7065, 7105), it may be desirable to ensure such electrical components are suitably removed from shrouds (7012, 7014, 7042, 7044, 7062, 7064, 7102, 7104) before shrouds (7012, 7014, 7042, 7044, 7062, 7064, 7102, 7104) are processed for reuse and/or remanufacturing. Ensuring electrical components are suitably removed before shrouds (7012, 7014, 7042, 7044, 7062, 7064, 7102, 7104) are processed may prevent such electrical components from being inadvertently exposed to substances used during processing that may damage electrical components and/or render them unsuitable for further use.

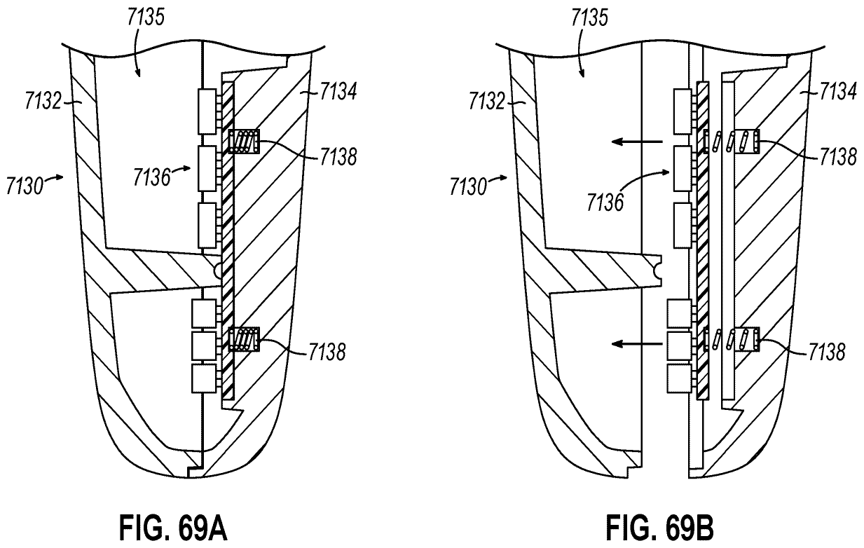

FIGS. 69A-69B show an exemplary proximal body (7130) that may be substantially similar to proximal body (7010, 7040, 7060, 7100) described above, with differences elaborated below. Therefore, proximal body (7130) includes a first shroud (7132) and a second shroud (7134) which may be substantially similar to shrouds (7012, 7014, 7042, 7044, 7062, 7064, 7102, 7104) described above, with differences elaborated below. Together, shrouds (7132, 7134) define a hollow interior (7135). Shroud (7134) includes biasing springs (7138) that are interposed between an interior surface of shroud (7134) and a suitable electrical component (7136). When shrouds (7132, 7134) are assembled, as shown in FIG. 69A, biasing springs (7138) bias electrical component (7136) against first shroud (7132), or any other suitable structure, thereby forcing electrical component (7136) into a suitable position within hollow interior (7135).

After exemplary use, shrouds (7132, 7134) may be disassembled for processing in accordance with the teachings herein. During disassembly, proximal body (7130) may be disassembled to remove electrical component (7136) from shroud (7134) such that electrical component (7136) does not inadvertently get processed with shroud (7134). As shown in FIG. 69B, once shrouds (7132, 7134) are disassembled, biasing springs (7138) drive electrical component (7136) away from shroud (7134) such that electrical component (7136) is prominently presented to the user disassembling proximal body (7130). Therefore, biasing springs (7138) help drive electrical component (7136) into an exposed position such that a person disassembling proximal body (7130) may be reminded to further remove electrical component (7136).

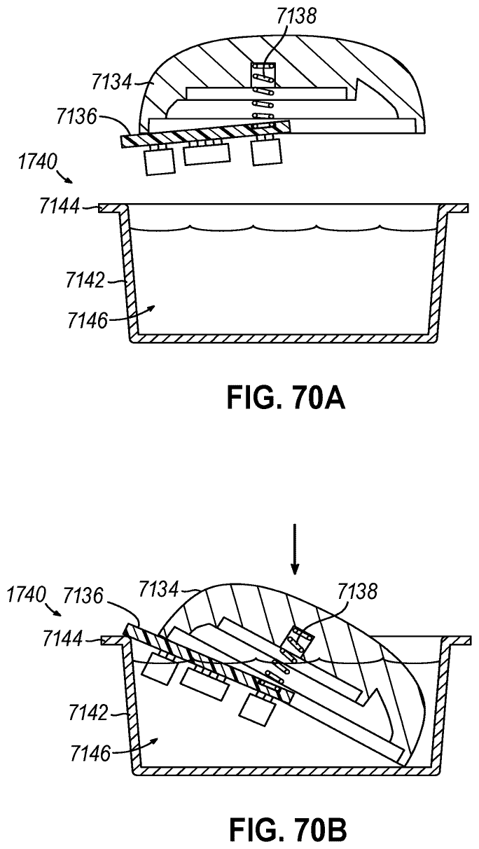

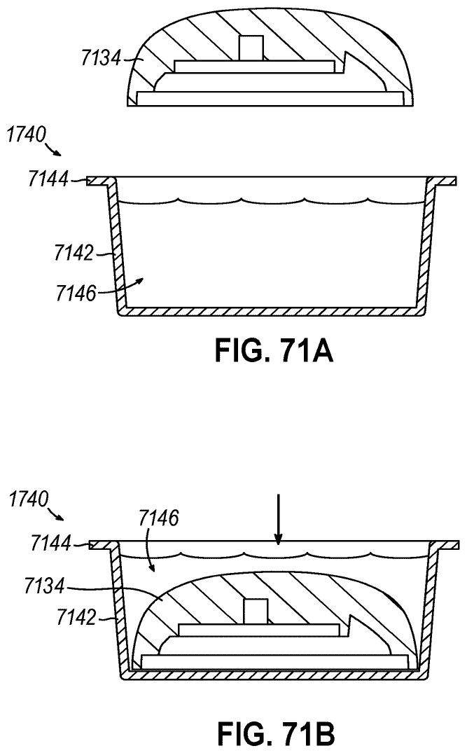

In some instances, it may be desirable to inhibit a user who is processing shroud (7132, 7134) for reuse and/remanufacturing from inadvertently dipping electrical component (7136) into a dip tray during processing. FIGS. 70A-71B show an exemplary dip tray (7140) that may be used to process shroud (7132, 7134) by exposing a used shroud (7132, 7134) in a suitable cleaning fluid to clean shroud (7132, 7134). Dip tray (7140) includes a body (7142) defining a reservoir (7146) that may house a suitable cleaning fluid. Dip tray (7140) also includes a perimeter (7144) that defines an opening dimensioned to receive shroud (7134) for processing. As shown in FIGS. 70A-70B, perimeter (7144) is dimensioned with a specific geometry that may inhibit suitably receiving shroud (7134) within reservoir (7146) if electrical component (7136) is not removed from shroud (7134). As shown in FIGS. 71A-71B, perimeter (7144) is also dimensioned with a specific geometry that suitably receives shroud (7134) within reservoir (7146) if electrical component (7136) is removed from shroud (7134). Therefore, if a user inadvertently keeps electrical component (7136) attached to shroud (7134), user may be reminded to removed electrical component (7136) when attempting to insert shroud (7134) within dip tray (7140), as shroud (7143) will not suitably fit within dip tray (7140).

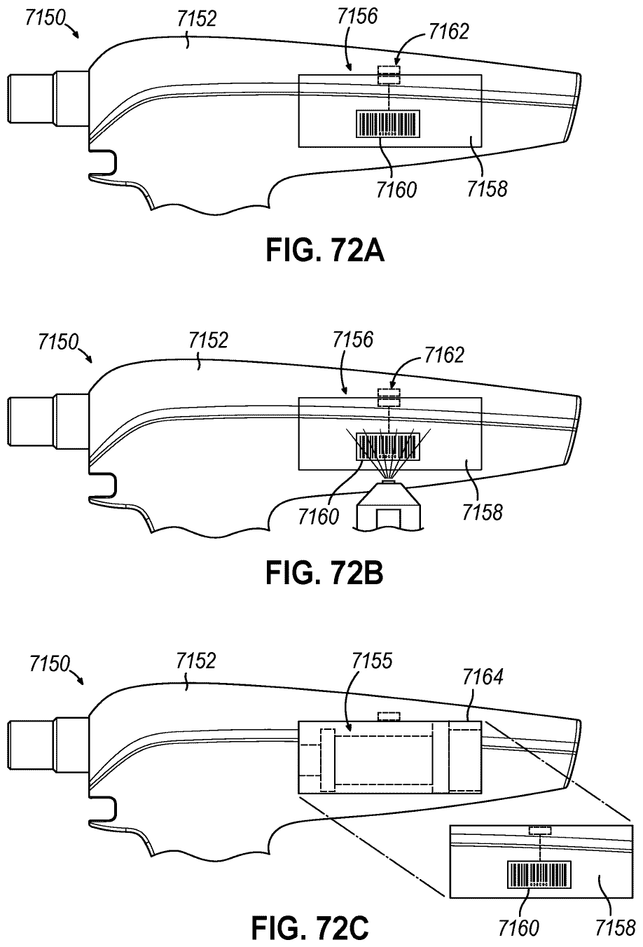

FIGS. 72A-72C show another exemplary proximal body (7150) that may be substantially similar to proximal body (7010, 7040, 7060, 7100, 7130) described above, with differences elaborated below. Therefore, proximal body (7150) includes sheath (7152) that may define a hollow interior (7155). Sheath (7152) includes a hatch assembly (7156) configured to provide suitable access to hollow interior (7155) in accordance with the description herein.

Hatch assembly (7156) includes a hatch door (7158) removably coupled to an opening (7164) defined by sheath (7152), a bar code (7160), and an electric latch assembly (7162). Latch assembly (7162) is configured to lock hatch door (7158) such that hatch door (7158) is inhibited from removal from opening (7164) unless bar code (7160) is suitably scanned. As shown in FIG. 72B, once a user desires to remove hatch door (7158), user may scan bar code (7160) with a suitable device. Scanning bar code (7160) may instruct latch assembly (7162) to unlock hatch door (7158) such that a user may remove hatch door (7158) to provide access to hollow interior (7155), as shown in FIG. 72C. Therefore, proximal body (7150) may be structurally robust during exemplary use, while still providing access to hollow interior (7155) for harvesting internal components.

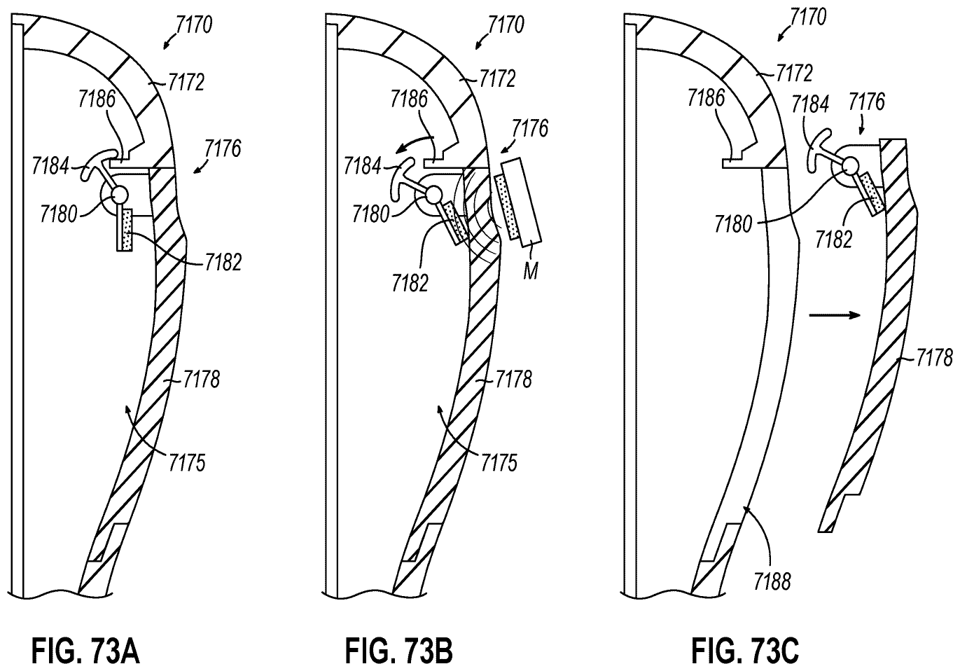

FIGS. 73A-73C show another exemplary proximal body (7170) that may be substantially similar to proximal body (7010, 7040, 7060, 7100, 7130, 7150) described above, with differences elaborated below. Therefore, proximal body (7170) includes sheath (7172) that may define a hollow interior (7165). Sheath (7172) includes a hatch assembly (7176) configured to provide suitable access to hollow interior (7175) in accordance with the description herein.

Hatch assembly (7176) includes a hatch door (7178) removably coupled to an opening (7188) defined by shroud (7172), a pivoting latch (7180) pivotally coupled with shroud (7172), and a locking protrusion (7186) within hollow interior (7175). Pivoting latch (7180) includes a magnet (7182) on one end and a latching body (7184) on the other end. Pivoting latch (7180) may be biased toward the locked position shown in FIG. 73A. In the locked position, pivoting latch (7180) may prevent hatch door (7178) from being removed from shroud (7172). If a user desires to remove hatch door (7178), user may wave a suitable magnet (M) over hatch door (7178), as shown in FIG. 73B. The magnetic attraction between magnet (M) and magnet (7182) may cause pivoting latch (7180) to pivot to an unlocked position, thereby allowing hatch door (7178) to be removed to provide access to hollow interior (7175), as shown in FIG. 73C. Therefore, proximal body (7170) may be structurally robust during exemplary use, while still providing access to hollow interior (7175) for harvesting internal components.

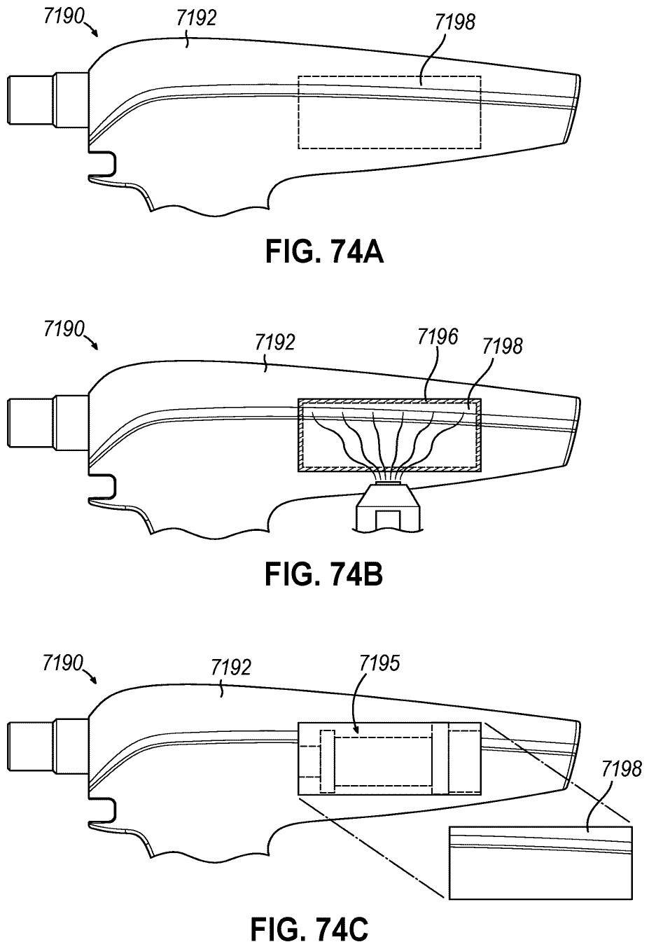

FIGS. 74A-74C show another exemplary proximal body (7190) that may be substantially similar to proximal body (7010, 7040, 7060, 7100, 7130, 7150, 7170) described above, with differences elaborated below. Therefore, proximal body (7190) includes sheath (7192) that may define a hollow interior (7195). Sheath (7192) includes a metal frame window (7196) configured to provide suitable access to hollow interior (7175) in accordance with the description herein. In particular, as shown in FIG. 74B, a user may expose metal frame window (7196) to a suitable heat source. Once metal frame window (7196) has a suitable amount of thermal energy, metal frame window (7196) may melt adjacent portions of sheath (7192), thereby creating a removable door (7198). As shown in FIG. 74C, door (7198) may then be removed to provide access to hollow interior (7195). Therefore, proximal body (7190) may be structurally robust during exemplary use, while still providing access to hollow interior (7195) for harvesting internal components.

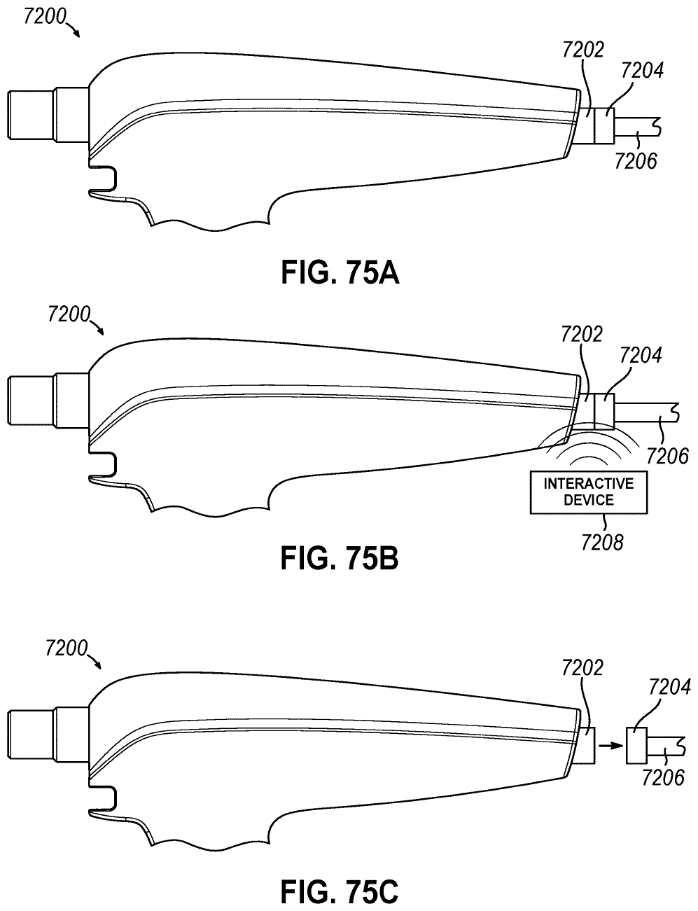

FIGS. 75A-75C show another exemplary proximal body (7200) that may be substantially similar to proximal body (7010, 7040, 7060, 7100, 7130, 7150, 7170, 7190) described above, with differences elaborated below. Proximal body (7200) also includes a power coupling (7202) which is configured to selectively electrically couple with a complementary power coupling feature (7204) of a power cord (7206). As best shown in FIGS. 75B-75C, power couplings (7202, 7204) are configured to remain coupled together unless the connection is severed with an interactive device (7208). Interactive device (7208) may be a physical key or an electrically activated release mechanism.

### XIV. EXEMPLARY DISPOSAL BAGS FOR PROCESSING FEATURES OF USED SURGICAL INSTRUMENTS

As mentioned above, objects leaving the sterile field after a surgical procedure often require special consideration when processing for disposal, reuse, or remanufacturing. In some instances, a used surgical instrument may be disassembled into various predetermined categories and inserted within suitable transportation bags in order to transport disassembled surgical features for suitable processing. It may be desirable for transportation bags to be easily fillable, and/or inhibit emitting/leaking/spilling/transmitting biohazardous material contained within transportation bags while filled with features of a used surgical instruments. Further, it may be desirable for transportation bags to facilitate a determination of whether or not stored components may be suitable for reuse and/or remanufacturing.

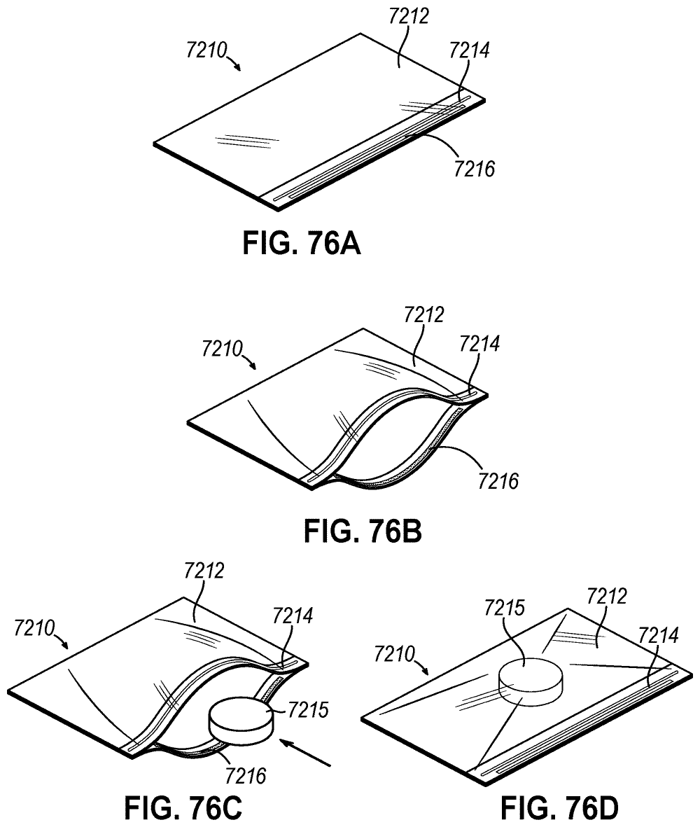

FIGS. 76A-76D show an exemplary processing bag assembly (7210) that may be utilized to transmit used surgical features for suitable processing. Processing bag assembly (7210) is formed of a suitable processing bag (7212) that is configured to seal off stored surgical components from the external environment. Therefore, an internal surface of bag (7212) may be isolated from an external surface of bag (7212). Bag (7212) defines a sealable opening (7218) that may be selectively opened in order to place used surgical components (7215) into the interior of bag (7212), and then subsequently closed to create a seal such that surgical component (7215) are suitably isolated from the external environment.

As shown in FIGS. 76A-76C, bag assembly (7210) includes a pre-applied, closable, adhesive and/or tie element (7216) that enables the opening of bag assembly (7210) such that surgical components (7215) may be deposited into bag via sealable opening (7218). As shown in FIGS. 76C and 76D, adhesive and/or tie element (7216) is configured to re-close opening (7218) of bag (7212) in order to subsequently re-seal the interior of bag (7212) from the external environment. Any suitable type of adhesive and/or tie element may be utilized as would be apparent to one skilled in the art in view of the teachings herein.

Bag assembly (7210) also includes a biasing means (7214) located on or within a portion of bag (7212) adjacent to opening (7218). Biasing means (7214) may help bias opening (7218) toward the open position shown in FIGS. 76B-76C such that a user within the sterile environment may easily deposit used surgical components within bag (7212) without having to touch and/or contaminate the external surface of bag (7212). Biasing means (7214) may be suitably overcome in order to close opening (7218). Adhesive and/or tie element (7216) is sufficiently strong enough to overcome the bias of biasing means (7214) to close and seal opening (7218) for suitable transportation in accordance with the description herein.

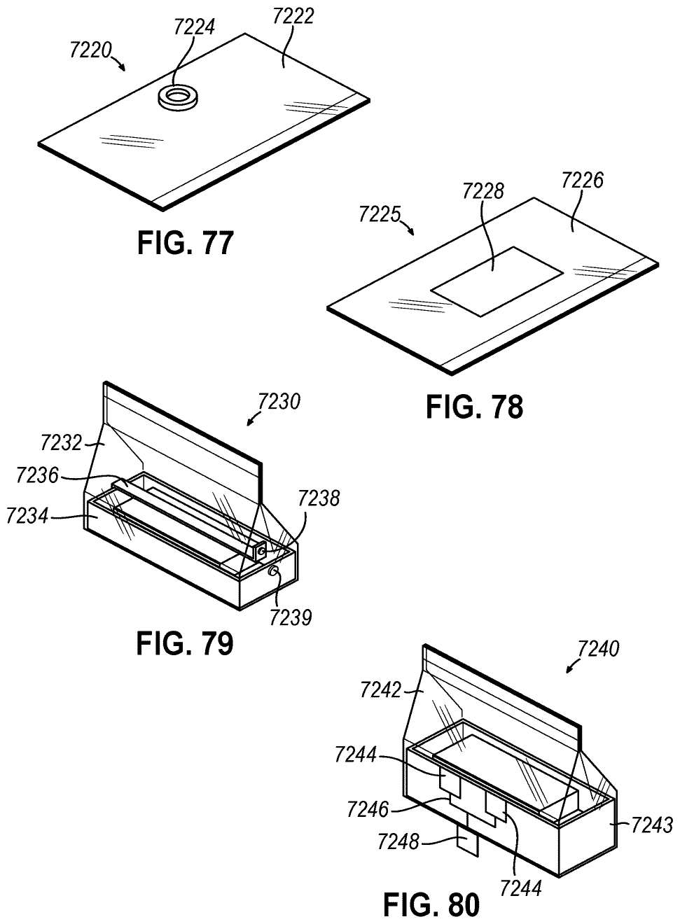

FIG. 77 shows another exemplary processing bag assembly (7220) that may be utilized to transmit used surgical features for suitable processing. Processing bag assembly (7220) may be substantially similar to processing bag assembly (7210) described above, with differences elaborated herein. Processing bag assembly (7220) includes a processing bag (7222) that may be substantially similar to processing bag (7212). Additionally, processing bag assembly (7220) includes a gas prevention/flammable control feature (7224) configured to fireproof and/or electrically isolate the interior of bag (7222). Gas prevention/flammable control feature (7224) may include any suitable structure as would be apparent to one skilled in the art in view of the teachings herein. For example, gas prevention/flammable control feature (7224) may include a valve configured to establish communication with a suction source to remove oxygen from the interior of bag (7222). As another example, gas prevention/flammable control feature (7224) may include a fire-retardant substance lining the interior of bag (7222).

FIG. 78 shows another exemplary processing bag assembly (7225) that may be utilized to transmit used surgical features for suitable processing. Processing bag assembly (7225) may be substantially similar to processing bag assembly (7210, 7220) described above, with differences elaborated herein. Processing bag assembly (7225) includes a processing bag (7225) that may be substantially similar to processing bag (7212, 7222). Additionally, processing bag assembly (7225) includes a set of instructions or indication of handling options (7228). For example, instructions or indication of handling options (7228) could include instructions for processing, or a warning of handling concerns for items that are intended to be sealed inside the bag. In some instances, bag (7226) could be color coded to indicate the processing path intended for items inside bag (7226).

FIG. 79 shows another exemplary processing bag assembly (7230) that may be utilized to transmit used surgical features for suitable processing. Processing bag assembly (7230) may be substantially similar to processing bag assembly (7210, 7220, 7225) described above, with differences elaborated herein. Processing bag assembly (7230) includes a processing bag (7232) that may be substantially similar to processing bag (7212, 7222, 7225). Additionally, processing bag assembly (7230) includes a rigid tray (7234) fixed within the interior of bag (7332). Tray (7234) may include complementary recesses dimensioned to receive specific portions of a used surgical instrument. Therefore, bag assembly (7230) may act as both a discrete holding frame and a sealing bag. Additionally, tray (7234) includes restraint snaps (7236) and restraining means (7238) that may be configured to further fix specific portions of used surgical instrument housed within tray (7234).

FIG. 80 shows another exemplary processing bag assembly (7240) that may be utilized to transmit used surgical features for suitable processing. Processing bag assembly (7240) may be substantially similar to processing bag assembly (7210, 7220, 7225, 7230) described above, with differences elaborated herein. Processing bag assembly (7240) includes a processing bag (7242) that may be substantially similar to processing bag (7212, 7222, 7225, 7232). Processing bag assembly (7240) also includes a tray (7243) that may be substantially similar to tray (7243) described above. Additionally, tray (7243) includes electrical connectors (7244) that are configured to establish electrical communication with specific portions of used surgical instrument housed within tray (7243). Electrical connectors (7244) are in communication with an external electrical coupling (7248) fixed to an exterior of bag (7242). After suitably loading portions of the used surgical instruments into tray (7243), a user may use a suitable electrical instrument to provide a small voltage or power to packaging via external electrical coupling (7248). The small voltage or power may travel to the electrical feature of used surgical instrument in communication with electrical connectors (7244) to check at least one functional aspect of the electrical feature to determine if there is capacity for reusing such an electrical feature.

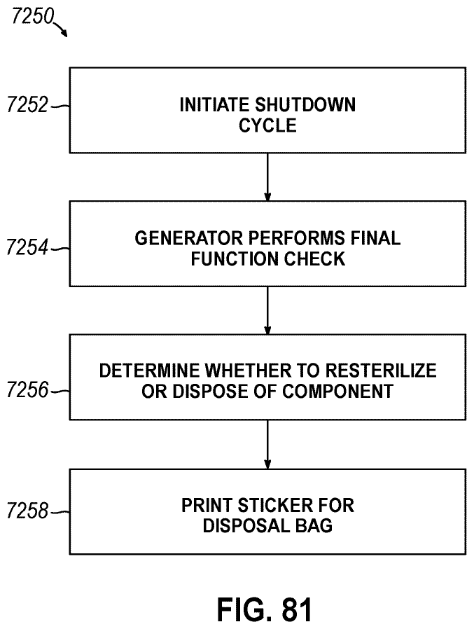

FIG. 81 shows an exemplary shutdown cycle (7250) that may be performed by generator module (140) or electronics of surgical instrument/tool (112, 117, 152, 154, 156) to check for potential recovery capacity and generate a sticker or label to be attached to processing bag (7212, 7222, 7225, 7232, 7242). First, after a user is finished using surgical instrument/tool (112, 117, 152, 154, 156) in accordance with the description herein, user may initiate (7252) the shutdown cycle. This initiation (7252) may be done using any suitable means as would be apparent to one skilled in the art in view of the teachings herein. Next, generator model (140) or electronics of surgical instrument/tool (112, 117, 152, 154, 156) may perform a final function check (7254) to determine if suitable components of surgical instrument/tool (112, 117, 152, 154, 156) have capacity for reuse. Next, generator model (140) or electronics of surgical instrument/tool (112, 117, 152, 154, 156) then determines (7256) whether features of surgical instrument/tool (112, 117, 152, 154, 156) should be sterilized or disposed of. Utilizing such determination, a printer may then print (7258) a sticker to be placed on processing bag (7212, 7222, 7225, 7232, 7242) which indicates the determination made above. Therefore, after exemplary use of surgical instrument/tool (112, 117, 152, 154, 156), a sticker may be generated and placed on processing bag (7212, 7222, 7225, 7232, 7242) to indicate the intended processing path for features of surgical instrument/tool (112, 117, 152, 154, 156).

### XV. EXEMPLARY SURGICAL VISUALIZATION SYSTEM

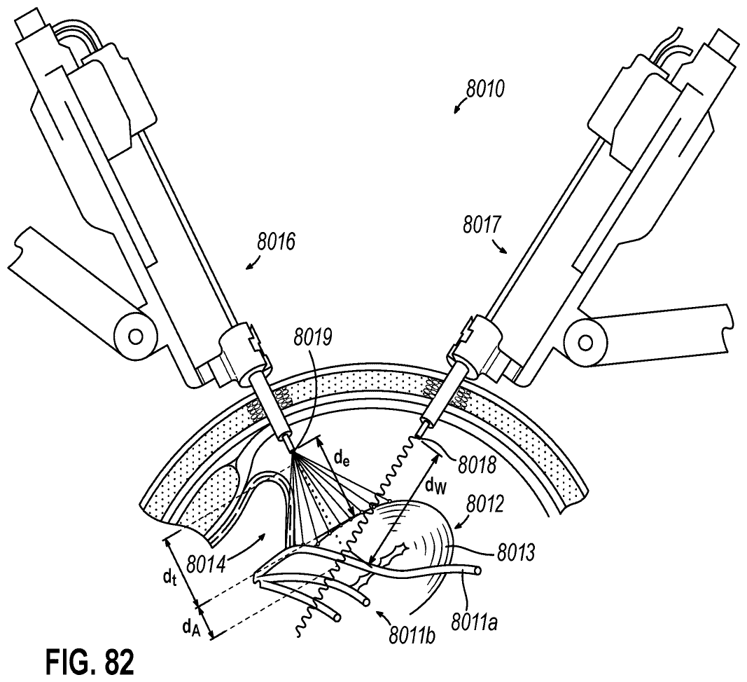

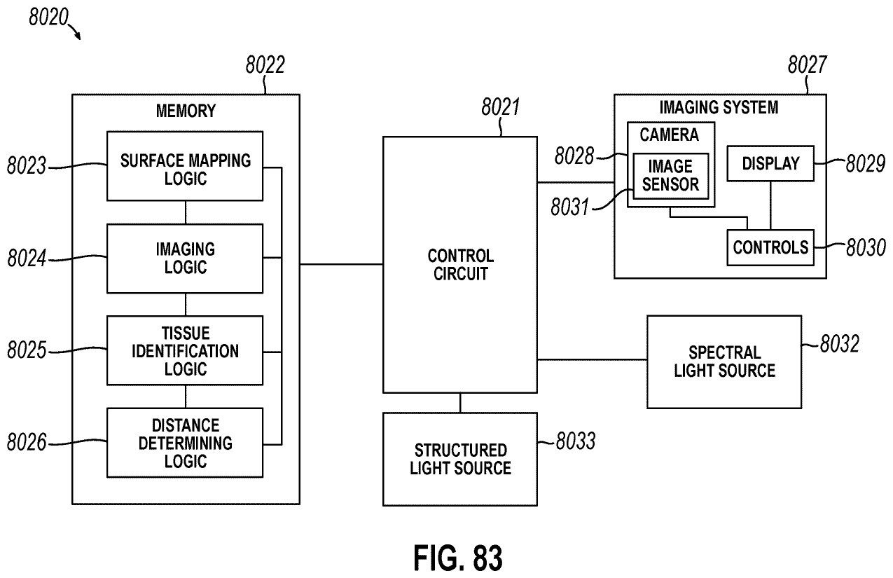

FIGS. 82-83 depicts a schematic view of a surgical visualization system (8010) and a schematic diagram of a control system (8020) that may be used in conjunction with each other, according to at least one aspect of the present disclosure. Surgical visualization system (8010) and control system (8020) may be readily incorporated into computer-implemented interactive surgical system (100) described above. For example, surgical visualization system (8010) may be used in replacement of imaging device (124) described above; while control system (8020) may be used in replacement of imaging module (138) described above. Surgical visualization system (8010) may create a visual representation of a critical structure (8011a, 8011b) within an anatomical field.

Critical structures (8011a, 8011b) may be any anatomical structures of interest or any foreign structure in the anatomical field. In one aspect, a critical structure (8011a, 8011b) may be embedded in tissue. Stated differently, a critical structure (8011a, 8011b) may be positioned below a surface of the tissue. In such instances, the tissue conceals the critical structure (8011a, 8011b) from the clinician's view. A critical structure (8011a, 8011b) may also be obscured from the view of an imaging device by the tissue. The tissue may be fat, connective tissue, adhesions, and/or organs, for example. In other instances, a critical structure (8011a, 8011b) may be partially obscured from view. Surgical visualization system (8010) is shown being utilized intraoperatively to identify and facilitate avoidance of certain critical structures, such as a ureter (8011a) and vessels (8011b) in an organ (8012) (the uterus in this example), that are not visible on a surface (8013) of the organ (8012).

With continuing reference to FIG. 82, surgical visualization system (8010) incorporates tissue identification and geometric surface mapping in combination with a distance sensor system (8014). In combination, these features of surgical visualization system (8010) may determine a position of critical structure (8011a, 8011b) within the anatomical field and/or the proximity of a surgical device (8016) to surface (8013) of the visible tissue and/or to critical structure (8011a, 8011b). Surgical device (8016) may be substantially similar to surgical instrument/tool (112, 117, 152, 154, 156) described herein. As also described herein, surgical visualization system (8010) may be configured to achieve identification of one or more critical structures (8011a, 8011b) and/or the proximity of surgical device (8016) to critical structure(s) (8011a, 8011b).

The depicted surgical visualization system (8010) includes an imaging system that includes an imaging device (8017), such as a camera of a scope, for example, that is configured to provide real-time views of the surgical site. In various instances, imaging device (8017) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from imaging device (8017) may be provided to a clinician; and, in various aspects of the present disclosure, may be augmented with additional information based on the tissue identification, landscape mapping, and input from a distance sensor system (8014). In such instances, a surgical visualization system (8010) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

Imaging device (8017) of the present example includes an emitter (8018), which is configured to emit spectral light in a plurality of wavelengths to obtain a spectral image of hidden structures, for example. Imaging device (8017) may also include a three-dimensional camera and associated electronic processing circuits in various instances. In one aspect, emitter (8018) is an optical waveform emitter that is configured to emit electromagnetic radiation (e.g., near-infrared radiation (NIR) photons) that may penetrate surface (8013) of tissue (8012) and reach critical structure(s) (8011a, 8011b). Imaging device (8017) and optical waveform emitter (8018) thereon may be positionable by surgical arms (123) (see FIG. 2) or a surgeon manually operating imaging device (8017). A corresponding waveform sensor (e.g., an image sensor, spectrometer, or vibrational sensor, etc.) on imaging device (8017) may be configured to detect the effect of the electromagnetic radiation received by the waveform sensor.

The wavelengths of the electromagnetic radiation emitted by optical waveform emitter (8018) may be configured to enable the identification of the type of anatomical and/or physical structure, such as critical structure(s) (8011a, 8011b). The identification of critical structure(s) (8011a, 8011b) may be accomplished through spectral analysis, photo-acoustics, fluorescence detection, and/or ultrasound, for example. In one aspect, the wavelengths of the electromagnetic radiation may be variable. The waveform sensor and optical waveform emitter (8018) may be inclusive of a multispectral imaging system and/or a selective spectral imaging system, for example. In other instances, the waveform sensor and optical waveform emitter (8018) may be inclusive of a photoacoustic imaging system, for example. In other instances, optical waveform emitter (8018) may be positioned on a separate surgical device from imaging device (8017).

The depicted surgical visualization system (8010) also includes an emitter (8019), which is configured to emit a pattern of light, such as stripes, grid lines, and/or dots, to enable the determination of the topography or landscape of surface (8013). For example, projected light arrays may be used for three-dimensional scanning and registration on surface (8013). The projected light arrays may be emitted from emitter (8019) located on surgical device (8016) and/or imaging device (8017), for example. In one aspect, the projected light array is employed to determine the shape defined by surface (8013) of the tissue (8012) and/or the motion of surface (8013) intraoperatively. Imaging device (8017) is configured to detect the projected light arrays reflected from surface (8013) to determine the topography of surface (8013) and various distances with respect to surface (8013).

The depicted surgical visualization system (8010) also includes distance sensor system (8014) configured to determine one or more distances at the surgical site. In one aspect, distance sensor system (8014) may include a time-of-flight distance sensor system that includes an emitter, such as structured light emitter (8019); and a receiver (not shown), which may be positioned on surgical device (8016). In other instances, the time-of-flight emitter may be separate from structured light emitter (8019). In one general aspect, the emitter portion of time-of-flight distance sensor system (8014) may include a laser source and the receiver portion of time-of-flight distance sensor system (8014) may include a matching sensor. Time-of-flight distance sensor system (8014) may detect the “time of flight,” or how long the laser light emitted by structured light emitter (8019) has taken to bounce back to the sensor portion of the receiver. Use of a very narrow light source in structured light emitter (8019) may enable distance sensor system (8014) to determine the distance to surface (8013) of the tissue (8012) directly in front of distance sensor system (8014).

Referring still to FIG. 82, distance sensor system (8014) may be employed to determine an emitter-to-tissue distance (de) from structured light emitter (8019) to surface (8013) of the tissue (12). A device-to-tissue distance (dt) from the distal end of surgical device (8016) to surface (8013) of the tissue (12) may be obtainable from the known position of emitter (8019) on the shaft of surgical device (8016) relative to the distal end of surgical device (8016). In other words, when the distance between emitter (8019) and the distal end of surgical device (8016) is known, the device-to-tissue distance (dt) may be determined from the emitter-to-tissue distance (de). In certain instances, the shaft of surgical device (8016) may include one or more articulation joints; and may be articulatable with respect to emitter (8019) and the jaws. The articulation configuration may include a multi-joint vertebrae-like structure, for example. In certain instances, a three-dimensional camera may be utilized to triangulate one or more distances to surface (8013).

As described above, surgical visualization system (8010) may be configured to determine the emitter-to-tissue distance (de) from emitter (8019) on surgical device (8016) to surface (8013) of a uterus (12) via structured light. Surgical visualization system (8010) is configured to extrapolate a device-to-tissue distance (dt) from surgical device (8016) to the surface (13) of the uterus (12) based on emitter-to-tissue distance (de). Surgical visualization system (10) is also configured to determine a tissue-to-ureter distance (d_{A}) from a ureter (11a) to the surface (13) and a camera-to-ureter distance (d_{w}), from imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (da) (or depth) based on other distances and/or the surface mapping logic described herein.

FIG. 83 is a schematic diagram of a control system (8020), which may be utilized with a surgical visualization system (8010). The depicted control system (8020) includes a control circuit (8021) in signal communication with a memory (8022). Memory (8022) stores instructions executable by control circuit (8021) to determine and/or recognize critical structures (e.g., critical structures (8011a, 8011b) depicted in FIG. 82), determine and/or compute one or more distances and/or three-dimensional digital representations, and to communicate certain information to one or more clinicians. For example, memory (8022) stores surface mapping logic (8023), imaging logic (8024), tissue identification logic (8025), or distance determining logic (8026) and/or any combinations of logic (8023, 8024, 8025, 8026). Control system (8020) also includes an imaging system (8027) having one or more cameras (8028) (like imaging device (8017) depicted in FIG. 82), one or more displays (8029), one or more controls (8030) or any combinations of these elements. The one or more cameras (8028) may include one or more image sensors (8031) to receive signals from various light sources emitting light at various visible and invisible spectra (e.g., visible light, spectral imagers, three-dimensional lens, among others). Display (8029) may include one or more screens or monitors for depicting real, virtual, and/or virtually-augmented images and/or information to one or more clinicians.

In various aspects, a main component of camera (8028) includes an image sensor (8031). Image sensor (8031) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. Image sensor (8031) may also include any suitable number of chips.

Control system (8020) also includes a spectral light source (8032) and a structured light source (8033). In certain instances, a single source may be pulsed to emit wavelengths of light in spectral light source (8032) range and wavelengths of light in structured light source (8033) range. Alternatively, a single light source may be pulsed to provide light in the invisible spectrum (e.g., infrared spectral light) and wavelengths of light on the visible spectrum. Spectral light source (8032) may include a hyperspectral light source, a multispectral light source, a fluorescence excitation light source, and/or a selective spectral light source, for example. In various instances, tissue identification logic (8025) may identify critical structure(s) via data from a spectral light source (8032) received by the image sensor (8031) portion of camera (8028). Surface mapping logic (8023) may determine the surface contours of the visible tissue based on reflected structured light. With time-of-flight measurements, distance determining logic (8026) may determine one or more distance(s) to the visible tissue and/or critical structure(s) (8011a, 8011b). One or more outputs from surface mapping logic (8023), tissue identification logic (8025), and/or distance determining logic (8026) may be provided to imaging logic (8024) and combined, blended, and/or overlaid to be conveyed to a clinician via display (8029) of imaging system (8027).

Surgical visualization system (8010) and control system (8020) may include the teachings of U.S. patent application Ser. No. 17/373,593, entitled “Endoscope with Synthetic Aperture Multispectral Camera Array,” filed on Aug. 14, 2021, the disclosure of which is incorporated by reference herein.

### XVI. EXEMPLARY METHOD FOR INSTRUMENT ASSESSMENT OF RECOVERY CAPACITY

As mentioned above, in some instances after exemplary use, features of surgical instrument/tool (112, 117, 152, 154, 156, 8016) may be processed for disposal, reuse, and/or remanufacturing. Suitable reuse and/or remanufacturing of features of surgical instrument/tool (112, 117, 152, 154, 156, 8016) may be referred to as recovering features of instrument/tool (112, 117, 152, 154, 156, 8016). In some instances, recovered features of surgical instrument/tool (112, 117, 152, 154, 156, 8016) may be reused by being incorporated into rebuilding a reprocessed product.

Processing features of a used surgical instrument/tool (112, 117, 152, 154, 156, 8016) for suitable recovery (i.e., reuse and/or remanufacturing) in accordance with the description herein may require significant resources (e.g., effort, time, and money). Additionally, in some instances, features of a used surgical instrument/tool (112, 117, 152, 154, 156, 8016) that are originally intended for suitable recovery may experience an undesirable amount of damage and/or performance degradation during exemplary use such that recovering a specific feature of surgical instrument/tool (112, 117, 152, 154, 156, 8016) is no longer feasible. When features originally intended for suitable recovery experience an undesirable amount of damage and/or performance degradation, significant resources may be utilized in an unsuccessful attempt to recover such features. Therefore, it may be desirable to determine if at least some features of a specific used surgical instrument/tool (112, 117, 152, 154, 156, 8016) have the capacity of being suitably recovered after exemplary use, but prior to investing significant resources.

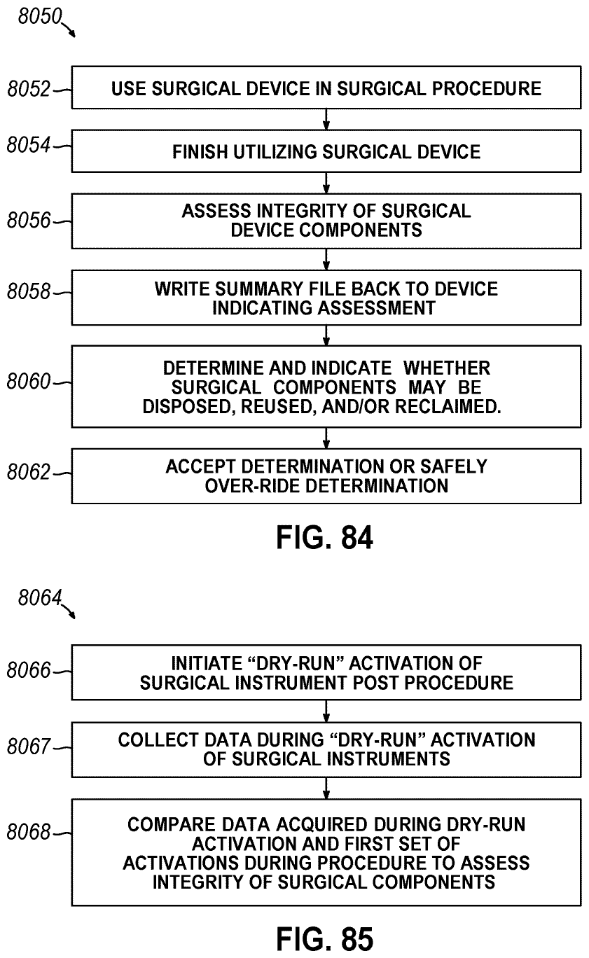

FIG. 84 shows an exemplary method (8050) of determining recovery capacity for at least some features of a surgical instrument/tool (112, 117, 152, 154, 156, 8016). In some non-limiting aspects of the disclosure, features of surgical instrument/tool (112, 117, 152, 154, 156, 8016) that method (8050) may be used to determine the recovery capacity of include, but are not limited to: suitable electrical components, handpiece (160, 176, 185), shaft assembly (164, 178, 186), end effector (166, 180, 188), ultrasonic blade (168, 190), clamp arm (170, 181, 182, 192), trigger (183, 194), toggle button (173, 174, 175, 195, 196, 197), suitable portions of the above mentioned components, or any other suitable components as would be apparent to one skilled in the art in view of the teachings herein.

First, an operator may use (8052) surgical instrument/tool (112, 117, 152, 154, 156, 8016) in order to perform a suitable medical procedure on a patient in accordance with the description herein. As mentioned above, during use (8052), surgical instrument/tool (112, 117, 152, 154, 156, 8016) may be suitably coupled to, and in communication with, hub (106), generator module (140), patient side cart (120), surgical robot hub (122), surgical visualization system (8010), control system (8020), etc. Therefore, suitable devices of computer-implemented interactive surgical system (100) may be able to measure, collect, and/or store various data related to the operation of surgical instrument/tool (112, 117, 152, 154, 156, 8016) in accordance with the description herein.

Next, the operator may finish (8054) utilizing surgical instrument/tool (112, 117, 152, 154, 156, 8016). The operator may interact with suitable components of interactive surgical system (100) in order to indicate that surgical instrument/tool (112, 117, 152, 154, 156, 8016) is finished being utilized for a specific surgical procedure. In some non-limiting aspects of the disclosure, the operator (or any other suitable individual) may press a button of surgical system (100) in order to indicate that that surgical instrument/tool (112, 117, 152, 154, 156, 8016) is finished being used and/or the medical procedure has been completed. For example, such a button may be located on generator module (140). Of course, any other suitable means may be utilized in order to indicate that surgical instrument/tool (112, 117, 152, 154, 156, 8016) is finished being used as would be apparent to one skilled in the art in view of the teachings herein.

As mentioned above, suitable components of surgical system (100) may measure, collect, and/or store various data related to the operation of surgical instrument/tool (112, 117, 152, 154, 156, 8016). Once surgical instrument/tool (112, 117, 152, 154, 156, 8016) is finished (8054) being utilized, generator module (140), hub (106), control system (8020), or any other suitable component(s) may be utilized in order to assess (8056) the integrity and/or recovery capacity of at least one feature of surgical instrument/tool (112, 117, 152, 154, 156, 8016). Any suitable components and/or methods may be utilized to assess (8056) the integrity and/or recovery capacity of at least one feature of surgical instrument/tool (112, 117, 152, 154, 156, 8016) as would be apparent to one skilled in the art in view of the teachings herein.

In some aspects of the disclosure, after assessing (8056) the integrity and/or recovery capacity of at least one feature of surgical instrument/tool (112, 117, 152, 154, 156, 8016), generator module (140), hub (106), control system (8020), or any other suitable component(s) may write (8058) a summary file of the assessment (8056) back to suitable electrical components of surgical instrument/tool (112, 117, 152, 154, 156, 8016). Additionally, or alternatively, generator module (140), hub (106), control system (8020), or any other suitable component(s) may also determine and indicate (8060) whether specific features of surgical instrument/tool (112, 117, 152, 154, 156, 8016) have the capacity of being recovered or if such features should be discarded/disposed. Therefore, in some instances, a user may directly view on generator module (140), hub (106), control system (8020), or any other suitable component(s) whether certain features of surgical instrument/tool (112, 117, 152, 154, 156, 8016) have the capacity to be recovered. In some instances, generator module (140), hub (106), control system (8020), etc., may then be used to then retrieve and display proper disposal/recovery instructions.

In instances where a summary file is written (8058) back to surgical instrument/tool (112, 117, 152, 154, 156, 8016), such a summary file may include a synopsis of the assessment (8056) and a recommendation on whether features of surgical instrument/tool (112, 117, 152, 154, 156, 8016) should be disposed or recovered. The summary file written (8058) back to surgical instrument/tool (112, 117, 152, 154, 156, 8016) may be utilized during the disassembly process as would be apparent to one skilled in the art in view of the teachings herein. For example, in instances where an electronic disassembly assistance device is utilized, such as a smart phone or a tablet, such a device may communicate with surgical instrument/tool (112, 117, 152, 154, 156, 8016) in order to retrieve the summary file. The electronic disassembly assistance device may then utilize the summary file to retrieve or generate instructions related to proper recovery and/or disposal. In some instances, the electronic disassembly assistance device may communicate with generator module (140), hub (106), control system (8020), etc., in order to retrieve or generate such instructions.

In some instances, a user may be provided with the option to accept or safely over-ride (8062) the determination (8060) made as to whether surgical instrument/tool (112, 117, 152, 154, 156, 8016) should be disposed or recovered. In instances where a user decides to over-ride a determination (8060) that a feature of surgical instrument/tool (112, 117, 152, 154, 156, 8016) should be disposed, electronic disassembly assistance device or any other feature configured to display instructions may then display recovery instructions rather than disposal instructions. Additionally, electronic disassembly assistance device or any other suitable display feature may also display the implication and/or potential consequences over-riding (8062) the system's determination (8060).

However, in some instance where the assessed (8056) damage to features of surgical instrument/tool (112, 117, 152, 154, 156, 8016) exceeds a predetermined, ultimate limit, the disposal determination (8060) of such a feature may become an unassailable determination that is not over-ridable. For example, in instances where the assessed (8060) feature is ultrasonic blade (168), if assessment (8060) determines blade damage is coming to a threshold that, if exceeded, the instrument will fail in an unsafe manner; the system could prevent the user from over-riding (8062) the determination (8060) and also prevent further use of the specific ultrasound blade (168). In some instances, the system could prevent over-riding (8062) in a limiting manner rather than an absolute manner such that portions of features are prevented from operating or features are prevented from being used at a predetermined, maximum power level.

Assessment (8056) may be performed at any suitable time after surgical instrument/tool (112, 117, 152, 154, 156, 8016) is finished being utilized as would be apparent to one skilled in the art in view of the teachings herein. Therefore, method (8050) may be utilized within the surgical theater in order to assess if specific features of surgical instrument/tool (112, 117, 152, 154, 156, 8016) should be discarded immediately or if such features show any potential for being recovered.

As mentioned above, any suitable components and/or methods may be utilized to assess (8056) the integrity and/or recovery capacity of at least one feature of surgical instrument/tool (112, 117, 152, 154, 156, 8016) as would be apparent to one skilled in the art in view of the teachings herein. FIG. 85 shows one exemplary assessment method (8064) that may be used to assess the recovery capacity of suitable features of surgical instrument/tool (112, 117, 152, 154, 156, 8016). Therefore, it should be understood that assessment method (8064) may be readily incorporated into method (8050) described above.

First, a user may initiate a “dry-run” activation (8066) of surgical instrument/tool (112, 117, 152, 154, 156, 8016) after surgical instrument/tool (112, 117, 152, 154, 156, 8016) is finished being utilized (e.g., post procedure). A “dry-run” activation (8066) may include activating an empty end effector (i.e., an end effector not significantly grasping other material) with suitable energy required to sever and/or seal tissue for a suitable amount of time, and then allow end effector to suitably deenergize (e.g., an activation cycle). Of course, “dry-run” activation (8066) may include activating end effector with any suitable level of energy as would be apparent to one skilled in the art in view of the teachings here. Further, “dry-run” activation (8066) may include any suitable type of energized activation (e.g., ultrasonic, RF energy, etc.,) as would be apparent to one skilled in the art in view of the teachings herein.

Surgical instrument/tool (112, 117, 152, 154, 156, 8016) may include any suitable number of sensors configured to measure suitable characteristics during the operation of surgical instrument/tool (112, 117, 152, 154, 156, 8016). Because surgical instrument/tool (112, 117, 152, 154, 156, 8016) is in communication with other suitable components of computer-implemented interactive surgical system (100), such components of system (100) may then collect/measure (8067) data (e.g., temperature, time, and energy output, etc.) during the post procedure activation (8066). While temperature, time, and energy output are measured in the current example, any other suitable variables may be measured as would be apparent to one skilled in the art in view of the teachings herein.

Next, suitable components of system (100) (such as hub (106), generator module (140), etc.) may then compare (8068) data acquired during the dry-run with specified parameters, such as data acquired during the first activation of the actual surgical procedure. If the comparison (8068) verifies that the measured variables from the dry-run activation (8066) are within specified parameters, the assessment method (8064) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8064) may correspond to a determination that specific features of surgical instrument/tool (112, 117, 152, 154, 156, 8016) should be disposed.

Determining the range of specified parameters may be achieved using any suitable means as would be apparent to one skilled in the art in view of the teachings herein. In the current example shown in FIG. 85, system (100) may store and utilize variable measurements taken during a single activation (e.g., the first activation) of surgical instrument/tool (112, 117, 152, 154, 156, 8016) during a surgical procedure and utilized those measurements to generate the range of specified parameters. As another example, system (100) may store and utilize variable measurements taken during multiple activations of surgical instrument/tool (112, 117, 152, 154, 156, 8016) throughout a surgical procedure and utilized those measurements to generate the range of specified parameters. As yet another example, the range of specified parameters may be predetermined.

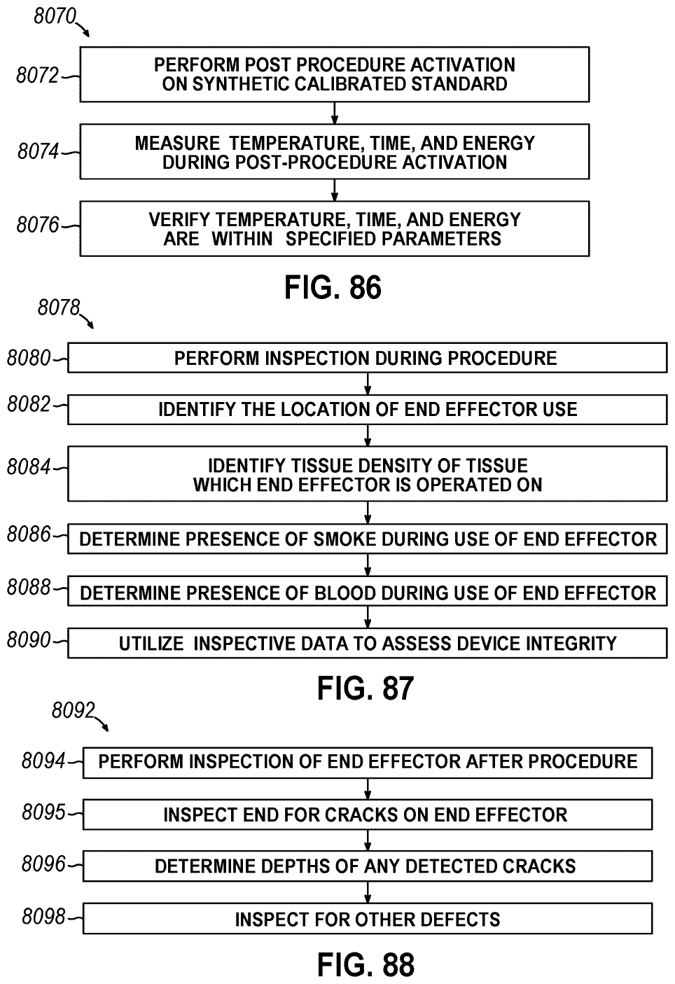

FIG. 86 shows another exemplary assessment method (8070) that may be used to assess the recovery capacity of suitable features of surgical instrument/tool (112, 117, 152, 154, 156, 8016). Therefore, it should be understood that assessment method (8070) may be readily incorporated into method (8050) described above. As will be described below, assessment method (8070) may be substantially similar to assessment method (8064) described above, except that method (8070) utilizes a synthetic calibrated standard (e.g., synthetic tissue) during a post procedure activation (8072) instead of a dry-run.

First, a user may perform a post procedure activation (8072) of surgical instrument/tool (112, 117, 152, 154, 156, 8016) on a piece of synthetic tissue (e.g., a calibrated standard). During post procedure activation (8072), end effector of surgical instrument/tool (112, 117, 152, 154, 156, 8016) may grasp the synthetic tissue and suitably apply energy to synthetic tissue in a similar fashion to exemplary use of end effector with patient tissue during a surgical procedure. Such a piece of synthetic tissue may be provided with surgical instrument/tool (112, 117, 152, 154, 156, 8016) in a surgical kit. Such a piece of synthetic tissue may be a 3D printed “vessel” like structure that surgical instrument/tool (112, 117, 152, 154, 156, 8016) may grasp and “seal” during activation.

Surgical instrument/tool (112, 117, 152, 154, 156, 8016) may include any suitable number of sensors configured to measure suitable characteristics during the operation of surgical instrument/tool (112, 117, 152, 154, 156, 8016). Because surgical instrument/tool (112, 117, 152, 154, 156, 8016) is in communication with other suitable components of computer-implemented interactive surgical system (100), such components of system (100) may then measure (8074) temperature, time, and energy output during the post procedure activation (8072). While temperature, time, and energy output are measured in the current example, any other suitable variables may be measured as would be apparent to one skilled in the art in view of the teachings herein.

Next, suitable components of system (100) (such as hub (106), generator module (140), etc.) may then verify (8076) that the measured variables from the post procedure activation (8072) are within specified parameters, while the user may visually verify end effector suitably applied energy to the synthetic standard (e.g., visually confirm a seal was made). If the measured variables are within the specified parameters and end effector suitably applied energy to the synthetic standard, the assessment method (8070) may correspond to a determination that specific features of surgical instrument/tool (112, 117, 152, 154, 156, 8016) may be recovered, or at least have some capacity for being recovered. Conversely, if the measured variables are not within the specified parameters and/or end effector did not suitably applied energy to the synthetic standard, the assessment method (8070) may correspond to a determination that specific features of surgical instrument/tool (112, 117, 152, 154, 156, 8016) should be disposed.

Determining the range of specified parameters may be achieved using any suitable means as would be apparent to one skilled in the art in view of the teachings herein. In the current example shown in FIG. 86, system (100) may store and utilize variable measurements taken during a single activation (e.g., the first activation) of surgical instrument/tool (112, 117, 152, 154, 156, 8016) during a surgical procedure and utilize those measurements to generate the range of specified parameters. As another example, system (100) may store and utilize variable measurements taken during multiple activations of surgical instrument/tool (112, 117, 152, 154, 156, 8016) throughout a surgical procedure and utilized those measurements to generate the range of specified parameters. As yet another example, the range of specified parameters may be predetermined.

FIG. 87 shows another exemplary assessment method (8078) that may be used to assess the recovery capacity of suitable features of surgical instrument/tool (112, 117, 152, 154, 156, 8016). Therefore, it should be understood that assessment method (8078) may be readily incorporated into method (8050) described above. In the current example, assessment method (8078) may utilize suitable components of surgical visualization system (8010) and/or control system (8020) in order to generate the necessary inspection data described below to determine if features of surgical instrument/tool (112, 117, 152, 154, 156, 8016) have any recovery capacity or should be discarded.

Assessment method (8078) includes utilizing surgical visualization system (8010) to perform inspection (8080) during a surgical procedure. Data acquired during inspection (8080) may be communicated and stored on suitable components of system (100), such as hub (106). During the procedure, various features of visualization system (8010) may be utilized to identify (8082) the anatomical location where end effector of surgical instrument/tool (112, 117, 152, 154, 156, 8016) is being used; and communicate that data to suitable components of system (100). Further, during the procedure, various features of visualization system (8010) may be utilized to identify (8084) the tissue density of tissue which end effector of surgical instrument/tool (112, 117, 152, 154, 156, 8016) is operating on; and communicate that data to suitable components of system (100). Further, during the procedure, various features of visualization system (8010) may be utilized to determine (8086) the presence of smoke during use tissue density of surgical instrument/tool (112, 117, 152, 154, 156, 8016); and communicate that data to suitable components of system (100). Further, during the procedure, various features of visualization system (8010) may be utilized to determine (8088) the presence of blood during use tissue density of surgical instrument/tool (112, 117, 152, 154, 156, 8016); and communicate that data to suitable components of system (100).

Finally, system (100) may utilize (8090) the above acquired inspection data in order to assess the recovery capacity of at least one feature of surgical instrument/tool (112, 117, 152, 154, 156, 8016). If the measured variables are within the specified parameters, the assessment method (8078) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8078) may correspond to a determination that specific features of surgical instrument/tool (112, 117, 152, 154, 156, 8016) should be disposed.

FIG. 88 shows another exemplary assessment method (8092) that may be used to assess the recovery capacity of suitable features of surgical instrument/tool (112, 117, 152, 154, 156, 8016). Therefore, it should be understood that assessment method (8092) may be readily incorporated into method (8050) described above. In the current example, assessment method (8092) may utilize suitable components of surgical visualization system (8010) and/or control system (8020) in order to generate the necessary inspection data described below to determine if features of surgical instrument/tool (112, 117, 152, 154, 156, 8016) have any recovery capacity or should be discarded.

Assessment method (8092) includes utilizing surgical visualization system (8010) to perform inspection (8094) after a surgical procedure. Data acquired during inspection (8092) may be communicated and stored on suitable components of system (100), such as hub (106). After a procedure, various features of visualization system (8010) may be utilized to inspect (8095) end effector of surgical instrument/tool (112, 117, 152, 154, 156, 8016) for cracks; and communicate that data to suitable components of system (100). Further, after a procedure, various features of visualization system (8010) may be utilized to determine (8096) the depths of any crack detected in end effector of surgical instrument/tool (112, 117, 152, 154, 156, 8016) and inspect (8098) for any other defects; and communicate that data to suitable components of system (100).

Finally, system (100) may utilize the above acquired inspection data in order to assess the recovery capacity of at least one feature of surgical instrument/tool (112, 117, 152, 154, 156, 8016). If the measured variables are within the specified parameters, the assessment method (8092) may correspond to a determination that specific features of surgical instrument/tool (112, 117, 152, 154, 156, 8016) may be recovered, or at least have some capacity for being recovered. Conversely, if the measured variables are not within the specified parameters, the assessment method (8092) may correspond to a determination that specific features of surgical instrument/tool (112, 117, 152, 154, 156, 8016) should be disposed.

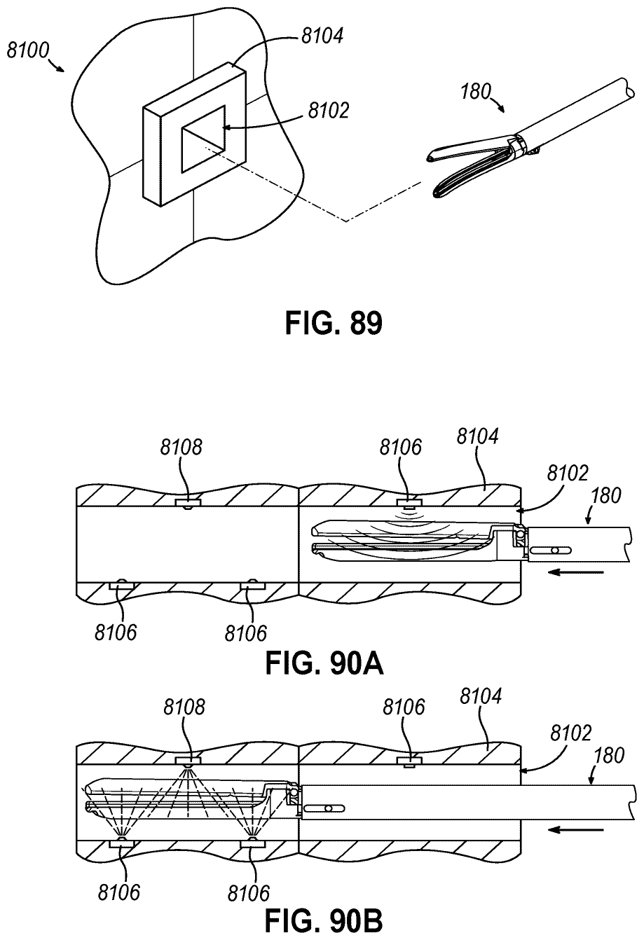

FIGS. 89-90B show an exemplary assessment and cleaning port (8100) that may be readily incorporated into hub (106) and/or a console of visualization system (8010) in order to perform assessment method (8092) described above, and also clean end effector.

Assessment and cleaning port (8100) includes a channel (8102) extending from an exterior portal (8104). Exterior portal (8104) may be accessible for an end effector, such as end effector (180) in the current example, to be easily inserted into channel (8102). Likewise, channel (8102) is dimensioned to suitably receive an end effector, such as end effector (180) in the current example.

As shown in FIGS. 90A-90B, an interior surface of port (8100) defining channel (8102) includes at least one visual inspector (8106) and at least one cleaning assembly (8108). In the current example, cleaning assemblies (8108) and visual inspector (8106) are longitudinally offset from each other. However, this is merely optional. During use, end effector (180) may be inserted into channel (8102) such that end effector (180) is adjacent to visual inspector (8106). Visual inspector (8106) may then perform assessment method (8092) described above and determine if features of end effector (180) are recoverable. If recoverable, as shown in FIG. 90B, end effector (180) may be placed adjacent to cleaning assembly (8108) such that cleaning assembly (8108) may apply cleaning materials onto end effector (180). Any suitable cleaning materials may be applied as would be apparent to one skilled in the art in view of the teachings herein.

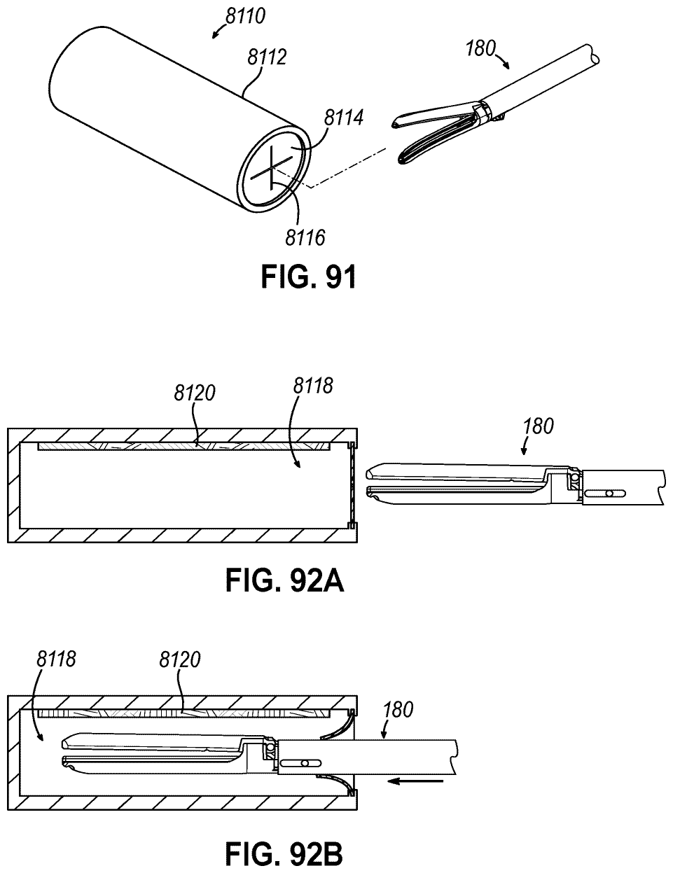

FIGS. 91-92B show an exemplary cleaning sheath (8110) that may be used to cover an end effector, such as end effector (180) shown. As will be described in greater detail below, cleaning sheath (8110) may be applied to end effector (180) after exemplary use in order to prevent other objects, besides end effector (180) and the interior of sheath (8110), from being exposed to biohazard materials accumulated on end effector (180) during exemplary use.

Cleaning sheath (8110) includes a hollow body (8112) defining an interior (8118), a seal (8112) located at an open end of hollow body (8112), and a cleaning material (8120) located within interior (8118) of hollow body (8112). Seal (8112) define an expandable opening (8116) configured to suitably receive end effector (180) such that end effector (180) may be inserted within interior (8118) of hollow body (8112), as shown in FIGS. 92A-92B. In some instances a robotic arm may be configured to attach sheath (8110) with end effector (180).

Seal (8112) may sufficiently engage portions of shaft assembly and/or end effector (180) when suitably inserted into interior (8118) such that seal (8112) inhibits any materials located within interior (8118) from escaping hollow body (8112).

Cleaning material (8120) may include chemicals or other materials that may clean and/or preserve end effector (180) while contained within interior (8118). Cleaning materials (8120) may be configured to clean off debris accumulated on end effector (180) from exemplary use of end effector (180). Cleaning material (8120) may contain any suitable cleaning materials as would be apparent to one skilled in the art in view of the teachings herein. For example, cleaning materials (8120) may include frozen C02 pellets that may configured to “grit” blast eschar accumulated on end effector (180) from exemplary use.

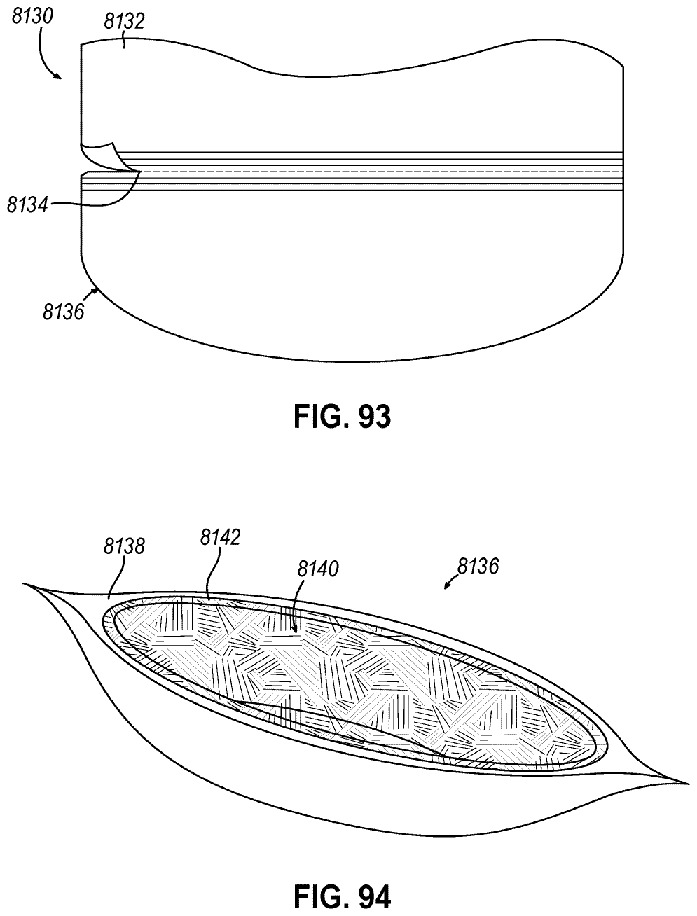

FIG. 93 shows an exemplary surgical instrument packaging (8130) that may be used to transport surgical instrument/tool (112, 117, 152, 154, 156, 8016) into the surgical theater while keeping surgical instrument/tool (112, 117, 152, 154, 156, 8016) suitably sterile. Additionally, as will be described in greater detail below, packaging (8130) includes a removable cleaning kit (8136) that may be utilized in the surgical theater, post procedure or mid-procedure, to clean features of surgical instrument/tool (112, 117, 152, 154, 156, 8016) for purposes of recovery or continued use.

Packaging (8130) includes a primary packaging (8132) configured to store suitable components of a surgical kit used to form surgical instrument/tool (112, 117, 152, 154, 156, 8016). Additionally, packaging (8130) includes removable cleaning kit (8136) that is removably attached to primary packaging (8132) via a perforated section (8134). Therefore, when a user desires to use cleaning kit (8136), they may remove cleaning kit (8136) from primary packaging (8132) via tearing perforated section (8134).

Turning to FIG. 94, in the current example, removable cleaning kit (8136) includes a container (8138) (e.g., a bowl) defining an opening (8140) and a cleaning material (8142) accessible via opening (8140). Cleaning material (8142) may also include a suitable applicator, such as a sponge soaked in a cleaning material. Cleaning material (8142) may include a gritty material or pellets. Cleaning material (8142) may include a bottle brush cleaner, leaflets, or fingers for the user to clean end effector (180) with a cleaning material.

Container (8138) may be moldered into a type of bowl dimensioned to receive liquid cleaning materials (8142). Therefore, container (8138) may act as a basin. The portion of container (8128) forming basin may be lined with a cleaning material that, upon contacting liquid, will self-mix with the liquid. Container (8138) may include a sponge or have a base of sponge molded into it for cleaning purpose. The sponge may be soaked in cleaning fluid to assist in debris removal. The sponge may contain dry chemicals that are activated when becoming wet.

### XVII. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of disassembling a surgical instrument, the surgical instrument including (a) a shaft assembly extending along a longitudinal axis; (b) an end effector distally extending from the shaft assembly; (c) a body assembly proximally extending from the shaft assembly and including: (i) a first shroud portion, (ii) a second shroud portion, and (iii) a shroud coupling configured to removably affix the first shroud portion to the second shroud portion in a connected state, wherein the shroud coupling is further configured to detach the first shroud portion from the second shroud portion in a disconnected state; (d) an internal assembly housed within the body assembly including a first internal component operably connected to a second internal component, wherein the first and second shroud portions in the connected state enclose and inhibit access to an internal component for containment therein, the method comprising: (a) removing the first shroud portion; (b) breaking the first internal component from the second internal component; and (c) removing the first internal component from the body assembly for disassembly thereof.

### Example 2

The method of Example 1, further comprising removing the second internal component from the body assembly.

### Example 3

The method of any one or more of Examples 1 through 2, further comprising placing the first internal component in a first waste stream and placing the second internal component in a second waste stream, wherein the second waste stream is different than the first waste stream.

### Example 4

The method of any one or more of Examples 1 through 3, wherein the surgical instrument further includes a cable removably connected to the body assembly, where the method further comprises (a) disconnecting the cable from the body assembly; and (b) processing the cable for reuse.

### Example 5

The method of Example 4, wherein the surgical instrument further includes (a) the cable configured to communicate an electrical energy therethrough; and (b) a first adapter extending along a longitudinal axis from the cable and configured to releasably connect to at least one of an instrument adapter of a surgical instrument or a generator adapter of a generator, the first adapter including: (i) an adapter body, (ii) a first electrical contact electrically connected to the cable, and (iii) an engagement assembly, including: (A) a latch coupling having a latch portion selectively movable relative to the adapter body between a locked position and an unlocked position, wherein the latch portion includes at least lease one catch member angularly surrounding at least a majority of the longitudinal axis or a sleeve rotatable about the longitudinal axis, wherein the latch portion in the locked position is configured to capture the at least one of the instrument adapter or the generator adapter for retaining electrical connection between the first electrical contact and the at least one of the instrument adapter or the generator adapter, and wherein the latch portion in the unlocked position is configured to release the at least one of the instrument adapter or the generator adapter for uncoupling electrical connection between the first electrical contact and the at least one of the instrument adapter or the generator adapter, or (B) a communication coupling supported by the adapter body and configured to resiliently bias engagement between the first electrical contact and the at least one of the instrument adapter or the generator adapter for urging contact therebetween.

### Example 6

The method of any one or more of Examples 4 through 5, wherein the surgical instrument further includes (a) the cable configured to communicate an electrical energy therethrough; and (b) a first adapter extending from the cable and configured to releasably connect to at least one of an instrument adapter of a surgical instrument or a generator adapter of a generator, the first adapter including: (i) an adapter body, (ii) a first electrical contact electrically connected to the cable, and (iii) an engagement assembly, including: (A) a latch coupling selectively movable relative to the adapter body between a locked position and an unlocked position, wherein the latch coupling in the locked position is configured to capture the at least one of the instrument adapter or the generator adapter for retaining electrical connection between the first electrical contact and the at least one of the instrument adapter or the generator adapter, and wherein the latch coupling in the unlocked position is configured to release the at least one of the instrument adapter or the generator adapter for uncoupling electrical connection between the first electrical contact and the at least one of the instrument adapter or the generator adapter, wherein the latch coupling includes a sleeve operatively connected to the adapter body, wherein the sleeve is biased toward the locked position and configured to selectively move between the locked and unlocked positions, and (B) a communication coupling having a biasing element supported by the adapter body and configured to resiliently bias engagement between the first electrical contact and the at least one of the instrument adapter or the generator adapter for urging contact therebetween.

### Example 7

The method of any one or more of Examples 1 through 6, further comprising selectively engaging a predetermined access portion of the surgical instrument and thereby at least partially removing the predetermined access portion to access an interior of the surgical instrument.

### Example 8

The method of Example 7, further comprising a surgical kit including the surgical instrument, a tool body, a torque wrench operatively connected to the tool body, and a removal portion operatively connected to the tool body, the method further comprising engaging the predetermined access portion with the removal portion and thereby at least partially removing the predetermined access portion to access the interior of the surgical instrument.

The method of any one or more of Examples 1 through 8, further comprising a controller, first and second robotic arms, and a tool, wherein the first robotic arm is operatively coupled with the surgical instrument, wherein the tool is in communication with the controller, the method further comprising disassembling a portion of the surgical instrument that is operatively coupled with the first robotic arm using a disassembly feature of the tool that is operatively coupled with second robotic arm.

### Example 9

### Example 10

The method of Example 9, further comprising inserting at least a portion of the surgical instrument into a package.

### Example 11

A method of determining a disposal methodology of a surgical kit for a surgical system, the method comprising: (a) identifying a geographical location of use for the surgical kit; (b) extracting a set of disposal instructions from a lookup table based on the identified geographical location of use for the surgical kit; and (c) displaying the disposal instructions.

### Example 12

The method of Example 11, wherein the surgical system further includes a generator and the surgical kit is configured to be assembled into a surgical instrument, wherein the surgical instrument is configured to be disassembled into a first set of used components and a second set of used components.

### Example 13

The method of Example 12, wherein the surgical system further includes an identification feature associated with either the generator of the surgical kit and a disassembly assistant device configured to display a set of instructions on how to disassemble the surgical instrument into the first set of used components and the second set of used components, wherein the set of instructions is dependent on data obtained from the identification feature.

The method of any one or more of Examples 11 through 13 further comprising reclaiming at least a portion of a surgical instrument of the surgical system, wherein reclaiming the at least the portion of the surgical instrument further includes inserting the portion of the surgical instrument into a return packaging and enclosing the return packaging.

### Example 14

### Example 15

The method of Example 14, wherein the surgical system further includes a outer packaging and the return packaging, wherein the outer packaging defines an interior and an exterior in a closed configuration wherein the surgical instrument is disposed within the interior of the outer packaging or coupled with the outer packaging in the closed configuration, wherein the outer packaging is configured to be opened by a user to expose the interior in an open configuration to allow for access of the surgical instrument during a surgical procedure, and wherein the return packaging is configured to receive the surgical instrument after the surgical procedure for reduced cross-contamination.

### Example 16

The method of Example 15, wherein the surgical system further includes machine-readable code and a code reader, wherein the machine-readable code is coupled with at least one of the surgical instrument, the outer packaging, or the return packaging, and wherein the code reader configured to read the machine-readable code.

### Example 17

The method of any one or more of Examples 11 through 16, wherein the surgical kit is configured to be assembled into a surgical instrument, wherein the surgical instrument includes (a) an end effector configured to transition between a deactivated configuration and an activated configuration, wherein the end effector is configured to transmit energy to tissue in the activated configuration; and (b) a proximal body operably attached to the end effector, wherein the proximal body comprises: (i) an electrical component configured to assist the end effector, (ii) a first shroud, (iii) a second shroud configured to couple with the first shroud to cooperatively define a hollow interior, wherein the electrical component is housed within the hollow interior, (iv) a first restraining feature associated with the first shroud, and (v) a second restraining feature associated with the second shroud, wherein the first and second restraining features are configured to couple together to cooperatively align the first shroud and the second shroud, wherein the first restraining feature and the second restraining feature are configured to selectively disengage to allow the first shroud and the second shroud to decouple form each other and expose the electrical component within the hollow interior.

### Example 18

The method of any one or more of Examples 11 through 17 further comprising determining a recovery capacity of at least one feature of a surgical instrument of the surgical system.

### Example 19

The method of Example 18, wherein the surgical system further includes (a) the surgical instrument comprising an end effector; (b) a hub configured to establish communication with the surgical instrument and assist the surgical instrument during a procedure; and (c) an assessment port associated with the hub, wherein the assessment port defines a channel dimensioned to receive the end effector, wherein the assessment port comprises a visualization system located within the channel, wherein the visualization system is configured to inspect the end effector for a defect and communicate the defect to the hub.

A method of reclaiming at least a portion of a surgical instrument, comprising: (a) verifying reuse capacity of the portion of the surgical instrument; (b) determining a waste stream for the portion of the surgical instrument; and (c) disassembling the at least the portion of the surgical instrument from a remainder of the surgical instrument at a predetermined region of the surgical instrument thereby reclaiming the at least a portion of the surgical instrument according to the waste stream.

### Example 20

### XVIII. MISCELLANEOUS

Versions of the devices described above may have application in conventional medical treatments and procedures conducted by a medical professional, as well as application in robotic-assisted medical treatments and procedures.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

In addition to the foregoing, the teachings herein may be readily combined with the teachings of U.S. patent application Ser. No. 17/854,050, entitled “Surgical Instrument with Predetermined Separation Features for Waste Stream Utilization and Related Methods,” filed on Jun. 30, 2022, published as U.S. Pub. No. 2024/0000474 on Jan. 4, 2024, the disclosure of which is incorporated by reference herein. Various suitable ways in which the teachings herein may be combined with the teachings of U.S. patent application Ser. No. 17/854,050 will be apparent to those of ordinary skill in the art in view of the teachings herein.

In addition to the foregoing, the teachings herein may be readily combined with the teachings of U.S. patent application Ser. No. 17/854,166, entitled “Surgical Instrument with Removable Cable and Associated Couplings,” filed on Jun. 30, 2022, published as U.S. Pub. No. 2024/0006810 on Jan. 4, 2024, issued as U.S. Pat. No. 12,218,459 on Feb. 4, 2025, the disclosure of which is incorporated by reference herein. Various suitable ways in which the teachings herein may be combined with the teachings of U.S. patent application Ser. No. 17/854,166 will be apparent to those of ordinary skill in the art in view of the teachings herein.

In addition to the foregoing, the teachings herein may be readily combined with the teachings of U.S. patent application Ser. No. 17/854,641, entitled “Surgical System and Methods of Assembly and Disassembly of Surgical Instrument,” filed on Jun. 30, 2022, published as U.S. Pub. No. 2024/0000476, the disclosure of which is incorporated by reference herein. Various suitable ways in which the teachings herein may be combined with the teachings of U.S. patent application Ser. No. 17/854,641 will be apparent to those of ordinary skill in the art in view of the teachings herein.

In addition to the foregoing, the teachings herein may be readily combined with the teachings of U.S. patent application Ser. No. 17/854,104, entitled “Robotic Surgical System with Removable Portion and Method of Disassembling Same,” filed on Jun. 30, 2022, published as U.S. Pub. No. 2024/0000526 on Jan. 1, 2024, the disclosure of which is incorporated by reference herein. Various suitable ways in which the teachings herein may be combined with the teachings of U.S. patent application Ser. No. 17/854,104 will be apparent to those of ordinary skill in the art in view of the teachings herein.

In addition to the foregoing, the teachings herein may be readily combined with the teachings of U.S. patent application Ser. No. 17/854,110, entitled “System for Determining Disposal of Surgical Instrument and Related Methods,” filed on Jun. 30, 2022, published as U.S. Pub. No. 2024/0001416 on Jan. 1, 2024, the disclosure of which is incorporated by reference herein. Various suitable ways in which the teachings herein may be combined with the teachings of U.S. patent application Ser. No. 17/854,110 will be apparent to those of ordinary skill in the art in view of the teachings herein.

In addition to the foregoing, the teachings herein may be readily combined with the teachings of U.S. patent application Ser. No. 17/854,114, entitled “Reclamation Packaging for Surgical Instrument and Related Methods,” filed on Jun. 30, 2022, published as U.S. Pub. No. 2024/0000491 on Jan. 1, 2024, the disclosure of which is incorporated by reference herein. Various suitable ways in which the teachings herein may be combined with the teachings of U.S. patent application Ser. No. 17/854,114 will be apparent to those of ordinary skill in the art in view of the teachings herein.

In addition to the foregoing, the teachings herein may be readily combined with the teachings of U.S. patent application Ser. No. 17/854,120, entitled “Surgical Instrument with Various Alignment Features and Methods for Improved Disassembly and Assembly,” filed on Jun. 30, 2022, published as U.S. Pub. No. 2024/0000475 on Jan. 1, 2024, the disclosure of which is incorporated by reference herein. Various suitable ways in which the teachings herein may be combined with the teachings of U.S. patent application Ser. No. 17/854,120 will be apparent to those of ordinary skill in the art in view of the teachings herein.

In addition to the foregoing, the teachings herein may be readily combined with the teachings of U.S. patent application Ser. No. 17/854,127, entitled “Surgical System and Methods for Instrument Assessment and Cleaning,” filed on Jun. 30, 2022, published as U.S. Pub. No. 2024/0003820 on Jan. 1, 2024, the disclosure of which is incorporated by reference herein. Various suitable ways in which the teachings herein may be combined with the teachings of U.S. patent application Ser. No. 17/854,127 will be apparent to those of ordinary skill in the art in view of the teachings herein.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of determining a recovery capacity of at least one feature of a surgical instrument, the method comprising:
(i) establishing communication with the surgical instrument;
(ii) assisting operation of the surgical instrument during a procedure;
(iii) obtaining data related to the surgical instrument;
(iv) evaluating the data obtained related to the surgical instrument to determine a digital assessment of an impact on performance of the at least one feature of the surgical instrument; and
(v) determining, based on the digital assessment, a capacity of recovery for the at least one feature of the surgical instrument.

2. The method of claim 1, wherein the capacity of recovery relates to the ability of the at least one feature to withstand a procedural aspect required to reuse the at least one feature of the surgical instrument.

3. The method of claim 2, wherein the procedural aspect comprises cleaning the at least one feature of the surgical instrument.

4. The method of claim 2, wherein the procedural aspect comprises remanufacturing the at least one feature of the surgical instrument.

5. The method of claim 1, further comprising providing a recommendation, based on the capacity of recovery.

6. The method of claim 5, wherein the recommendation comprises displaying instructions to discard the at least one feature or displaying instructions to recover the at least one feature.

7. The method of claim 5, further comprising providing a user feedback option to over-ride the recommendation.

8. The method of claim 1, wherein obtaining data occurs concurrently with the procedure.

9. The method of claim 8, wherein obtaining data comprises identifying the location, within a patient, of an end effector of the surgical instrument.

10. The method of claim 8, wherein obtaining data comprises identifying tissue density which an end effector of the surgical instrument is operating on.

11. The method of claim 8, wherein obtaining data comprises defining the presence of smoke or blood during use of an end effector of the surgical instrument.

12. The method of claim 1, wherein obtaining data occurs after the procedure.

13. The method of claim 12, wherein obtaining data comprises inspecting an end effector of the surgical instrument for cracks.

14. The method of claim 12, wherein obtaining data comprises performing a dry-run activation of an end effector of the surgical instrument.

15. The method of claim 12, wherein obtaining data comprises performing a test activation of an end effector of the surgical instrument on a synthetic calibration piece.

16. A surgical system comprising:
(a) a surgical instrument comprising an end effector;
(b) a hub configured to establish communication with the surgical instrument and assist the surgical instrument during a procedure;
(c) an assessment port associated with the hub, wherein the assessment port defines a channel dimensioned to receive the end effector, wherein the assessment port comprises a visualization system located within the channel, wherein the visualization system is configured to inspect the end effector to defects and communicate the results to the hub.

17. The surgical system of claim 16, wherein the assessment port further comprises a cleaning assembly located within the channel.

18. The surgical system of claim 17, wherein the cleaning assembly is configured to clean the end effector in response to the inspection of the visualization system.

19. The surgical system of claim 16, wherein the hub further comprises a generator module configured to power the surgical instrument.

20. A method of determining a recovery capacity of at least one feature of a surgical instrument, the method comprising:
(i) establishing communication with the surgical instrument;
(ii) assisting operation of the surgical instrument during a procedure;
(iii) obtaining a first set of data about the operation of the surgical instrument during the procedure;
(iv) obtaining a second set of data about the operation of the surgical instrument after the procedure;
(v) comparing the first set of data with the second set of data to determine a digital assessment of an impact on performance of the at least one feature of the surgical instrument; and
(vi) determining, based on the digital assessment, a capacity of recovery for the at least one feature of the surgical instrument.
